(19) United States
(12) Patent Application Publication
(10) Pub. No.: US 2026/0337153 A1
(43) Pub. Date: Oct. 29, 2026

(54) IONIZABLE AMINE LIPIDS

(71) Applicant: Intellia Therapeutics, Inc., Cambridge, MA (US)

(72) Inventors: Ramsey Nabil Majzoub, Cambridge, MA (US); Micah Maetani, Cambridge, MA (US); Siyeon Im, Cambridge, MA (US)

(21) Appl. No.: 19/485,395

(22) PCT Filed: May 17, 2024
(86) PCT No.: PCT/US2024/029927

§ 371 Date: Nov. 18, 2025

Related U.S. Application Data
(60) Provisional application No. 63/467,691, filed on May 19, 2023.
(60) Provisional application No. 63/610,628, filed on Dec. 15, 2023.
(60) Provisional application No. 63/648,469, filed on May 16, 2024.

(51) Int. Cl. C07C 271/20; A61K 9/51; A61K 31/7105; A61K 38/46; A61K 48/00; C07D 295/13

(52) U.S. Cl. CPC C07C 271/20; A61K 9/5123; A61K 31/7105; A61K 38/465; A61K 48/0033; A61K 48/0041; A61K 48/005; C07D 295/13

(57) ABSTRACT

The disclosure provides ionizable lipids and lipid nanoparticle (LNP) compositions comprising ionizable lipids, helper lipids, neutral lipids, and PEG lipids useful for the delivery of biologically active agents, for example delivering biologically active agents to cells to prepare engineered cells. The LNP compositions disclosed herein are useful in methods of gene editing and methods of delivering a biologically active agent and methods of modifying or cleaving DNA.

### CROSS-REFERENCE TO RELATED APPLICATIONS

This application claims the benefit of priority to U.S. Provisional Application Nos. 63/467,691, filed May 19, 2023, 63/610,628, filed Dec. 15, 2023, and 63/648,469, filed May 16, 2024, the entire contents of each of which are incorporated by reference herein.

### BACKGROUND

Lipid nanoparticles formulated with ionizable lipids can serve as cargo vehicles for delivery of biologically active agents, in particular polynucleotides, such as polynucleotides for RNA interference, RNAi therapy, mRNA therapy, RNA drugs, antisense therapy, gene therapy, and nucleic acid vaccines (e.g., RNA vaccines). The lipid nanoparticles can include one or more small nucleic acid molecules, RNAi agents, short interfering nucleic acid (siNA), messenger ribonucleic acid (messenger RNA, mRNA), short interfering RNA (siRNA), double-stranded RNA (dsRNA), micro-RNA (miRNA), and short hairpin RNA (shRNA) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), aiRNA (asymmetrical interfering RNA), and siRNA with 1, 2 or more mismatches between the sense and anti-sense strand to relevant cells and/or tissues, such as in a cell culture, subject or organism. Of particular interest is delivery of an mRNA and/or guide RNA. The LNP compositions containing ionizable lipids can facilitate delivery of oligonucleotide agents across cell membranes, and can be used to introduce components and compositions for gene editing into living cells. Biologically active agents that are particularly difficult to deliver to cells include proteins, nucleic acid-based drugs, and derivatives thereof, particularly drugs that include relatively large oligonucleotides, such as mRNA. Compositions for delivery of promising gene editing technologies into cells, such as for delivery of CRISPR/Cas9 system components, are of particular interest (e.g., mRNA encoding a nuclease and associated guide RNA (gRNA)).

There is a need for compositions for improved delivery of nucleic acids, such as RNAs, in vivo and in vitro. As an example, compositions for delivery of the components of CRISPR/Cas to a eukaryotic cell, such as a human cell, are needed. In particular, compositions for delivering mRNA encoding the CRISPR protein component, and for delivering CRISPR gRNAs are of particular interest. Compositions with useful properties for in vitro and in vivo delivery that can stabilize and deliver RNA components, are also of particular interest.

### BRIEF SUMMARY

In some embodiments, the present disclosure relates to a compound represented by structural Formula I

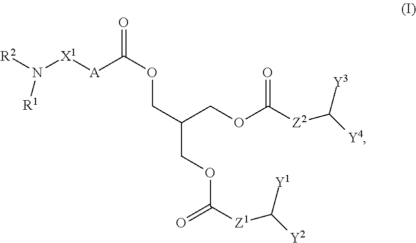

or a salt thereof, wherein:
A is O or NH,
X¹ is a C₁₋₅ alkylene,
R¹ and R² is each independently a C₁₋₃ alkyl, or
R¹ taken together with R² and the nitrogen atom to which they are attached form a 5, 6-, or 7-membered ring, and
Z¹ is a C₁₋₅ alkylene,
Y¹ and Y² is each independently a C₃₋₁₀ alkoxyl or O(C₃₋₁₀ alkynyl),
Z² is a C₁₋₅ alkylene or a direct bond, and
Y³ and Y⁴ is each independently a C₃₋₁₀ alkoxyl or O(C₃₋₁₀ alkynyl), or
   Y³ and Y⁴ is each independently a C₃₋₁₀ alkyl or C₃₋₁₀ alkynyl,
   provided that if Y¹, Y², Y³, and Y⁴ is each independently a C₃₋₁₀ alkoxy, then R¹ and R² are not C₂ alkyl, and R¹ taken together with R² and the nitrogen atom to which they are attached do not form a 6-membered ring.

In some embodiments, the present disclosure relates to a compound represented by structural Formula II,

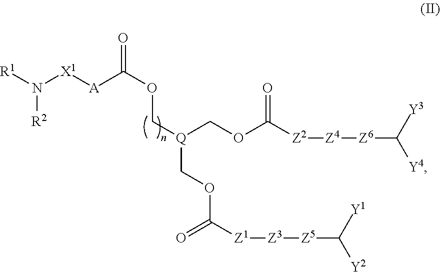

or a salt thereof,
wherein,
Q is CR³ or

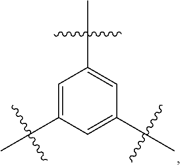 [SMILES: *c1cc(*)cc(*)c1]

A is O, NH, or a direct bond,
X¹ is a C₁₋₅ alkylene,
R¹ and R² is each independently a C₁₋₃ alkyl, or
R¹ taken together with the nitrogen atom to which it is attached and 1-3 carbon atoms of X¹ form a 4-, 5-, or 6-membered ring, or
R¹ taken together with R² and the nitrogen atom to which they are attached form a 5-, 6-, or 7-membered ring, and
R³ is H or C₁₋₃ alkyl,
Z¹ and Z² is each independently a C₁₋₅ alkylene,
Z³ and Z⁴ is each independently a —C(═O)O— in either direction,
Z⁵ and Z⁶ is each independently a direct bond or a C₁₋₃ alkylene,
Y¹ is selected from H, a C₁₋₁₀ alkyl, C₃₋₁₀ alkenyl, and C₃₋₁₀ alkynyl,
Y², Y³, and Y⁴ is each independently selected from a C₃₋₁₀ alkyl, C₃₋₁₀ alkenyl, and C₃₋₁₀ alkynyl, and
n is 0 or 1.

In some embodiments the present disclosure relates to a compound represented by structural Formula III,

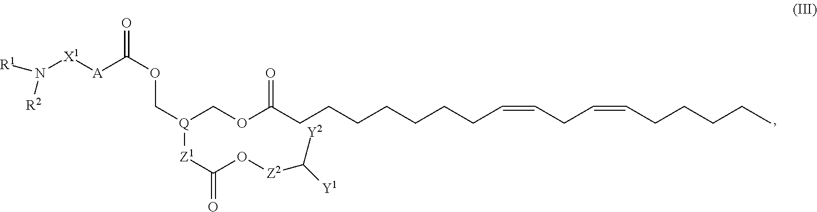

or a salt thereof,
wherein:
Q is CH or

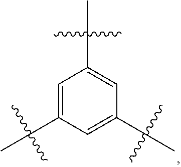 [SMILES: *c1cc(*)cc(*)c1]

A is O or NH,
X¹ is a C₁₋₅ alkylene,
R¹ and R² is each independently a C₁₋₃ alkyl, or
R¹ taken together with the nitrogen atom to which it is attached and 1-3 carbon atoms of X¹ form a 4-, 5-, or 6-membered ring, or
R¹ taken together with R² and the nitrogen atom to which they are attached form a 5-, 6-, or 7-membered ring, and
Z¹ is a C₂₋₉ alkylene,
Z² is a C₁₋₃ alkylene or a direct bond, and
Y¹ and Y² is each independently selected from a C₃₋₁₀ alkyl, C₃₋₁₀ alkenyl, and C₃₋₁₀ alkynyl.

In some embodiments the present disclosure relates to a compound represented by structural Formula IV,

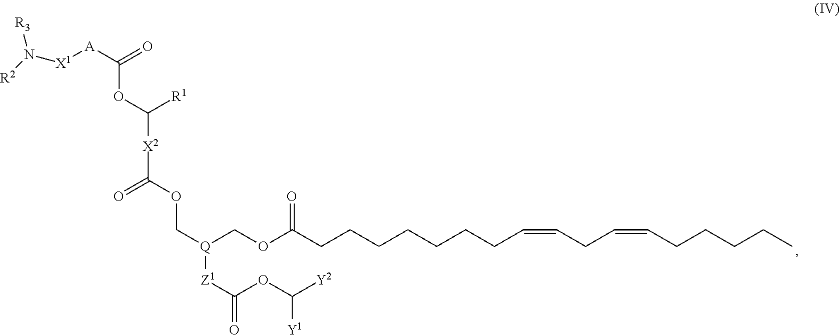

or a salt thereof,
wherein:
Q is CH or

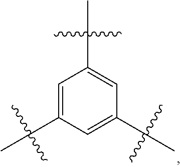 [SMILES: *c1cc(*)cc(*)c1]

A is O, NH, or a direct bond,
X¹ and X² is each independently a C₁₋₅ alkylene,
R¹ is selected from a C₃₋₉ alkyl, C₃₋₉ alkenyl, and C₃₋₉ alkynyl,
R² and R³ is each independently a C₁₋₃ alkyl, or
R² taken together with R³ and the nitrogen atom to which they are attached form a 5-, 6-, or 7-membered ring, and
Z¹ is a C₆₋₁₀ alkylene, and
Y¹ and Y² is each independently selected from a C₃₋₁₀ alkyl, C₃₋₁₀ alkenyl, and C₃₋₁₀ alkynyl.

In some embodiments, the present disclosure relates to a compound represented by one of the following structural formulas:

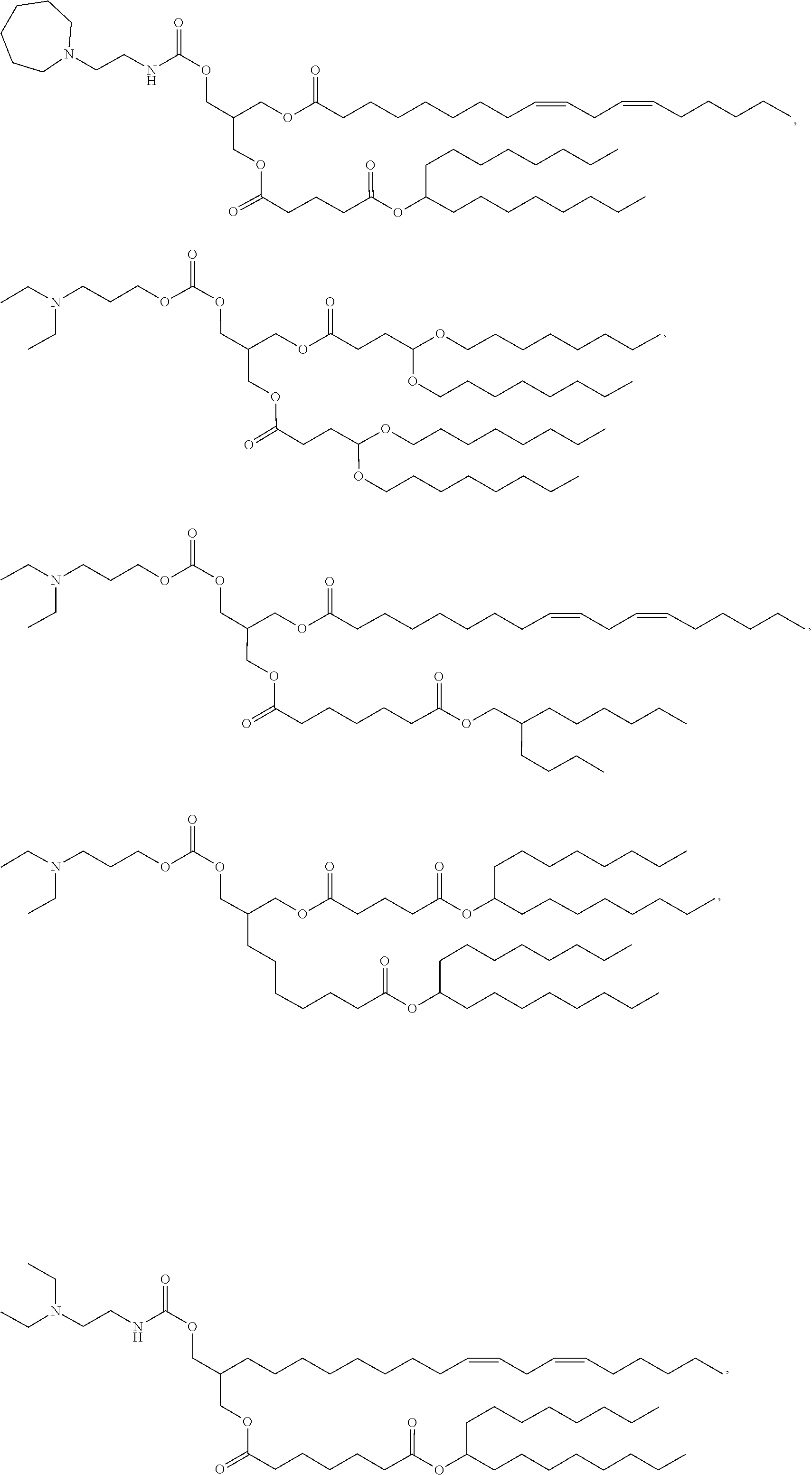

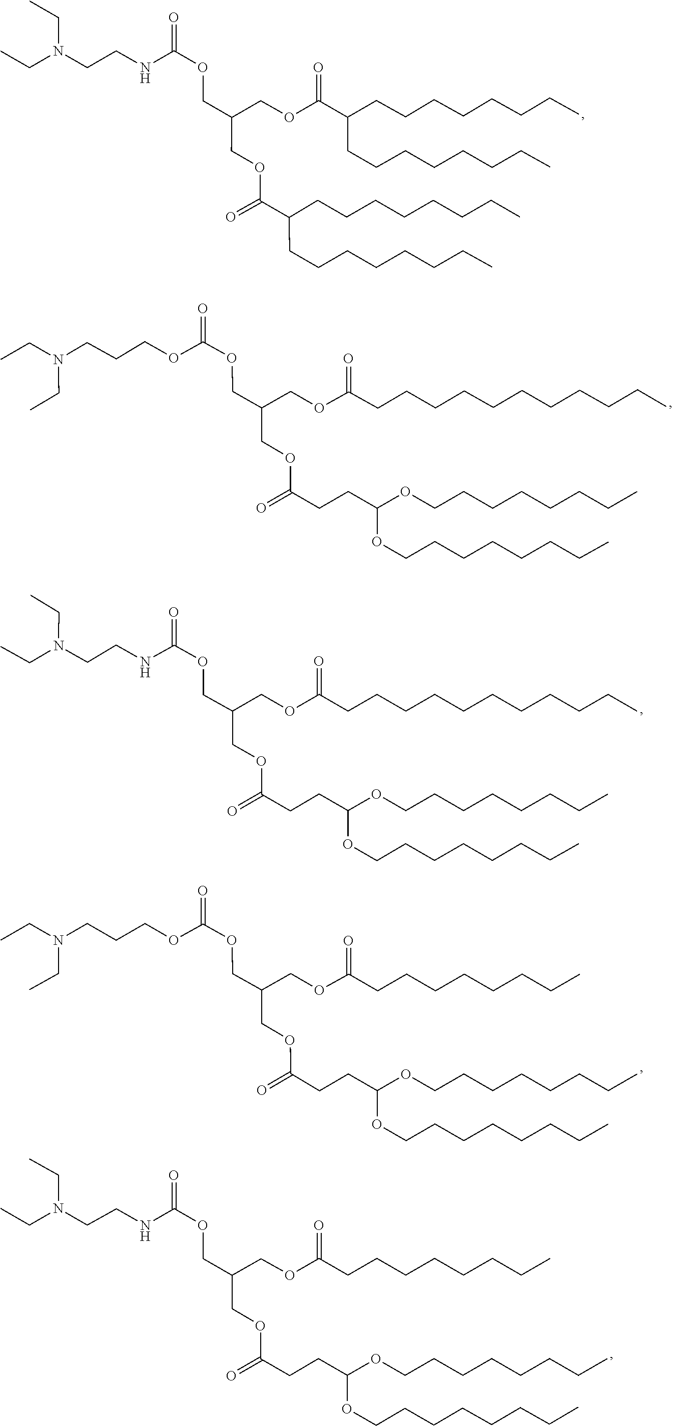

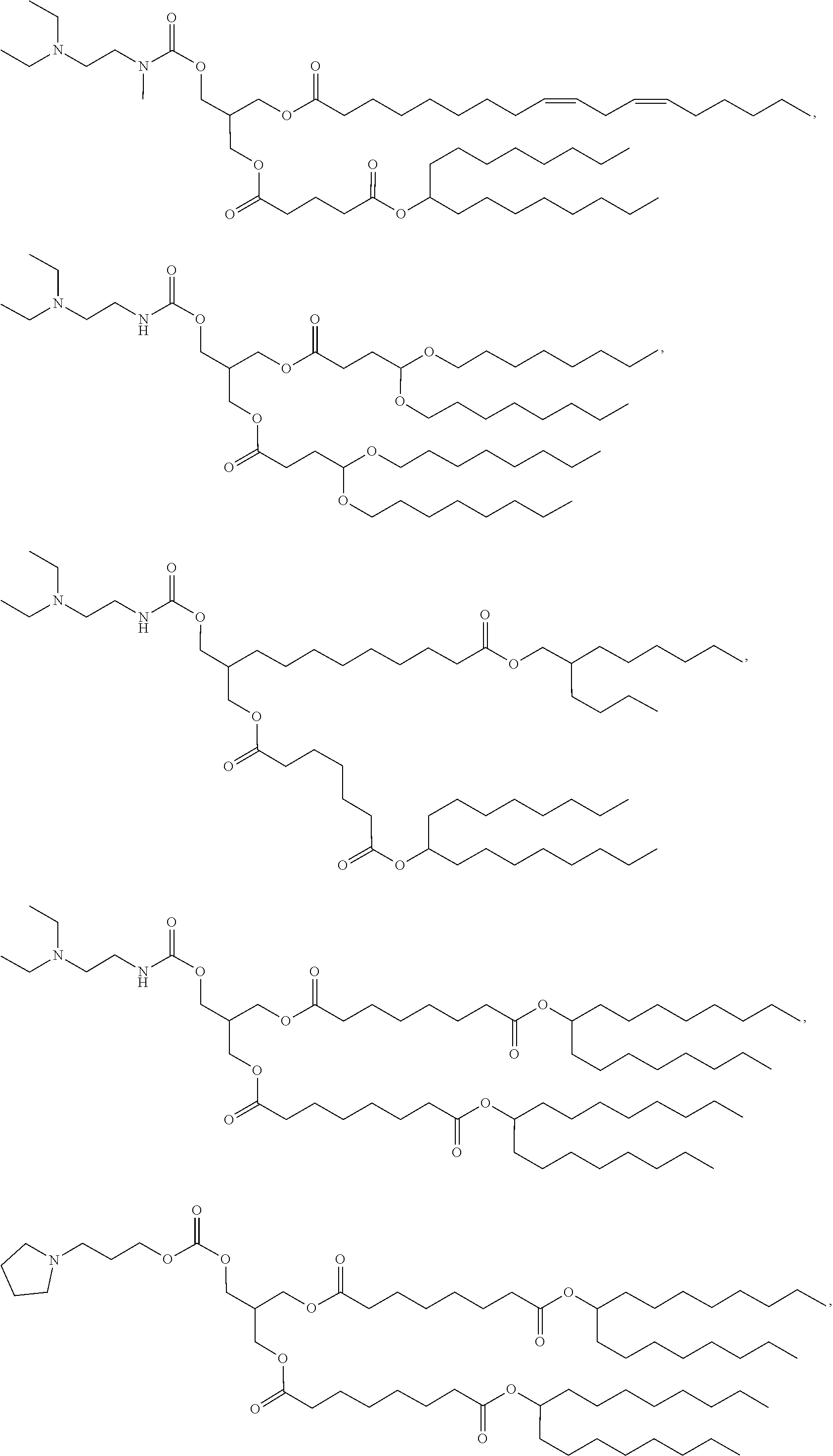

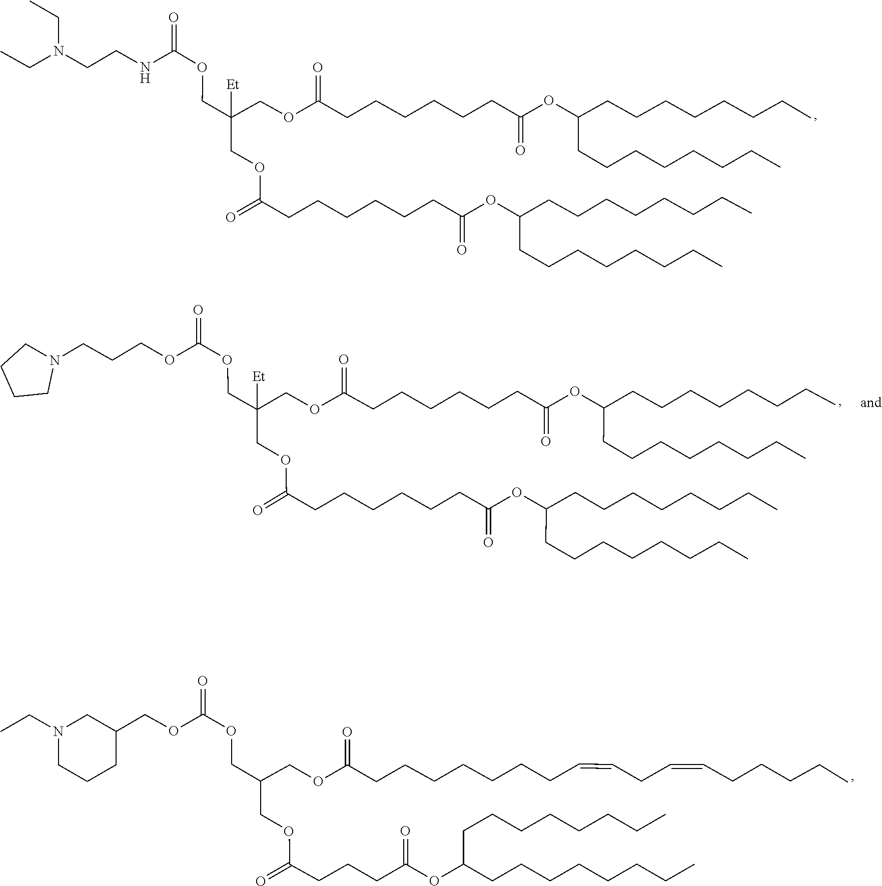

or a salt thereof.

In some embodiments, the present disclosure relates to a compound represented by one of the following structural formulas:

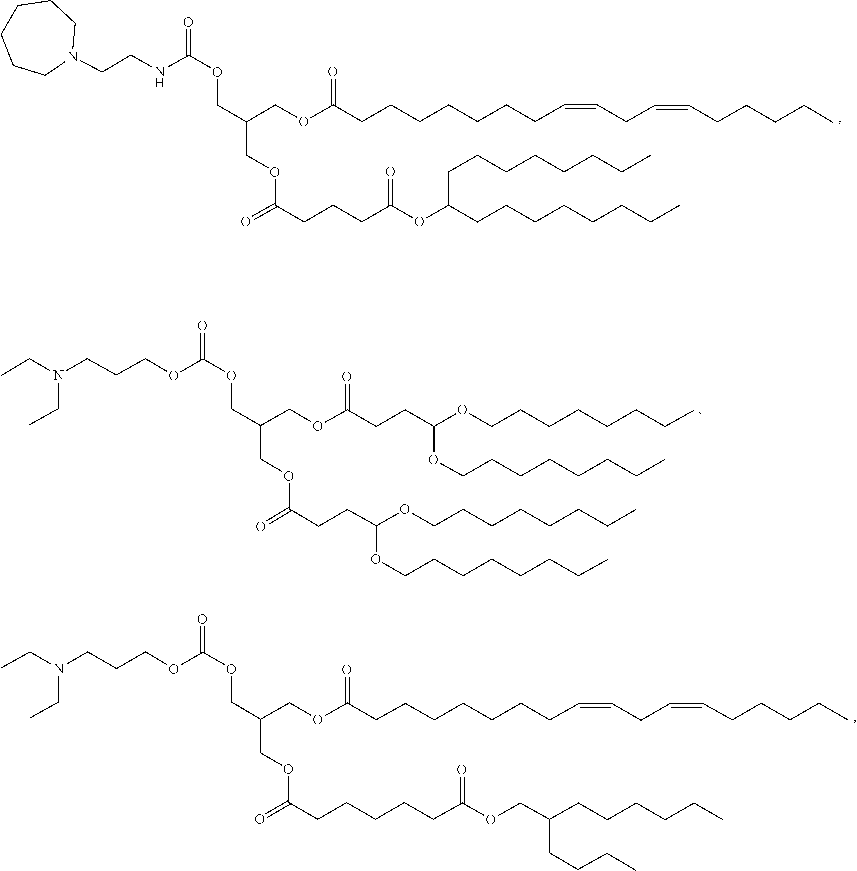

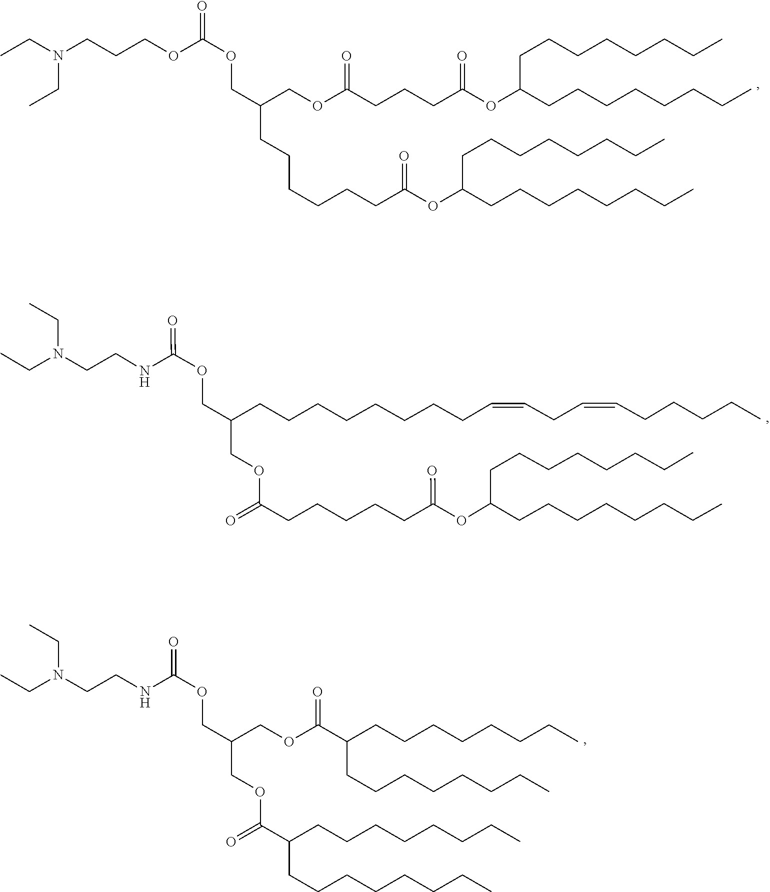

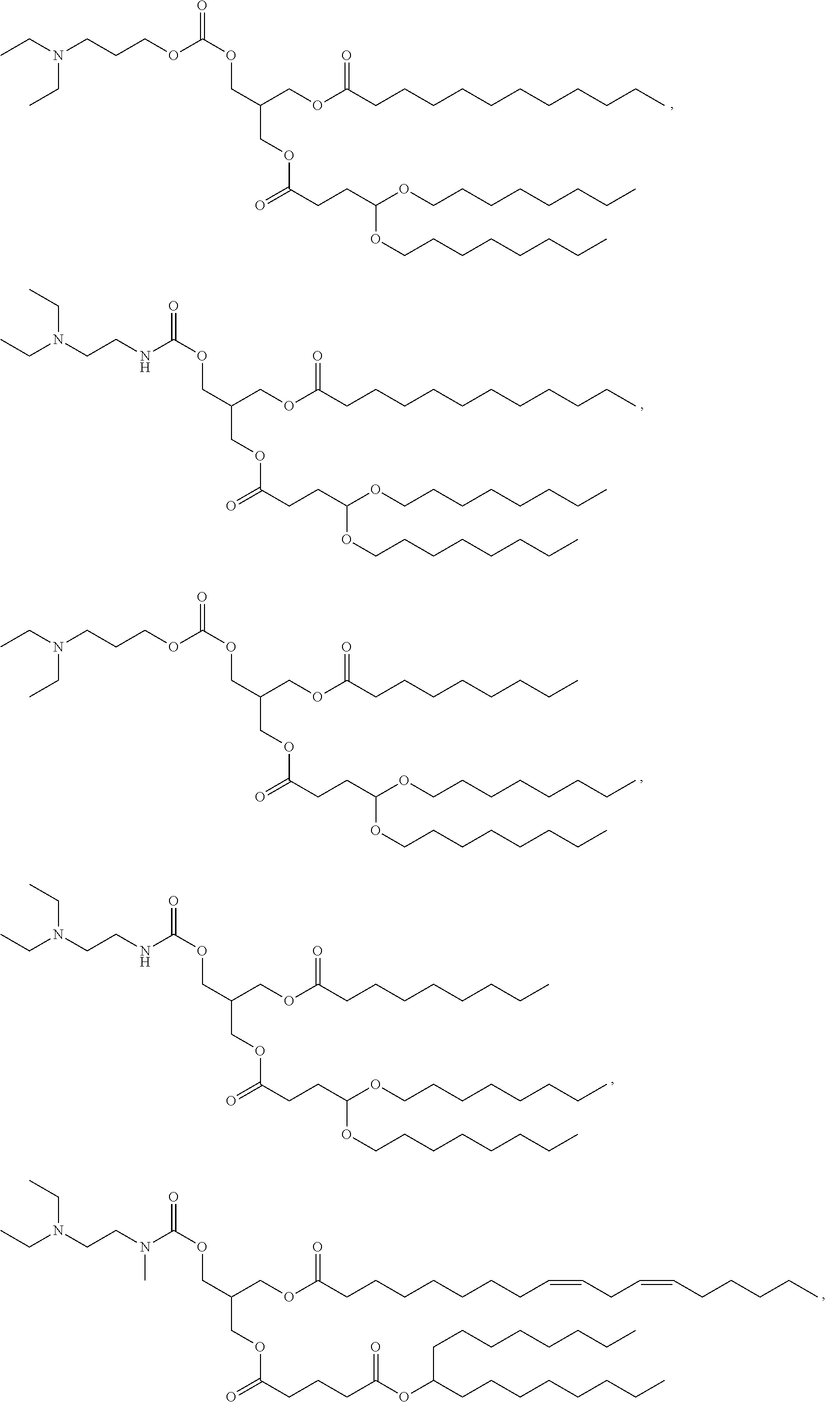

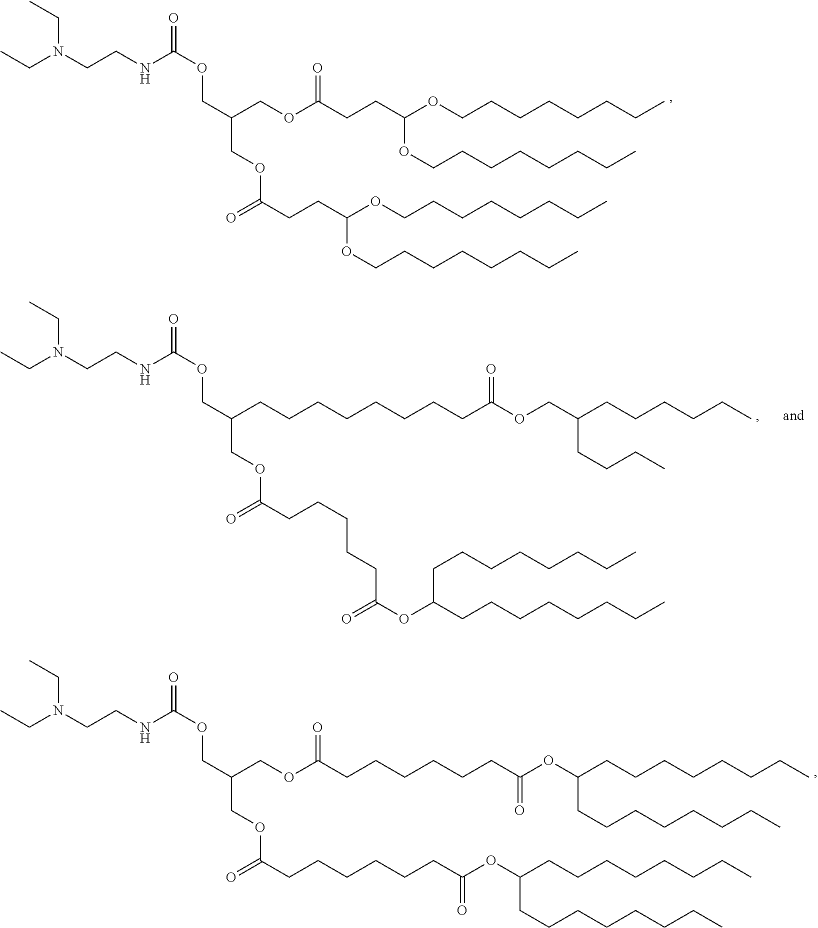

or a salt thereof.

In certain embodiments, the invention relates to a composition comprising a compound of Formula (I)-(IV) or Table 1 and a lipid component.

In some embodiments, the present disclosure relates to a method of cleaving a DNA, comprising contacting a cell with a composition as described herein.

In some embodiments, the present disclosure relates to a method of gene editing, comprising contacting a cell with a composition as described herein.

### BRIEF DESCRIPTION OF DRAWINGS

FIG. 1A is a graph demonstrating percentages of editing of TTR in rat liver after delivery using LNPs comprising Compound 53, Compound 54, Compound 58, or Compound 59 (Experiment 1).

FIG. 1B is a graph demonstrating serum TTR (μg/mL) after delivery using LNPs comprising Compound 53, Compound 54, Compound 58, or Compound 59 (Experiment 1).

FIG. 2A is a graph demonstrating percentages of editing of TTR in rat liver after delivery using LNPs comprising Compound 53, Compound 56, Compound 57, Compound 2, Compound 3, Compound 4, Compound 5, Compound 58, or Compound 59 (Experiment 2).

FIG. 2B is a graph demonstrating serum TTR (μg/mL) after delivery using LNPs comprising Compound 53, Compound 56, Compound 57, Compound 2, Compound 3, Compound 4, Compound 5, Compound 58, or Compound 59 (Experiment 2).

FIG. 3 is a graph demonstrating percentages of editing of TTR in rat liver after delivery using LNPs comprising Compound 53, Compound 1, Compound 7, Compound 4, Compound 9, Compound 57, Compound 8, or Compound 2 (Experiment 3).

FIG. 4 is a graph demonstrating percentages of editing of TTR in rat liver after delivery using LNPs comprising Compound 53, Compound 9, Compound 11, Compound 10, Compound 14, Compound 13, or Compound 12 (Experiment 4).

FIG. 5A is a graph demonstrating percentages of editing of TTR in rat liver after delivery using LNPs comprising Compound 53, Compound 24, Compound 25, Compound 26, or Compound 27 (Experiment 5).

FIG. 5B is a graph demonstrating serum TTR (μg/mL) after delivery using LNPs comprising Compound 53, Compound 24, Compound 25, Compound 26, or Compound 27 (Experiment 5).

FIG. 6 is a graph demonstrating percentages of editing of TTR in rat liver after delivery using LNPs comprising Compound 53 or Compound 23 (Experiment 6).

FIG. 7 is a graph demonstrating percentages of editing of TTR in rat liver after delivery using LNPs comprising Compound 53, Compound 41, Compound 43, Compound 45, Compound 47, Compound 42, Compound 44, Compound 46, or Compound 48 (Experiment 7).

FIG. 8 is a graph demonstrating percentages of editing of TTR in rat liver after delivery using LNPs comprising Compound 53, or Compound 22 (Experiment 8).

FIG. 9 is a graph demonstrating percentages of editing of TTR in rat liver after delivery using LNPs comprising Compound 53, Compound 9, Compound 15, Compound 16, Compound 17, Compound 18, Compound 19, or Compound 20 (Experiment 9).

FIG. 10 is a graph demonstrating percentages of editing of TTR in rat liver after delivery using LNPs comprising Compound 53, Compound 56, Compound 58, Compound 49, or Compound 50 (Experiment 10).

FIG. 11 is a graph demonstrating percentages of editing of TTR in rat liver after delivery using LNPs comprising Compound 53, Compound 55, Compound 23, or Compound 51 (Experiment 11).

FIG. 12 is a graph demonstrating percentages of editing of TTR in rat liver after delivery using LNPs comprising Compound 6, Compound 28, Compound 29, Compound 30, Compound 31, Compound 32, Compound 33, Compound 36, or Compound 37 (Experiment 12).

FIG. 13 is a graph demonstrating percentages of editing of TTR in rat liver after delivery using LNPs comprising Compound 53, Compound 6, Compound 60, Compound 52, or Compound 61 (Experiment 13).

FIG. 14 is a graph demonstrating percentages of editing of TTR in rat liver after delivery using LNPs comprising Compound 6, Compound 2, Compound 35, Compound 39, Compound 40, Compound 38, or Compound 34 (Experiment 14).

FIG. 15 is a graph demonstrating percentages of editing of TTR in rat liver after delivery using LNPs comprising one of Compound 53, Compound 6, or Compound 60, and one of PEG2K-DMG, C13 Ether, or C14 Ether.

FIG. 16 is a graph demonstrating percentages of editing of TTR in rat liver after delivery using LNPs comprising Compound 6, Compound 52, Compound 62, or Compound 63.

FIG. 17 is a graph demonstrating editing efficiency in rat liver, measured by % editing, after delivery of LNPs comprising Compound 53, Compound 6, Compound 68, Compound 66, Compound 33, Compound 69, Compound 64, Compound 65, Compound 67, or Compound 52.

### DETAILED DESCRIPTION

The present disclosure provides lipids, particularly ionizable lipids, and lipid compositions useful for delivering biologically active agents, including nucleic acids, such as CRISPR/Cas component RNAs (mRNA and/or gRNA) (the “cargo”), to a cell, and methods for preparing and using such lipids and compositions. The lipid compositions include an ionizable lipid, a neutral lipid, a PEG lipid, and a helper lipid. In some embodiments, the ionizable lipid is a compound of Formula (I)-(IV) or a compound selected from the compounds of Table 1, or a salt thereof, such as a pharmaceutically acceptable salt thereof, as defined herein.

TABLE 1

| Compound | Structure |
|---|---|
| 1 | 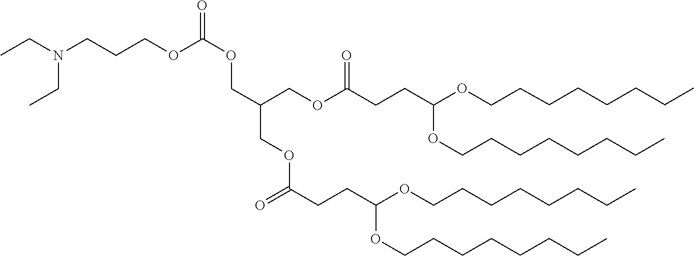 [SMILES: CCCCCCCCOC(CCC(=O)OCC(COC(=O)CCC(OCCCCCCCC)OCCCCCCCC)COC(=O)OCCCN(CC)CC)OCCCCCCCC] |
| 2 | 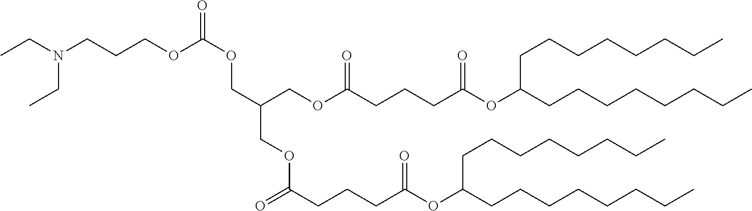 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(CC)CC] |
| 3 | 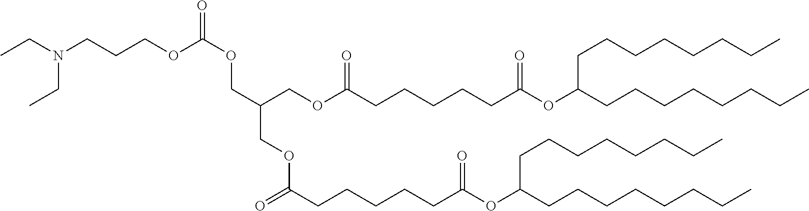 [SMILES: CCCCCCCCC(CCCCCCCC)OC(=O)CCCCCC(=O)OCC(COC(=O)CCCCCC(=O)OC(CCCCCCCC)CCCCCCCC)COC(=O)OCCCN(CC)CC] |
| 4 | 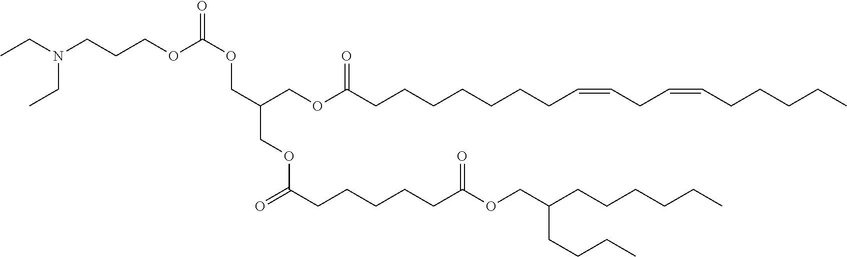 [SMILES: CCCCC/C=C\C/C=C\CCCCCCCC(=O)OCC(COC(=O)CCCCCC(=O)OCC(CCCC)CCCCCC)COC(=O)OCCCN(CC)CC] |
| 5 | 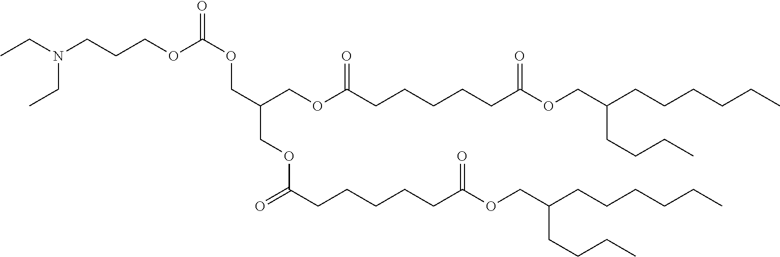 [SMILES: CCCCCCC(CCCC)COC(=O)CCCCCC(=O)OCC(COC(=O)CCCCCC(=O)OCC(CCCC)CCCCCC)COC(=O)OCCCN(CC)CC] |
| 6 | 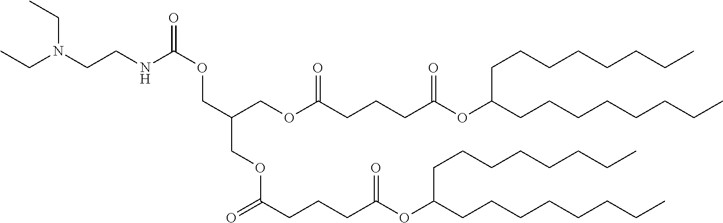 [SMILES: CCCCCCCCC(CCCCCCCC)OC(=O)CCCC(=O)OCC(COC(=O)CCCC(=O)OC(CCCCCCCC)CCCCCCCC)COC(=O)NCCN(CC)CC] |
| 7 | 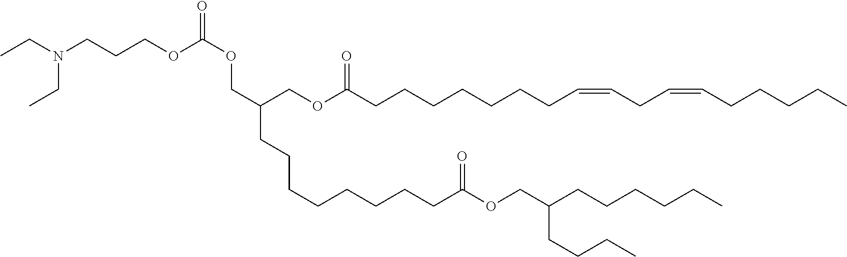 [SMILES: CCCCC/C=C\C/C=C\CCCCCCCC(=O)OCC(CCCCCCCCC(=O)OCC(CCCC)CCCCCC)COC(=O)OCCCN(CC)CC] |
| 8 | 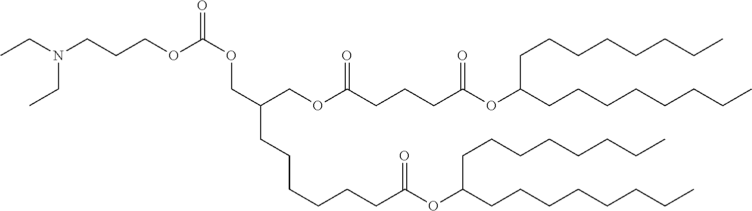 [SMILES: CCCCCCCCC(CCCCCCCC)OC(=O)CCCCCCC(COC(=O)CCCC(=O)OC(CCCCCCCC)CCCCCCCC)COC(=O)OCCCN(CC)CC] |
| 9 | 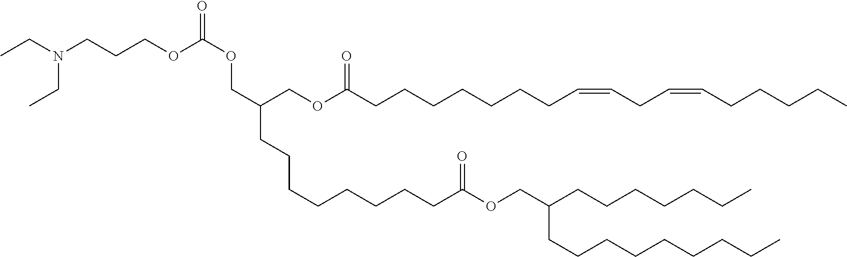 [SMILES: CCCCC/C=C\C/C=C\CCCCCCCC(=O)OCC(CCCCCCCCC(=O)OCC(CCCCCCC)CCCCCCCCC)COC(=O)OCCCN(CC)CC] |
| 10 | 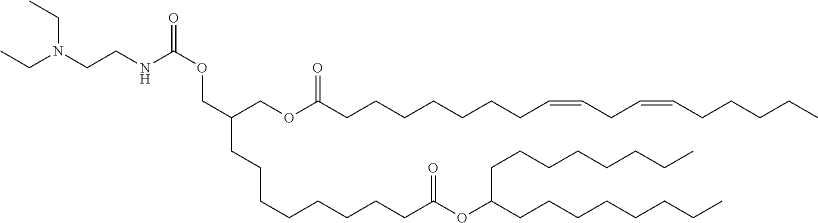 [SMILES: CCCCC/C=C\C/C=C\CCCCCCCC(=O)OCC(CCCCCCCCC(=O)OC(CCCCCCCC)CCCCCCCC)COC(=O)NCCN(CC)CC] |
| 11 | 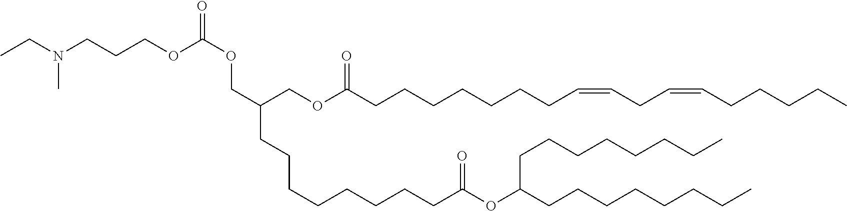 [SMILES: CCCCC/C=C\C/C=C\CCCCCCCC(=O)OCC(CCCCCCCCC(=O)OC(CCCCCCCC)CCCCCCCC)COC(=O)OCCCN(C)CC] |
| 12 | 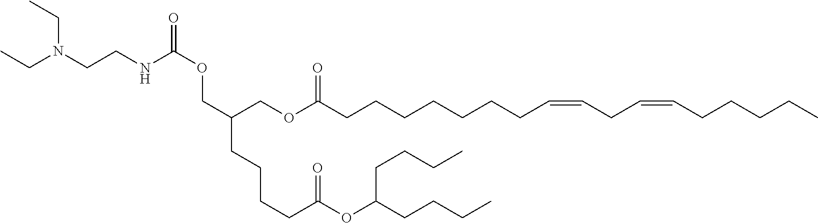 [SMILES: CCCCC/C=C\C/C=C\CCCCCCCC(=O)OCC(CCCCC(=O)OC(CCCC)CCCC)COC(=O)NCCN(CC)CC] |
| 13 | 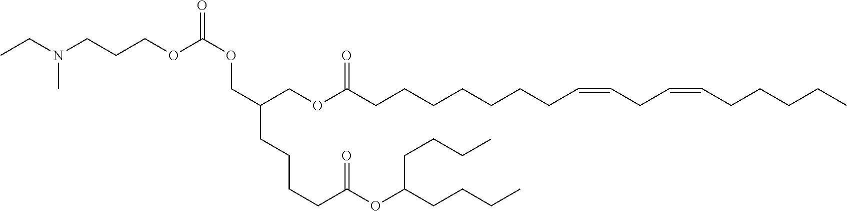 [SMILES: CCCCC/C=C\C/C=C\CCCCCCCC(=O)OCC(CCCCC(=O)OC(CCCC)CCCC)COC(=O)OCCCN(C)CC] |
| 14 | 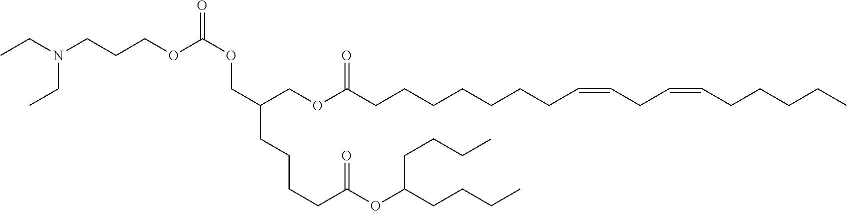 [SMILES: CCCCC/C=C\C/C=C\CCCCCCCC(=O)OCC(CCCCC(=O)OC(CCCC)CCCC)COC(=O)OCCCN(CC)CC] |
| 15 | 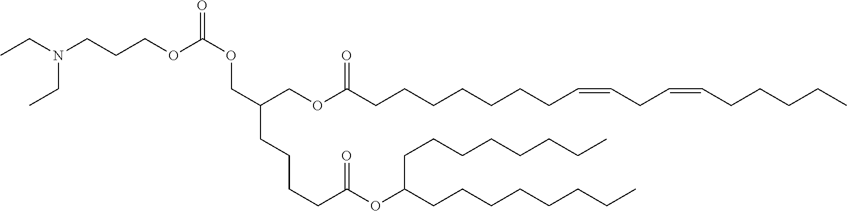 [SMILES: CCCCC/C=C\C/C=C\CCCCCCCC(=O)OCC(CCCCC(=O)OC(CCCCCCCC)CCCCCCCC)COC(=O)OCCCN(CC)CC] |
| 16 | 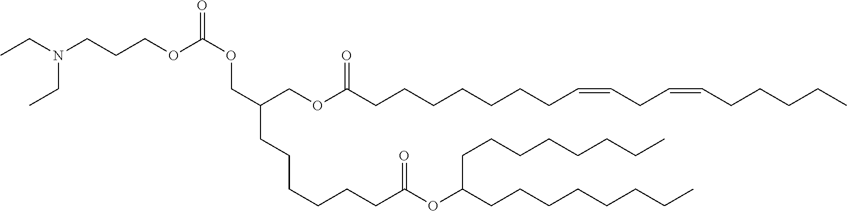 [SMILES: CCCCC/C=C\C/C=C\CCCCCCCC(=O)OCC(CCCCCCC(=O)OC(CCCCCCCC)CCCCCCCC)COC(=O)OCCCN(CC)CC] |
| 17 | 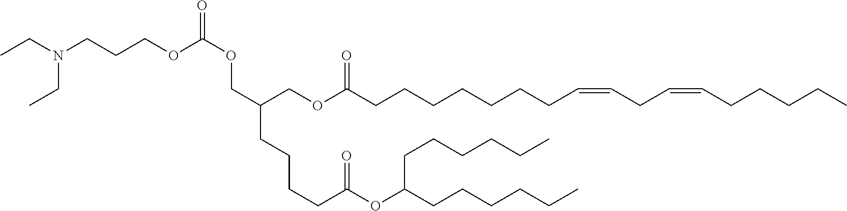 [SMILES: CCCCC/C=C\C/C=C\CCCCCCCC(=O)OCC(CCCCC(=O)OC(CCCCCC)CCCCCC)COC(=O)OCCCN(CC)CC] |
| 18 | 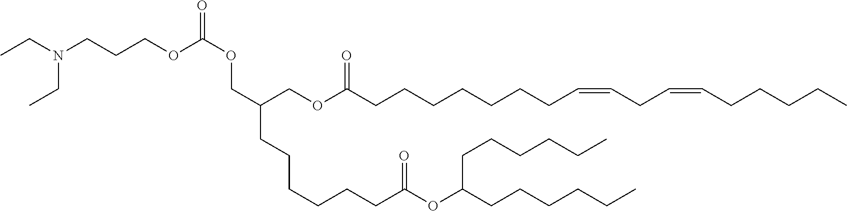 [SMILES: CCCCC/C=C\C/C=C\CCCCCCCC(=O)OCC(CCCCCCC(=O)OC(CCCCCC)CCCCCC)COC(=O)OCCCN(CC)CC] |
| 19 | 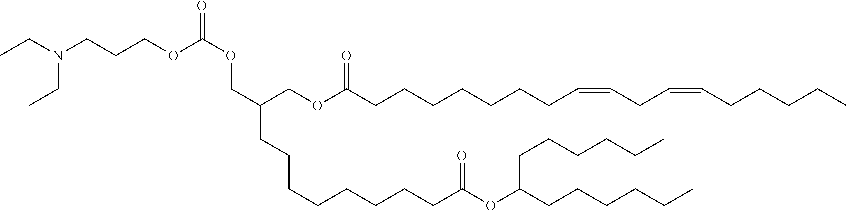 [SMILES: CCCCC/C=C\C/C=C\CCCCCCCC(=O)OCC(CCCCCCCCC(=O)OC(CCCCCC)CCCCCC)COC(=O)OCCCN(CC)CC] |
| 20 | 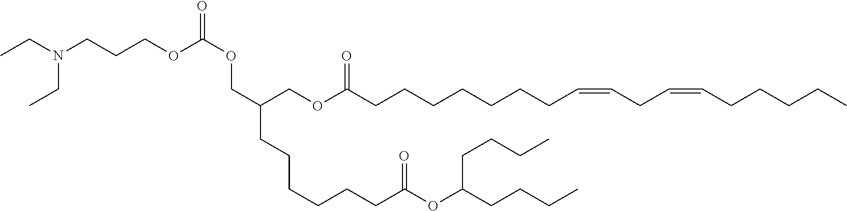 [SMILES: CCCCC/C=C\C/C=C\CCCCCCCC(=O)OCC(CCCCCCC(=O)OC(CCCC)CCCC)COC(=O)OCCCN(CC)CC] |
| 21 | 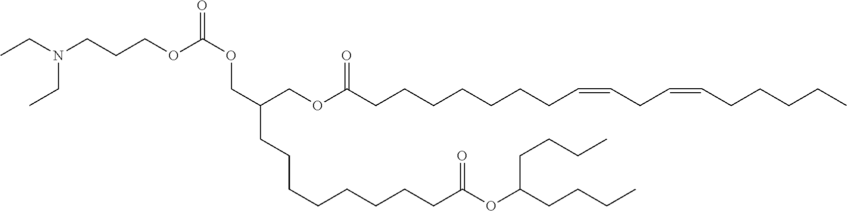 [SMILES: CCCCC/C=C\C/C=C\CCCCCCCC(=O)OCC(CCCCCCCCC(=O)OC(CCCC)CCCC)COC(=O)OCCCN(CC)CC] |
| 22 | 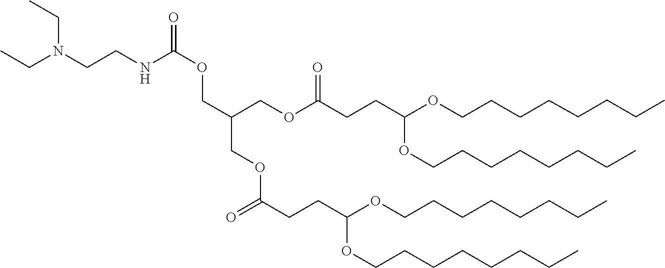 [SMILES: CCCCCCCCOC(CCC(=O)OCC(COC(=O)CCC(OCCCCCCCC)OCCCCCCCC)COC(=O)NCCN(CC)CC)OCCCCCCCC] |
| 23 | 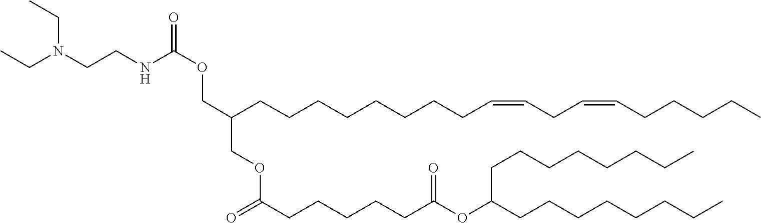 [SMILES: CCCCC/C=C\C/C=C\CCCCCCCCC(COC(=O)CCCCCC(=O)OC(CCCCCCCC)CCCCCCCC)COC(=O)NCCN(CC)CC] |
| 24 | 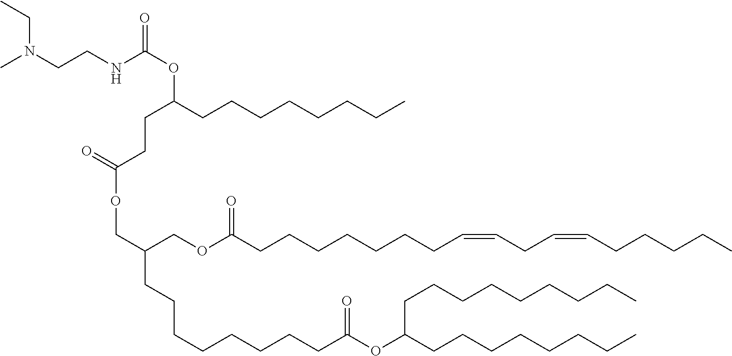 [SMILES: CCCCC/C=C\C/C=C\CCCCCCCC(=O)OCC(CCCCCCCCC(=O)OC(CCCCCCCC)CCCCCCCCC)COC(=O)CCC(CCCCCCCC)OC(=O)NCCN(C)CC] |
| 25 | 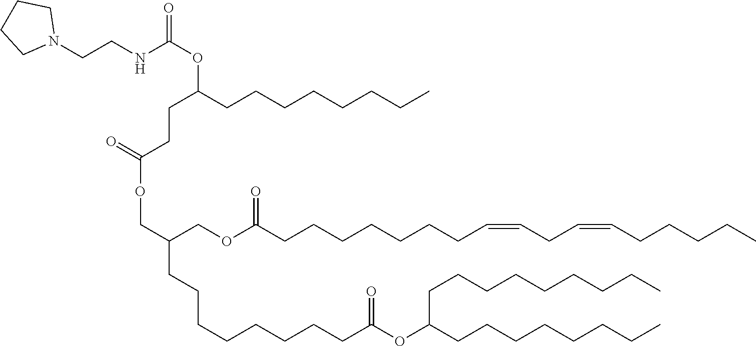 [SMILES: CCCCC/C=C\C/C=C\CCCCCCCC(=O)OCC(CCCCCCCCC(=O)OC(CCCCCCCC)CCCCCCCCC)COC(=O)CCC(CCCCCCCC)OC(=O)NCCN1CCCC1] |
| 26 | 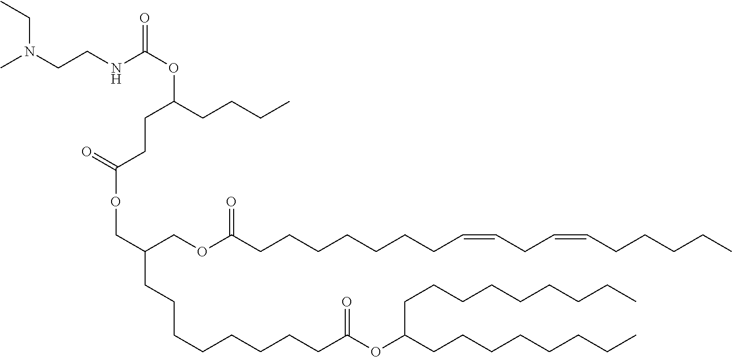 [SMILES: CCCCC/C=C\C/C=C\CCCCCCCC(=O)OCC(CCCCCCCCC(=O)OC(CCCCCCCC)CCCCCCCCC)COC(=O)CCC(CCCC)OC(=O)NCCN(C)CC] |
| 27 | 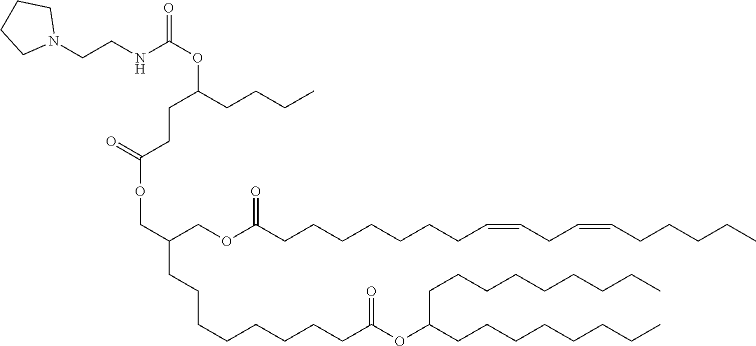 [SMILES: CCCCC/C=C\C/C=C\CCCCCCCC(=O)OCC(CCCCCCCCC(=O)OC(CCCCCCCC)CCCCCCCCC)COC(=O)CCC(CCCC)OC(=O)NCCN1CCCC1] |
| 28 | 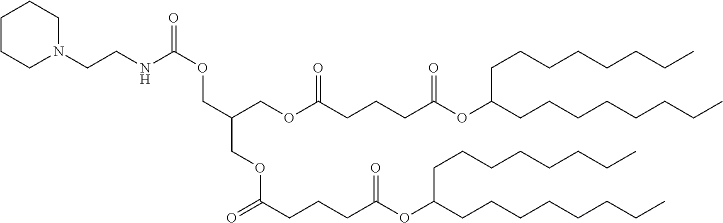 [SMILES: CCCCCCCCC(CCCCCCCC)OC(=O)CCCC(=O)OCC(COC(=O)CCCC(=O)OC(CCCCCCCC)CCCCCCCC)COC(=O)NCCN1CCCCC1] |
| 29 | 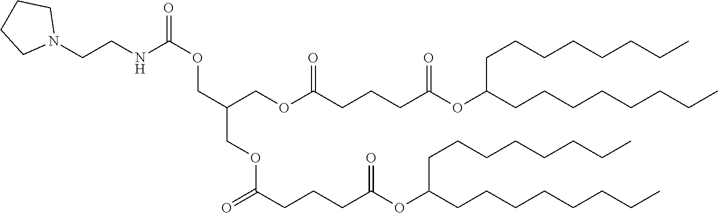 [SMILES: CCCCCCCCC(CCCCCCCC)OC(=O)CCCC(=O)OCC(COC(=O)CCCC(=O)OC(CCCCCCCC)CCCCCCCC)COC(=O)NCCN1CCCC1] |
| 30 | 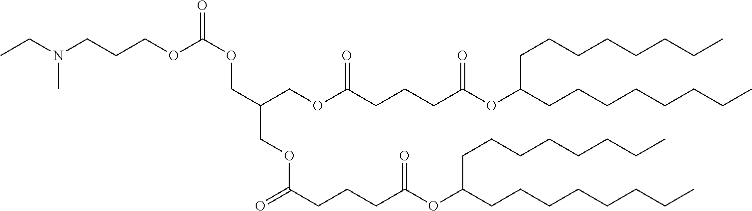 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(C)CC] |
| 31 | 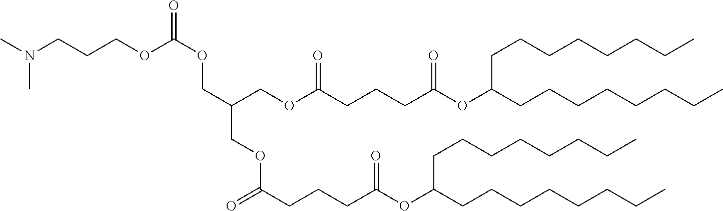 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(C)C] |
| 32 | 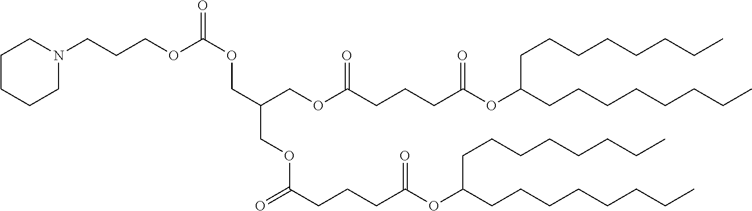 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN1CCCCC1] |
| 33 | 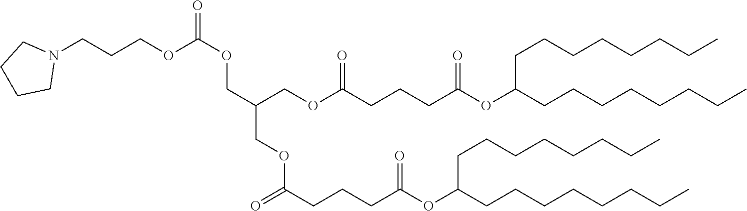 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN1CCCC1] |
| 34 | 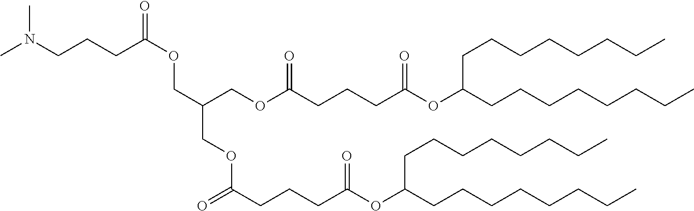 [SMILES: CCCCCCCCC(CCCCCCCC)OC(=O)CCCC(=O)OCC(COC(=O)CCCC(=O)OC(CCCCCCCC)CCCCCCCC)COC(=O)CCCN(C)C] |
| 35 | 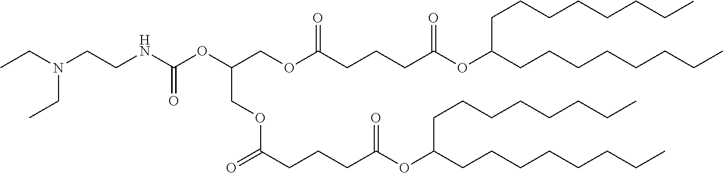 [SMILES: CCCCCCCCC(CCCCCCCC)OC(=O)CCCC(=O)OCC(COC(=O)CCCC(=O)OC(CCCCCCCC)CCCCCCCC)OC(=O)NCCN(CC)CC] |
| 36 | 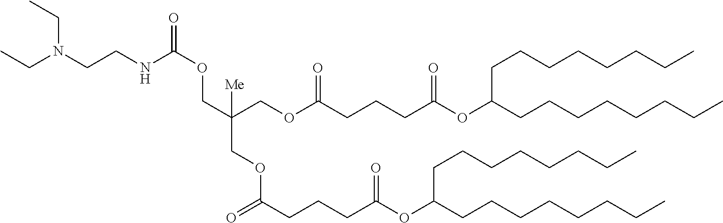 [SMILES: CCCCCCCCC(CCCCCCCC)OC(=O)CCCC(=O)OCC(C)(COC(=O)CCCC(=O)OC(CCCCCCCC)CCCCCCCC)COC(=O)NCCN(CC)CC] |
| 37 | 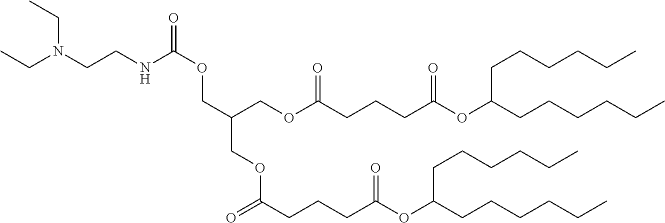 [SMILES: CCCCCCC(CCCCCC)OC(=O)CCCC(=O)OCC(COC(=O)CCCC(=O)OC(CCCCCC)CCCCCC)COC(=O)NCCN(CC)CC] |
| 38 | 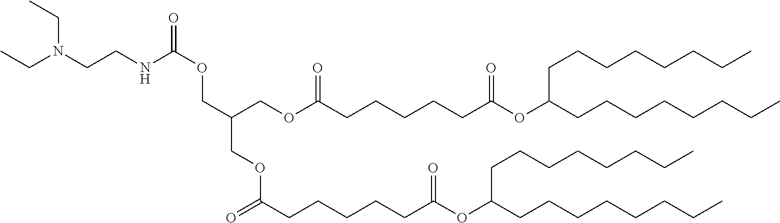 [SMILES: CCCCCCCCC(CCCCCCCC)OC(=O)CCCCCC(=O)OCC(COC(=O)CCCCCC(=O)OC(CCCCCCCC)CCCCCCCC)COC(=O)NCCN(CC)CC] |
| 39 | 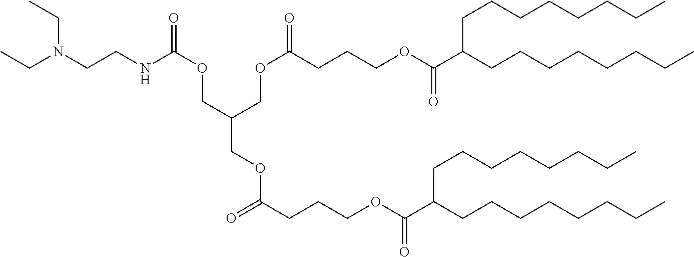 [SMILES: CCCCCCCCC(CCCCCCCC)C(=O)OCCCC(=O)OCC(COC(=O)CCCOC(=O)C(CCCCCCCC)CCCCCCCC)COC(=O)NCCN(CC)CC] |
| 40 | 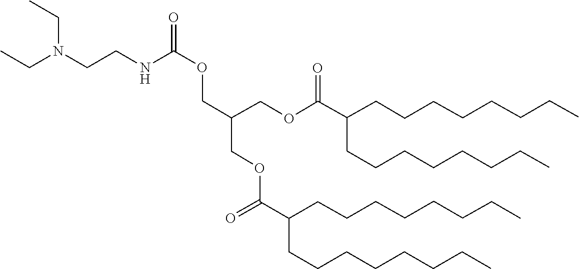 [SMILES: CCCCCCCCC(CCCCCCCC)C(=O)OCC(COC(=O)NCCN(CC)CC)COC(=O)C(CCCCCCCC)CCCCCCCC] |
| 41 | 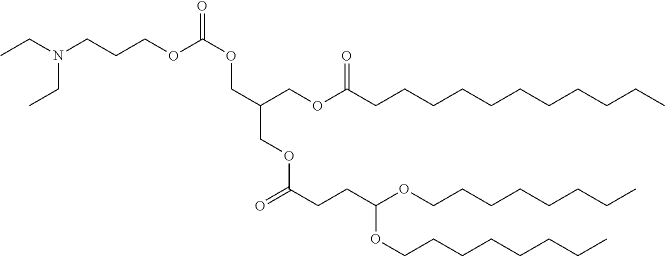 [SMILES: CCCCCCCCCCCC(=O)OCC(COC(=O)CCC(OCCCCCCCC)OCCCCCCCC)COC(=O)OCCCN(CC)CC] |
| 42 | 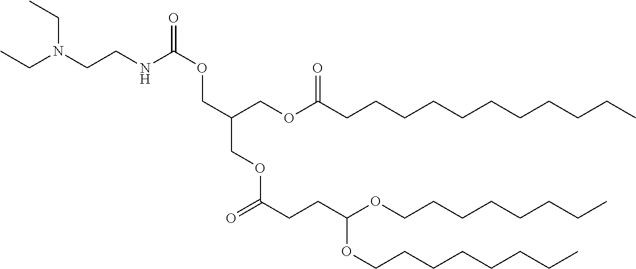 [SMILES: CCCCCCCCCCCC(=O)OCC(COC(=O)CCC(OCCCCCCCC)OCCCCCCCC)COC(=O)NCCN(CC)CC] |
| 43 | 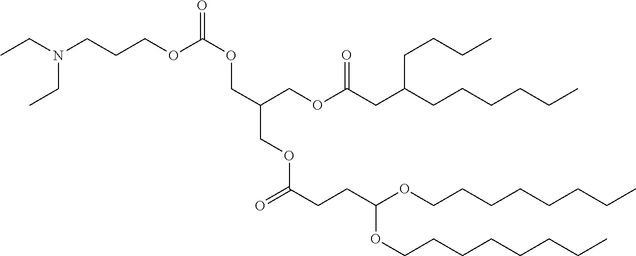 [SMILES: CCCCCCCCOC(CCC(=O)OCC(COC(=O)CC(CCCC)CCCCCC)COC(=O)OCCCN(CC)CC)OCCCCCCCC] |
| 44 | 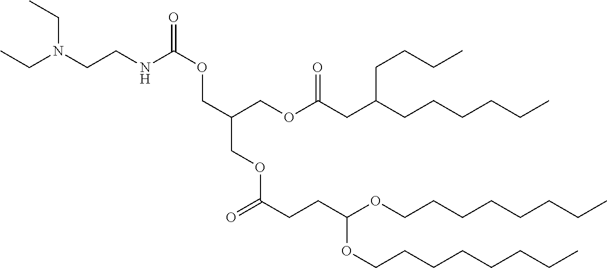 [SMILES: CCCCCCCCOC(CCC(=O)OCC(COC(=O)CC(CCCC)CCCCCC)COC(=O)NCCN(CC)CC)OCCCCCCCC] |
| 45 | 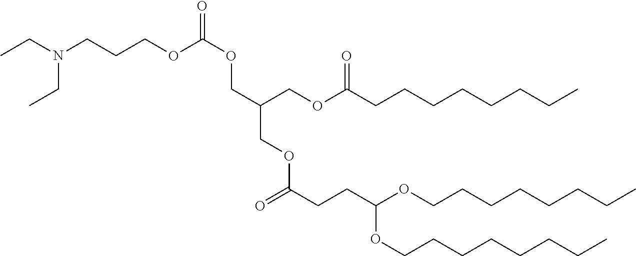 [SMILES: CCCCCCCCOC(CCC(=O)OCC(COC(=O)CCCCCCCC)COC(=O)OCCCN(CC)CC)OCCCCCCCC] |
| 46 | 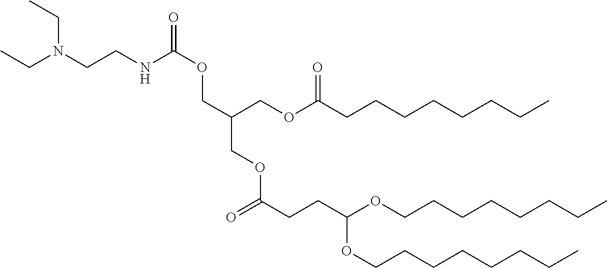 [SMILES: CCCCCCCCOC(CCC(=O)OCC(COC(=O)CCCCCCCC)COC(=O)NCCN(CC)CC)OCCCCCCCC] |
| 47 | 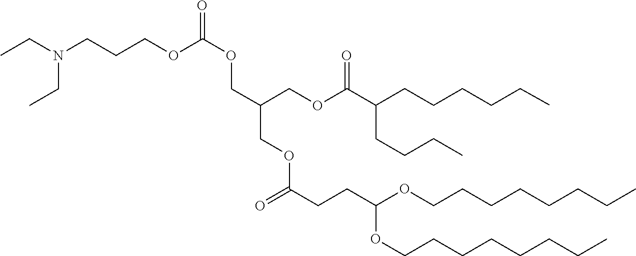 [SMILES: CCCCCCCCOC(CCC(=O)OCC(COC(=O)OCCCN(CC)CC)COC(=O)C(CCCC)CCCCCC)OCCCCCCCC] |
| 48 | 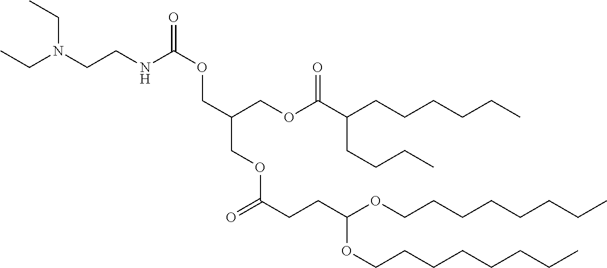 [SMILES: CCCCCCCCOC(CCC(=O)OCC(COC(=O)NCCN(CC)CC)COC(=O)C(CCCC)CCCCCC)OCCCCCCCC] |
| 49 | 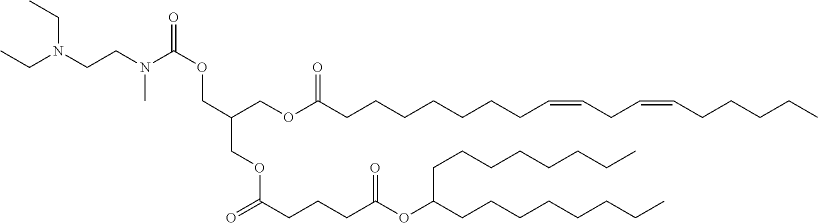 [SMILES: CCCCC/C=C\C/C=C\CCCCCCCC(=O)OCC(COC(=O)CCCC(=O)OC(CCCCCCCC)CCCCCCCC)COC(=O)N(C)CCN(CC)CC] |
| 50 | 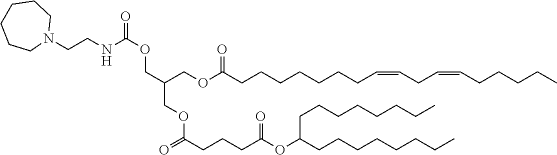 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1] |
| 51 | 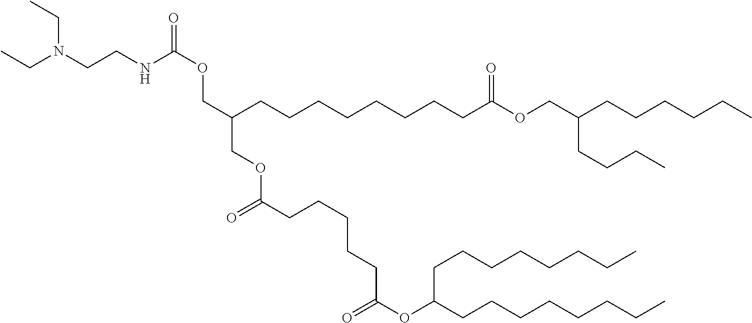 [SMILES: CCCCCCCCC(CCCCCCCC)OC(=O)CCCCCC(=O)OCC(CCCCCCCCC(=O)OCC(CCCC)CCCCCC)COC(=O)NCCN(CC)CC] |
| 52 | 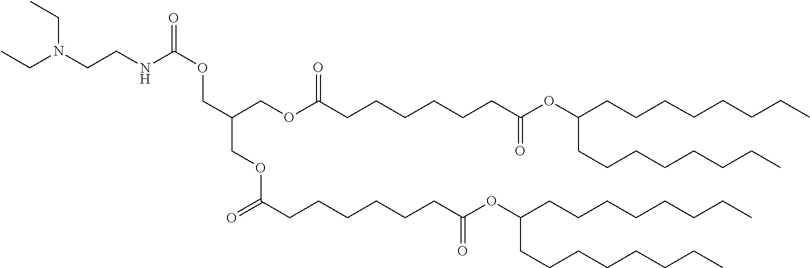 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)NCCN(CC)CC] |
| 62 | 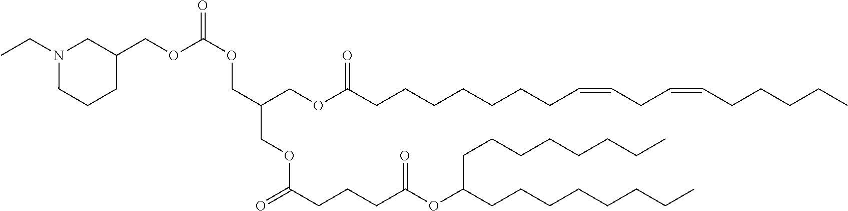 [SMILES: CCCCC/C=C\C/C=C\CCCCCCCC(=O)OCC(COC(=O)CCCC(=O)OC(CCCCCCCC)CCCCCCCC)COC(=O)OCC1CCCN(CC)C1] |
| 63 | 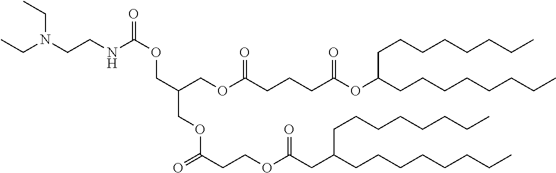 [SMILES: CCCCCCCCC(CCCCCCCC)CC(=O)OCCC(=O)OCC(COC(=O)CCCC(=O)OC(CCCCCCCC)CCCCCCCC)COC(=O)NCCN(CC)CC] |
| 64 | 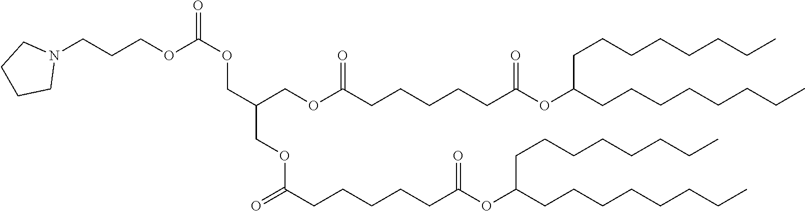 [SMILES: CCCCCCCCC(CCCCCCCC)OC(=O)CCCCCC(=O)OCC(COC(=O)CCCCCC(=O)OC(CCCCCCCC)CCCCCCCC)COC(=O)OCCCN1CCCC1] |
| 65 | 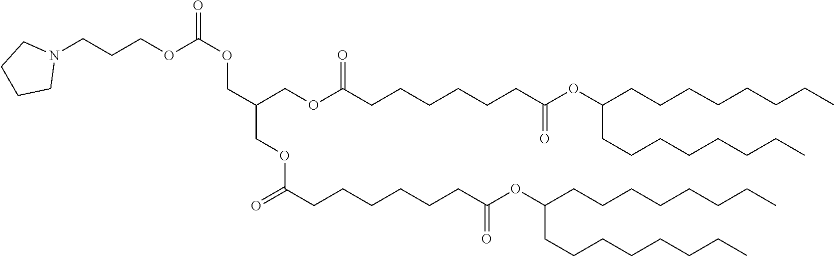 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCCC1] |
| 66 | 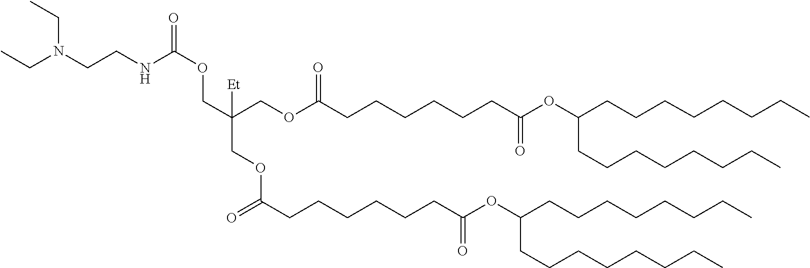 [SMILES: CCCCCCCCC(CCCCCCCC)OC(=O)CCCCCCC(=O)OCC(CC)(COC(=O)CCCCCCC(=O)OC(CCCCCCCC)CCCCCCCC)COC(=O)NCCN(CC)CC] |
| 67 | 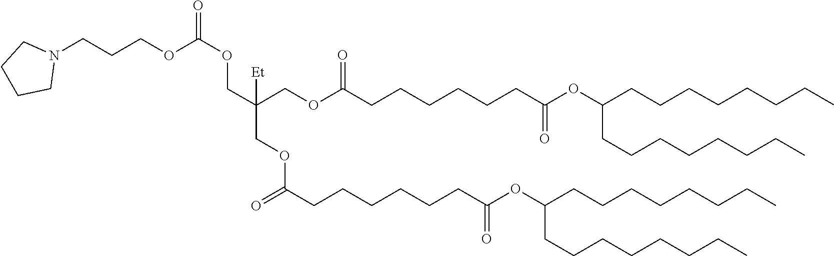 [SMILES: CCCCCCCCC(CCCCCCCC)OC(=O)CCCCCCC(=O)OCC(CC)(COC(=O)CCCCCCC(=O)OC(CCCCCCCC)CCCCCCCC)COC(=O)OCCCN1CCCC1] |
| 68 | 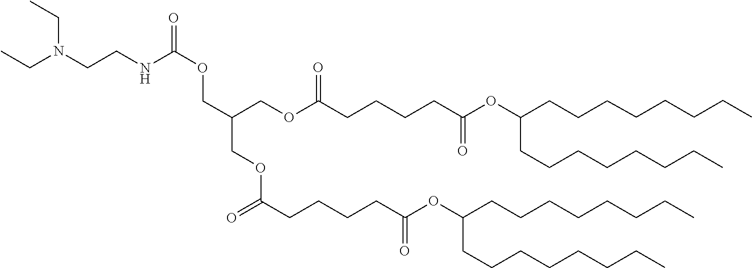 [SMILES: CCCCCCCCC(CCCCCCCC)OC(=O)CCCCC(=O)OCC(COC(=O)CCCCC(=O)OC(CCCCCCCC)CCCCCCCC)COC(=O)NCCN(CC)CC] |
| 69 | 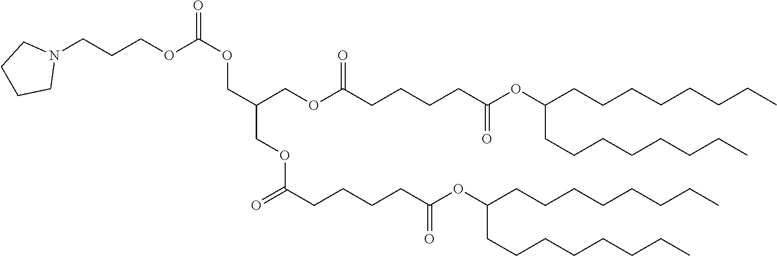 [SMILES: CCCCCCCCC(CCCCCCCC)OC(=O)CCCCC(=O)OCC(COC(=O)CCCCC(=O)OC(CCCCCCCC)CCCCCCCC)COC(=O)OCCCN1CCCC1] |

In certain embodiments, the lipid compositions may comprise a biologically active agent, e.g. an RNA component. In certain embodiments, the RNA component includes an mRNA. In some embodiments, the mRNA is an mRNA encoding a Class 2 Cas nuclease. In certain embodiments, the RNA component includes a gRNA and optionally an mRNA encoding a Class 2 Cas nuclease. In some embodiments, the lipid compositions are lipid nanoparticle (LNP) compositions. “Lipid nanoparticle” or “LNP” refers to, without limiting the meaning, a particle that comprises a plurality of (i.e., more than one) lipid components physically associated with each other by intermolecular forces.

Methods of gene editing and methods of making engineered cells using these lipid compositions are also provided. In some embodiments, LNP compositions may be used to deliver a biologically active agent to a cell, a tissue, or an animal. In some embodiments, the cell is a eukaryotic cell, and in particular a human cell. In some embodiments, the cell is a liver cell. In some embodiments, the cell is a type of cell useful in a therapy, for example, adoptive cell therapy (ACT), such as autologous and allogeneic cell therapies. In some embodiments, the cell is a stem cell, such as a hematopoietic stem cell, an induced pluripotent stem cell, or another multipotent or pluripotent cell. In some embodiments, the cell is a stem cell, for example, a mesenchymal stem cell that can develop into a bone, cartilage, muscle, or fat cell. In some embodiments, the stem cells comprise ocular stem cells. In certain embodiments, the cell is selected from mesenchymal stem cells, hematopoietic stem cells (HSCs), mononuclear cells, endothelial progenitor cells (EPCs), neural stem cells (NSCs), limbal stem cells (LSCs), tissue-specific primary cells or cells derived therefrom (TSCs), induced pluripotent stem cells (iPSCs), ocular stem cells, pluripotent stem cells (PSCs), embryonic stem cells (ESCs), and cells for organ or tissue transplantations.

In some embodiments, the cell is an immune cell, such as a leukocyte or a lymphocyte. In preferred embodiments, the immune cell is a lymphocyte. In certain embodiments, the lymphocyte is a T cell, a B cell, or an NK cell. In preferred embodiments, the lymphocyte is a T cell. In certain embodiments, the lymphocyte is an activated T cell. In certain embodiments, the lymphocyte is a non-activated T cell.

### Ionizable Lipids

The disclosure provides ionizable lipids that can be used in LNP compositions.

The compounds of Formula (I)-(IV) or Table 1 of the present disclosure may form salts depending upon the pH of the medium they are in. For example, in a slightly acidic medium, the compounds of Formula (I)-(IV) or Table 1 may be protonated and thus bear a positive charge. Conversely, in a slightly basic medium, such as, for example, blood where pH is approximately 7.35, the compounds of Formula (I)-(IV) or Table 1 may not be protonated and thus bear no charge. In some embodiments, the compounds of Formula (I)-(IV) or Table 1 of the present disclosure may be predominantly protonated at a pH of at least about 9. In some embodiments, the compounds of Formula (I)-(IV) or Table 1 of the present disclosure may be predominantly protonated at a pH of at least about 10.

The pH at which a compound of Formula (I)-(IV) or Table 1 is predominantly protonated is related to its intrinsic pKa. In some embodiments, a salt of a compound of Formula (I)-(IV) or Table 1 of the present disclosure has a pKa in the range of from about 5.1 to about 8.0, even more preferably from about 5.5 to about 7.6. In some embodiments, a salt of a compound of Formula (I)-(IV) or Table 1 of the present disclosure has a pKa in the range of from about 5.7 to about 8, from about 5.7 to about 7.6, from about 6 to about 8, from about 6 to about 7.5, from about 6 to about 7, from about 6 to about 6.9, from about 6 to about 6.5, from about 6.1 to about 6.9, or from about 6 to about 6.85. In some embodiments, a salt of a compound of Formula (I)-(IV) or Table 1 of the present disclosure has a pKa of about 6.0, about 6.1, about 6.1, about 6.2, about 6.3, about 6.4, about 6.6, about 6.7, about 6.8, or about 6.9. Alternatively, a salt of a compound of Formula (I)-(IV) or Table 1 of the present disclosure has a pKa in the range of from about 6 to about 8. The pKa of a salt of a compound of Formula (I)-(IV) or Table 1 can be an important consideration in formulating LNPs, as it has been found that LNPs formulated with certain lipids having a pKa ranging from about 5.5 to about 7.0 are effective for delivery of cargo in vivo, e.g. to the liver. Further, it has been found that LNPs formulated with certain lipids having a pKa ranging from about 5.3 to about 6.4 are effective for delivery in vivo, e.g. to tumors. See, e.g., WO 2014/136086. In some embodiments, the ionizable lipids are positively charged at an acidic pH but neutral in the blood.

### Additional Lipids

“Neutral lipids” suitable for use in a lipid composition of the disclosure include, for example, a variety of neutral, uncharged or zwitterionic lipids. Examples of neutral phospholipids suitable for use in the present disclosure include, but are not limited to, dipalmitoylphosphatidylcholine (DPPC), distearoylphosphatidylcholine (DSPC), dioleoylphosphocholine (DOPC), dimyristoylphosphatidylcholine (DMPC), 1-palmitoyl-2-linoleoyl-sn-glycero-3-phosphatidylcholine (PLPC), 1,2-diarachidoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-dibehenoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), lysophosphatidylethanolamine and combinations thereof. In certain embodiments, the neutral phospholipid may be selected from distearoylphosphatidylcholine (DSPC) and dimyristoyl phosphatidyl ethanolamine (DMPE), preferably distearoylphosphatidylcholine (DSPC).

“Helper lipids” include steroids, sterols, and alkyl resorcinols. Helper lipids suitable for use in the present disclosure include, but are not limited to, cholesterol, 5-heptadecylresorcinol, and cholesterol hemisuccinate. In certain embodiments, the helper lipid may be cholesterol or a derivative thereof, such as cholesterol hemisuccinate.

In some embodiments, the LNP compositions include polymeric lipids, such as PEG lipids which can affect the length of time the nanoparticles can exist in vivo or ex vivo (e.g., in the blood or medium). PEG lipids may assist in the formulation process by, for example, reducing particle aggregation and controlling particle size. PEG lipids used herein may modulate pharmacokinetic properties of the LNPs. Typically, the PEG lipid comprises a lipid moiety and a polymer moiety based on PEG (sometimes referred to as poly(ethylene oxide)) (a PEG moiety). PEG lipids suitable for use in a lipid composition with a compound of Formula (I)-(IV) or Table 1 of the present disclosure and information about the biochemistry of such lipids can be found in Romberg et al., Pharmaceutical Research 25(1), 2008, pp. 55-71 and Hoekstra et al., Biochimica et Biophysica Acta 1660 (2004) 41-52. Additional suitable PEG lipids are disclosed, e.g., in WO 2015/095340 (p. 31, line 14 to p. 37, line 6), WO 2006/007712, and WO 2011/076807 (“stealth lipids”), each of which is incorporated by reference in its entirety.

In some embodiments, the lipid moiety may be derived from diacylglycerol or diacylglycamide, including those comprising a dialkylglycerol or dialkylglycamide group having alkyl chain length independently comprising from about C4 to about C40 saturated or unsaturated carbon atoms, wherein the chain may comprise one or more functional groups such as, for example, an amide or ester. In some embodiments, the alkyl chain length comprises about C10 to C20. The dialkylglycerol or dialkylglycamide group can further comprise one or more substituted alkyl groups. The chain lengths may be symmetrical or asymmetric.

Unless otherwise indicated, the term “PEG” as used herein means any polyethylene glycol or other polyalkylene ether polymer, such as an optionally substituted linear or branched polymer of ethylene glycol or ethylene oxide. In certain embodiments, the PEG moiety is unsubstituted. Alternatively, the PEG moiety may be substituted, e.g., by one or more alkyl, alkoxy, acyl, hydroxy, or aryl groups. For example, the PEG moiety may comprise a PEG copolymer such as PEG-polyurethane or PEG-polypropylene (see, e.g., J. Milton Harris, Poly(ethylene glycol) chemistry: biotechnical and biomedical applications (1992)); alternatively, the PEG moiety may be a PEG homopolymer. In certain embodiments, the PEG moiety has a molecular weight of from about 130 to about 50,000, such as from about 150 to about 30,000, or even from about 150 to about 20,000. Similarly, the PEG moiety may have a molecular weight of from about 150 to about 15,000, from about 150 to about 10,000, from about 150 to about 6,000, or even from about 150 to about 5,000. In certain preferred embodiments, the PEG moiety has a molecular weight of from about 150 to about 4,000, from about 150 to about 3,000, from about 300 to about 3,000, from about 1,000 to about 3,000, or from about 1,500 to about 2,500.

In certain preferred embodiments, the PEG moiety is a “PEG-2K,” also termed “PEG 2000” or “PEG2K”, which has an average molecular weight of about 2,000 daltons. PEG-2K is represented herein by the following formula (III),

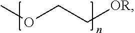

wherein n is about 45, meaning that the number averaged degree of polymerization comprises about 45 subunits. However, other PEG embodiments known in the art may be used, including, e.g., those where the number-averaged degree of polymerization comprises about 23 subunits (n=23), and/or 68 subunits (n=68). In some embodiments, n may range from about 30 to about 60. In some embodiments, n may range from about 35 to about 55. In some embodiments, n may range from about 40 to about 50. In some embodiments, n may range from about 42 to about 48. In some embodiments, n may be 45. In some embodiments, R may be selected from H, substituted alkyl, and unsubstituted alkyl. In some embodiments, R may be unsubstituted alkyl, such as methyl.

In any of the embodiments described herein, the PEG lipid may be selected from PEG-dilauroylglycerol, PEG-dimyristoylglycerol (PEG-DMG) (e.g., 1,2-dimyristoyl-rac-glycero-3-methoxypolyethylene glycol-2000 (PEG2K-DMG) (e.g., catalog #GM-020 from NOF, Tokyo, Japan)), PEG-dipalmitoylglycerol, PEG-distearoylglycerol (PEG-DSPE) (e.g., catalog #DSPE-020CN, NOF, Tokyo, Japan)), PEG-dilaurylglycamide, PEG-dimyristylglycamide, PEG-dipalmitoylglycamide, and PEG-distearoylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-ditetradecoxylbenzyl-[omega]-methyl-poly(ethylene glycol)ether), 1,2-dimyristoyl-sn-glycero-3-phosphoethanolamine-N-[methoxy(polyethylene glycol)-2000] (PEG2K-DMPE), 1,2-distearoyl-sn-glycero-3-phosphoethanolamine-N-[methoxy(polyethylene glycol)-2000] (PEG2K-DSPE) (e.g., cat. #880120C from Avanti Polar Lipids, Alabaster, Alabama, USA), 1,2-distearoyl-sn-glycerol, methoxypolyethylene glycol (PEG2K-DSG; GS-020, NOF Tokyo, Japan), poly(ethylene glycol)-2000-dimethacrylate (PEG2K-DMA), 1,2-distearyloxypropyl-3-amine-N-[methoxy(polyethylene glycol)-2000] (PEG2K-DSA), and methoxy-PEG2000-carbamoyl-1,2-tridecyoxypropylamine (C13 Ether), and methoxy-PEG2000-carbamoyl-1,2-tetradecyoxypropylamine (C14 Ether). In certain such embodiments, the PEG lipid may be PEG2K-DMG. In some embodiments, the PEG lipid may be PEG2K-DSG. In other embodiments, the PEG lipid may be PEG2K-DSPE. In some embodiments, the PEG lipid may be PEG2K-DMA. In yet other embodiments, the PEG lipid may be PEG2K-C-DMA. In certain embodiments, the PEG lipid may be compound S027, disclosed in WO2016/010840 (paragraphs [00240] to [00244]), which is incorporated herein by reference in its entirety. In some embodiments, the PEG lipid may be PEG2K-DSA. In other embodiments, the PEG lipid may be PEG2K-C11. In some embodiments, the PEG lipid may be PEG2K-C14. In some embodiments, the PEG lipid may be PEG2K-C16. In some embodiments, the PEG lipid may be PEG2K-C18.

In preferred embodiments, the PEG lipid includes a glycerol group. In preferred embodiments, the PEG lipid includes a dimyristoylglycerol (DMG) group. In preferred embodiments, the PEG lipid comprises PEG2K. In preferred embodiments, the PEG lipid is a PEG-DMG. In preferred embodiments, the PEG lipid is a PEG2K-DMG. In preferred embodiments, the PEG lipid is 1,2-dimyristoyl-rac-glycero-3-methoxypolyethylene glycol-2000. In preferred embodiments, the PEG2K-DMG is 1,2-dimyristoyl-rac-glycero-3-methoxypolyethylene glycol-2000.

### Lipid Compositions

Described herein are lipid compositions comprising at least one compound of Formula (I)-(IV) or Table 1, or a salt thereof (e.g., a pharmaceutically acceptable salt thereof), at least one helper lipid, at least one neutral lipid, and at least one polymeric lipid. In some embodiments, the lipid composition comprises at least one compound of Formula (I)-(IV) or Table 1, or a salt thereof, at least one neutral lipid, at least one helper lipid, and at least one PEG lipid. In some embodiments, the neutral lipid is DSPC or DMPE. In some embodiments, the helper lipid is cholesterol, 5-heptadecylresorcinol, or cholesterol hemisuccinate.

In some embodiments, the neutral lipid is DSPC. In some embodiments, the helper lipid is cholesterol. In some embodiments, the PEG lipid is PEG2K-DMG, C13 ether, or C14 ether. In some embodiments, the PEG lipid is 1,2-dimyristoyl-rac-glycero-3-methoxypolyethylene glycol-2000, methoxy-PEG2000-carbamoyl-1,2-tridecyoxypropylamine, or methoxy-PEG2000-carbamoyl-1,2-tetradecyoxypropylamine.

In some embodiments, the lipid composition further comprises one or more additional lipid components.

In some embodiments, the lipid composition is in the form of a liposome. In preferred embodiments, the lipid composition is in the form of a lipid nanoparticle (LNP). In certain embodiments the lipid composition is suitable for delivery in vivo. In certain embodiments the lipid composition is suitable for delivery to an organ, such as the liver. In certain embodiments the lipid composition is suitable for delivery to a tissue ex vivo. In certain embodiments the lipid composition is suitable for delivery to a cell in vitro.

Lipid compositions comprising lipids of Formula (I)-(IV) or Table 1, or a pharmaceutically acceptable salt thereof, may be in various forms, including, but not limited to, particle forming delivery agents including microparticles, nanoparticles and transfection agents that are useful for delivering various molecules to cells. Specific compositions are effective at transfecting or delivering biologically active agents. Preferred biologically active agents are nucleic acids such as RNAs. In further embodiments, the biologically active agent is chosen from mRNA and gRNA. The gRNA may be a dgRNA or an sgRNA. In certain embodiments, the cargo includes an mRNA encoding an RNA-guided DNA-binding agent (e.g. a Cas nuclease, a Class 2 Cas nuclease, or Cas9), a gRNA or a nucleic acid encoding a gRNA, or a combination of mRNA and gRNA.

The compositions will generally, but not necessarily, include one or more pharmaceutically acceptable excipients. The term “excipient” includes any ingredient other than the compound(s) of the disclosure, the other lipid component(s) and the biologically active agent. An excipient may impart either a functional (e.g. drug release rate controlling) and/or a non-functional (e.g. processing aid or diluent) characteristic to the compositions. The choice of excipient will to a large extent depend on factors such as the particular mode of administration, the effect of the excipient on solubility and stability, and the nature of the dosage form.

Parenteral formulations are typically aqueous or oily solutions or suspensions. Where the formulation is aqueous, excipients such as sugars (including but not restricted to glucose, mannitol, sorbitol, etc.) salts, carbohydrates and buffering agents (preferably to a pH of from 3 to 9), but, for some applications, they may be more suitably formulated with a sterile non-aqueous solution or as a dried form to be used in conjunction with a suitable vehicle such as sterile, pyrogen-free water (WFI).

### LNP Compositions

The lipid compositions may be provided as LNP compositions, and LNP compositions described herein may be provided as lipid compositions. Lipid nanoparticles may be, e.g., microspheres (including unilamellar and multilamellar vesicles, e.g. “liposomes”-lamellar phase lipid bilayers that, in some embodiments are substantially spherical, and, in more particular embodiments can comprise an aqueous core, e.g., comprising a substantial portion of RNA molecules), a dispersed phase in an emulsion, micelles or an internal phase in a suspension.

Described herein are LNP compositions comprising at least one compound of Formula (I)-(IV) or Table 1, or a salt thereof (e.g., a pharmaceutically acceptable salt thereof), at least one helper lipid, at least one neutral lipid, and at least one polymeric lipid. In some embodiments, the LNP composition comprises at least one compound of Formula (I)-(IV) or Table 1, or a pharmaceutically acceptable salt thereof, at least one neutral lipid, at least one helper lipid, and at least one PEG lipid. In some embodiments, the neutral lipid is DSPC or DPME. In some embodiments, the helper lipid is cholesterol, 5-heptadecylresorcinol, or cholesterol hemisuccinate.

Embodiments of the present disclosure provide lipid compositions described according to the respective molar ratios of the component lipids in the composition. All mol % numbers are given as a fraction of the lipid component of the lipid composition or, more specifically, the LNP compositions. In some embodiments, the lipid mol % of a lipid relative to the lipid component will be ±30%, ±25%, ±20%, ±15%, ±10%, ±5%, or ±2.5% of the specified, nominal, or actual mol % of the lipid. In some embodiments, the lipid mol % of a lipid relative to the lipid component will be ±4 mol %, ±3 mol %, ±2 mol %, ±1.5 mol %, ±1 mol %, ±0.5 mol %, ±0.25 mol %, or ±0.05 mol % of the specified, nominal, or actual mol % of the lipid component. In certain embodiments, the lipid mol % will vary by less than 15%, less than 10%, less than 5%, less than 1%, or less than 0.5% from the specified, nominal, or actual mol % of the lipid. In some embodiments, the mol % numbers are based on nominal concentration. As used herein, “nominal concentration” refers to concentration based on the input amounts of substances combined to form a resulting composition. For example, if 100 mg of solute is added to 1 L water, the nominal concentration is 100 mg/L. In some embodiments, the mol % numbers are based on actual concentration, e.g., concentration determined by an analytic method. In some embodiments, actual concentration of the lipids of the lipid component may be determined, for example, from chromatography, such as liquid chromatography, followed by a detection method, such as charged aerosol detection. In some embodiments, actual concentration of the lipids of the lipid component may be characterized by lipid analysis, AF4-MALS, NTA, and/or cryo-EM. All mol % numbers are given as a percentage of the lipids of the lipid component.

Embodiments of the present disclosure provide LNP compositions described according to the respective molar ratios of the lipids of the lipid component. In certain embodiments, the amount of the ionizable lipid is from about 25 mol % to about 75 mol %; the amount of the neutral lipid is from about 5 mol % to about 20 mol %; the amount of the helper lipid is from about 25 mol % to about 50 mol %; and the amount of the PEG lipid is from about 1.5 mol % to about 4 mol %. In certain embodiments, the amount of the ionizable lipid is from about 30-60 mol % of the lipid component; the amount of the neutral lipid is from about 7.5-15 mol % of the lipid component; the amount of the helper lipid is from about 30-45 mol % of the lipid component; and the amount of the PEG lipid is from about 2.3-3.5 mol % of the lipid component. In certain embodiments, the amount of the ionizable lipid is from about 45-55 mol % of the lipid component; the amount of the neutral lipid is from about 8-12 mol % of the lipid component; the amount of the helper lipid is from about 35-42 mol % of the lipid component; and the amount of the PEG lipid is from about 2.5-3.5 mol % of the lipid component. In certain embodiments, the amount of the ionizable lipid is from about 47-52 mol % of the lipid component; the amount of the neutral lipid is from about 8-10 mol % of the lipid component; the amount of the helper lipid is from about 37-40 mol % of the lipid component; and the amount of the PEG lipid is from about 2.7-3.3 mol % of the lipid component. In certain embodiments, the ionizable lipid is about 50 mol % of the lipid component; the amount of the neutral lipid is about 9 mol % of the lipid component; the amount of the helper lipid is about 38 mol % of the lipid component; and the amount of the PEG lipid is about 3 mol % of the lipid component.

In certain embodiments, the amount of the ionizable lipid is about 25-75 mol %, about 25-70 mol %, about 25-65 mol %, about 25-60 mol %, about 25-55 mol %, about 25-50 mol %, about 30-75 mol %, about 30-70 mol %, about 30-65 mol %, about 30-60 mol %, about 30-55 mol %, about 30-50 mol %, about 35-75 mol %, about 35-60 mol %, about 35-65 mol %, about 35-60 mol %, about 35-65 mol %, about 35-55 mol %, about 40-75 mol %, about 40-65 mol %, about 40-60 mol %, about 40-70 mol %, about 40-55 mol %, about 45-55 mol %, or about 45-60 mol %. In some embodiments, the mol % of the ionizable lipid may be about 40 mol %, about 41 mol %, about 42 mol %, about 43 mol %, about 44 mol %, about 45 mol %, about 46 mol %, about 47 mol %, about 48 mol %, about 49 mol %, about 50 mol %, about 51 mol %, about 52 mol %, about 53 mol %, about 54 mol %, 55 mol %, about 56 mol %, about 57 mol %, about 58 mol %, about 59 mol %, or about 60 mol %. In some embodiments, the ionizable lipid mol % relative to the lipid component will be ±30%, ±25%, ±20%, ±15%, ±10%, ±5%, or ±2.5% of the specified, nominal, or actual mol %. In some embodiments, the ionizable lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability of the ionizable lipid mol % will be less than 15%, less than 10% or less than 5%. In some embodiments, the mol % numbers are based on nominal concentration. In some embodiments, the mol % numbers are based on actual concentration.

In certain embodiments, the amount of the neutral lipid is about 5-20 mol %, about 5-15 mol %, about 5-10 mol %, about 7-10 mol %, or about 9 mol %. In additional embodiments, the amount of the neutral lipid may be about 5-30 mol %, about 5-28 mol %, about 5-25 mol %, about 5-23 mol %, about 5-20 mol %, about 5-18 mol %, about 5-23 mol %, about 5-20 mol %, about 5-18 mol %, about 5-15 mol %, about 5-13 mol %, or about 5-10 mol %. In some embodiments, the mol % of the neutral lipid may be about 5 mol %, about 6 mol %, about 7 mol %, about 8 mol %, about 9 mol %, about 10 mol %, about 11 mol %, about 12 mol %, about 13 mol %, about 14 mol %, about 15 mol %, about 16 mol %, about 17 mol %, about 18 mol %, about 19 mol %, or about 20 mol %. In some embodiments, the neutral lipid mol % relative to the lipid component will be ±30%, ±25%, ±20%, ±15%, ±10%, ±5%, or ±2.5% of the specified, nominal, or actual neutral lipid mol %. In some embodiments, the neutral lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the amount of the helper lipid is about 30-50 mol %, about 30-65 mol %, about 30-55 mol %, about 33-50 mol %, about 32-55 mol %, about 32-65 mol %, about 35-50 mol %, about 35-55 mol %, about 35-40 mol %, about 35-45 mol %, or about 38 mol %. In additional embodiments, the amount of the helper lipid may be about 25-65 mol %, about 28-65 mol %, about 30-65 mol %, about 32-65 mol %, about 35-65 mol %, about 25-62 mol %, about 28-62 mol %, about 30-62 mol %, about 32-62 mol %, about 35-62 mol %, about 38-62 mol %, about 40-62 mol %, about 42-62 mol %, about 45-62 mol %, about 48-62 mol %, about 50-62 mol %, about 52-62 mol %, about 55-62 mol %, about 58-62 mol %, about 60-62 mol %, about 25-60 mol %, about 28-60 mol %, about 30-60 mol %, about 32-60 mol %, about 35-60 mol %, about 38-60 mol %, about 40-60 mol %, about 42-60 mol %, about 45-60 mol %, about 48-60 mol %, about 50-60 mol %, about 52-60 mol %, about 55-60 mol %, about 58-60 mol %, about 25-58 mol %, about 28-58 mol %, about 30-58 mol %, about 32-58 mol %, about 35-58 mol %, about 38-58 mol %, about 40-58 mol %, about 42-58 mol %, about 45-58 mol %, about 48-58 mol %, about 50-58 mol %, about 52-58 mol %, about 55-58 mol %, about 25-55 mol %, about 28-55 mol %, about 30-55 mol %, about 32-55 mol %, about 35-55 mol %, about 38-55 mol %, about 40-55 mol %, about 42-55 mol %, about 45-55 mol %, about 48-55 mol %, about 50-55 mol %, about 52-55 mol %, about 25-53 mol %, about 28-53 mol %, about 30-53 mol %, about 32-53 mol %, about 35-53 mol %, about 38-53 mol %, about 40-53 mol %, about 42-53 mol %, about 45-53 mol %, about 48-53 mol %, about 50-53 mol %, about 25-50 mol %, about 28-50 mol %, about 30-50 mol %, about 32-50 mol %, about 35-50 mol %, about 38-50 mol %, about 40-50 mol %, about 42-50 mol %, about 45-50 mol %, about 48-50 mol %, about 25-48 mol %, about 28-48 mol %, about 30-48 mol %, about 32-48 mol %, about 35-48 mol %, about 38-48 mol %, about 40-48 mol %, about 42-48 mol %, about 45-48 mol %, about 25-45 mol %, about 28-45 mol %, about 30-45 mol %, about 32-45 mol %, about 35-45 mol %, about 38-45 mol %, about 40-45 mol %, about 42-45 mol %, about 25-43 mol %, about 28-43 mol %, about 30-43 mol %, about 32-43 mol %, about 35-43 mol %, about 38-43 mol %, about 40-43 mol %, about 25-40 mol %, about 28-40 mol %, about 30-40 mol %, about 32-40 mol %, about 35-40 mol %, about 38-40 mol %, about 25-38 mol %, about 28-38 mol %, about 30-38 mol %, about 32-38 mol %, about 35-38 mol %, about 25-35 mol %, about 28-35 mol %, about 30-35 mol %, about 32-35 mol %, about 25-33 mol %, about 28-33 mol %, about 30-33 mol %, about 35-45 mol %, or about 35-40 mol %. In certain embodiments, the amount of the helper lipid is adjusted based on the amounts of the ionizable lipid, the neutral lipid, and/or the PEG lipid to bring the lipid component to about 100 mol %. In some embodiments, the helper lipid mol % relative to the lipid component will be ±30%, ±25%, ±20%, ±15%, ±10%, ±5%, or 2.5% of the specified, nominal, or actual helper lipid mol %. In some embodiments, the helper lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the amount of the PEG lipid is about 1.5-3.5 mol %, about 2.0-2.7 mol %, about 2.0-3.5 mol %, about 2.3-3.5 mol %, about 2.3-2.7 mol %, about 2.5-3.5 mol %, about 2.5-2.7 mol %, about 2.9-3.5 mol %, or about 2.7 mol %. In additional embodiments, the amount of the PEG lipid may be about 1.0-4.0 mol %, about 1.2-4.0 mol %, about 1.4-4.0 mol %, about 1.5-4.0 mol %, about 1.6-4.0 mol %, about 1.7-4.0 mol %, about 1.8-4.0 mol %, about 1.9-4.0 mol %, about 2.0-4.0 mol %, about 2.1-4.0 mol %, about 2.2-4.0 mol %, about 2.3-4.0 mol %, about 2.4-4.0 mol %, about 2.5-4.0 mol %, about 2.6-4.0 mol %, about 2.7-4.0 mol %, about 2.8-4.0 mol %, about 2.9-4.0 mol %, about 3.0-4.0 mol %, about 3.1-4.0 mol %, about 3.2-4.0 mol %, about 3.3-4.0 mol %, about 3.4-4.0 mol %, about 3.5-4.0 mol %, about 3.7-4.0 mol %, 1.0-3.7 mol %, about 1.2-3.7 mol %, about 1.4-3.7 mol %, about 1.5-3.7 mol %, about 1.6-3.7 mol %, about 1.7-3.7 mol %, about 1.8-3.7 mol %, about 1.9-3.7 mol %, about 2.0-3.7 mol %, about 2.1-3.7 mol %, about 2.2-3.7 mol %, about 2.3-3.7 mol %, about 2.4-3.7 mol %, about 2.5-3.7 mol %, about 2.6-3.7 mol %, about 2.7-3.7 mol %, about 2.8-3.7 mol %, about 2.9-3.7 mol %, about 3.0-3.7 mol %, about 3.1-3.7 mol %, about 3.2-3.7 mol %, about 3.3-3.7 mol %, about 3.4-3.7 mol %, about 3.5-3.7 mol %, 1.0-3.5 mol %, about 1.2-3.5 mol %, about 1.4-3.5 mol %, about 1.5-3.5 mol %, about 1.6-3.5 mol %, about 1.7-3.5 mol %, about 1.8-3.5 mol %, about 1.9-3.5 mol %, about 2.0-3.5 mol %, about 2.1-3.5 mol %, about 2.2-3.5 mol %, about 2.3-3.5 mol %, about 2.4-3.5 mol %, about 2.5-3.5 mol %, about 2.6-3.5 mol %, about 2.7-3.5 mol %, about 2.8-3.5 mol %, about 2.9-3.5 mol %, about 3.0-3.5 mol %, about 3.1-3.5 mol %, about 3.2-3.5 mol %, about 3.3-3.5 mol %, about 3.4-3.5 mol %, 1.0-3.4 mol %, about 1.2-3.4 mol %, about 1.4-3.4 mol %, about 1.5-3.4 mol %, about 1.6-3.4 mol %, about 1.7-3.4 mol %, about 1.8-3.4 mol %, about 1.9-3.4 mol %, about 2.0-3.4 mol %, about 2.1-3.4 mol %, about 2.2-3.4 mol %, about 2.3-3.4 mol %, about 2.4-3.4 mol %, about 2.5-3.4 mol %, about 2.6-3.4 mol %, about 2.7-3.4 mol %, about 2.8-3.4 mol %, about 2.9-3.4 mol %, about 3.0-3.4 mol %, about 3.1-3.4 mol %, about 3.2-3.4 mol %, about 3.3-3.4 mol %, 1.0-3.3 mol %, about 1.2-3.3 mol %, about 1.4-3.3 mol %, about 1.5-3.3 mol %, about 1.6-3.3 mol %, about 1.7-3.3 mol %, about 1.8-3.3 mol %, about 1.9-3.3 mol %, about 2.0-3.3 mol %, about 2.1-3.3 mol %, about 2.2-3.3 mol %, about 2.3-3.3 mol %, about 2.4-3.3 mol %, about 2.5-3.3 mol %, about 2.6-3.3 mol %, about 2.7-3.3 mol %, about 2.8-3.3 mol %, about 2.9-3.3 mol %, about 3.0-3.3 mol %, about 3.1-3.3 mol %, about 3.2-3.3 mol %, 1.0-3.2 mol %, about 1.2-3.2 mol %, about 1.4-3.2 mol %, about 1.5-3.2 mol %, about 1.6-3.2 mol %, about 1.7-3.2 mol %, about 1.8-3.2 mol %, about 1.9-3.2 mol %, about 2.0-3.2 mol %, about 2.1-3.2 mol %, about 2.2-3.2 mol %, about 2.3-3.2 mol %, about 2.4-3.2 mol %, about 2.5-3.2 mol %, about 2.6-3.2 mol %, about 2.7-3.2 mol %, about 2.8-3.2 mol %, about 2.9-3.2 mol %, about 3.0-3.2 mol %, about 3.1-3.2 mol %, 1.0-3.1 mol %, about 1.2-3.1 mol %, about 1.4-3.1 mol %, about 1.5-3.1 mol %, about 1.6-3.1 mol %, about 1.7-3.1 mol %, about 1.8-3.1 mol %, about 1.9-3.1 mol %, about 2.0-3.1 mol %, about 2.1-3.1 mol %, about 2.2-3.1 mol %, about 2.3-3.1 mol %, about 2.4-3.1 mol %, about 2.5-3.1 mol %, about 2.6-3.1 mol %, about 2.7-3.1 mol %, about 2.8-3.1 mol %, about 2.9-3.1 mol %, about 3.0-3.1 mol %, 1.0-3.0 mol %, about 1.2-3.0 mol %, about 1.4-3.0 mol %, about 1.5-3.0 mol %, about 1.6-3.0 mol %, about 1.7-3.0 mol %, about 1.8-3.0 mol %, about 1.9-3.0 mol %, about 2.0-3.0 mol %, about 2.1-3.0 mol %, about 2.2-3.0 mol %, about 2.3-3.0 mol %, about 2.4-3.0 mol %, about 2.5-3.0 mol %, about 2.6-3.0 mol %, about 2.7-3.0 mol %, about 2.8-3.0 mol %, about 2.9-3.0 mol %, 1.0-2.9 mol %, about 1.2-2.9 mol %, about 1.4-2.9 mol %, about 1.5-2.9 mol %, about 1.6-2.9 mol %, about 1.7-2.9 mol %, about 1.8-2.9 mol %, about 1.9-2.9 mol %, about 2.0-2.9 mol %, about 2.1-2.9 mol %, about 2.2-2.9 mol %, about 2.3-2.9 mol %, about 2.4-2.9 mol %, about 2.5-2.9 mol %, about 2.6-2.9 mol %, about 2.7-2.9 mol %, about 2.8-2.9 mol %, 1.0-2.8 mol %, about 1.2-2.8 mol %, about 1.4-2.8 mol %, about 1.5-2.8 mol %, about 1.6-2.8 mol %, about 1.7-2.8 mol %, about 1.8-2.8 mol %, about 1.9-2.8 mol %, about 2.0-2.8 mol %, about 2.1-2.8 mol %, about 2.2-2.8 mol %, about 2.3-2.8 mol %, about 2.4-2.8 mol %, about 2.5-2.8 mol %, about 2.6-2.8 mol %, about 2.7-2.8 mol %, 1.0-2.7 mol %, about 1.2-2.7 mol %, about 1.4-2.7 mol %, about 1.5-2.7 mol %, about 1.6-2.7 mol %, about 1.7-2.7 mol %, about 1.8-2.7 mol %, about 1.9-2.7 mol %, about 2.0-2.7 mol %, about 2.1-2.7 mol %, about 2.2-2.7 mol %, about 2.3-2.7 mol %, about 2.4-2.7 mol %, about 2.5-2.7 mol %, about 2.6-2.7 mol %, 1.0-2.6 mol %, about 1.2-2.6 mol %, about 1.4-2.6 mol %, about 1.5-2.6 mol %, about 1.6-2.6 mol %, about 1.7-2.6 mol %, about 1.8-2.6 mol %, about 1.9-2.6 mol %, about 2.0-2.6 mol %, about 2.1-2.6 mol %, about 2.2-2.6 mol %, about 2.3-2.6 mol %, about 2.4-2.6 mol %, about 2.5-2.6 mol %, 1.0-2.5 mol %, about 1.2-2.5 mol %, about 1.4-2.5 mol %, about 1.5-2.5 mol %, about 1.6-2.5 mol %, about 1.7-2.5 mol %, about 1.8-2.5 mol %, about 1.9-2.5 mol %, about 2.0-2.5 mol %, about 2.1-2.5 mol %, about 2.2-2.5 mol %, about 2.3-2.5 mol %, about 2.4-2.5 mol %, 1.0-2.4 mol %, about 1.2-2.4 mol %, about 1.4-2.4 mol %, about 1.5-2.4 mol %, about 1.6-2.4 mol %, about 1.7-2.4 mol %, about 1.8-2.4 mol %, about 1.9-2.4 mol %, about 2.0-2.4 mol %, about 2.1-2.4 mol %, about 2.2-2.4 mol %, about 2.3-2.4 mol %, 1.0-2.3 mol %, about 1.2-2.3 mol %, about 1.4-2.3 mol %, about 1.5-2.3 mol %, about 1.6-2.3 mol %, about 1.7-2.3 mol %, about 1.8-2.3 mol %, about 1.9-2.3 mol %, about 2.0-2.3 mol %, about 2.1-2.3 mol %, about 2.2-2.3 mol %, 1.0-2.2 mol %, about 1.2-2.2 mol %, about 1.4-2.2 mol %, about 1.5-2.2 mol %, about 1.6-2.2 mol %, about 1.7-2.2 mol %, about 1.8-2.2 mol %, about 1.9-2.2 mol %, about 2.0-2.2 mol %, about 2.1-2.2 mol %, about 2.2-2.2 mol %, about 2.3-2.2 mol %, about 2.4-2.2 mol %, 1.0-2.1 mol %, about 1.2-2.1 mol %, about 1.4-2.1 mol %, about 1.5-2.1 mol %, about 1.6-2.1 mol %, about 1.7-2.1 mol %, about 1.8-2.1 mol %, about 1.9-2.1 mol %, about 2.0-2.1 mol %, 1.0-2.0 mol %, about 1.2-2.0 mol %, about 1.4-2.0 mol %, about 1.5-2.0 mol %, about 1.6-2.0 mol %, about 1.7-2.0 mol %, about 1.8-2.0 mol %, about 1.9-2.0 mol %, 1.0-1.9 mol %, about 1.2-1.9 mol %, about 1.4-1.9 mol %, about 1.5-1.9 mol %, about 1.6-1.9 mol %, about 1.7-1.9 mol %, about 1.8-1.9 mol %, 1.0-1.8 mol %, about 1.2-1.8 mol %, about 1.4-1.8 mol %, about 1.5-1.8 mol %, about 1.6-1.8 mol %, about 1.7-1.8 mol %, 1.0-1.7 mol %, about 1.2-1.7 mol %, about 1.4-1.7 mol %, about 1.5-1.7 mol %, about 1.6-1.7 mol %, 1.0-1.6 mol %, about 1.2-1.6 mol %, about 1.4-1.6 mol %, about 1.5-1.6 mol %, 1.0-1.5 mol %, about 1.2-1.5 mol %, about 1.4-1.5 mol %, about 1.5-1.5 mol %, about 1.6-1.5 mol %, about 1.7-1.5 mol %, about 1.8-1.5 mol %, about 1.9-1.5 mol %, 1.0-1.4 mol %, about 1.2-1.4 mol %, or 1.0-1.2 mol %. In some embodiments, the mol % of the PEG lipid may be about 1.5 mol %, about 1.6 mol %, about 1.7 mol %, about 1.8 mol %, about 1.9 mol %, about 2.0 mol %, about 2.1 mol %, about 2.2 mol %, about 2.3 mol %, about 2.4 mol %, about 2.5 mol %, about 2.6 mol %, about 2.7 mol %, about 2.8 mol %, about 2.9 mol %, about 3.0 mol %, about 3.1 mol %, about 3.2 mol %, about 3.3 mol %, about 3.4 mol %, or about 3.5 mol %. In some embodiments, the PEG lipid mol % relative to the lipid component will be ±30%, ±25%, ±20%, ±15%, ±10%, ±5%, or ±2.5% of the specified, nominal, or actual PEG lipid mol %. In some embodiments, the PEG lipid mol % relative to the lipid component will be ±4 mol %, ±3 mol %, ±2 mol %, ±1.5 mol %, ±1 mol %, ±0.5 mol %, or ±0.25 mol % of the specified, nominal, or actual mol %. In certain embodiments, LNP inter-lot variability will be less than 15%, less than 10% or less than 5%. In some embodiments, the mol % numbers are based on nominal concentration. In some embodiments, the mol % numbers are based on actual concentration.

In certain embodiments, the amount of the helper lipid is about 30-40 mol % and the amount of the PEG lipid is about 2.5-3.5 mol %; the amount of the helper lipid is about 35-40 mol % and the amount of the PEG lipid is about 2.9-3.3 mol %; or the amount of the helper lipid is about 38 mol % and the amount of the PEG lipid is about 3 mol %. In certain embodiments, the amount of the helper lipid is about 37-47 mol % and the amount of the PEG lipid is about 2.0-3.0 mol %; the amount of the helper lipid is about 40-45 mol % and the amount of the PEG lipid is about 2.2-2.8 mol %; or the amount of the helper lipid is about 42 mol %, and the amount of the PEG lipid is about 2.2-2.8 mol %. In certain embodiments, the amount of the helper lipid is about 47-57 mol % and the amount of the PEG lipid is about 2.0-3.0 mol %; the amount of the helper lipid is about 50-55 mol % and the amount of the PEG lipid is about 2.3-2.7 mol %; or the amount of the helper lipid is about 52 mol %, and the amount of the PEG lipid is about 2.3-2.7 mol %.

In certain embodiments, the lipid compositions, such as LNP compositions, comprise a lipid component and a nucleic acid component (also referred to as an aqueous component), e.g. an RNA component and the molar ratio of compound of Formula (I)-(IV) or Table 1 to nucleic acid can be measured. Embodiments of the present disclosure also provide lipid compositions having a defined molar ratio between the positively charged amine groups of pharmaceutically acceptable salts of the compounds of Formula (I)-(IV) or Table 1 (N) and the negatively charged phosphate groups (P) of the nucleic acid to be encapsulated. This may be mathematically represented by the equation N/P. In some embodiments, a lipid composition, such as an LNP composition, may comprise a lipid component that comprises a compound of Formula (I)-(IV) or Table 1 or a pharmaceutically acceptable salt thereof; and a nucleic acid component, wherein the N/P ratio is about 3 to 10. In some embodiments, an LNP composition may comprise a lipid component that comprises a compound of Formula (I)-(IV) or Table 1 or a pharmaceutically acceptable salt thereof; and an RNA component, wherein the N/P ratio is about 3 to 10. For example, the N/P ratio may be about 4-7, about 5-7, or about 6 to 7. In some embodiments, the N/P ratio may about 6, e.g., 6±1, or 6±0.5. In some embodiments, the N/P ratio may about 7, e.g., 7±1, or 7±0.5.

In some embodiments, the aqueous component comprises a biologically active agent. In some embodiments, the aqueous component comprises a polypeptide, optionally in combination with a nucleic acid. In some embodiments, the aqueous component comprises a nucleic acid, such as an RNA. In some embodiments, the aqueous component is a nucleic acid component. In some embodiments, the nucleic acid component comprises DNA and it can be called a DNA component. In some embodiments, the nucleic acid component comprises RNA. In some embodiments, the aqueous component, such as an RNA component may comprise an mRNA, such as an mRNA encoding an RNA-guided DNA-binding agent. In some embodiments, the RNA-guided DNA-binding agent is a Cas nuclease. In certain embodiments, aqueous component may comprise an mRNA that encodes a Cas nuclease, such as Cas9. In certain embodiments, the biologically active agent is a Cas nuclease mRNA. In certain embodiments, the biologically active agent is a Class 2 Cas nuclease mRNA. In certain embodiments, the biologically active agent is a Cas9 nuclease mRNA. In certain embodiments, the aqueous component may comprise a modified RNA. In some embodiments, the aqueous component may comprise a guide RNA nucleic acid. In certain embodiments, the aqueous component may comprise a gRNA. In certain embodiments, the aqueous component may comprise a dgRNA. In certain embodiments, the aqueous component may comprise a modified gRNA. In some compositions comprising an mRNA encoding an RNA-guided DNA-binding agent, the composition further comprises a gRNA nucleic acid, such as a gRNA. In some embodiments, the aqueous component comprises an RNA-guided DNA-binding agent and a gRNA. In some embodiments, the aqueous component comprises a Cas nuclease mRNA and a gRNA. In some embodiments, the aqueous component comprises a Class 2 Cas nuclease mRNA and a gRNA.

In certain embodiments, a lipid composition, such as an LNP composition, may comprise an mRNA encoding a Cas nuclease such as a Class 2 Cas nuclease, a compound of Formula (I)-(IV) or Table 1 or a pharmaceutically acceptable salt thereof, a helper lipid, optionally a neutral lipid, and a PEG lipid. In certain compositions comprising an mRNA encoding a Cas nuclease such as a Class 2 Cas nuclease, the helper lipid is cholesterol. In other compositions comprising an mRNA encoding a Cas nuclease such as a Class 2 Cas nuclease, the neutral lipid is DSPC. In additional embodiments comprising an mRNA encoding a Cas nuclease such as a Class 2 Cas nuclease, e.g. Cas9, the PEG lipid is PEG2K-DMG. In specific compositions comprising an mRNA encoding a Cas nuclease such as a Class 2 Cas nuclease, and a compound of Formula (I)-(IV) or Table 1 or a pharmaceutically acceptable salt thereof. In certain compositions, the composition further comprises a gRNA, such as a dgRNA or an sgRNA.

In some embodiments, a lipid composition, such as an LNP composition, may comprise a gRNA. In certain embodiments, a composition may comprise a compound of Formula (I)-(IV) or Table 1 or a pharmaceutically acceptable salt thereof, a gRNA, a helper lipid, optionally a neutral lipid, and a PEG lipid. In certain LNP compositions comprising a gRNA, the helper lipid is cholesterol. In some compositions comprising a gRNA, the neutral lipid is DSPC. In additional embodiments comprising a gRNA, the PEG lipid is PEG2K-DMG. In certain compositions, the gRNA is selected from dgRNA and sgRNA.

In certain embodiments, a lipid composition, such as an LNP composition, comprises an mRNA encoding an RNA-guided DNA-binding agent and a gRNA, which may be an sgRNA, in an aqueous component and a compound of Formula (I)-(IV) or Table 1 in a lipid component. For example, an LNP composition may comprise a compound of Formula (I)-(IV) or Table 1 or a pharmaceutically acceptable salt thereof, an mRNA encoding a Cas nuclease, a gRNA, a helper lipid, a neutral lipid, and a PEG lipid. In certain compositions comprising an mRNA encoding a Cas nuclease and a gRNA, the helper lipid is cholesterol. In some compositions comprising an mRNA encoding a Cas nuclease and a gRNA, the neutral lipid is DSPC. In additional embodiments comprising an mRNA encoding a Cas nuclease and a gRNA, the PEG lipid is PEG2K-DMG.

In certain embodiments, the lipid compositions, such as LNP compositions include an RNA-guided DNA-binding agent, such as a Class 2 Cas mRNA and at least one gRNA.

In some embodiments, the gRNA is a sgRNA. In some embodiments, the RNA-guided DNA-binding agent is a Cas9 mRNA In certain embodiments, the LNP composition includes a ratio of gRNA to RNA-guided DNA-binding agent mRNA, such as Class 2 Cas nuclease mRNA of about 1:1 or about 1:2. In some embodiments, the ratio of by weight is from about 25:1 to about 1:25, about 10:1 to about 1:10, about 8:1 to about 1:8, about 4:1 to about 1:4, about 2:1 to about 1:2, about 2:1 to 1:4 by weight, or about 1:1 to about 1:2.

The lipid compositions disclosed herein, such as LNP compositions, may be used in methods disclosed herein to deliver CRISPR/Cas9 components to insert a template nucleic acid, e.g., a DNA template. The template nucleic acid may be delivered separately from the lipid compositions comprising a compound of Formula (I)-(IV) or Table 1 or a pharmaceutically acceptable salt thereof. In some embodiments, the template nucleic acid may be single- or double-stranded, depending on the desired repair mechanism. The template may have regions of homology to the target DNA, e.g. within the target DNA sequence, and/or to sequences adjacent to the target DNA.

In some embodiments, LNP compositions are formed by mixing an aqueous RNA solution with an organic solvent-based lipid solution. Suitable solutions or solvents include or may contain: water, PBS, Tris buffer, NaCl, citrate buffer, acetate buffer, ethanol, chloroform, diethylether, cyclohexane, tetrahydrofuran, methanol, isopropanol. For example, the organic solvent may be 100% ethanol. A pharmaceutically acceptable buffer, e.g., for in vivo administration of LNP compositions, may be used. In certain embodiments, a buffer is used to maintain the pH of the composition comprising LNPs at or above pH 6.5. In certain embodiments, a buffer is used to maintain the pH of the composition comprising LNPs at or above pH 7.0. In certain embodiments, the composition has a pH ranging from about 7.2 to about 7.7. In additional embodiments, the composition has a pH ranging from about 7.3 to about 7.7 or ranging from about 7.4 to about 7.6. In further embodiments, the composition has a pH of about 7.2, 7.3, 7.4, 7.5, 7.6, or 7.7. The pH of a composition may be measured with a micro pH probe. In certain embodiments, a cryoprotectant is included in the composition. Non-limiting examples of cryoprotectants include sucrose, trehalose, glycerol, DMSO, and ethylene glycol. Exemplary compositions may include up to 10% cryoprotectant, such as, for example, sucrose. In certain embodiments, the composition may comprise tris saline sucrose (TSS). In certain embodiments, the LNP composition may include about 1, 2, 3, 4, 5, 6, 7, 8, 9, or 10% cryoprotectant. In certain embodiments, the LNP composition may include about 1, 2, 3, 4, 5, 6, 7, 8, 9, or 10% sucrose. In some embodiments, the LNP composition may include a buffer. In some embodiments, the buffer may comprise a phosphate buffer (PBS), a Tris buffer, a citrate buffer, and mixtures thereof. In certain exemplary embodiments, the buffer comprises NaCl. In certain embodiments, the buffer lacks NaCl. Exemplary amounts of NaCl may range from about 20 mM to about 45 mM. Exemplary amounts of NaCl may range from about 40 mM to about 50 mM. In some embodiments, the amount of NaCl is about 45 mM. In some embodiments, the buffer is a Tris buffer. Exemplary amounts of Tris may range from about 20 mM to about 60 mM. Exemplary amounts of Tris may range from about 40 mM to about 60 mM. In some embodiments, the amount of Tris is about 50 mM. In some embodiments, the buffer comprises NaCl and Tris. Certain exemplary embodiments of the LNP compositions contain 5% sucrose and 45 mM NaCl in Tris buffer. In other exemplary embodiments, compositions contain sucrose in an amount of about 5% w/v, about 45 mM NaCl, and about 50 mM Tris at pH 7.5. The salt, buffer, and cryoprotectant amounts may be varied such that the osmolality of the overall composition is maintained. For example, the final osmolality may be maintained at less than 450 mOsm/L. In further embodiments, the osmolality is between 350 and 250 mOsm/L. Certain embodiments have a final osmolality of 300+/−20 mOsm/L or 310+/−40 mOsm/L.

In some embodiments, microfluidic mixing, T-mixing, or cross-mixing of the aqueous RNA solution and the lipid solution in an organic solvent is used. In certain aspects, flow rates, junction size, junction geometry, junction shape, tube diameter, solutions, and/or RNA and lipid concentrations may be varied. LNPs or LNP compositions may be buffer exchanged, concentrated or purified, e.g., via dialysis, centrifugal filter, tangential flow filtration, chromatography, or gravity size exclusion chromatography. The LNP compositions may be stored as a suspension, an emulsion, or a lyophilized powder, for example. In some embodiments, an LNP composition is stored at 2-8° C., in certain aspects, the LNP compositions are stored at room temperature. In additional embodiments, an LNP composition is stored frozen, for example at −20° C. or −80° C. In other embodiments, an LNP composition is stored at a temperature ranging from about 0° C. to about −80° C. Frozen LNP compositions may be thawed before use, for example on ice, at room temperature, or at 25° C., preferably at room temperature.

Preferred lipid compositions, such as LNP compositions, are biodegradable, for example, in that they do not accumulate to cytotoxic levels in vivo at a therapeutically effective dose. In some embodiments, the compositions do not cause an innate immune response that leads to substantial adverse effects at a therapeutic dose level. In some embodiments, the compositions provided herein do not cause toxicity at a therapeutic dose level.

In some embodiments, the concentration of the LNPs in the LNP composition is about 1-10 μg/mL, about 2-10 μg/mL, about 2.5-10 μg/mL, about 1-5 μg/mL, about 2-5 μg/mL, about 2.5-5 μg/mL, about 0.04 μg/mL, about 0.08 μg/mL, about 0.16 μg/mL, about 0.25 μg/mL, about 0.63 μg/mL, about 1.25 μg/mL, about 2.5 μg/mL, or about 5 μg/mL.

In some embodiments, Dynamic Light Scattering (“DLS”) may be used to characterize the polydispersity index (PDI) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

In some embodiments, the LNPs disclosed herein have a PDI from about 0.005 to about 0.75. In some embodiments, the LNPs disclosed herein have a PDI from about 0.005 to about 0.1. In some embodiments, the LNPs disclosed herein have a PDI from about 0.005 to about 0.09, about 0.005 to about 0.08, about 0.005 to about 0.07, or about 0.006 to about 0.05. In some embodiments, the LNP have a PDI from about 0.01 to about 0.5. In some embodiments, the LNP have a PDI from about zero to about 0.4. In some embodiments, the LNP have a PDI from about zero to about 0.35. In some embodiments, the LNP PDI may range from about zero to about 0.3. In some embodiments, the LNP have a PDI that may range from about zero to about 0.25. In some embodiments, the LNP PDI may range from about zero to about 0.2. In some embodiments, the LNP have a PDI from about zero to about 0.05. In some embodiments, the LNP have a PDI from about zero to about 0.01. In some embodiments, the LNP have a PDI less than about 0.01, about 0.02, about 0.05, about 0.08, about 0.1, about 0.15, about 0.2, or about 0.4.

LNP size may be measured by various analytical methods known in the art. In some embodiments, LNP size may be measured using Asymmetric-Flow Field Flow Fractionation-Multi-Angle Light Scattering (AF4-MALS). In certain embodiments, LNP size may be measured by separating particles in the composition by hydrodynamic radius, followed by measuring the molecular weights, hydrodynamic radii and root mean square radii of the fractionated particles. In some embodiments, LNP size and particle concentration may be measured by nanoparticle tracking analysis (NTA, Malvern Nanosight). In certain embodiments, LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. Such methods may also count the number of individual particles to give particle concentration. In some embodiments, LNP size, morphology, and structural characteristics may be determined by cryo-electron microscopy (“cryo-EM”).

The LNPs of the LNP compositions disclosed herein have a size (e.g. Z-average diameter or number-average diameter) of about 1 to about 250 nm. In some embodiments, the LNPs have a size of about 10 to about 200 nm. In further embodiments, the LNPs have a size of about 20 to about 150 nm. In some embodiments, the LNPs have a size of about 50 to about 150 nm or about 70 to 130 nm. In some embodiments, the LNPs have a size of about 50 to about 100 nm. In some embodiments, the LNPs have a size of about 50 to about 120 nm. In some embodiments, the LNPs have a size of about 60 to about 100 nm. In some embodiments, the LNPs have a size of about 75 to about 150 nm. In some embodiments, the LNPs have a size of about 75 to about 120 nm. In some embodiments, the LNPs have a size of about 75 to about 100 nm. In some embodiments, the LNPs have a size of about 50 to about 145 nm, about 50 to about 120 nm, about 50 to about 120 nm, about 50 to about 115 nm, about 50 to about 100 nm, about 60 to about 145 nm, about 60 to about 120 nm, about 60 to about 115 nm, or about 60 to about 100 nm. In some embodiments, the LNPs have a size of less than about 145 nm, less than about 120 nm, less than about 115 nm, less than about 100 nm, or less than about 80 nm. In some embodiments, the LNPs have a size of greater than about 50 nm or greater than about 60 nm. In some embodiments, the particle size is a Z-average particle size. In some embodiments, the particle size is a number-average particle size. In some embodiments, the particle size is the size of an individual LNP. Unless indicated otherwise, all sizes referred to herein are the average sizes (diameters) of the fully formed nanoparticles, as measured by dynamic light scattering on a Malvern Zetasizer or Wyatt NanoStar. The nanoparticle sample is diluted in phosphate buffered saline (PBS) so that the count rate is approximately 200-400 kcps.

In some embodiments, the LNP compositions are formed with an average encapsulation efficiency ranging from about 50% to about 100%. In some embodiments, the LNP compositions are formed with an average encapsulation efficiency ranging from about 50% to about 95%. In some embodiments, the LNP compositions are formed with an average encapsulation efficiency ranging from about 70% to about 90%. In some embodiments, the LNP compositions are formed with an average encapsulation efficiency ranging from about 90% to about 100%. In some embodiments, the LNP compositions are formed with an average encapsulation efficiency ranging from about 75% to about 95%. In some embodiments, the LNP compositions are formed with an average encapsulation efficiency ranging from about 90% to about 100%. In some embodiments, the LNP compositions are formed with an average encapsulation efficiency ranging from about 95% to about 100%. In some embodiments, the LNP compositions are formed with an average encapsulation efficiency ranging from about 98% to about 100%. In some embodiments, the LNP compositions are formed with an average encapsulation efficiency ranging from about 99% to about 100%.

### Cargo

The cargo delivered via an LNP composition described herein includes a biologically active agent. The biologically active agent may be a nucleic acid, such as an mRNA or gRNA. In certain embodiments, the cargo is or comprises one or more biologically active agent, such as mRNA, gRNA, expression vector, RNA-guided DNA-binding agent, antibody (e.g., monoclonal, chimeric, humanized, nanobody, and fragments thereof etc.), cholesterol, hormone, peptide, protein, chemotherapeutic and other types of antineoplastic agent, low molecular weight drug, vitamin, co-factor, nucleoside, nucleotide, oligonucleotide, enzymatic nucleic acid, antisense nucleic acid, triplex forming oligonucleotide, antisense DNA or RNA composition, chimeric DNA:RNA composition, allozyme, aptamer, ribozyme, decoys and analogs thereof, plasmid and other types of vectors, and small nucleic acid molecule, RNAi agent, short interfering nucleic acid (siNA), short interfering RNA (siRNA), double-stranded RNA (dsRNA), micro-RNA (miRNA), short hairpin RNA (shRNA) and “self-replicating RNA” (encoding a replicase enzyme activity and capable of directing its own replication or amplification in vivo) molecules, peptide nucleic acid (PNA), a locked nucleic acid ribonucleotide (LNA), morpholino nucleotide, threose nucleic acid (TNA), glycol nucleic acid (GNA), sisiRNA (small internally segmented interfering RNA), and iRNA (asymmetrical interfering RNA). The above list of biologically active agents is exemplary only, and is not intended to be limiting. Such compounds may be purified or partially purified, and may be naturally occurring or synthetic, and may be chemically modified.

The cargo delivered via LNP composition may be an RNA, such as an mRNA molecule encoding a protein of interest. For example, an mRNA for expressing a protein such as green fluorescent protein (GFP), an RNA-guided DNA-binding agent, or a Cas nuclease is included. LNP compositions that include a Cas nuclease mRNA, for example a Class 2 Cas nuclease mRNA that allows for expression in a cell of a Class 2 Cas nuclease such as a Cas9 or Cpf1 (also referred to as Cas12a) protein are provided. Further, the cargo may contain one or more gRNAs or nucleic acids encoding gRNAs. A template nucleic acid, e.g., for repair or recombination, may also be included with the compositions or a template nucleic acid may be used in the methods described herein. In a sub-embodiment, the cargo comprises an mRNA that encodes a Streptococcus pyogenes Cas9, optionally and an S. pyogenes gRNA. In a further sub-embodiment, the cargo comprises an mRNA that encodes a Neisseria meningitidis Cas9, optionally and an Nme (Neisseria meningitidis) gRNA.

“mRNA” refers to a polynucleotide and comprises an open reading frame that can be translated into a polypeptide (i.e., can serve as a substrate for translation by a ribosome and amino-acylated tRNAs). mRNA can comprise a phosphate-sugar backbone including ribose residues or analogs thereof, e.g., 2′-methoxy ribose residues. In some embodiments, the sugars of an mRNA phosphate-sugar backbone consist essentially of ribose residues, 2′-methoxy ribose residues, or a combination thereof. In general, mRNAs do not contain a substantial quantity of thymidine residues (e.g., 0 residues or fewer than 30, 20, 10, 5, 4, 3, or 2 thymidine residues; or less than 10%, 9%, 8%, 7%, 6%, 5%, 4%, 4%, 3%, 2%, 1%, 0.5%, 0.2%, or 0.1% thymidine content). An mRNA can contain modified uridines at some or all of its uridine positions.

### Genome Editing Tools

In some embodiments, the LNP composition is a lipid nucleic acid assembly, also referred to as a lipid nucleic acid composition. In some embodiments, the lipid nucleic acid composition or LNP composition comprises a genome editing tool or a nucleic acid encoding the same. As used herein, the term “genome editing tool” (or “gene editing tool”) is any component of “genome editing system” (or “gene editing system”) necessary or helpful for producing an edit in the genome of a cell. In some embodiments, the present disclosure provides for methods of delivering genome editing tools of a genome editing system (for example a zinc finger nuclease system, a TALEN system, a meganuclease system or a CRISPR/Cas system) to a cell (or population of cells). Genome editing tools include, for example, nucleases capable of making single or double strand break in the DNA or RNA of a cell, e.g., in the genome of a cell. The genome editing tools, e.g. nucleases, may optionally modify the genome of a cell without cleaving the nucleic acid, or nickases. A genome editing nuclease or nickase may be encoded by an mRNA. Such nucleases include, for example, RNA-guided DNA binding agents, and CRISPR/Cas components. Genome editing tools include fusion proteins, including e.g., a nickase fused to an effector domain such as an editor domain. Genome editing tools include any item necessary or helpful for accomplishing the goal of a genome edit, such as, for example, guide RNA, sgRNA, dgRNA, donor nucleic acid, and the like.

Various suitable gene editing systems comprising genome editing tools for delivery with the lipid nucleic acid assembly compositions are described herein, including but not limited to the CRISPR/Cas system; zinc finger nuclease (ZFN) system; and the transcription activator-like effector nuclease (TALEN) system. Generally, the gene editing systems involve the use of engineered cleavage systems to induce a double strand break (DSB) or a nick (e.g., a single strand break, or SSB) in a target DNA sequence. Cleavage or nicking can occur through the use of specific nucleases such as engineered ZFN, TALENs, or using the CRISPR/Cas system with an engineered guide RNA to guide specific cleavage or nicking of a target DNA sequence. Further, targeted nucleases are being developed based on the Argonaute system (e.g., from T. thermophilus, known as ‘TtAgo’, see Swarts et al (2014) Nature 507(7491): 258-261), which also may have the potential for uses in genome editing and gene therapy.

In certain embodiments, the disclosed compositions comprise one or more DNA modifying agents, such as a DNA cutting agent. A variety of DNA modifying agents may be included in the LNP compositions described herein. For example, DNA modifying agents include nucleases (both sequence-specific and non-specific), topoisomerases, methylases, acetylases, chemicals, pharmaceuticals, and other agents. In some embodiments, proteins that bind to a given DNA sequence or set of sequences may be employed to induce DNA modification such as strand breakage. Proteins can either be modified by many means, such as incorporation of ¹²⁵I, the radioactive decay of which would cause strand breakage, or modifying cross-linking reagents such as 4-azidophenacylbromide which form a cross-link with DNA on exposure to UV-light. Such protein-DNA cross-links can subsequently be converted to a double-stranded DNA break by treatment with piperidine. Yet another approach to DNA modification involves antibodies raised against specific proteins bound at one or more DNA sites, such as transcription factors or architectural chromatin proteins, and used to isolate the DNA from nucleoprotein complexes.

In certain embodiments, the disclosed compositions comprise one or more DNA cutting agents. DNA cutting agents include technologies such as Zinc-Finger Nucleases (ZFN), Transcription Activator-Like Effector Nucleases (TALEN), mito-TALEN, and meganuclease systems. TALEN and ZFN technologies use a strategy of tethering endonuclease catalytic domains to modular DNA binding proteins for inducing targeted DNA double-stranded breaks (DSB) at specific genomic loci. Additional DNA cutting agents include small interfering RNA, micro RNA, anti-microRNA, antagonist, small hairpin RNA, and aptamers (RNA, DNA or peptide based (including affimers)).

In some embodiments, the gene editing system is a TALEN system. Transcription activator-like effector nucleases (TALEN) are restriction enzymes that can be engineered to cut specific sequences of DNA. They are made by fusing a TAL effector DNA-binding domain to a DNA cleavage domain (a nuclease which cuts DNA strands). Transcription activator-like effectors (TALEs) can be engineered to bind to a desired DNA sequence, to promote DNA cleavage at specific locations (see, e.g., Boch, 2011, Nature Biotech). The restriction enzymes can be introduced into cells, for use in gene editing or for genome editing in situ, a technique known as genome editing with engineered nucleases. Such methods and compositions for use therein are known in the art. See, e.g., WO2019147805, WO2014040370, WO2018073393, the contents of which are hereby incorporated in their entireties.

In some embodiments, the gene editing system is a zinc-finger system. Zinc-finger nucleases (ZFNs) are artificial restriction enzymes generated by fusing a zinc finger DNA-binding domain to a DNA-cleavage domain. Zinc finger domains can be engineered to target specific desired DNA sequences to enables zinc-finger nucleases to target unique sequences within complex genomes. The non-specific cleavage domain from the type Us restriction endonuclease FokI is typically used as the cleavage domain in ZFNs. Cleavage is repaired by endogenous DNA repair machinery, allowing ZFN to precisely alter the genomes of higher organisms. Such methods and compositions for use therein are known in the art. See, e.g., WO2011091324, the contents of which are hereby incorporated in their entireties.

In preferred embodiments, the disclosed compositions comprise an mRNA encoding an RNA-guided DNA-binding agent, such as a Cas nuclease. In particular embodiments, the disclosed compositions comprise an mRNA encoding a Class 2 Cas nuclease, such as S. pyogenes Cas9.

As used herein, an “RNA-guided DNA-binding agent” means a polypeptide or complex of polypeptides having RNA and DNA-binding activity, or a DNA-binding subunit of such a complex, wherein the DNA-binding activity is sequence-specific and depends on the sequence of the RNA. Exemplary RNA-guided DNA-binding agents include Cas cleavases/nickases and inactivated forms thereof (“dCas DNA-binding agents”). “Cas nuclease”, as used herein, encompasses Cas cleavases, Cas nickases, and dCas DNA-binding agents. Cas cleavases/nickases and dCas DNA-binding agents include a Csm or Cmr complex of a type III CRISPR system, the Cas10, Csm1, or Cmr2 subunit thereof, a Cascade complex of a type I CRISPR system, the Cas3 subunit thereof, and Class 2 Cas nucleases. As used herein, a “Class 2 Cas nuclease” is a single-chain polypeptide with RNA-guided DNA-binding activity. Class 2 Cas nucleases include Class 2 Cas cleavases/nickases (e.g., H840A, D10A, or N863A variants), which further have RNA-guided DNA cleavases or nickase activity, and Class 2 dCas DNA-binding agents, in which cleavase/nickase activity is inactivated. Class 2 Cas nucleases that may be used with the LNP compositions described herein include, for example, Cas9, Cpf1, C2c1, C2c2, C2c3, HF Cas9 (e.g., N497A, R661A, Q695A, Q926A variants), HypaCas9 (e.g., N692A, M694A, Q695A, H698A variants), eSPCas9(1.0) (e.g, K810A, K1003A, R1060A variants), and eSPCas9(1.1) (e.g., K848A, K1003A, R1060A variants) proteins and modifications thereof. Cpf1 protein, Zetsche et al., Cell, 163: 1-13 (2015), is homologous to Cas9, and contains a RuvC-like nuclease domain. Cpf1 sequences of Zetsche are incorporated by reference in their entirety. See, e.g., Zetsche, Tables 2 and 4. See, e.g., Makarova et al., Nat Rev Microbiol, 13(11): 722-36 (2015); Shmakov et al., Molecular Cell, 60:385-397 (2015).

Non-limiting exemplary species that the Cas nuclease can be derived from include Streptococcus pyogenes, Streptococcus thermophilus, Streptococcus sp., Staphylococcus aureus, Listeria innocua, Lactobacillus gasseri, Francisella novicida, Wolinella succinogenes, Sutterella wadsworthensis, Gammaproteobacterium, Neisseria meningitidis, Campylobacter jejuni, Pasteurella multocida, Fibrobacter succinogene, Rhodospirillum rubrum, Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Lactobacillus buchneri, Treponema denticola, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, Streptococcus pasteurianus, Neisseria cinerea, Campylobacter lari, Parvibaculum lavamentivorans, Corynebacterium diphtheria, Acidaminococcus sp., Lachnospiraceae bacterium ND2006, and Acaryochloris marina.

In some embodiments, the Cas nuclease is the Cas9 nuclease from Streptococcus pyogenes. In other embodiments, the Cas nuclease is the Cas9 nuclease from Streptococcus thermophilus. In still other embodiments, the Cas nuclease is the Cas9 nuclease from Neisseria meningitidis. In some embodiments, the Cas nuclease is the Cas9 nuclease is from Staphylococcus aureus. In some embodiments, the Cas nuclease is the Cpf1 nuclease from Francisella novicida. In other embodiments, the Cas nuclease is the Cpf1 nuclease from Acidaminococcus sp. In still other embodiments, the Cas nuclease is the Cpf1 nuclease from Lachnospiraceae bacterium ND2006. In further embodiments, the Cas nuclease is the Cpf1 nuclease from Francisella tularensis, Lachnospiraceae bacterium, Butyrivibrio proteoclasticus, Peregrinibacteria bacterium, Parcubacteria bacterium, Smithella, Acidaminococcus, Candidatus Methanoplasma termitum, Eubacterium eligens, Moraxella bovoculi, Leptospira inadai, Porphyromonas crevioricanis, Prevotella disiens, or Porphyromonas macacae. In some embodiments, the Cas nuclease is a Cpf1 nuclease from an Acidaminococcus or Lachnospiraceae. Wild type Cas9 has two nuclease domains: RuvC and HNH. The RuvC domain cleaves the non-target DNA strand, and the HNH domain cleaves the target strand of DNA. In some embodiments, the Cas9 nuclease comprises more than one RuvC domain and/or more than one HNH domain. In some embodiments, the Cas9 nuclease is a wild type Cas9. In some embodiments, the Cas9 is capable of inducing a double strand break in target DNA. In other embodiments, the Cas nuclease may cleave dsDNA, it may cleave one strand of dsDNA, or it may not have DNA cleavase or nickase activity.

In some embodiments, chimeric Cas nucleases are used, where one domain or region of the protein is replaced by a portion of a different protein. In some embodiments, a Cas nuclease domain may be replaced with a domain from a different nuclease such as Fok1. In some embodiments, a Cas nuclease may be a modified nuclease.

In other embodiments, the Cas nuclease or Cas nickase may be from a Type-I CRISPR/Cas system. In some embodiments, the Cas nuclease may be a component of the Cascade complex of a Type-I CRISPR/Cas system. In some embodiments, the Cas nuclease may be a Cas3 protein. In some embodiments, the Cas nuclease may be from a Type-III CRISPR/Cas system. In some embodiments, the Cas nuclease may have an RNA cleavage activity.

In some embodiments, the RNA-guided DNA-binding agent has single-strand nickase activity, i.e., can cut one DNA strand to produce a single-strand break, also known as a “nick.” In some embodiments, the RNA-guided DNA-binding agent comprises a Cas nickase. A nickase is an enzyme that creates a nick in dsDNA, i.e., cuts one strand but not the other of the DNA double helix. In some embodiments, a Cas nickase is a version of a Cas nuclease (e.g., a Cas nuclease discussed above) in which an endonucleolytic active site is inactivated, e.g., by one or more alterations (e.g., point mutations) in a catalytic domain. See, e.g., U.S. Pat. No. 8,889,356 for discussion of Cas nickases and exemplary catalytic domain alterations. In some embodiments, a Cas nickase such as a Cas9 nickase has an inactivated RuvC or HNH domain. In some embodiments, the RNA-guided DNA-binding agent is modified to contain only one functional nuclease domain. For example, the agent protein may be modified such that one of the nuclease domains is mutated or fully or partially deleted to reduce its nucleic acid cleavage activity. In some embodiments, a nickase is used having a RuvC domain with reduced activity. In some embodiments, a nickase is used having an inactive RuvC domain. In some embodiments, a nickase is used having an HNH domain with reduced activity. In some embodiments, a nickase is used having an inactive HNH domain.

In some embodiments, a conserved amino acid within a Cas protein nuclease domain is substituted to reduce or alter nuclease activity. In some embodiments, a Cas nuclease may comprise an amino acid substitution in the RuvC or RuvC-like nuclease domain. Exemplary amino acid substitutions in the RuvC or RuvC-like nuclease domain include D10A (based on the S. pyogenes Cas9 protein). See, e.g., Zetsche et al. (2015) Cell October 22:163(3): 759-771. In some embodiments, the Cas nuclease may comprise an amino acid substitution in the HNH or HNH-like nuclease domain. Exemplary amino acid substitutions in the HNH or HNH-like nuclease domain include E762A, H840A, N863A, H983A, and D986A (based on the S. pyogenes Cas9 protein). See, e.g., Zetsche et al. (2015). Further exemplary amino acid substitutions include D917A, E1006A, and D1255A (based on the Francisella novicida U112 Cpf1 (FnCpf1) sequence (UniProtKB—A0Q7Q2 (CPF1_FRATN)).

In some embodiments, an mRNA encoding a nickase is provided in combination with a pair of guide RNAs that are complementary to the sense and antisense strands of the target sequence, respectively. In this embodiment, the guide RNAs direct the nickase to a target sequence and introduce a DSB by generating a nick on opposite strands of the target sequence (i.e., double nicking). In some embodiments, use of double nicking may improve specificity and reduce off-target effects. In some embodiments, a nickase is used together with two separate guide RNAs targeting opposite strands of DNA to produce a double nick in the target DNA. In some embodiments, a nickase is used together with two separate guide RNAs that are selected to be in close proximity to produce a double nick in the target DNA.

In some embodiments, the RNA-guided DNA-binding agent lacks cleavase and nickase activity. In some embodiments, the RNA-guided DNA-binding agent comprises a dCas DNA-binding polypeptide. A dCas polypeptide has DNA-binding activity while essentially lacking catalytic (cleavase/nickase) activity. In some embodiments, the dCas polypeptide is a dCas9 polypeptide. In some embodiments, the RNA-guided DNA-binding agent lacking cleavase and nickase activity or the dCas DNA-binding polypeptide is a version of a Cas nuclease (e.g., a Cas nuclease discussed above) in which its endonucleolytic active sites are inactivated, e.g., by one or more alterations (e.g., point mutations) in its catalytic domains. See, e.g., US 2014/0186958 A1; US 2015/0166980 A1.

In some embodiments, the RNA-guided DNA binding agent comprises a APOBEC3 deaminase. In some embodiments, an APOBEC3 deaminase is an APOBEC3A (A3A). In some embodiments, the A3A is a human A3A. In some embodiments, the A3A is a wild-type A3A.

In some embodiments, the RNA-guided DNA binding agent comprises an editor. An exemplary editor comprises a human A3A fused to S. pyogenes D10A Cas9 nickase. In some embodiments the editor comprises a human A3A fused to a N. meningitidis D16A nickase. In some embodiments, the editor is provided with a uracil glycosylase inhibitor (“UGI”). In some embodiments, the editor is fused to the UGI. In some embodiments the UGI is not fused to the editor. In some embodiments, the mRNA encoding the editor and an mRNA encoding the UGI are formulated together in an LNP. In other embodiments, the editor and UGI are provided in separate LNPs.

In some embodiments, the RNA-guided DNA-binding agent comprises one or more heterologous functional domains (e.g., is or comprises a fusion polypeptide).

In some embodiments, the heterologous functional domain may facilitate transport of the RNA-guided DNA-binding agent into the nucleus of a cell. For example, the heterologous functional domain may be a nuclear localization signal (NLS).

In some embodiments, the heterologous functional domain may be capable of modifying the intracellular half-life of the RNA-guided DNA binding agent. In some embodiments, the half-life of the RNA-guided DNA binding agent may be increased. In some embodiments, the half-life of the RNA-guided DNA-binding agent may be reduced. In some embodiments, the heterologous functional domain may be capable of increasing the stability of the RNA-guided DNA-binding agent. In some embodiments, the heterologous functional domain may be capable of reducing the stability of the RNA-guided DNA-binding agent. In some embodiments, the heterologous functional domain may act as a signal peptide for protein degradation. In some embodiments, the protein degradation may be mediated by proteolytic enzymes, such as, for example, proteasomes, lysosomal proteases, or calpain proteases. In some embodiments, the heterologous functional domain may comprise a PEST sequence. In some embodiments, the RNA-guided DNA-binding agent may be modified by addition of ubiquitin or a polyubiquitin chain. In some embodiments, the ubiquitin may be a ubiquitin like protein (UBL). Non-limiting examples of ubiquitin-like proteins include small ubiquitin like modifier (SUMO), ubiquitin cross-reactive protein (UCRP, also known as interferon stimulated gene-15 (ISG15)), ubiquitin-related modifier-1 (URM1), neuronal-precursor-cell expressed developmentally downregulated protein-8 (NEDD8, also called Rub1 in S. cerevisiae), human leukocyte antigen F-associated (FAT10), autophagy-8 (ATG8) and -12 (ATG12), Fau ubiquitin-like protein (FUB1), membrane-anchored UBL (MUB), ubiquitin fold-modifier-1 (UFM1), and ubiquitin-like protein-5 (UBL5).

In some embodiments, the heterologous functional domain may be a marker domain. Non-limiting examples of marker domains include fluorescent proteins, purification tags, epitope tags, and reporter gene sequences. In some embodiments, the marker domain may be a fluorescent protein. Non-limiting examples of suitable fluorescent proteins include green fluorescent proteins (e.g., GFP, GFP-2, tagGFP, turboGFP, sfGFP, EGFP, Emerald, Azami Green, Monomeric Azami Green, CopGFP, AceGFP, ZsGreen1), yellow fluorescent proteins (e.g., YFP, EYFP, Citrine, Venus, YPet, PhiYFP, ZsYellow1), blue fluorescent proteins (e.g., EBFP, EBFP2, Azurite, mKalamal, GFPuv, Sapphire, T-sapphire,), cyan fluorescent proteins (e.g., ECFP, Cerulean, CyPet, AmCyan1, Midoriishi-Cyan), red fluorescent proteins (e.g., mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain may be a purification tag and/or an epitope tag. Non-limiting exemplary tags include glutathione-S-transferase (GST), chitin binding protein (CBP), maltose binding protein (MBP), thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, 8×His, biotin carboxyl carrier protein (BCCP), poly-His, and calmodulin. Non-limiting exemplary reporter genes include glutathione-S-transferase (GST), horseradish peroxidase (HRP), chloramphenicol acetyltransferase (CAT), beta-galactosidase, beta-glucuronidase, luciferase, or fluorescent proteins.

In additional embodiments, the heterologous functional domain may target the RNA-guided DNA-binding agent to a specific organelle, cell type, tissue, or organ. In some embodiments, the heterologous functional domain may target the RNA-guided DNA-binding agent to mitochondria.

In further embodiments, the heterologous functional domain may be an effector domain such as an editor domain. When the RNA-guided DNA-binding agent is directed to its target sequence, e.g., when a Cas nuclease is directed to a target sequence by a gRNA, the effector domain such as an editor domain may modify or affect the target sequence. In some embodiments, the effector domain such as an editor domain may be chosen from a nucleic acid binding domain, a nuclease domain (e.g., a non-Cas nuclease domain), an epigenetic modification domain, a transcriptional activation domain, or a transcriptional repressor domain. In some embodiments, the heterologous functional domain is a nuclease, such as a FokI nuclease. See, e.g., U.S. Pat. No. 9,023,649. In some embodiments, the heterologous functional domain is a transcriptional activator or repressor. See, e.g., Qi et al., “Repurposing CRISPR as an RNA-guided platform for sequence-specific control of gene expression,” Cell 152:1173-83 (2013); Perez-Pinera et al., “RNA-guided gene activation by CRISPR-Cas9-based transcription factors,” Nat. Methods 10:973-6 (2013); Mali et al., “CAS9 transcriptional activators for target specificity screening and paired nickases for cooperative genome engineering,” Nat. Biotechnol. 31:833-8 (2013); Gilbert et al., “CRISPR-mediated modular RNA-guided regulation of transcription in eukaryotes,” Cell 154:442-51 (2013). As such, the RNA-guided DNA-binding agent essentially becomes a transcription factor that can be directed to bind a desired target sequence using a guide RNA. In some embodiments, the DNA modification domain is a methylation domain, such as a demethylation or methyltransferase domain. In some embodiments, the effector domain is a DNA modification domain, such as a base-editing domain. In particular embodiments, the DNA modification domain is a nucleic acid editing domain that introduces a specific modification into the DNA, such as a deaminase domain. See, e.g., WO 2015/089406; US 2016/0304846. The nucleic acid editing domains, deaminase domains, and Cas9 variants described in WO 2015/089406 and U.S. 2016/0304846, each of which is hereby incorporated by reference in its entirety.

The nuclease may comprise at least one domain that interacts with a guide RNA (“gRNA”). Additionally, the nuclease may be directed to a target sequence by a gRNA. In Class 2 Cas nuclease systems, the gRNA interacts with the nuclease as well as the target sequence, such that it directs binding to the target sequence. In some embodiments, the gRNA provides the specificity for the targeted cleavage, and the nuclease may be universal and paired with different gRNAs to cleave different target sequences. Class 2 Cas nuclease may pair with a gRNA scaffold structure of the types, orthologs, and exemplary species listed above.

As used herein, “ribonucleoprotein” (RNP) or “RNP complex” refers to a gRNA together with an RNA-guided DNA-binding agent, such as a Cas nuclease, e.g., a Cas cleavase, Cas nickase, or dCas DNA-binding agent (e.g., Cas9). In some embodiments, the gRNA guides the RNA-guided DNA-binding agent such as Cas9 to a target sequence, and the gRNA hybridizes with and the agent binds to the target sequence; in cases where the agent is a cleavase or nickase, binding can be followed by cleaving or nicking.

In some embodiments of the present disclosure, the cargo for the LNP composition includes at least one gRNA comprising guide sequences that direct an RNA-guided DNA-binding agent, which can be a nuclease (e.g., a Cas nuclease such as Cas9), to a target DNA. The gRNA may guide the Cas nuclease or Class 2 Cas nuclease to a target sequence on a target nucleic acid molecule. In some embodiments, a gRNA binds with and provides specificity of cleavage by a Class 2 Cas nuclease. In some embodiments, the gRNA and the Cas nuclease may form a ribonucleoprotein (RNP), e.g., a CRISPR/Cas complex such as a CRISPR/Cas9 complex. In some embodiments, the CRISPR/Cas complex may be a Type-II CRISPR/Cas9 complex. In some embodiments, the CRISPR/Cas complex may be a Type-V CRISPR/Cas complex, such as a Cpf1/gRNA complex. Cas nucleases and cognate gRNAs may be paired. The gRNA scaffold structures that pair with each Class 2 Cas nuclease vary with the specific CRISPR/Cas system.

“Guide RNA”, “gRNA”, and simply “guide” are used herein interchangeably to refer to a cognate guide nucleic acid for an RNA-guided DNA-binding agent. Guide RNAs can include modified RNAs as described herein. A gRNA may be, for example, either a single guide RNA, or the combination of a crRNA and a trRNA (also known as tracrRNA). The crRNA and trRNA may be associated as a single RNA molecule (as a single guide RNA, sgRNA) or, for example, in two separate RNA strands (dual guide RNA, dgRNA). In some systems a gRNA may be a crRNA (also known as a CRISPR RNA). “Guide RNA” or “gRNA” refers to each type. The trRNA may be a naturally-occurring sequence, or a trRNA sequence with modifications or variations compared to naturally-occurring sequences.

In some embodiments, an mRNA encoding a RNA-guided DNA binding agent is formulated in a first LNP composition and a gRNA nucleic acid is formulated in a second LNP composition. In some embodiments, the first and second LNP compositions are administered simultaneously. In other embodiments, the first and second LNP compositions are administered sequentially. In some embodiments, the first and second LNP compositions are combined prior to the preincubation step. In other embodiments, the first and second LNP compositions are preincubated separately.

In some embodiments, the cargo may comprise a DNA molecule. In some embodiments, the nucleic acid may comprise a nucleotide sequence encoding a crRNA. In some embodiments, the nucleotide sequence encoding the crRNA comprises a targeting sequence flanked by all or a portion of a repeat sequence from a naturally occurring CRISPR/Cas system. In some embodiments, the nucleic acid may comprise a nucleotide sequence encoding a tracr RNA. In certain embodiments, the crRNA and the tracr RNA may be encoded by two separate nucleic acids. In other embodiments, the crRNA and the tracr RNA may be encoded by a single nucleic acid. In some embodiments, the crRNA and the tracr RNA may be encoded by opposite strands of a single nucleic acid. In other embodiments, the crRNA and the tracr RNA may be encoded by the same strand of a single nucleic acid. In some embodiments, the gRNA nucleic acid encodes an sgRNA. In some embodiments, the gRNA nucleic acid encodes a Cas9 nuclease sgRNA. In come embodiments, the gRNA nucleic acid encodes a Cpf1 nuclease sgRNA.

The nucleotide sequence encoding the guide RNA may be operably linked to at least one transcriptional or regulatory control sequence, such as a promoter, a 3′ UTR, or a 5′ UTR. In one example, the promoter may be a tRNA promoter, e.g., tRNALys3, or a tRNA chimera. See Mefferd et al., RNA. 2015 21:1683-9; Scherer et al., Nucleic Acids Res. 2007 35: 2620-2628. In some embodiments, the promoter may be recognized by RNA polymerase III (Pol III). Non-limiting examples of Pol III promoters also include U6 and H1 promoters. In some embodiments, the nucleotide sequence encoding the guide RNA may be operably linked to a mouse or human U6 promoter. In some embodiments, the gRNA nucleic acid is a modified nucleic acid. In some embodiments, the gRNA nucleic acid includes a modified nucleoside or nucleotide. In some embodiments, the gRNA nucleic acid includes a 5′ end modification, for example a modified nucleoside or nucleotide to stabilize and prevent integration of the nucleic acid. In other embodiments, the gRNA nucleic acid comprises a double-stranded DNA having a 5′ end modification on each strand. In some embodiments, the gRNA nucleic acid includes an inverted dideoxy-T or an inverted abasic nucleoside or nucleotide as the 5′ end modification. In some embodiments, the gRNA nucleic acid includes a label such as biotin, desthiobiotin-TEG, digoxigenin, and fluorescent markers, including, for example, FAM, ROX, TAMRA, and AlexaFluor.

As used herein, a “guide sequence” refers to a sequence within a gRNA that is complementary to a target sequence and functions to direct a gRNA to a target sequence for binding and/or modification (e.g., cleavage) by an RNA-guided DNA-binding agent. A “guide sequence” may also be referred to as a “targeting sequence,” or a “spacer sequence.” A guide sequence can be 20 base pairs in length, e.g., in the case of Streptococcus pyogenes (i.e., Spy Cas9) and related Cas9 homologs/orthologs. Shorter or longer sequences can also be used as guides, e.g., 15-, 16-, 17-, 18-, 19-, 21-, 22-, 23-, 24-, or 25-nucleotides in length. In some embodiments, the target sequence is in a gene or on a chromosome, for example, and is complementary to the guide sequence. In some embodiments, the degree of complementarity or identity between a guide sequence and its corresponding target sequence may be about or at least 75%, 80%, 85%, 90%, 95%, 96%, 97%, 98%, 99%, or 100%. In some embodiments, the guide sequence and the target region may be 100% complementary or identical over a region of at least 15, 16, 17, 18, 19, or 20 contiguous nucleotides. In other embodiments, the guide sequence and the target region may contain at least one mismatch. For example, the guide sequence and the target sequence may contain 1, 2, 3, or 4 mismatches, where the total length of the target sequence is at least 17, 18, 19, 20 or more base pairs. In some embodiments, the guide sequence and the target region may contain 1-4 mismatches where the guide sequence comprises at least 17, 18, 19, 20 or more nucleotides. In some embodiments, the guide sequence and the target region may contain 1, 2, 3, or 4 mismatches where the guide sequence comprises 20 nucleotides.

In certain embodiments, multiple LNP compositions may be used collaboratively and/or for separate purposes. In some embodiments, a cell may be contacted with first and second LNP compositions described herein. In some embodiments, the first and second LNP compositions each independently comprise one or more of an mRNA, a gRNA, and a guide RNA nucleic acid. In some embodiments, the first and second LNP compositions are administered simultaneously. In some embodiments, the first and second LNP compositions are administered sequentially.

In some embodiments, a method of producing multiple genome edits in a cell is provided (sometimes referred to herein and elsewhere as “multiplexing” or “multiplex gene editing” or “multiplex genome editing”). The ability to engineer multiple attributes into a single cell depends on the ability to perform edits in multiple targeted genes efficiently, including knockouts and in locus insertions, while retaining viability and the desired cell phenotype. In some embodiments, the method comprises culturing a cell in vitro, contacting the cell with two or more lipid nucleic acid assembly compositions, wherein each lipid nucleic acid assembly composition comprises a nucleic acid genome editing tool capable of editing a target site, and expanding the cell in vitro. The method results in a cell having more than one genome edit, wherein the genome edits differ. In certain embodiments, the first LNP composition comprises a first gRNA and the second LNP composition comprises a second gRNA, wherein the first and second gRNAs comprise different guide sequences that are complementary to different targets. In such embodiments, the LNP compositions may allow for multiplex gene editing. In some embodiments, the cell is contacted with 1, 2, 3, 4, 5, 6, 7, 8, 9, 10, or 11 lipid nucleic acid assembly compositions. In some embodiments, the cell is contacted with at least 6 lipid nucleic acid assembly compositions.

Target sequences for RNA-guided DNA-binding proteins such as Cas proteins include both the positive and negative strands of genomic DNA (i.e., the sequence given and the sequence's reverse complement), as a nucleic acid substrate for a Cas protein is a double stranded nucleic acid. Accordingly, where a guide sequence is said to be “complementary to a target sequence”, it is to be understood that the guide sequence may direct a gRNA to bind to the reverse complement of a target sequence. Thus, in some embodiments, where the guide sequence binds the reverse complement of a target sequence, the guide sequence is identical to certain nucleotides of the target sequence (e.g., the target sequence not including the PAM) except for the substitution of U for T in the guide sequence.

In some embodiments, methods are provided for producing multiple genome edits in an in vitro-cultured cell, comprising the steps of: a) contacting the cell in vitro with at least a first lipid composition comprising a first nucleic acid, thereby producing a contacted cell; b) contacting the cell in vitro with at least a second lipid composition comprising a second nucleic acid, wherein the second nucleic acid is different from the first nucleic acid; and c) expanding the cell in vitro. In certain embodiments, methods are provided for producing multiple genome edits in an in vitro-cultured cell, comprising the steps of: a) contacting the cell in vitro with at least a first lipid composition comprising a first nucleic acid, thereby producing a contacted cell; b) culturing the contacted cell in vitro, thereby producing a cultured contacted cell; c) contacting the cultured contacted cell in vitro with at least a second lipid composition comprising a second nucleic acid, wherein the second nucleic acid is different from the first nucleic acid; and d) expanding the cell in vitro. In further embodiments, the methods further comprise contacting the cell in vitro with at least a third lipid composition comprising a third nucleic acid, wherein the third nucleic acid is different from the first and second nucleic acids. In still further embodiments, the methods further comprise contacting the cell in vitro with at least a fourth lipid composition comprising a fourth nucleic acid, wherein the fourth nucleic acid is different from the first second, and third nucleic acids. In still yet further embodiments, the methods further comprise contacting the cell in vitro with at least a fifth lipid composition comprising a fifth nucleic acid, wherein the fifth nucleic acid is different from the first second, third, and fourth nucleic acids. In additional embodiments, the methods further comprise contacting the cell in vitro with at least a sixth lipid composition comprising a sixth nucleic acid, wherein the sixth nucleic acid is different from the first second, third, fourth, and fifth nucleic acids. In certain embodiments, at least two of the lipid compositions are administered sequentially. In some embodiments, at least two of the lipid compositions are administered simultaneously. In some embodiments, the expanded cell exhibits increased survival.

In certain embodiments, at least one of the foregoing lipid compositions comprises a nucleic acid genome editing tool as described herein. In some embodiments, a further lipid composition comprises an RNA-guided DNA binding agent. In some embodiments, the RNA-guided DNA binding agent is Cas9.

In some embodiments, the methods of the present disclosure further comprise contacting the cell with a donor nucleic acid. In some embodiments, a further lipid composition comprises a donor nucleic acid. The donor nucleic acid may be inserted in a target sequence. In some embodiments, a donor nucleic acid sequence is provided as a vector. In some embodiments, the donor nucleic acid encodes a targeting receptor. In certain embodiments, the donor nucleic acid comprises regions having homology with corresponding regions of a T cell receptor sequence. A “targeting receptor” is a polypeptide present on the surface of a cell, e.g., a T cell, to permit binding of the cell to a target site, e.g., a specific cell or tissue in an organism. In some embodiments, the targeting receptor is a CAR. In some embodiments, the targeting receptor is a universal CAR (UniCAR). In some embodiments, the targeting receptor is a TCR. In some embodiments, the targeting receptor is a T cell receptor fusion construct (TRuC). In some embodiments, the targeting receptor is a B cell receptor (BCR) (e.g., expressed on a B cell). In some embodiments, the targeting receptor is chemokine receptor. In some embodiments, the targeting receptor is a cytokine receptor.

The length of the targeting sequence may depend on the CRISPR/Cas system and components used. For example, different Class 2 Cas nucleases from different bacterial species have varying optimal targeting sequence lengths. Accordingly, the targeting sequence may comprise 5, 6, 7, 8, 9, 10, 11, 12, 13, 14, 15, 16, 17, 18, 19, 20, 21, 22, 23, 24, 25, 26, 27, 28, 29, 30, 35, 40, 45, 50, or more than 50 nucleotides in length. In some embodiments, the targeting sequence length is 0, 1, 2, 3, 4, or 5 nucleotides longer or shorter than the guide sequence of a naturally-occurring CRISPR/Cas system. In certain embodiments, the Cas nuclease and gRNA scaffold will be derived from the same CRISPR/Cas system. In some embodiments, the targeting sequence may comprise or consist of 18-24 nucleotides. In some embodiments, the targeting sequence may comprise or consist of 19-21 nucleotides. In some embodiments, the targeting sequence may comprise or consist of 20 nucleotides.

In some embodiments, the sgRNA is a “Cas9 sgRNA” capable of mediating RNA-guided DNA cleavage by a Cas9 protein. In some embodiments, the sgRNA is a “Cpf1 sgRNA” capable of mediating RNA-guided DNA cleavage by a Cpf1 protein. In certain embodiments, the gRNA comprises a crRNA and tracr RNA sufficient for forming an active complex with a Cas9 protein and mediating RNA-guided DNA cleavage. In certain embodiments, the gRNA comprises a crRNA sufficient for forming an active complex with a Cpf1 protein and mediating RNA-guided DNA cleavage. See Zetsche 2015.

Certain embodiments also provide nucleic acids, e.g., expression cassettes, encoding the gRNA described herein. A “guide RNA nucleic acid” is used herein to refer to a gRNA (e.g. an sgRNA or a dgRNA) and a gRNA expression cassette, which is a nucleic acid that encodes one or more gRNAs.

### Modified RNAs

In certain embodiments, the lipid compositions, such as LNP compositions comprise modified nucleic acids, including modified RNAs.

Modified nucleosides or nucleotides can be present in an RNA, for example a gRNA or mRNA. A gRNA or mRNA comprising one or more modified nucleosides or nucleotides, for example, is called a “modified” RNA to describe the presence of one or more non-naturally and/or naturally occurring components or configurations that are used instead of or in addition to the canonical A, G, C, and U residues. In some embodiments, a modified RNA is synthesized with a non-canonical nucleoside or nucleotide, here called “modified.”

Modified nucleosides and nucleotides can include one or more of: (i) alteration, e.g., replacement, of one or both of the non-linking phosphate oxygens and/or of one or more of the linking phosphate oxygens in the phosphodiester backbone linkage (an exemplary backbone modification); (ii) alteration, e.g., replacement, of a constituent of the ribose sugar, e.g., of the 2′ hydroxyl on the ribose sugar (an exemplary sugar modification); (iii) wholesale replacement of the phosphate moiety with “dephospho” linkers (an exemplary backbone modification); (iv) modification or replacement of a naturally occurring nucleobase, including with a non-canonical nucleobase (an exemplary base modification); (v) replacement or modification of the ribose-phosphate backbone (an exemplary backbone modification); (vi) modification of the 3′ end or 5′ end of the polynucleotide, e.g., removal, modification or replacement of a terminal phosphate group or conjugation of a moiety, cap or linker (such 3′ or 5′ cap modifications may comprise a sugar and/or backbone modification); and (vii) modification or replacement of the sugar (an exemplary sugar modification). Certain embodiments comprise a 5′ end modification to an mRNA, gRNA, or nucleic acid. Certain embodiments comprise a modification to an mRNA, gRNA, or nucleic acid. Certain embodiments comprise a 3′ end modification to an mRNA, gRNA, or nucleic acid. A modified RNA can contain 5′ end and 3′ end modifications. A modified RNA can contain one or more modified residues at non-terminal locations. In certain embodiments, a gRNA includes at least one modified residue. In certain embodiments, an mRNA includes at least one modified residue. In certain embodiments, the modified gRNA comprises a modification at one or more of the first five nucleotides at a 5′ end. In certain embodiments, the modified gRNA comprises a modification at one or more of the first five nucleotides at a 5′ end. In certain embodiments, wherein the modified gRNA comprises a modification at one or more of the last five nucleotides at a 3′ end.

Unmodified nucleic acids can be prone to degradation by, e.g., intracellular nucleases or those found in serum. For example, nucleases can hydrolyze nucleic acid phosphodiester bonds. Accordingly, in one aspect the RNAs (e.g. mRNAs, gRNAs) described herein can contain one or more modified nucleosides or nucleotides, e.g., to introduce stability toward intracellular or serum-based nucleases. In some embodiments, the modified RNA molecules described herein can exhibit a reduced innate immune response when introduced into a population of cells, both in vivo and ex vivo. The term “innate immune response” includes a cellular response to exogenous nucleic acids, including single stranded nucleic acids, which involves the induction of cytokine expression and release, particularly the interferons, and cell death.

Accordingly, in some embodiments, an RNA or nucleic acid comprises at least one modification which confers increased or enhanced stability to the nucleic acid, including, for example, improved resistance to nuclease digestion in vivo. As used herein, the terms “modification” and “modified” as such terms relate to the nucleic acids provided herein, include at least one alteration which preferably enhances stability and renders the RNA or nucleic acid more stable (e.g., resistant to nuclease digestion) than the wild-type or naturally occurring version of the RNA or nucleic acid. As used herein, the terms “stable” and “stability” and such terms relate to the nucleic acids described herein, and particularly with respect to the RNA, refer to increased or enhanced resistance to degradation by, for example nucleases (i.e., endonucleases or exonucleases) which are normally capable of degrading such RNA. Increased stability can include, for example, less sensitivity to hydrolysis or other destruction by endogenous enzymes (e.g., endonucleases or exonucleases) or conditions within the target cell or tissue, thereby increasing or enhancing the residence of such RNA or nucleic acid in the target cell, tissue, subject and/or cytoplasm. The stabilized RNA or nucleic acid molecules provided herein demonstrate longer half-lives relative to their naturally occurring, unmodified counterparts (e.g. the wild-type version of the molecule). Also contemplated by the terms “modification” and “modified” as such terms related to the mRNA of the LNP compositions disclosed herein are alterations which improve or enhance translation of mRNA nucleic acids, including for example, the inclusion of sequences which function in the initiation of protein translation (e.g., the Kozak consensus sequence). (Kozak, M., Nucleic Acids Res 15 (20): 8125-48 (1987)).

In some embodiments, the RNA or nucleic acid has undergone a chemical or biological modification to render it more stable. Exemplary modifications to an RNA or nucleic acid include the depletion of a base (e.g., by deletion or by the substitution of one nucleotide for another) or modification of a base, for example, the chemical modification of a base. The phrase “chemical modifications” as used herein, includes modifications which introduce chemistries which differ from those seen in naturally occurring RNA or nucleic acids, for example, covalent modifications such as the introduction of modified nucleotides, (e.g., nucleotide analogs, or the inclusion of pendant groups which are not naturally found in such RNA, such as a deoxynucleoside, or nucleic acid molecules).

In some embodiments of a backbone modification, the phosphate group of a modified residue can be modified by replacing one or more of the oxygens with a different substituent. Further, the modified residue, e.g., modified residue present in a modified nucleic acid, can include the wholesale replacement of an unmodified phosphate moiety with a modified phosphate group as described herein. In some embodiments, the backbone modification of the phosphate backbone can include alterations that result in either an uncharged linker or a charged linker with unsymmetrical charge distribution.

Examples of modified phosphate groups include, phosphorothioate, phosphoroselenates, borano phosphates, borano phosphate esters, hydrogen phosphonates, phosphoroamidates, alkyl or aryl phosphonates and phosphotriesters. The phosphorous atom in an unmodified phosphate group is achiral. However, replacement of one of the non-bridging oxygens with one of the above atoms or groups of atoms can render the phosphorous atom chiral. The stereogenic phosphorous atom can possess either the “R” configuration (herein Rp) or the “S” configuration (herein Sp). The backbone can also be modified by replacement of a bridging oxygen, (i.e., the oxygen that links the phosphate to the nucleoside), with nitrogen (bridged phosphoroamidates), sulfur (bridged phosphorothioates) and carbon (bridged methylenephosphonates). The replacement can occur at either linking oxygen or at both of the linking oxygens. The phosphate group can be replaced by non-phosphorus containing connectors in certain backbone modifications. In some embodiments, the charged phosphate group can be replaced by a neutral moiety. Examples of moieties which can replace the phosphate group can include, without limitation, e.g., methyl phosphonate, hydroxylamino, siloxane, carbonate, carboxymethyl, carbamate, amide, thioether, ethylene oxide linker, sulfonate, sulfonamide, thioformacetal, formacetal, oxime, methyleneimino, methylenemethylimino, methylenehydrazo, methylenedimethylhydrazo and methyleneoxymethylimino.

mRNAs

In some embodiments, a composition or formulation disclosed herein comprises an mRNA comprising an open reading frame (ORF) encoding an RNA-guided DNA-binding agent, such as a Cas nuclease, or Class 2 Cas nuclease as described herein. In some embodiments, an mRNA comprising an ORF encoding an RNA-guided DNA-binding agent, such as a Cas nuclease or Class 2 Cas nuclease, is provided, used, or administered. An mRNA may comprise one or more of a 5′ cap, a 5′ untranslated region (UTR), a 3′ UTRs, and a polyadenine tail. The mRNA may comprise a modified open reading frame, for example to encode a nuclear localization sequence or to use alternate codons to encode the protein.

The mRNA in the disclosed LNP compositions may encode a cell surface or intracellular polypeptide. The mRNA in the disclosed LNP compositions may encode, for example, a secreted hormone, enzyme, receptor, polypeptide, peptide or other protein of interest that is normally secreted. In some embodiments, the mRNA may optionally have chemical or biological modifications which, for example, improve the stability and/or half-life of such mRNA or which improve or otherwise facilitate protein production.

In addition, suitable modifications include alterations in one or more nucleotides of a codon such that the codon encodes the same amino acid but is more stable than the codon found in the wild-type version of the mRNA. For example, an inverse relationship between the stability of RNA and a higher number cytidines (C's) and/or uridines (U's) residues has been demonstrated, and RNA devoid of C and U residues have been found to be stable to most RNases (Heidenreich, et al. J Biol Chem 269, 2131-8 (1994)). In some embodiments, the number of C and/or U residues in an mRNA sequence is reduced. In another embodiment, the number of C and/or U residues is reduced by substitution of one codon encoding a particular amino acid for another codon encoding the same or a related amino acid. Contemplated modifications to the mRNA nucleic acids also include the incorporation of pseudouridines. In some embodiments, a modified uridine is a pseudouridine modified at the 1 position, e.g., with a halogen, methyl, or ethyl. The modified uridine can be, for example, pseudouridine, N1-methyl-pseudouridine, 5-methoxyuridine, 5-iodouridine, or a combination thereof. In some embodiments, the modified uridine is 5-methoxyuridine. In some embodiments, the modified uridine is 5-iodouridine. In some embodiments, the modified uridine is pseudouridine. In some embodiments, the modified uridine is N1-methyl-pseudouridine. In some embodiments, the modified uridine is a combination of pseudouridine and N1-methyl-pseudouridine. In some embodiments, the modified uridine is a combination of pseudouridine and 5-methoxyuridine. In some embodiments, the modified uridine is a combination of N1-methyl pseudouridine and 5-methoxyuridine. In some embodiments, the modified uridine is a combination of 5-iodouridine and N1-methyl-pseudouridine. In some embodiments, the modified uridine is a combination of pseudouridine and 5-iodouridine. In some embodiments, the modified uridine is a combination of 5-iodouridine and 5-methoxyuridine. The incorporation of pseudouridines into the mRNA nucleic acids may enhance stability and translational capacity, as well as diminishing immunogenicity in vivo. See, e.g., Karikó, K., et al., Molecular Therapy 16 (11): 1833-1840 (2008). Substitutions and modifications to the mRNA may be performed by methods readily known to one or ordinary skill in the art.

The constraints on reducing the number of C and U residues in a sequence will likely be greater within the coding region of an mRNA, compared to an untranslated region, (i.e., it will likely not be possible to eliminate all of the C and U residues present in the message while still retaining the ability of the message to encode the desired amino acid sequence). The degeneracy of the genetic code, however presents an opportunity to allow the number of C and/or U residues that are present in the sequence to be reduced, while maintaining the same coding capacity (i.e., depending on which amino acid is encoded by a codon, several different possibilities for modification of RNA sequences may be possible). The term modification also includes, for example, the incorporation of non-nucleotide linkages or modified nucleotides into the mRNA sequences (e.g., modifications to one or both the 3′ and 5′ ends of an mRNA molecule encoding a functional secreted protein or enzyme). Such modifications include the addition of bases to an mRNA sequence (e.g., the inclusion of a poly A tail or a longer poly A tail), the alteration of the 3′ UTR or the 5′ UTR, complexing the mRNA with an agent (e.g., a protein or a complementary nucleic acid molecule), and inclusion of elements which change the structure of an mRNA molecule (e.g., which form secondary structures).

The poly A tail is thought to stabilize natural messengers. Therefore, a long poly A tail may be added to an mRNA molecule thus rendering the mRNA more stable. Poly A tails can be added using a variety of art-recognized techniques. For example, long poly A tails can be added to synthetic or in vitro transcribed mRNA using poly A polymerase (Yokoe, et al. Nature Biotechnology. 1996; 14: 1252-1256). A transcription vector can also encode long poly A tails. In addition, poly A tails can be added by transcription directly from PCR products. In some embodiments, the length of the poly A tail is at least about 90, 200, 300, 400 at least 500 nucleotides. In certain embodiments, the length of the poly A tail is adjusted to control the stability of a modified mRNA molecule and, thus, the transcription of protein. For example, since the length of the poly A tail can influence the half-life of an mRNA molecule, the length of the poly A tail can be adjusted to modify the level of resistance of the mRNA to nucleases and thereby control the time course of protein expression in a cell. In some embodiments, the stabilized mRNA molecules are sufficiently resistant to in vivo degradation (e.g., by nucleases), such that they may be delivered to the target cell without a transfer vehicle.

In certain embodiments, an mRNA can be modified by the incorporation 3′ and/or 5′ untranslated (UTR) sequences which are not naturally found in the wild-type mRNA. In some embodiments, 3′ and/or 5′ flanking sequence which naturally flanks an mRNA and encodes a second, unrelated protein can be incorporated into the nucleotide sequence of an mRNA molecule encoding a therapeutic or functional protein in order to modify it. For example, 3′ or 5′ sequences from mRNA molecules which are stable (e.g., globin, actin, GAPDH, tubulin, histone, or citric acid cycle enzymes) can be incorporated into the 3′ and/or 5′ region of a sense mRNA nucleic acid molecule to increase the stability of the sense mRNA molecule. See, e.g., US2003/0083272.

More detailed descriptions of the mRNA modifications can be found in US2017/0210698A1, at pages 57-68, the contents of which are incorporated herein.

### Template Nucleic Acid

The methods disclosed herein may include using a template nucleic acid. The template may be used to alter or insert a nucleic acid sequence at or near a target site for an RNA-guided DNA-binding protein such as a Cas nuclease, e.g., a Class 2 Cas nuclease. In some embodiments, the methods comprise introducing a template to the cell. In some embodiments, a single template may be provided. In other embodiments, two or more templates may be provided such that editing may occur at two or more target sites. For example, different templates may be provided to edit a single gene in a cell, or two different genes in a cell.

In some embodiments, the template may be used in homologous recombination. In some embodiments, the homologous recombination may result in the integration of the template sequence or a portion of the template sequence into the target nucleic acid molecule. In other embodiments, the template may be used in homology-directed repair, which involves DNA strand invasion at the site of the cleavage in the nucleic acid. In some embodiments, the homology-directed repair may result in including the template sequence in the edited target nucleic acid molecule. In yet other embodiments, the template may be used in gene editing mediated by non-homologous end joining. In some embodiments, the template sequence has no similarity to the nucleic acid sequence near the cleavage site. In some embodiments, the template or a portion of the template sequence is incorporated. In some embodiments, the template includes flanking inverted terminal repeat (ITR) sequences.

In some embodiments, the template sequence may correspond to, comprise, or consist of an endogenous sequence of a target cell. It may also or alternatively correspond to, comprise, or consist of an exogenous sequence of a target cell. As used herein, the term “endogenous sequence” refers to a sequence that is native to the cell. The term “exogenous sequence” refers to a sequence that is not native to a cell, or a sequence whose native location in the genome of the cell is in a different location. In some embodiments, the endogenous sequence may be a genomic sequence of the cell. In some embodiments, the endogenous sequence may be a chromosomal or extrachromosomal sequence. In some embodiments, the endogenous sequence may be a plasmid sequence of the cell.

In some embodiments, the template contains ssDNA or dsDNA containing flanking invert-terminal repeat (ITR) sequences. In some embodiments, the template is provided as a vector, plasmid, minicircle, nanocircle, or PCR product.

In some embodiments, the nucleic acid is purified. In some embodiments, the nucleic acid is purified using a precipitation method (e.g., LiCl precipitation, alcohol precipitation, or an equivalent method, e.g., as described herein). In some embodiments, the nucleic acid is purified using a chromatography-based method, such as an HPLC-based method or an equivalent method (e.g., as described herein). In some embodiments, the nucleic acid is purified using both a precipitation method (e.g., LiCl precipitation) and an HPLC-based method. In some embodiments, the nucleic acid is purified by tangential flow filtration (TFF).

### Cell Types

The ionizable lipid compounds and LNP compositions disclosed herein may be used for gene editing in vivo and in vitro. In one embodiment, one or more LNP compositions described herein may be administered to a subject in need thereof. In one embodiment, one or more LNP compositions described herein may contact a cell. In one embodiment, a therapeutically effective amount of a composition described herein may contact a cell of a subject in need thereof. In one embodiment, a genetically engineered cell may be produced by contacting a cell with an LNP composition described herein. In various embodiments, the methods comprise introducing a template nucleic acid to a cell or subject, as set forth above. In some embodiments, the cell is in vivo. In some embodiments, the cell is a liver cell. In preferred embodiments, the cell is a liver cell in vivo. In some embodiments, the cell is an immune cell. As used herein, “immune cell” refers to a cell of the immune system, including e.g., a lymphocyte (e.g., T cell, B cell, natural killer cell (“NK cell”, and NKT cell, or iNKT cell)), monocyte, macrophage, mast cell, dendritic cell, or granulocyte (e.g., neutrophil, eosinophil, and basophil). In some embodiments, the cell is a primary immune cell. In some embodiments, the immune system cell may be selected from CD3+, CD4+ and CD8+ T cells, regulatory T cells (Tregs), B cells, NK cells, and dendritic cells (DC). In some embodiments, the immune cell is allogeneic. In some embodiments, the cell is a lymphocyte. In some embodiments, the cell is an adaptive immune cell. In some embodiments, the cell is a T cell. In some embodiments, the cell is a B cell. In some embodiments, the cell is a NK cell.

While the inventions are described in conjunction with the illustrated embodiments, it is understood that they are not intended to limit the invention to those embodiments. On the contrary, the disclosure is intended to cover all alternatives, modifications, and equivalents, including equivalents of specific features, which may be included within the inventions as defined by the appended claims.

Both the foregoing general description and detailed description, as well as the following examples, are exemplary and explanatory only and are not restrictive of the teachings. The section headings used herein are for organizational purposes only and are not to be construed as limiting the desired subject matter in any way. In the event that any literature incorporated by reference contradicts any term defined in this specification, this specification controls. All ranges given in the application encompass the endpoints unless stated otherwise.

### Definitions

It should be noted that, as used in this application, the singular form “a”, “an” and “the” include plural references unless the context clearly dictates otherwise. Thus, for example, reference to “a composition” includes a plurality of compositions and reference to “a cell” includes a plurality of cells and the like. The use of “or” is inclusive and means “and/or” unless stated otherwise.

Unless specifically noted in the above specification, embodiments in the specification that recite “comprising” various components are also contemplated as “consisting of” or “consisting essentially of” the recited components; embodiments in the specification that recite “consisting of” various components are also contemplated as “comprising” or “consisting essentially of” the recited components; embodiments in the specification that recite “about” various components are also contemplated as “at” the recited components; and embodiments in the specification that recite “consisting essentially of” various components are also contemplated as “consisting of” or “comprising” the recited components (this interchangeability does not apply to the use of these terms in the claims).

Numeric ranges are inclusive of the numbers defining the range. Measured and measurable values are understood to be approximate, taking into account significant digits and the error associated with the measurement. As used in this application, the terms “about” and “approximately” have their art-understood meanings; use of one vs the other does not necessarily imply different scope. Unless otherwise indicated, numerals used in this application, with or without a modifying term such as “about” or “approximately”, should be understood to encompass normal divergence and/or fluctuations as would be appreciated by one of ordinary skill in the relevant art. In certain embodiments, the term “approximately” or “about” can refer to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.1%, or less in either direction (greater than or less than) of a stated reference value unless otherwise stated or otherwise evident from the context (except where such number would exceed 100% of a possible value).

As used herein, the term “contacting” means establishing a physical connection between two or more entities. For example, contacting a mammalian cell with a nanoparticle composition means that the mammalian cell and a nanoparticle are made to share a physical connection. Methods of contacting cells with external entities both in vivo and ex vivo are well known in the biological arts. For example, contacting a nanoparticle composition and a mammalian cell disposed within a mammal may be performed by varied routes of administration (e.g., intravenous, intramuscular, intradermal, and subcutaneous) and may involve varied amounts of nanoparticle compositions. Moreover, more than one mammalian cell may be contacted by a nanoparticle composition.

As used herein, the term “delivering” means providing an entity to a destination. For example, delivering a therapeutic and/or prophylactic to a subject may involve administering a nanoparticle composition including the therapeutic and/or prophylactic to the subject (e.g., by an intravenous, intramuscular, intradermal, or subcutaneous route). Administration of a nanoparticle composition to a mammal or mammalian cell may involve contacting one or more cells with the nanoparticle composition.

As used herein, “encapsulation efficiency” refers to the amount of a therapeutic and/or prophylactic that becomes part of a nanoparticle composition, relative to the initial total amount of therapeutic and/or prophylactic used in the preparation of a nanoparticle composition. For example, if 97 mg of therapeutic and/or prophylactic are encapsulated in a nanoparticle composition out of a total 100 mg of therapeutic and/or prophylactic initially provided to the composition, the encapsulation efficiency may be given as 97%. As used herein, “encapsulation” may refer to complete, substantial, or partial enclosure, confinement, surrounding, or encasement.

As used herein, the terms “editing efficiency”, “editing percentage”, “indel efficiency”, and “percent indels” refer to the total number of sequence reads with insertions or deletions relative to the total number of sequence reads. For example, editing efficiency at a target location in a genome may be measured by isolating and sequencing genomic DNA to identify the presence of insertions and deletions introduced by gene editing. In some embodiments, editing efficiency is measured as a percentage of cells that no longer contain a gene (e.g., CD3) after treatment, relative to the number of the cells that initially contained that gene (e.g., CD3+ cells).

As used herein, “knockdown” refers to a decrease in expression of a particular gene product (e.g., protein, mRNA, or both). Knockdown of a protein can be measured by detecting total cellular amount of the protein from a sample, such as a tissue, fluid, or cell population of interest. It can also be measured by measuring a surrogate, marker, or activity for the protein. Methods for measuring knockdown of mRNA are known and include sequencing of mRNA isolated from a sample of interest. In some embodiments, “knockdown” may refer to some loss of expression of a particular gene product, for example a decrease in the amount of mRNA transcribed or a decrease in the amount of protein expressed by a population of cells (including in vivo populations such as those found in tissues).

As used herein, “knockout” refers to a loss of expression from a particular gene or of a particular protein in a cell. Knockout can be measured by detecting total cellular amount of a protein in a cell, a tissue or a population of cells, for example. Knockout can also be detected at the genome or mRNA level, for example.

As used herein, the term “biodegradable” is used to refer to materials that, when introduced into cells, are broken down by cellular machinery (e.g., enzymatic degradation) or by hydrolysis into components that cells can either reuse or dispose of without significant toxic effect(s) on the cells. In certain embodiments, components generated by breakdown of a biodegradable material do not induce inflammation and/or other adverse effects in vivo. In some embodiments, biodegradable materials are enzymatically broken down. Alternatively or additionally, in some embodiments, biodegradable materials are broken down by hydrolysis.

As used herein, the “N/P ratio” is the molar ratio of ionizable nitrogen atom-containing lipid (e.g. Compound of Formula (I)-(IV)) to phosphate groups in RNA, e.g., in a nanoparticle composition including a lipid component and an RNA.

Compositions may also include salts of one or more compounds. Salts may be pharmaceutically acceptable salts. As used herein, “pharmaceutically acceptable salts” refers to derivatives of the disclosed compounds wherein the parent compound is altered by converting an existing acid or base moiety to its salt form (e.g., by reacting a free base group with a suitable organic acid). Examples of pharmaceutically acceptable salts include, but are not limited to, mineral or organic acid salts of basic residues such as amines; alkali or organic salts of acidic residues such as carboxylic acids; and the like. Representative acid addition salts include acetate, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like. The pharmaceutically acceptable salts of the present disclosure include the conventional non-toxic salts of the parent compound formed, for example, from non-toxic inorganic or organic acids. The pharmaceutically acceptable salts of the present disclosure can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418, Pharmaceutical Salts: Properties, Selection, and Use, P. H. Stahl and C. G. Wermuth (eds.), Wiley-VCH, 2008, and Berge et al., Journal of Pharmaceutical Science, 66, 1-19 (1977), each of which is incorporated herein by reference in its entirety.

As used herein, the “polydispersity index” is a ratio that describes the homogeneity of the particle size distribution of a system. A small value, e.g., less than 0.3, indicates a narrow particle size distribution. In some embodiments, the polydispersity index may be less than 0.1.

As used herein, “transfection” refers to the introduction of a species (e.g., an RNA) into a cell. Transfection may occur, for example, in vitro, ex vivo, or in vivo.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions, Wiley Interscience, New York, 1981; Wilen et al., Tetrahedron 33:2725 (1977); Eliel, E. L. Stereochemistry of Carbon Compounds, McGraw-Hill, NY, 1962; and Wilen, S. H., Tables of Resolving Agents and Optical Resolutions p. 268, E. L. Eliel, Ed., Univ. of Notre Dame Press, Notre Dame, IN 1972. The invention additionally encompasses compounds as individual isomers substantially free of other isomers, and alternatively, as mixtures of various isomers.

Unless otherwise stated, structures depicted herein are also meant to include all isomeric (e.g., enantiomeric, diastereomeric, and geometric (or conformational)) forms of the structure; for example, the R and S configurations for each stereocenter. Therefore, single stereochemical isomers as well as enantiomeric, diastereomeric, and geometric (or conformational) mixtures of the present compounds are within the scope of the invention. Unless otherwise stated, all tautomeric forms of the compounds of the invention are within the scope of the invention.

Unless otherwise stated, structures depicted herein are also meant to include compounds that differ only in the presence of one or more isotopically enriched atoms. For example, compounds having the present structures except for the replacement of hydrogen by deuterium or tritium, replacement of ¹⁹F with ¹⁸F, or the replacement of ¹²C with ¹³C or ¹⁴C are within the scope of the disclosure. Such compounds are useful, for example, as analytical tools or probes in biological assays.

When a range of values is listed, it is intended to encompass each value and sub-range within the range. For example, “C₁₋₆ alkyl” is intended to encompass C₁, C₂, C₃, C₄, C₅, C₆, C₁₋₆, C₁₋₅, C₁₋₄, C₁₋₃, C₁₋₂, C₂₋₆, C₂₋₃, C₂₋₄, C₂₋₃, C₃₋₆, C₃₋₅, C₃₋₄, C₄₋₆, C₄₋₅, and C₅₋₆ alkyl.

The term “alkyl” as used herein is a branched or unbranched saturated hydrocarbon group of 1 to 24 carbon atoms, such as methyl, ethyl, n-propyl, isopropyl, n-butyl, isobutyl, s-butyl, t-butyl, n-pentyl, isopentyl, s-pentyl, neopentyl, hexyl, heptyl, octyl, nonyl, decyl, dodecyl, tetradecyl, hexadecyl, eicosyl, tetracosyl, and the like. The alkyl group can be cyclic or acyclic. The alkyl group can be branched or unbranched (i.e., linear). The alkyl group can also be substituted or unsubstituted. For example, the alkyl group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein. A “lower alkyl” group is an alkyl group containing from one to six (e.g., from one to four) carbon atoms.

The term “alkenyl”, as used herein, refers to an aliphatic group containing at least one carbon-carbon double bond and 2 to 12 carbon atoms, and is intended to include both “unsubstituted alkenyls” and “substituted alkenyls”, the latter of which refers to alkenyl moieties having substituents replacing a hydrogen on one or more carbons of the alkenyl group. Such substituents may occur on one or more carbons that are included or not included in one or more double bonds. Moreover, such substituents include all those contemplated for alkyl groups, as discussed below, except where stability is prohibitive. For example, an alkenyl group may be substituted by one or more alkyl, carbocyclyl, aryl, heterocyclyl, or heteroaryl groups is contemplated. Exemplary alkenyl groups include, but are not limited to, vinyl (—CH═CH₂), allyl (—CH₂CH═CH₂), cyclopentenyl (—C5H7), and 5-hexenyl (—CH₂CH₂CH₂CH₂CH═CH₂). In some embodiments, a heteroalkenyl group has 3 to 10 carbon atoms and one or more carbon-carbon double bonds (e.g., 1, 2, 3, or 4 double bonds). In some embodiments, an alkenyl group has 3 to 9 carbon atoms (“C₃₋₉ alkenyl”). In some embodiments, an alkenyl group has 3 to 8 carbon atoms (“C₃₋₈ alkenyl”). In some embodiments, an alkenyl group has 3 to 7 carbon atoms (“C₃₋₇ alkenyl”). In some embodiments, an alkenyl group has 3 to 6 carbon atoms (“C₃₋₆ alkenyl”). In some embodiments, an alkenyl group has 3 to 5 carbon atoms (“C₃₋₅ alkenyl”). In some embodiments, an alkenyl group has 3 to 4 carbon atoms (“C₃₋₄ alkenyl”). In some embodiments, an alkenyl group has 3 carbon atoms (“C₃ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₃₋₁₀ alkenyl. In certain embodiments, the alkenyl group is a substituted C₃₋₁₀ alkenyl. In an alkenyl group, a C═C double bond for which the stereochemistry is not specified (e.g., —CH═CHCH₃ or

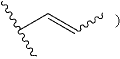

may be an (E)- or (Z)-double bond.

The term “alkynyl”, as used herein, refers to an aliphatic group containing at least one carbon-carbon triple bond and is intended to include both “unsubstituted alkynyls” and “substituted alkynyls”, the latter of which refers to alkynyl moieties having substituents replacing a hydrogen on one or more carbons of the alkynyl group. Such substituents may occur on one or more carbons that are included or not included in one or more double bonds. Moreover, such substituents include all those contemplated for alkyl groups, as discussed below, except where stability is prohibitive. For example, an alkynyl group may be substituted by one or more alkyl, carbocyclyl, aryl, heterocyclyl, or heteroaryl groups is contemplated. The term “alkynyl” refers to a radical of a straight-chain or branched hydrocarbon group having from 2 to 12 carbon atoms and one or more carbon-carbon triple bonds (e.g., 1, 2, 3, or 4 triple bonds) (“C₂₋₁₂ alkynyl”). In some embodiments, an alkynyl group has 3 to 10 carbon atoms (“C₃₋₁₀ alkynyl”). In some embodiments, an alkynyl group has 3 to 9 carbon atoms (“C₃₋₉ alkynyl”). In some embodiments, an alkynyl group has 3 to 8 carbon atoms (“C₃₋₈ alkynyl”). In some embodiments, an alkynyl group has 3 to 7 carbon atoms (“C₃₋₇ alkynyl”). In some embodiments, an alkynyl group has 3 to 6 carbon atoms (“C₃₋₆ alkynyl”). In some embodiments, an alkynyl group has 3 to 5 carbon atoms (“C₃₋₅ alkynyl”). In some embodiments, an alkynyl group has 3 to 4 carbon atoms (“C₃₋₄ alkynyl”). In some embodiments, an alkynyl group has 3 carbon atoms (“C₃ alkynyl”). The one or more carbon-carbon triple bonds can be internal (such as in 2-butynyl) or terminal (such as in 1-butynyl).

An “alkylene” group refers to a divalent alkyl radical, which may be branched or unbranched (i.e., linear). Any of the above mentioned monovalent alkyl groups may be converted to an alkylene by abstraction of a second hydrogen atom from the alkyl. Representative alkylenes include C₂₋₄ alkylene and C₂₋₃ alkylene. Typical alkylene groups include, but are not limited to —CH(CH₃)—, —C(CH₃)₂—, —CH₂CH₂—, —CH₂CH(CH₃)—, —CH₂C(CH₃)₂—, —CH₂CH₂CH₂—, —CH₂CH₂CH₂CH₂—, and the like. The alkylene group can also be substituted or unsubstituted. For example, the alkylene group can be substituted with one or more groups including, but not limited to, alkyl, aryl, heteroaryl, cycloalkyl, alkoxy, amino, ether, halide, hydroxy, nitro, silyl, sulfoxo, sulfonate, carboxylate, or thiol, as described herein.

The term “alkenylene” includes divalent, straight or branched, unsaturated, acyclic hydrocarbyl groups having at least one carbon-carbon double bond and, in one embodiment, no carbon-carbon triple bonds. Any of the above-mentioned monovalent alkenyl groups may be converted to an alkenylene by abstraction of a second hydrogen atom from the alkenyl. Representative alkenylenes include C₂₋₆alkenylenes.

The term “C_{x-y}” when used in conjunction with a chemical moiety, such as alkyl or alkylene, is meant to include groups that contain from x to y carbons in the chain. For example, the term “C_{x-y} alkyl” refers to substituted or unsubstituted saturated hydrocarbon groups, including straight-chain and branched-chain alkyl and alkylene groups that contain from x to y carbons in the chain.

The term “alkoxy” refers to an alkyl group, preferably a lower alkyl group, having an oxygen attached thereto. Representative alkoxy groups include methoxy, ethoxy, propoxy, tert-butoxy and the like.

In some embodiments, the present disclosure relates to a compound represented by structural Formula I

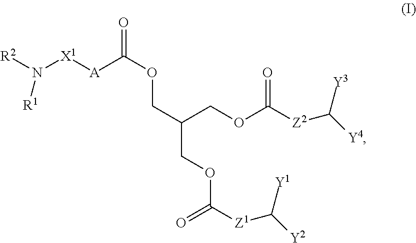

or a salt thereof, wherein:
A is O or NH,
X¹ is a C₁₋₅ alkylene,
R¹ and R² is each independently a C₁₋₃ alkyl, or
R¹ taken together with R² and the nitrogen atom to which they are attached form a 5-6-, or 7-membered ring, and
Z¹ is a C₁₋₅ alkylene,
Y¹ and Y² is each independently a C₃₋₁₀ alkoxyl or O(C₃₋₁₀ alkynyl),
Z² is a C₁₋₅ alkylene or a direct bond, and
Y³ and Y⁴ is each independently a C₃₋₁₀ alkoxyl or O(C₃₋₁₀ alkynyl), or
Y³ and Y⁴ is each independently a C₃₋₁₀ alkyl or C₃₋₁₀ alkynyl,
provided that if Y¹, Y², Y³, and Y⁴ is each independently a C₃₋₁₀ alkoxy, then R¹ and R² are not C₂ alkyl, and R¹ taken together with R² and the nitrogen atom to which they are attached do not form a 6-membered ring.

In some embodiments, A is O. Alternatively, A is NH.

In some embodiments, X¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, X¹ is a C₂₋₃ alkylene, such as a C₂ alkylene or a C₃ alkylene.

In some embodiments, Z¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, Z¹ is a C₂₋₃ alkylene, such as a C₂ alkylene or a C₃ alkylene.

In some embodiments, Z² is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, Z¹ is a C₂₋₃ alkylene, such as a C₂ alkylene or a C₃ alkylene. In some embodiments, Z² is a direct bond.

In some embodiments, Z¹ is a C₂₋₃ alkylene and Z² is a direct bond.

In some embodiments, Y¹ and Y² is each independently a C₃₋₉ alkoxyl. For example, Y¹ and Y² is each independently a C₄₋₉ alkoxyl, C₅₋₉ alkoxyl, C₆₋₉ alkoxyl, C₇₋₉ alkoxyl, C₈₋₉ alkoxyl, C₃₋₈ alkoxyl, C₃₋₇ alkoxyl, C₃₋₆ alkoxyl, C₃₋₅ alkoxyl, C₃₋₄ alkoxyl, C₄₋₈ alkoxyl, C₄₋₇ alkoxyl, C₄₋₆ alkoxyl, C₄₋₅ alkoxyl, C₅₋₈ alkoxyl, C₅₋₇ alkoxyl, C₅₋₆ alkoxyl, C₆₋₈ alkoxyl, C₆₋₇ alkoxyl, or C₇₋₈ alkoxy. For example, Y¹ and Y² is each independently a C₆₋₉ alkoxyl.

In some embodiments, Y³ and Y⁴ is each independently a C₃₋₉ alkoxyl. For example, Y³ and Y⁴ is each independently a C₄₋₉ alkoxyl, C₅₋₉ alkoxyl, C₆₋₉ alkoxyl, C₇₋₉ alkoxyl, C₈₋₉ alkoxyl, C₃₋₈ alkoxyl, C₃₋₇ alkoxyl, C₃₋₆ alkoxyl, C₃₋₅ alkoxyl, C₃₋₄ alkoxyl, C₄₋₈ alkoxyl, C₄₋₇ alkoxyl, C₄₋₆ alkoxyl, C₄₋₅ alkoxyl, C₅₋₈ alkoxyl, C₅₋₇ alkoxyl, C₅₋₆ alkoxyl, C₆₋₈ alkoxyl, C₆₋₇ alkoxyl, or C₇₋₈ alkoxy. For example, Y³ and Y⁴ is each independently a C₆₋₉ alkoxyl.

In some embodiments, Y³ and Y⁴ is each independently a C₃₋₉ alkyl. For example, Y³ and Y⁴ is each independently a C₄₋₉ alkyl, C₅₋₉ alkyl, C₆₋₉ alkyl, C₇₋₉ alkyl, C₈₋₉ alkyl, C₃₋₈ alkyl, C₃₋₇ alkyl, C₃₋₆ alkyl, C₃₋₅ alkyl, C₃₋₄ alkyl, C₄₋₈ alkyl, C₄₋₇ alkyl, C₄₋₆ alkyl, C₄₋₅ alkyl, C₅₋₈ alkyl, C₅₋₇ alkyl, C₅₋₆ alkyl, C₆₋₈ alkyl, C₆₋₇ alkyl, or C₇₋₈ alkyl. For example, Y³ is a C₆₋₉ alkyl and Y⁴ is a C₃₋₅ alkyl.

In some embodiments, R¹ and R² is each independently a C₁₋₃ alkyl. For example, R¹ and R² is each independently methyl ethyl, propyl, or isopropyl.

In some embodiments, the compound of Formula I is represented by one of the following structural formulas:

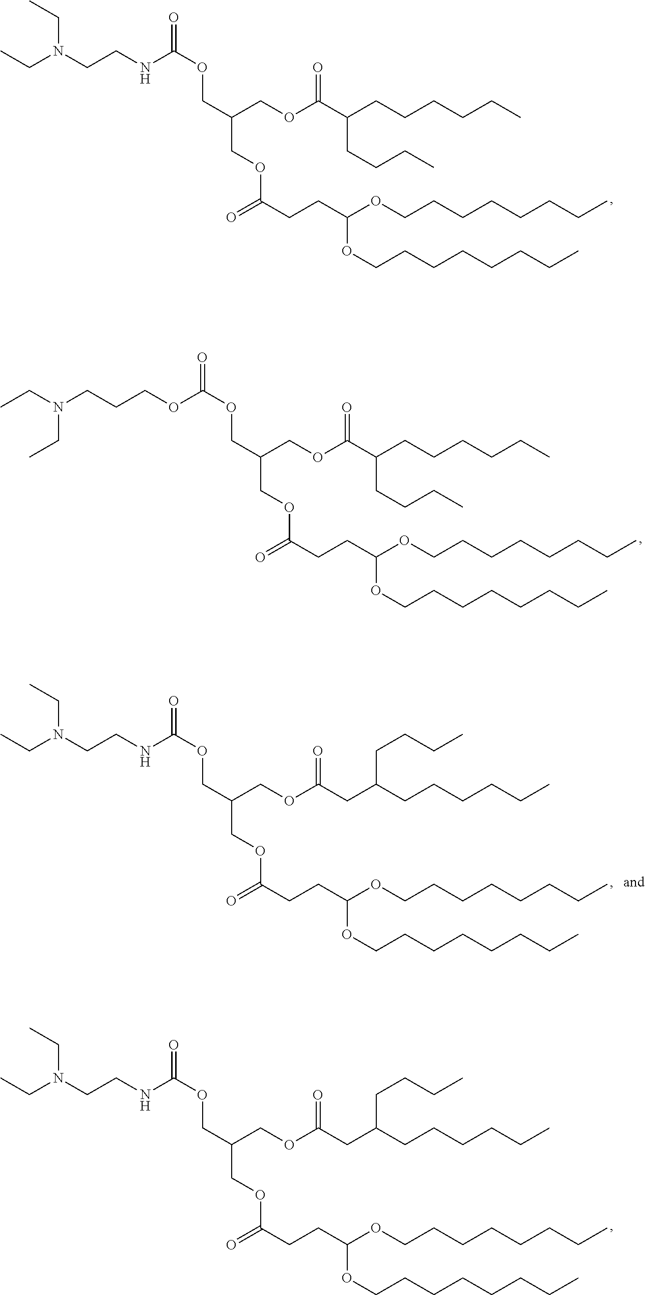

or a salt thereof.

In some embodiments, the present disclosure relates to compound of represented by structural Formula II,

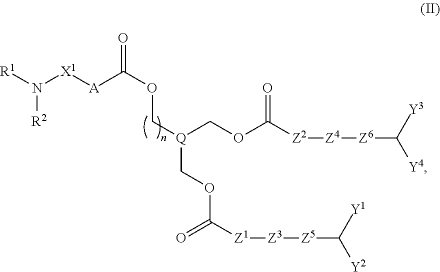

or a salt thereof,
wherein,
Q is CR³ or

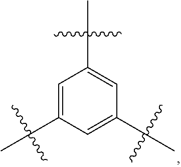 [SMILES: *c1cc(*)cc(*)c1]

A is O, NH, or a direct bond,
X¹ is a C₁₋₅ alkylene,
R¹ and R² is each independently a C₁₋₃ alkyl, or
R¹ taken together with the nitrogen atom to which it is attached and 1-3 carbon atoms of X¹ form a 4-, 5-, or 6-membered ring, or
R¹ taken together with R² and the nitrogen atom to which they are attached form a 5-, 6-, or 7-membered ring, and
R³ is H or C₁₋₃ alkyl,
Z¹ and Z² is each independently a C₁₋₅ alkylene,
Z³ and Z⁴ is each independently a —C(═O)O— in either direction,
Z⁵ and Z⁶ is each independently a direct bond or a C₁₋₃ alkylene,
Y¹ is selected from H, a C₁₋₁₀ alkyl, C₃₋₁₀ alkenyl, and C₃₋₁₀ alkynyl,
Y², Y³, and Y⁴ is each independently selected from a C₃₋₁₀ alkyl, C₃₋₁₀ alkenyl, and C₃₋₁₀ alkynyl, and
n is 0 or 1.

In some embodiments, Q is

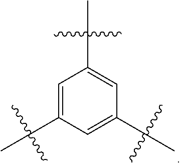 [SMILES: *c1cc(*)cc(*)c1]

In some embodiments, the compound of Formula II is represented by structural formula IIa,

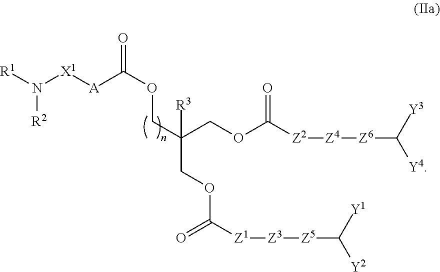

In some embodiments, n is 0. Alternatively, n is 1

In some embodiments, A is O. Alternatively, A is NH. Alternatively yet, A is a direct bond.

In some embodiments, X¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, X¹ is a C₂₋₃ alkylene, such as a C₂ alkylene or a C₃ alkylene. For example, X¹ is C₂ alkylene.

In some embodiments, Z¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, Z¹ is a C₂₋₃ alkylene, such as a C₂ alkylene or a C₃ alkylene.

In some embodiments, Z² is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, Z¹ is a C₂₋₃ alkylene, such as a C₂ alkylene or a C₃ alkylene.

In some embodiments, Z¹ and Z² is each independently C₃ alkylene. In some embodiments, Z¹ and Z² is each independently C₄ alkylene. In some embodiments, Z¹ and Z² is each independently C₅ alkylene.

In certain embodiments, Z¹ is C₂ alkylene; and Z² is C₃ alkylene.

In some embodiments, Z¹ and Z² is each independently C₃ alkylene, A is NH, and X¹ is a C₂ alkylene.

In some embodiments, Z³ and Z⁴ is each

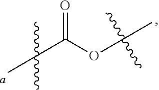 [SMILES: *OC(*)=O]

wherein a indicates the point of attachment to Z¹ and Z², respectively.

In some embodiments, Z³ is

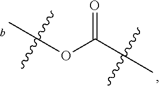 [SMILES: *OC(*)=O]

wherein b indicates the point of attachment to Z¹; and Z⁴ is

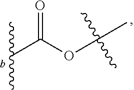 [SMILES: *OC(*)=O]

wherein b indicates the point of attachment to Z².

In some embodiments, Z⁵ and Z⁶ is each independently a direct bond.

In some embodiments, Z⁵ is C₁ alkylene; and Z⁶ is a direct bond.

In some embodiment, Y¹ is H.

In some embodiments, Y¹, Y², Y³, and Y⁴ is each independently a C₃₋₉ alkyl. For example, Y¹, Y², Y³, and Y⁴ is each independently a C₄₋₉ alkyl, C₅₋₉ alkyl, C₆₋₉ alkyl, C₇₋₉ alkyl, C₈₋₉ alkyl, C₃₋₈ alkyl, C₃₋₇ alkyl, C₃₋₆ alkyl, C₃₋₅ alkyl, C₃₋₄ alkyl, C₄₋₈ alkyl, C₄₋₇ alkyl, C₄₋₆ alkyl, C₄₋₅ alkyl, C₅₋₈ alkyl, C₅₋₇ alkyl, C₅₋₆ alkyl, C₆₋₈ alkyl, C₆₋₇ alkyl, or C₇₋₈ alkyl. For example, Y¹, Y², Y³, and Y⁴ is each independently a C₇₋₉ alkyl, such as C₈ alkyl.

In some embodiments, Y¹ and Y² is each independently a C₅₋₇ alkyl and Y³ and Y⁴ is each independently a C₃₋₅ alkyl.

In some embodiments, R¹ and R² is each independently a C₁₋₃ alkyl. For example, R¹ and R² is each independently methyl ethyl, propyl, or isopropyl. For example, R¹ and R² is each C₂ alkyl.

In some embodiments, R¹ taken together with R² and the nitrogen atom to which they are attached form a 5-, 6-, or 7-membered ring. For example, R¹ taken together with R² and the nitrogen atom to which they are attached forms a 5-membered ring. Alternatively, R¹ taken together with R² and the nitrogen atom to which they are attached forms a 6-membered ring. Alternatively yet, R¹ taken together with R² and the nitrogen atom to which they are attached forms a 7-membered ring.

In some embodiments, the compound of Formula II is represented by one of the following structural formulas:

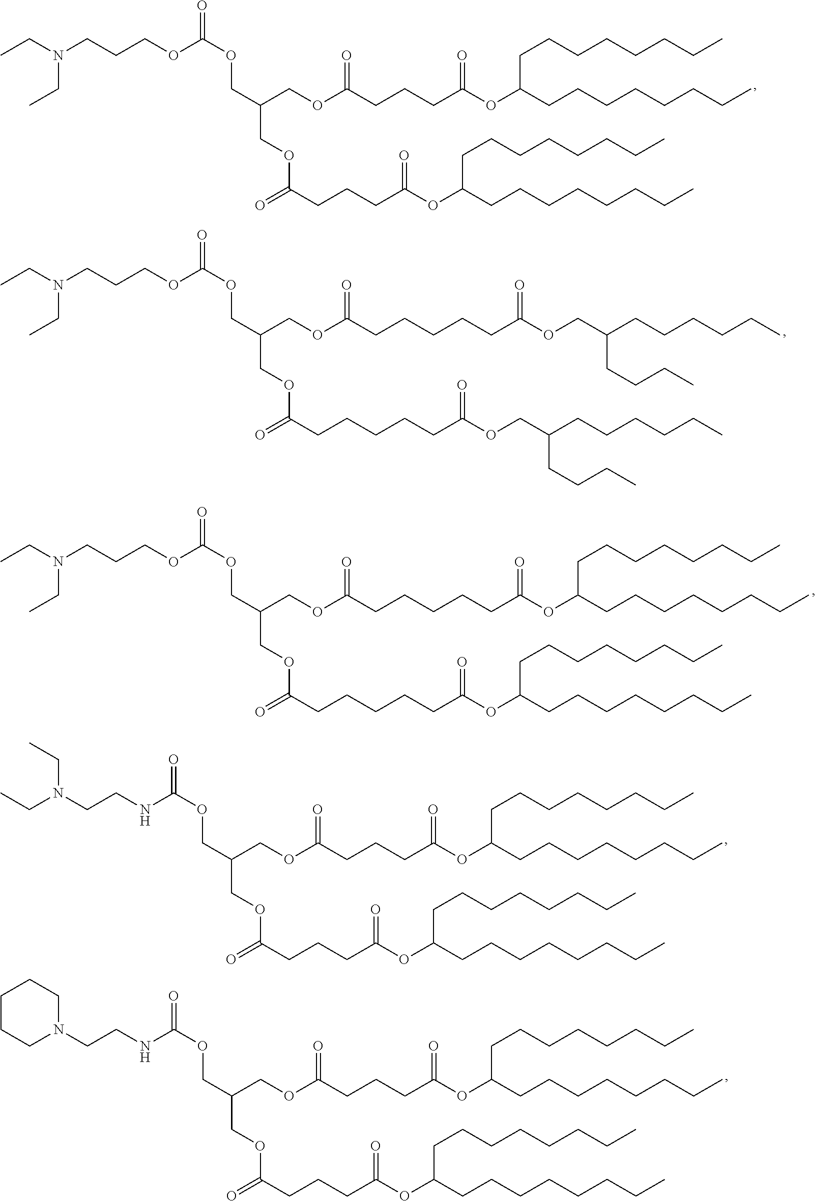

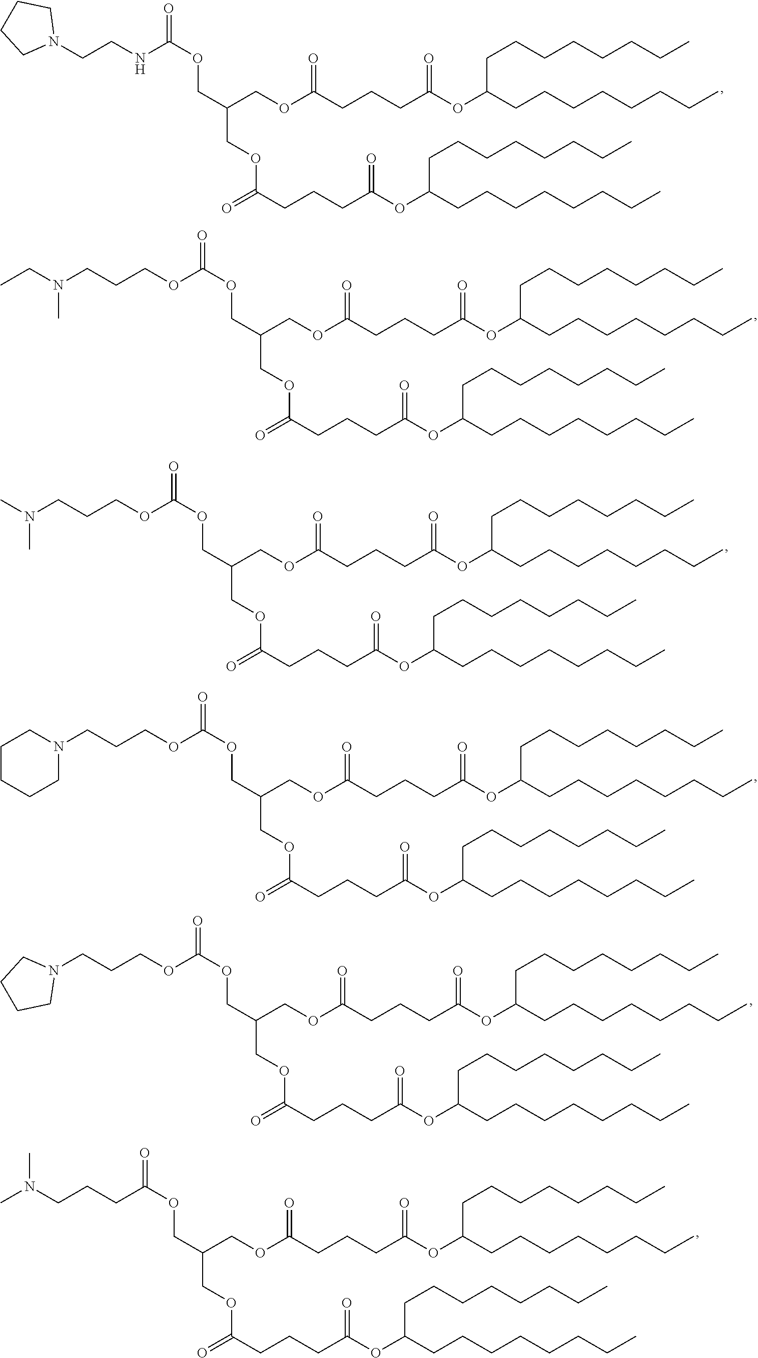

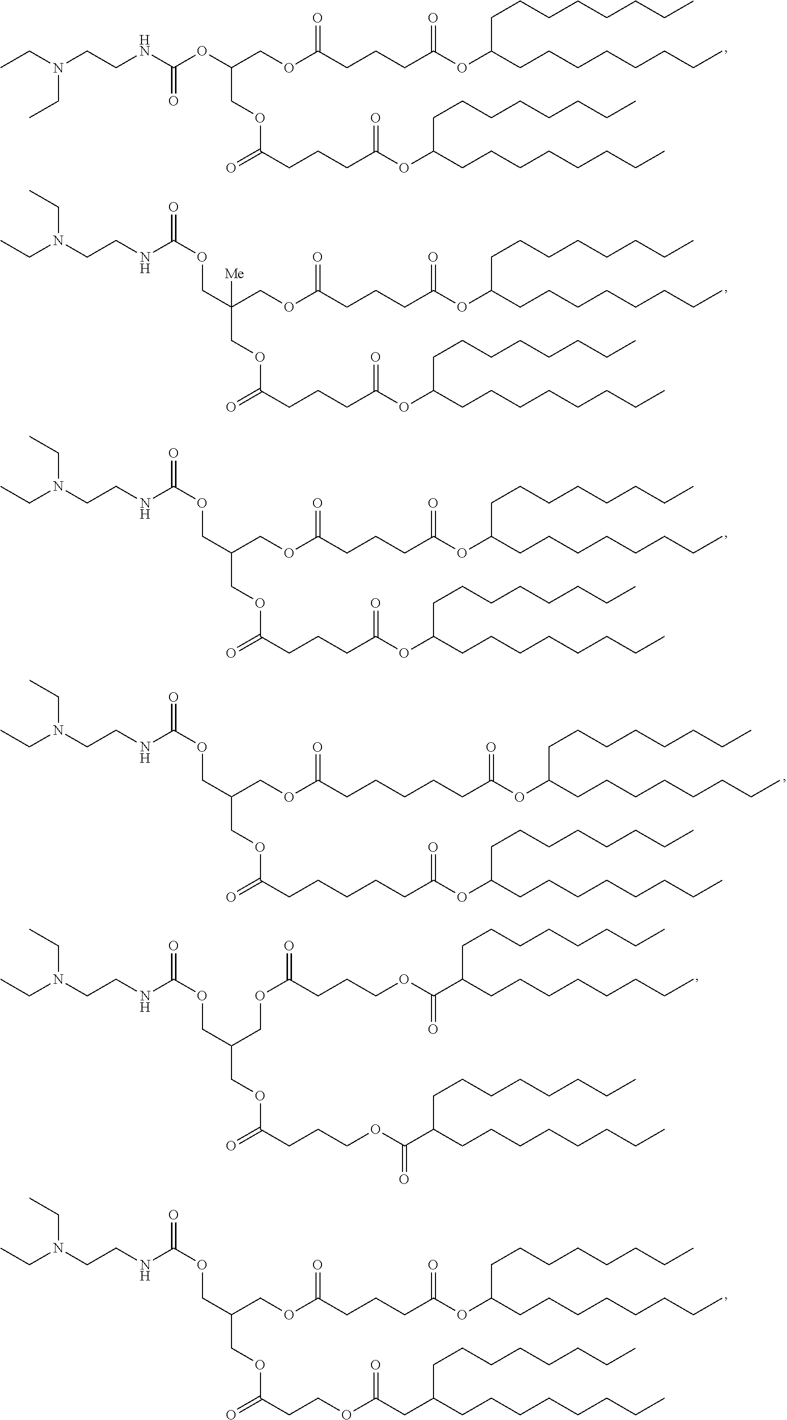

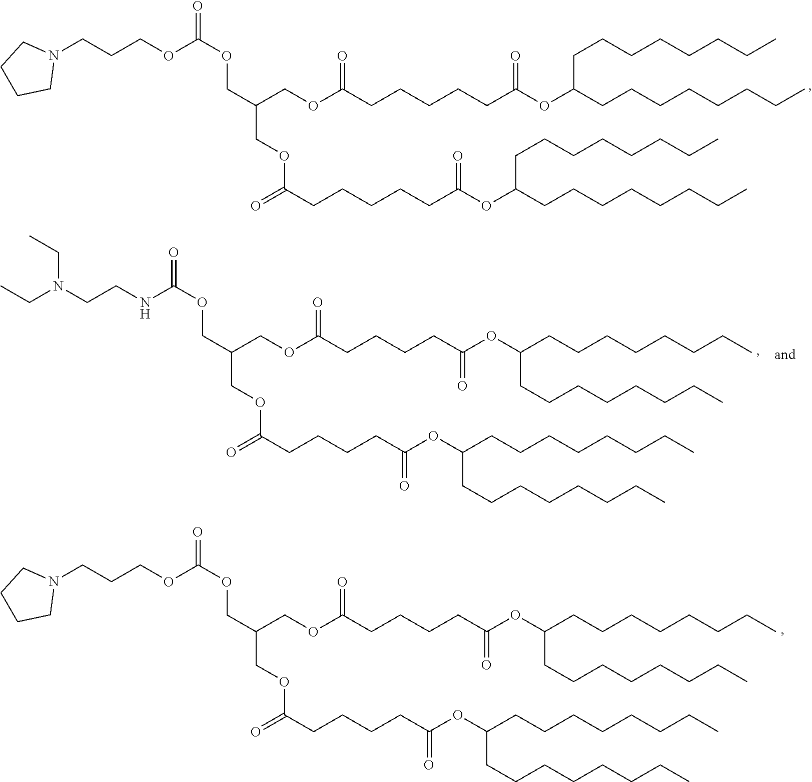

or a salt thereof.

In some embodiments, the compound of Formula II is represented by one of the following structural formulas:

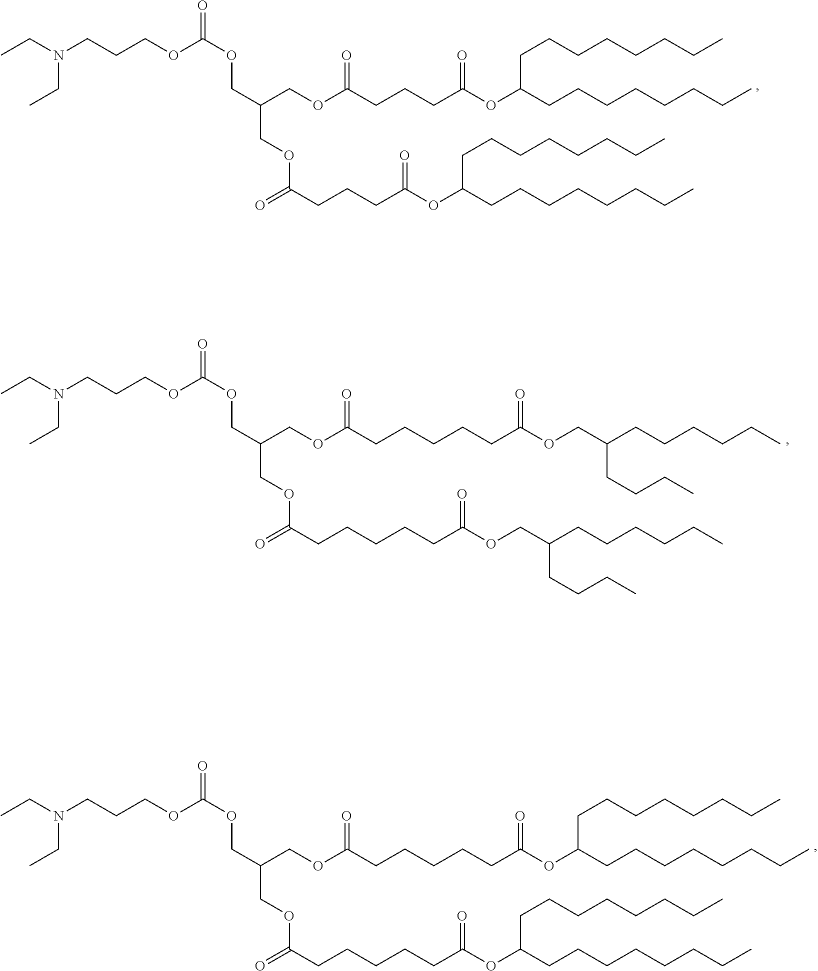

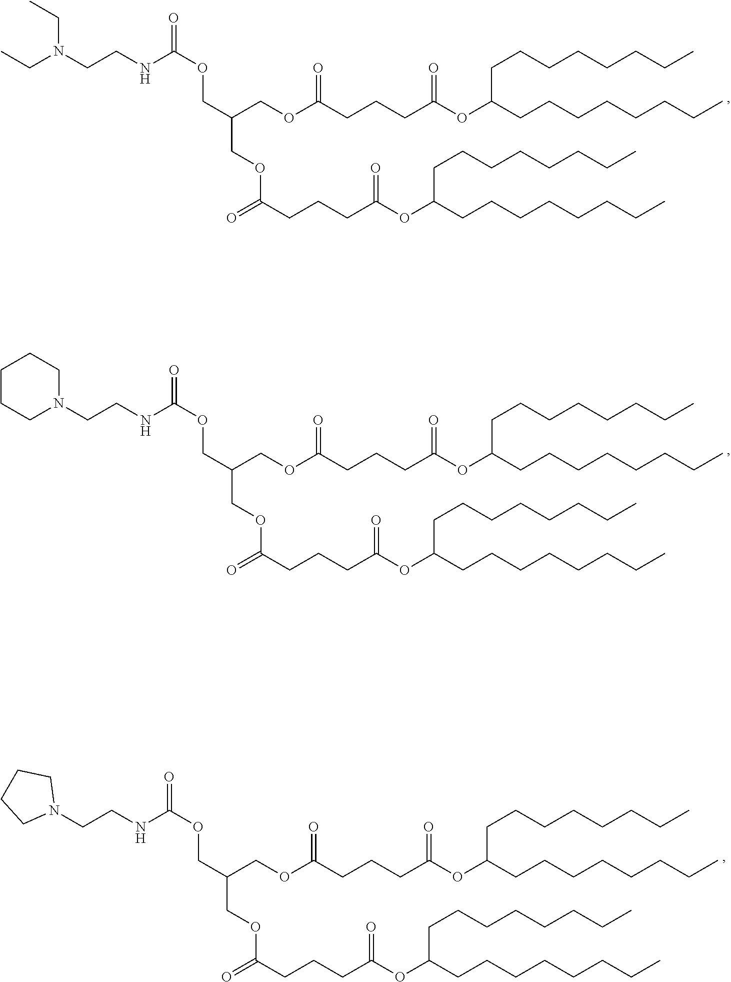 [SMILES: CCCCCCCCC(CCCCCCCC)OC(=O)CCCC(=O)OCC(COC(=O)CCCC(=O)OC(CCCCCCCC)CCCCCCCC)COC(=O)NCCN(CC)CC ; CCCCCCCCC(CCCCCCCC)OC(=O)CCCC(=O)OCC(COC(=O)CCCC(=O)OC(CCCCCCCC)CCCCCCCC)COC(=O)NCCN1CCCCC1 ; CCCCCCCCC(CCCCCCCC)OC(=O)CCCC(=O)OCC(COC(=O)CCCC(=O)OC(CCCCCCCC)CCCCCCCC)COC(=O)NCCN1CCCC1]

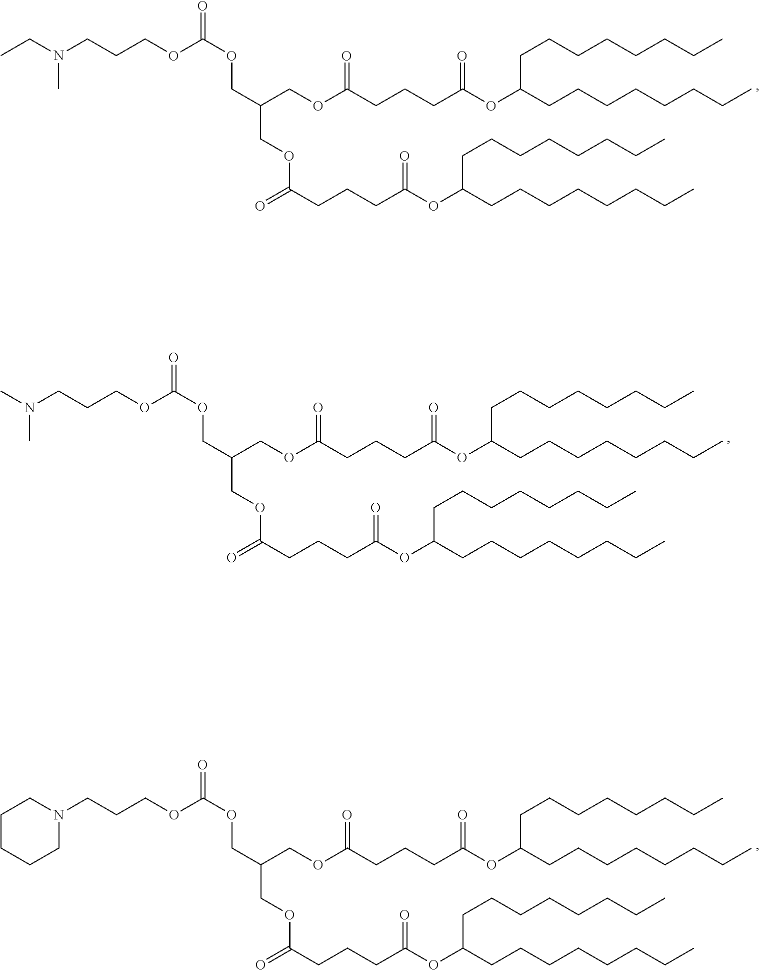

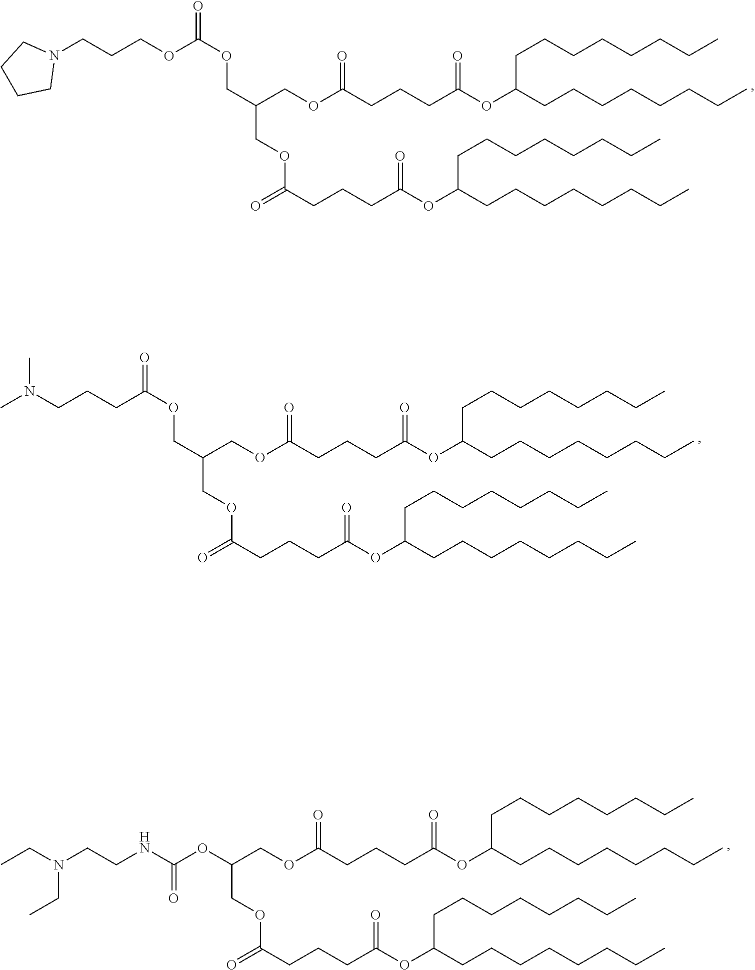

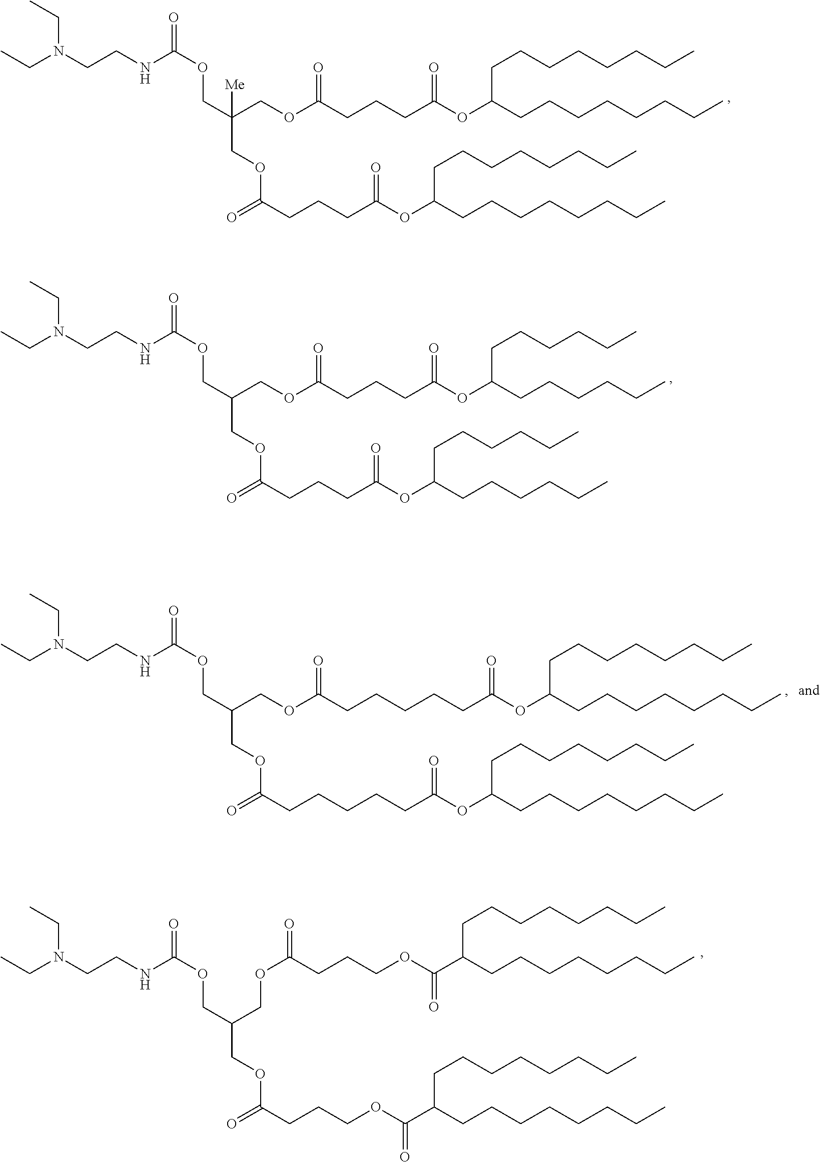

or a salt thereof.

In some embodiments the present disclosure relates to a compound of represented by structural Formula III,

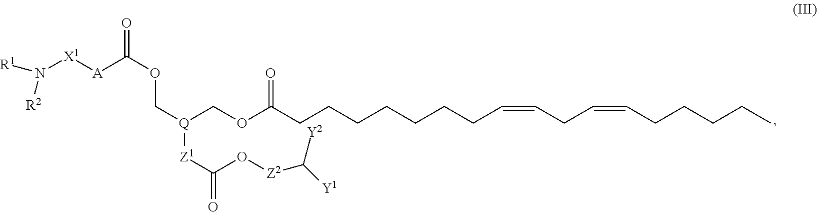

or a salt thereof,
wherein:
Q is CH or

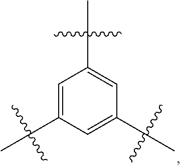 [SMILES: *c1cc(*)cc(*)c1]

A is O or NH,
X¹ is a C₁₋₅ alkylene,
R¹ and R² is each independently a C₁₋₃ alkyl, or
R¹ taken together with the nitrogen atom to which it is attached and 1-3 carbon atoms of X¹ form a 4-, 5-, or 6-membered ring, or
R¹ taken together with R² and the nitrogen atom to which they are attached form a 5-, 6-, or 7-membered ring, and
Z¹ is a C₂₋₉ alkylene,
Z² is a C₁₋₃ alkylene or a direct bond, and
Y¹ and Y² is each independently selected from a C₃₋₁₀ alkyl, C₃₋₁₀ alkenyl, and C₃₋₁₀ alkynyl.

In some embodiments, Q is

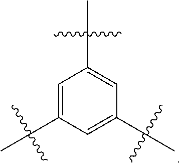 [SMILES: *c1cc(*)cc(*)c1]

In some embodiments, the compound of Formula III is represented by structural Formula IIIa,

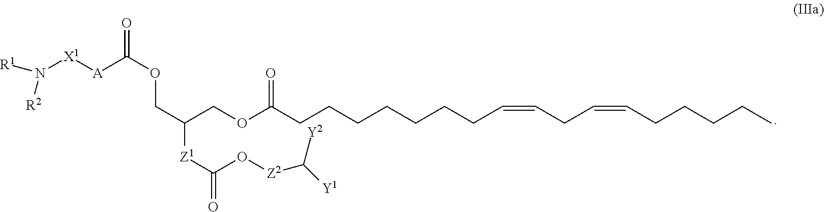

In some embodiments, A is O. Alternatively, A is NH.

In some embodiments, X¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, X¹ is a C₂₋₃ alkylene, such as a C₂ alkylene or a C₃ alkylene. For example, X¹ is a C₃ alkylene.

In some embodiments, Z¹ is a C₃₋₉ alkylene. For example, Z¹ is a C₄₋₉ alkylene, C₅₋₉ alkylene, C₆₋₉ alkylene, C₇₋₉ alkylene, C₈₋₉ alkylene, C₃₋₈ alkylene, C₃₋₇ alkylene, C₃₋₆ alkylene, C₃₋₅ alkylene, C₃₋₄ alkylene, C₄₋₈ alkylene, C₄₋₇ alkylene, C₄₋₆ alkylene, C₄₋₅ alkylene, C₅₋₈ alkylene, C₅₋₇ alkylene, C₅₋₆ alkylene, C₆₋₈ alkylene, C₆₋₇ alkylene, or C₇₋₈ alkoxy. For example, Z¹ is a C₃₋₅ alkylene, C₅₋₇ alkylene, or C₇₋₉ alkylene.

In some embodiment, Z² is a direct bond. In some embodiment, Z² is a C₁₋₃ alkylene.

In some embodiments, Y¹ and Y² is each independently a C₃₋₉ alkyl. For example, Y¹ and Y² is each independently a C₄₋₉ alkyl, C₅₋₉ alkyl, C₆₋₉ alkyl, C₇₋₉ alkyl, C₅₋₉ alkyl, C₃₋₈ alkyl, C₃₋₇ alkyl, C₃₋₆ alkyl, C₃₋₅ alkyl, C₃₋₄ alkyl, C₄₋₈ alkyl, C₄₋₇ alkyl, C₄₋₆ alkyl, C₄₋₅ alkyl, C₅₋₈ alkyl, C₅₋₇ alkyl, C₅₋₆ alkyl, C₆₋₈ alkyl, C₆₋₇ alkyl, or C₇₋₈ alkyl. In some embodiments, Y¹ and Y² is each independently a C₃₋₅ alkyl, C₅₋₇ alkyl, or C₇₋₉ alkyl.

In some embodiments, the compound of Formula III is represented by one of the following structural formulas:

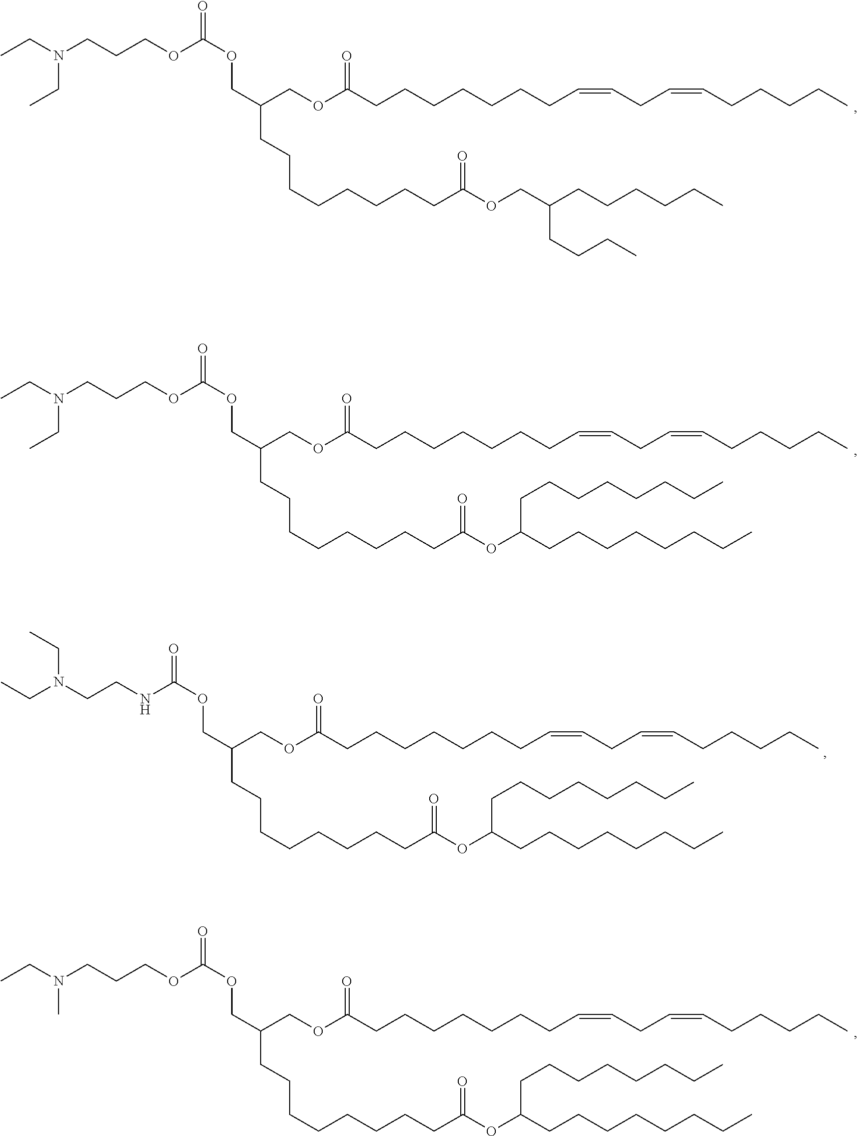

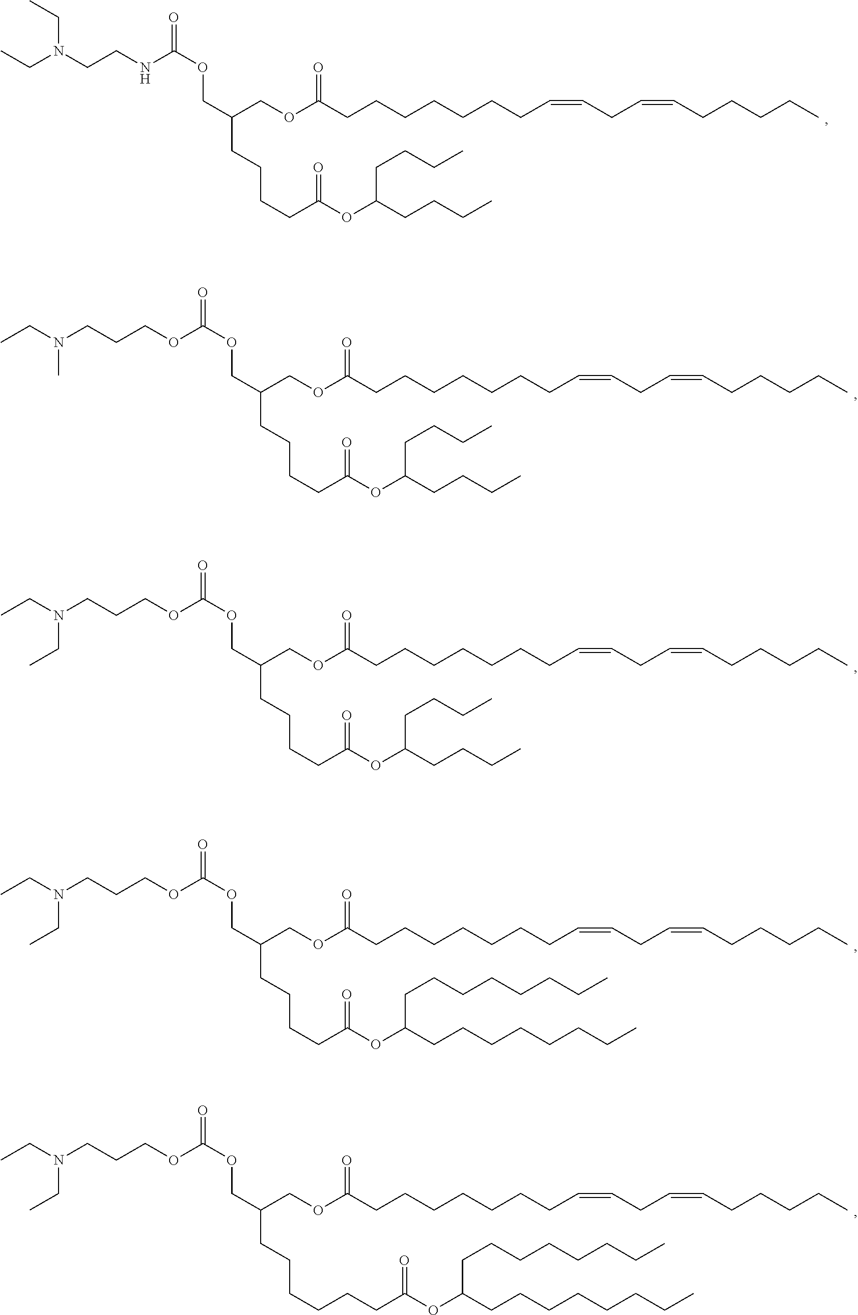

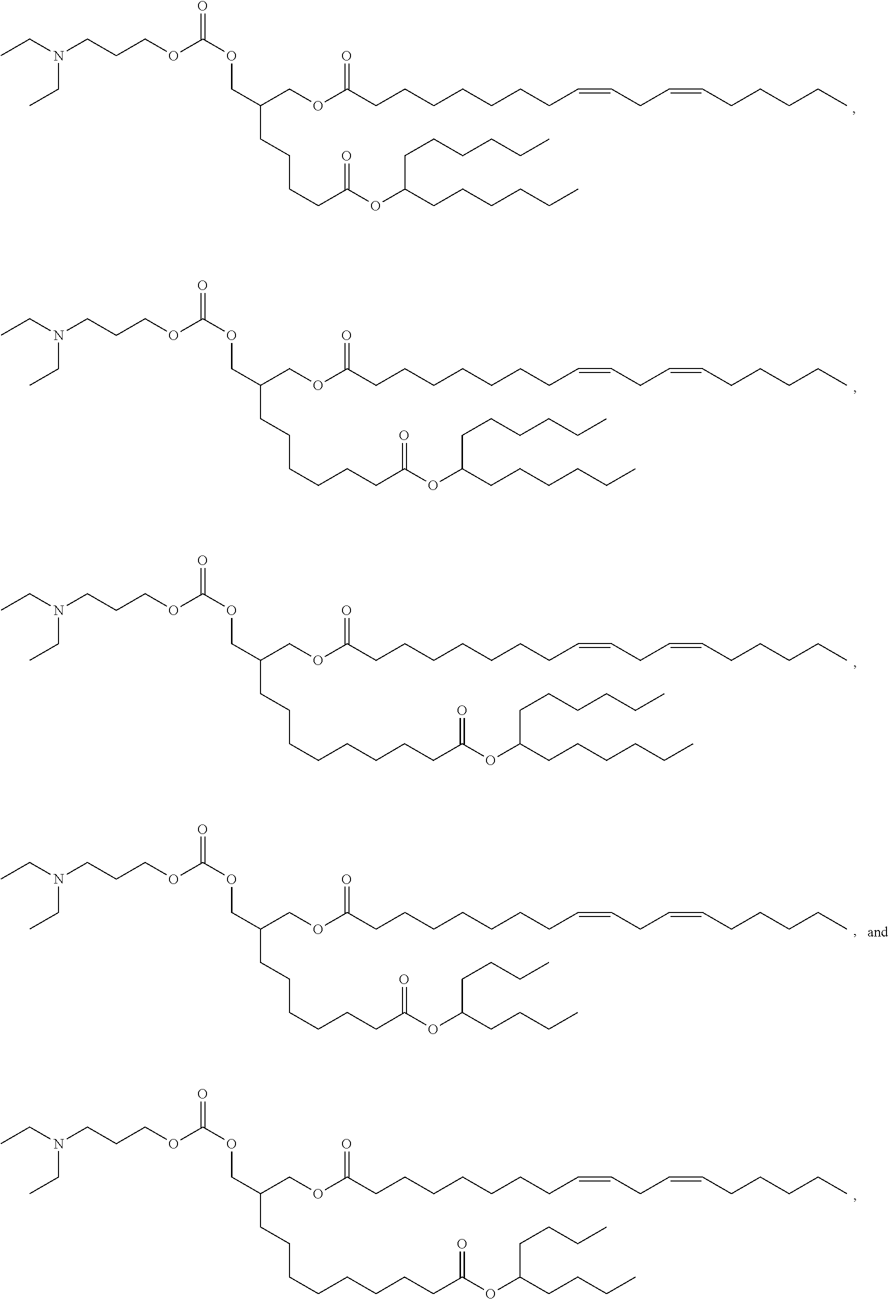

or a salt thereof.

In some embodiments the present disclosure relates to a compound of represented by structural Formula IV,

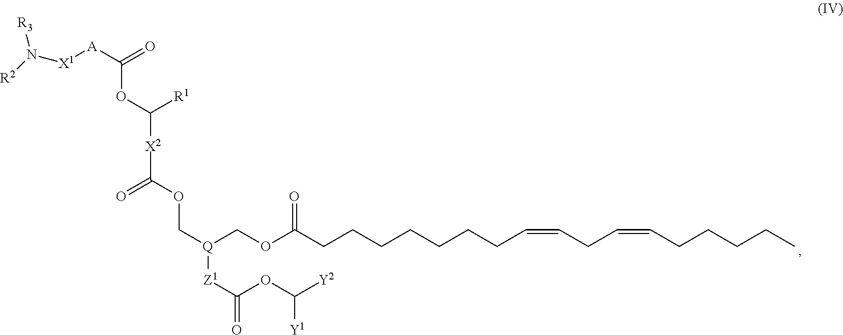

or a salt thereof,
wherein:
Q is CH or

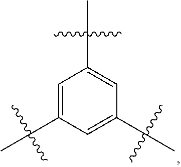 [SMILES: *c1cc(*)cc(*)c1]

A is O, NH, or a direct bond,
X¹ and X² is each independently a C₁₋₅ alkylene,
R¹ is selected from a C₃₋₉ alkyl, C₃₋₉ alkenyl, and C₃₋₉ alkynyl,
R² and R³ is each independently a C₁₋₃ alkyl, or
R² taken together with R³ and the nitrogen atom to which they are attached form a 5-, 6-, or 7-membered ring, and
Z¹ is a C₆₋₁₀ alkylene, and
Y¹ and Y² is each independently selected from a C₃₋₁₀ alkyl, C₃₋₁₀ alkenyl, and C₃₋₁₀ alkynyl.

In some embodiments, Q is

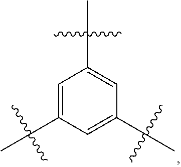 [SMILES: *c1cc(*)cc(*)c1]

In some embodiments, the compound of Formula IV is represented by structural formula IVa,

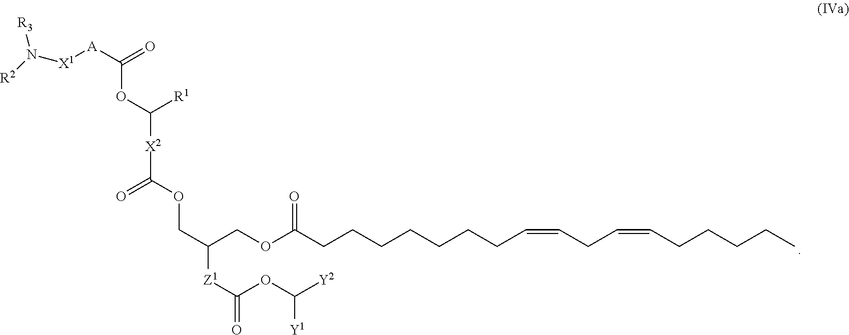

In some embodiments, A is O. Alternatively, A is NH. Alternatively yet, A is a direct bond.

In some embodiments, X¹ is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, X¹ is a C₂₋₃ alkylene, such as a C₂ alkylene or a C₃ alkylene.

In some embodiments, X² is a C₁₋₄ alkylene, C₁₋₃ alkylene, C₁₋₅ alkylene, C₁₋₂ alkylene, C₂₋₅ alkylene, C₂₋₄ alkylene, or C₂₋₃ alkylene. For example, X¹ is a C₂₋₃ alkylene, such as a C₂ alkylene or a C₃ alkylene.

In some embodiments, Z¹ is a C₃₋₉ alkylene. For example, Z¹ is a C₄₋₉ alkylene, C₅₋₉ alkylene, C₆₋₉ alkylene, C₇₋₉ alkylene, C₅₋₉ alkylene, C₃₋₈ alkylene, C₃₋₇ alkylene, C₃₋₆ alkylene, C₃₋₅ alkylene, C₃₋₄ alkylene, C₄₋₈ alkylene, C₄₋₇ alkylene, C₄₋₆ alkylene, C₄₋₅ alkylene, C₅₋₈ alkylene, C₅₋₇ alkylene, C₅₋₆ alkylene, C₆₋₈ alkylene, C₆₋₇ alkylene, or C₇₋₈ alkoxy. For example, Z¹ is a C₇₋₉ alkylene.

In some embodiments, Y¹ and Y² is each independently a C₃₋₁₀ alkyl. For example, Y¹ and Y² is each independently a C₄₋₁₀ alkyl, C₅₋₁₀ alkyl, C₆₋₁₀ alkyl, C₇₋₁₀ alkyl, C₈₋₁₀ alkyl, C₉₋₁₀ alkyl, C₄₋₉ alkyl, C₅₋₉ alkyl, C₆₋₉ alkyl, C₇₋₉ alkyl, C₈₋₉ alkyl, C₃₋₈ alkyl, C₃₋₇ alkyl, C₃₋₆ alkyl, C₃₋₅ alkyl, C₃₋₄ alkyl, C₄₋₈ alkyl, C₄₋₇ alkyl, C₄₋₆ alkyl, C₄₋₅ alkyl, C₅₋₈ alkyl, C₅₋₇ alkyl, C₅₋₆ alkyl, C₆₋₈ alkyl, C₆₋₇ alkyl, or C₇₋₈ alkyl. In some embodiments, Y¹ and Y² is each independently a C₈₋₁₀ alkyl.

In some embodiments, R¹ is a C₃₋₉ alkyl. For example, R¹ is a C₄₋₉ alkyl, C₅₋₉ alkyl, C₆₋₉ alkyl, C₇₋₉ alkyl, C₈₋₉ alkyl, C₃₋₈ alkyl, C₃₋₇ alkyl, C₃₋₆ alkyl, C₃₋₅ alkyl, C₃₋₄ alkyl, C₄₋₈ alkyl, C₄₋₇ alkyl, C₄₋₆ alkyl, C₄₋₅ alkyl, C₅₋₈ alkyl, C₅₋₇ alkyl, C₅₋₆ alkyl, C₆₋₈ alkyl, C₆₋₇ alkyl, or C₇₋₈ alkyl. In some embodiments, R¹ is a C₃₋₅ alkyl or C₇₋₉ alkyl.

In some embodiments, R² and R³ is each independently a C₁₋₃ alkyl. Alternatively, R² taken together with R³ and the nitrogen atom to which they are attached form a 5-, 6-, or 7-membered ring. For example, R² taken together with R³ and the nitrogen atom to which they are attached form a 5-membered ring. Alternatively, R² taken together with R³ and the nitrogen atom to which they are attached form a 6-membered ring. Alternatively yet, R² taken together with R³ and the nitrogen atom to which they are attached form a 7-membered ring.

In some embodiments, the compound of Formula IV is represented by one of the following structural formulas:

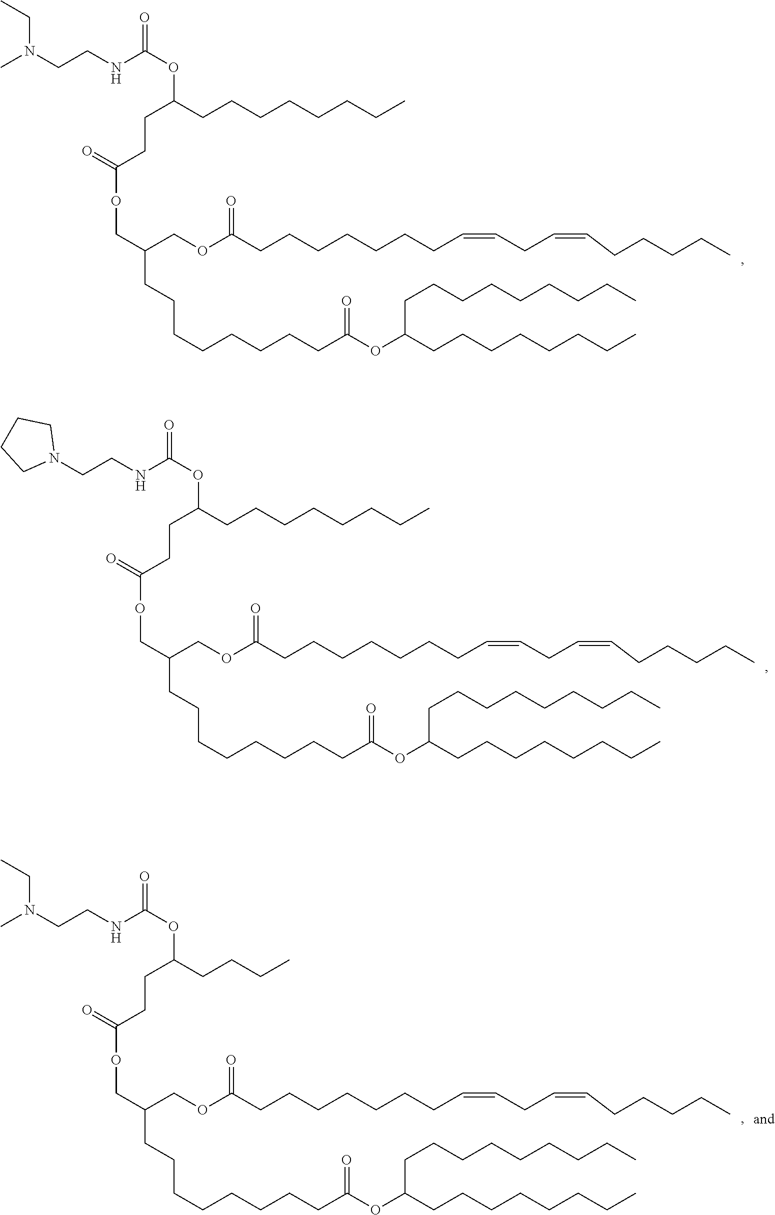

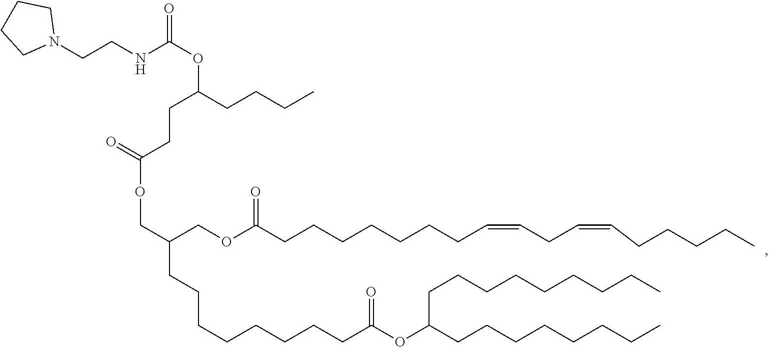 [SMILES: CCCCC/C=C\C/C=C\CCCCCCCC(=O)OCC(CCCCCCCCC(=O)OC(CCCCCCCC)CCCCCCCCC)COC(=O)CCC(CCCC)OC(=O)NCCN1CCCC1]

or a salt thereof.

In some embodiments, the present disclosure relates to a compound represented by one of the following structural formulas:

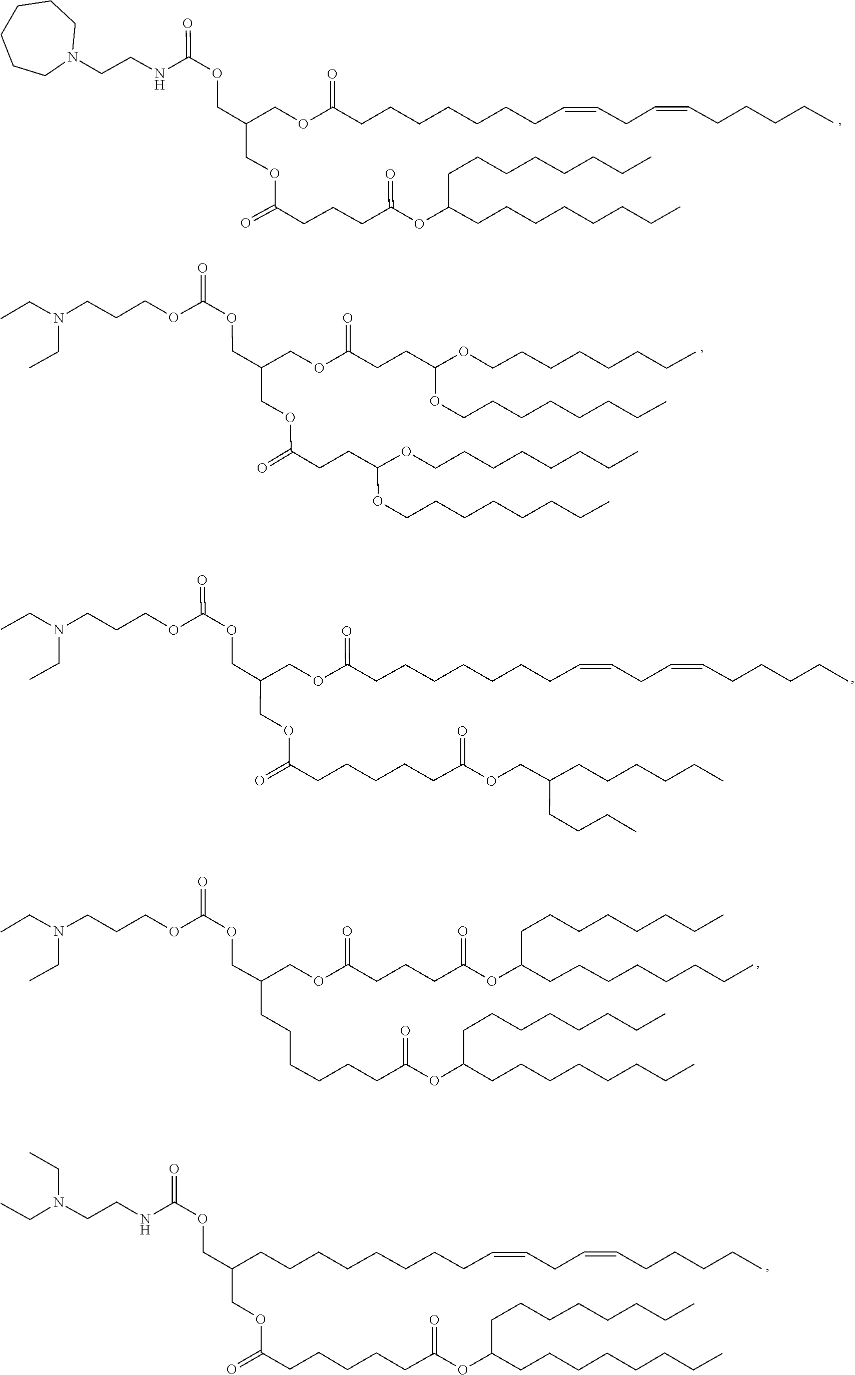

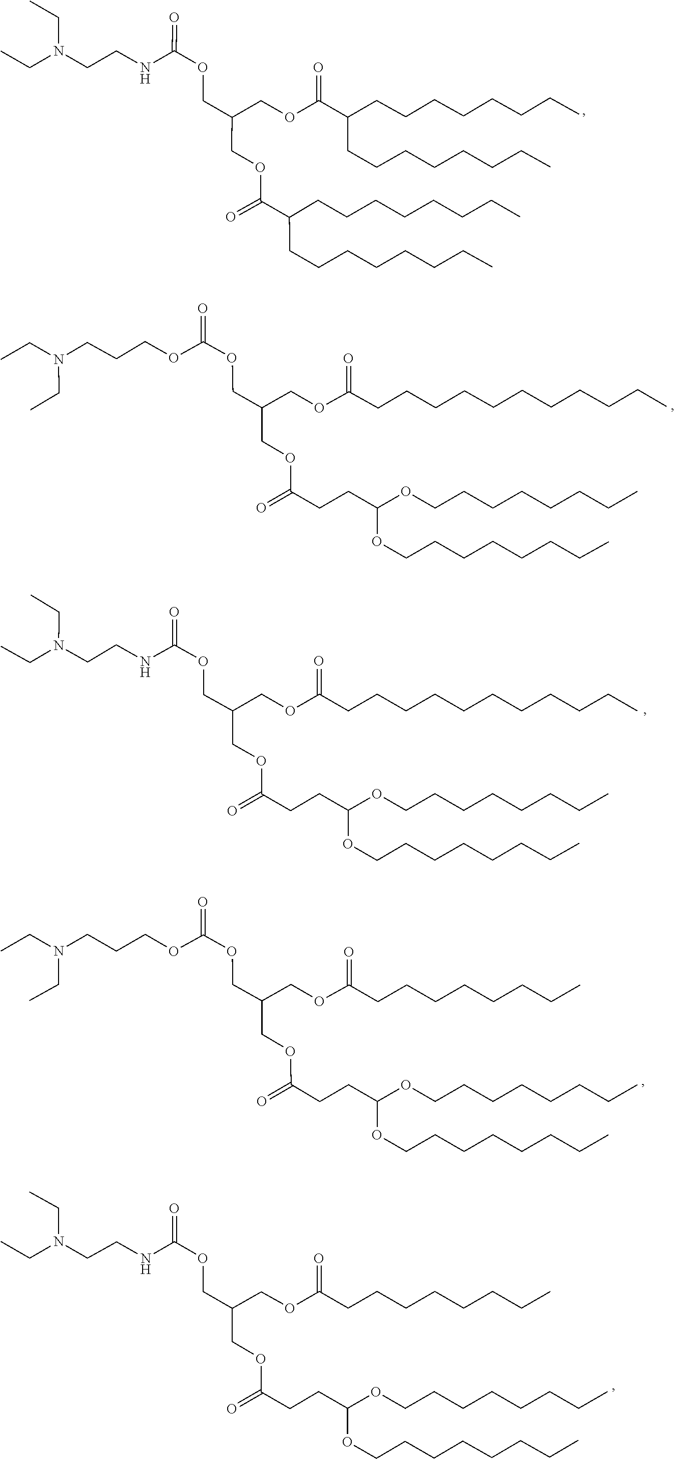

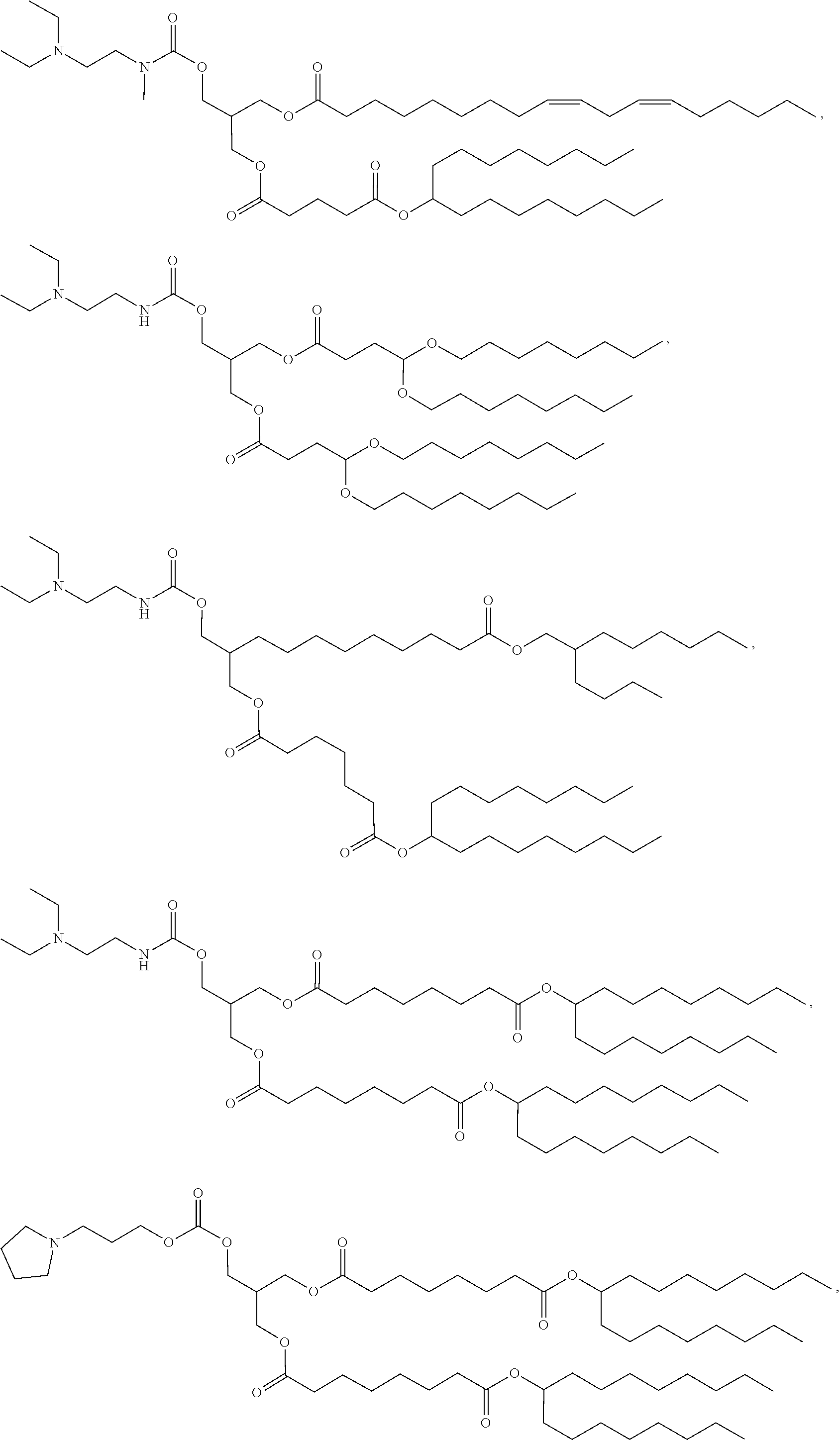

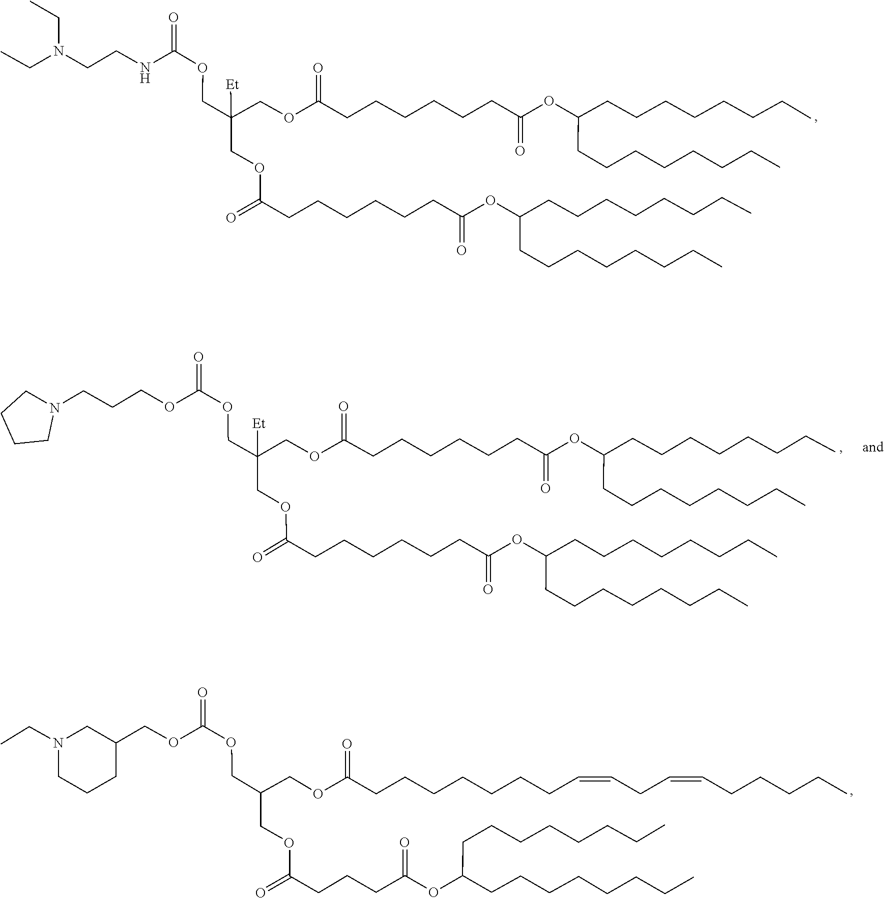

or a salt thereof.

In some embodiments, the present disclosure relates to a compound represented by one of the following structural formulas:

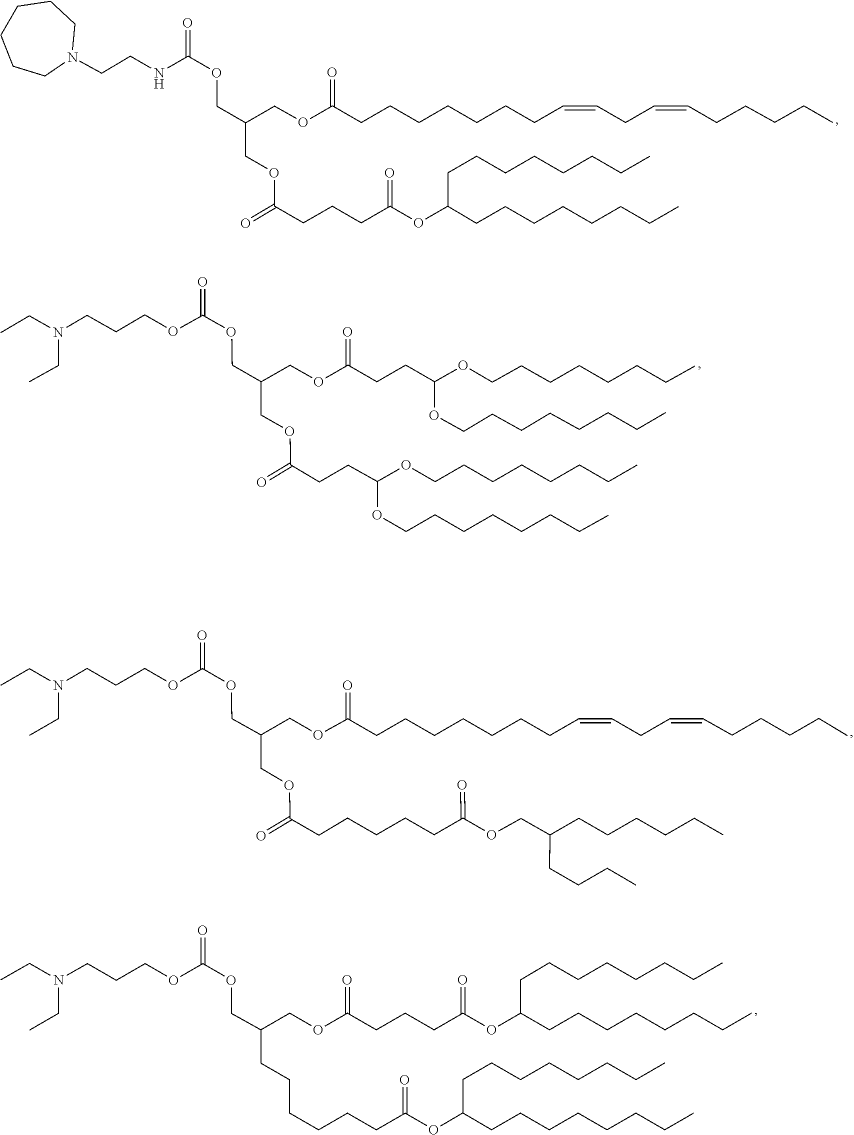

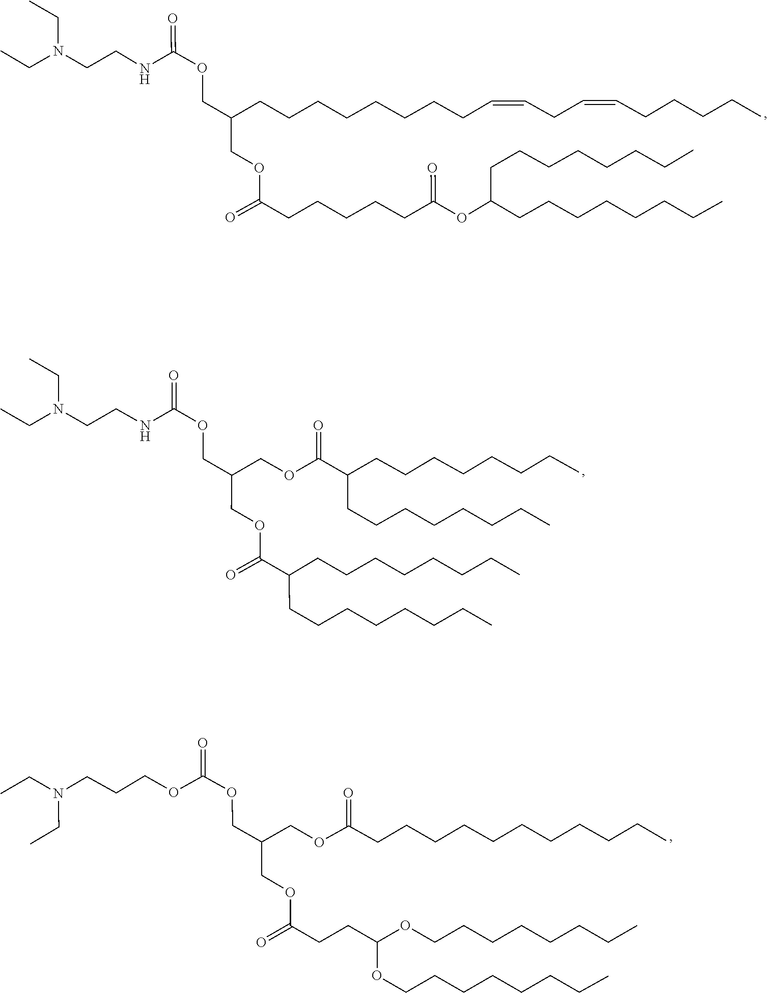

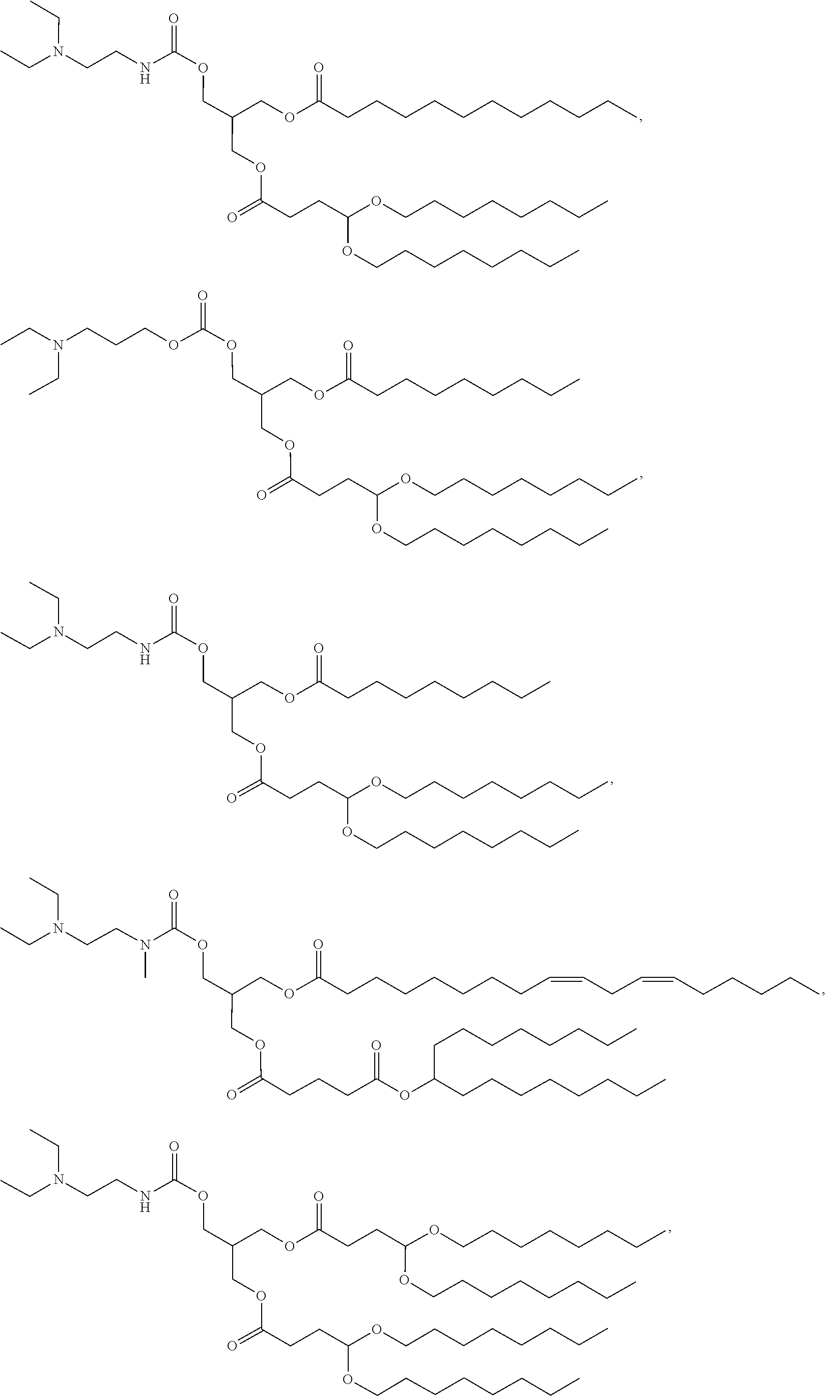

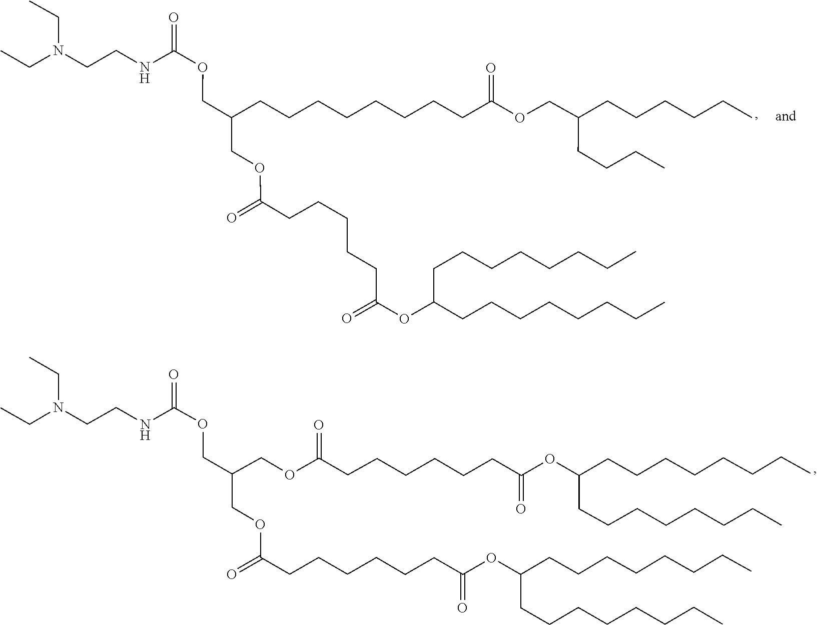

or a salt thereof.

In some embodiments, the salt is a pharmaceutically acceptable salt.

In certain embodiments, the invention relates to a composition comprising a compound of Formula (I)-(IV) or Table 1 and a lipid component.

In some embodiments, the lipid component further comprises a helper lipid and a PEG lipid.

In some embodiments, the lipid component further comprises a neutral lipid.

In some embodiments, the PEG lipid is selected from PEG-dilauroylglycerol, PEG-dimyristoylglycerol (PEG-DMG), PEG-dipalmitoylglycerol, PEG-distearoylglycerol (PEG-DSPE), PEG-dilaurylglycamide, PEG-dimyristylglycamide, PEG-dipalmitoylglycamide, PEG-distearoylglycamide, 1-[8′-(Cholest-5-en-3[beta]-oxy)carboxamido-3′,6′-dioxaoctanyl]carbamoyl-[omega]-methyl-poly(ethylene glycol) (PEG-cholesterol), 3,4-ditetradecoxylbenzyl-[omega]-methyl-poly(ethylene glycol)ether (PEG-DMB), 1,2-dimyristoyl-sn-glycero-3-phosphoethanolamine-N-[methoxy(polyethylene glycol)-2000] (PEG2K-DMPE), 1,2-dimyristoyl-rac-glycero-3-[methoxy(polyethylene glycol)-2000] (PEG2K-DMG), 1,2-distearoyl-sn-glycero-3-phosphoethanolamine-N-[methoxy(polyethylene glycol)-2000] (PEG2K-DSPE), 1,2-distearoyl-sn-glycerol-[methoxy(polyethylene glycol)-2000] (PEG2K-DSG), poly(ethylene glycol)-2000-dimethacrylate (PEG2K-DMA), 1,2-distearyloxypropyl-3-amine-N-[methoxy(polyethylene glycol)-2000] (PEG2K-DSA), and methoxy-PEG2000-carbamoyl-1,2-tridecyoxypropylamine (C13 Ether). For example, the PEG lipid is selected from PEG2K-DMG, C13 ether and C14 ether. For example, the PEG lipid comprises dimyristoylglycerol (DMG). Structures for C14 Ether, C13 Ether, and PEG2K-DMG are shown below:

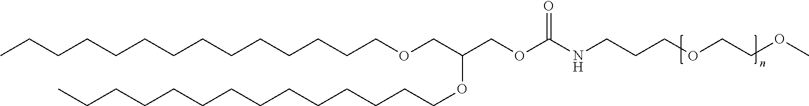

(where the number average n is about 45, C14 Ether);

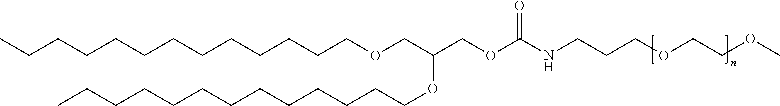

(where the number average n is about 45, C13 Ether);

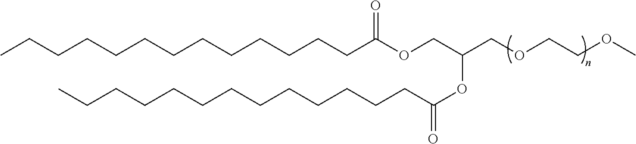

(where the number average n is about 45, PEG2K-DMG).

In some embodiments, the neutral lipid is selected from dipalmitoylphosphatidylcholine (DPPC), distearoylphosphatidylcholine (DSPC), dioleoylphosphocholine (DOPC), dimyristoylphosphatidylcholine (DMPC), 1-palmitoyl-2-linoleoyl-sn-glycero-3-phosphatidylcholine (PLPC), 1,2-diarachidoyl-sn-glycero-3-phosphocholine (DAPC), phosphatidylethanolamine (PE), egg phosphatidylcholine (EPC), dilauryloylphosphatidylcholine (DLPC), dimyristoylphosphatidylcholine (DMPC), 1-myristoyl-2-palmitoyl phosphatidylcholine (MPPC), 1-palmitoyl-2-myristoyl phosphatidylcholine (PMPC), 1-palmitoyl-2-stearoyl phosphatidylcholine (PSPC), 1,2-dibehenoyl-sn-glycero-3-phosphocholine (DBPC), 1-stearoyl-2-palmitoyl phosphatidylcholine (SPPC), 1,2-dieicosenoyl-sn-glycero-3-phosphocholine (DEPC), palmitoyloleoyl phosphatidylcholine (POPC), lysophosphatidyl choline, dioleoyl phosphatidylethanolamine (DOPE), dilinoleoylphosphatidylcholine, distearoylphosphatidylethanolamine (DSPE), dimyristoyl phosphatidylethanolamine (DMPE), dipalmitoyl phosphatidylethanolamine (DPPE), palmitoyloleoyl phosphatidylethanolamine (POPE), and lysophosphatidylethanolamine, or a combination thereof. For example, the neutral lipid is DSPC or DMPE. For example, the neutral lipid is DSPC.

In some embodiments, helper lipid is selected from cholesterol, 5-heptadecylresorcinol, and cholesterol hemisuccinate. For example, the helper lipid is cholesterol.

In some embodiments, the lipid component comprises 45-55 mol % of any one of the compounds represented by structural Formulas (I)-(IV) or selected from the compounds of Table 1, 35-40 mol % of the helper lipid, 7-10 mol % of the neutral lipid, and 1-5 mol % of the PEG lipid. For example, the lipid component comprises about 50 mol % of any one of the compounds represented by structural Formulas (I)-(IV) or selected from the compounds of Table 1, about 38 mol % of the helper lipid, about 9 mol % of the neutral lipid, and about 3 mol % of the PEG lipid.

In some embodiments, the composition further comprises a cryoprotectant.

In some embodiments, the composition further comprises a buffer.

In some embodiments, the composition further comprises an aqueous component comprising a biologically active agent. In some embodiments, the biologically active agent comprises a polypeptide. In some embodiments, the biologically active agent comprises or encodes a therapeutically active protein. In some embodiments, the biologically active agent comprises or encodes a genome-editing tool. In some embodiments, the biologically active agent comprises or encodes one or more nucleases capable of making single or double strand break in a DNA or an RNA.

In some embodiments, the biologically active agent comprises a nucleic acid. In some embodiments, the nucleic acid comprises RNA.

In some embodiments, the composition has an N/P ratio of from about 5 to about 7. For example, the N/P ratio is about 6 t 1. For example, the N/P ratio is about 6 t 0.5. For example, the N/P ratio is about 6.

In some embodiments, the composition further comprises an RNA component, wherein the RNA component comprises an mRNA. In some embodiments, the RNA component comprises a sequence encoding RNA-guided DNA binding agent, such as a Cas nuclease mRNA. In some embodiments, the RNA component comprises a Class 2 Cas nuclease mRNA. In some embodiments, the RNA component comprises a Cas9 nuclease mRNA.

In some embodiments, the RNA component comprises a modified RNA.

In some embodiments, the RNA component comprises a gRNA nucleic acid. In some embodiments, the gRNA nucleic acid is a gRNA. In some embodiments, the RNA component comprises a Class 2 Cas nuclease mRNA and a gRNA. In some embodiments, the gRNA nucleic acid is or encodes a dual-guide RNA (dgRNA). In some embodiments, the gRNA nucleic acid is or encodes a single-guide RNA (sgRNA).

In some embodiments, the gRNA is a modified gRNA. In some embodiments, the modified gRNA comprises a modification at one or more of the first five nucleotides at a 5′ end. In some embodiments, the modified gRNA comprises a modification at one or more of the last five nucleotides at a 3′ end.

In some embodiments, the composition comprises a guide RNA nucleic acid; the mRNA is a Class 2 Cas nuclease mRNA; and the ratio of the mRNA to the guide RNA nucleic acid is from about 2:1 to 1:4 by weight.

In some embodiments, the composition further comprises at least one template nucleic acid.

In some embodiments, the present disclosure relates to a method of cleaving a DNA, comprising contacting a cell with a composition as described herein.

In some embodiments, the present disclosure relates to a method of gene editing, comprising contacting a cell with a composition as described herein.

In some embodiments, the contacting step results in a single stranded DNA nick. In some embodiments, the contacting step results in a double-stranded DNA break.

In some embodiments, the composition comprises a Class 2 Cas mRNA and a gRNA nucleic acid.

In some embodiments, the method further comprises introducing at least one template nucleic acid into the cell. In some embodiments, the method comprises contacting the cell with a composition comprising a template nucleic acid.

In some embodiments, the method comprises administering the composition to an animal. In some embodiments, the method comprises administering the composition to a human. In some embodiments, the method comprises administering the composition to a cell. In some embodiments, the cell is a eukaryotic cell.

In some embodiments, the method comprises administering mRNA formulated in a first lipid nanoparticle (LNP) composition and a second LNP composition comprising one or more of an mRNA, a gRNA, a gRNA nucleic acid, and a template nucleic acid. In some embodiments, the first and second LNP compositions are administered simultaneously. In some embodiments, the first and second LNP compositions are administered sequentially.

In some embodiments, the method comprises administering the mRNA and the gRNA nucleic acid formulated in a single LNP composition.

In some embodiments, the gene editing results in a gene knockout. In some embodiments, the gene editing results in a gene correction.

In some embodiments, the cell is contacted with the lipid composition in vitro. In some embodiments, the cell is contacted with the lipid composition ex vivo.

In some embodiments, the method comprises contacting a tissue of an animal with the lipid composition. In some embodiments, the tissue is liver tissue.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference.

Applicant reserves the right to physically incorporate into this application any and all materials and information from any such articles, patents, patent applications, or other physical and electronic documents.

### EXAMPLES

### General Information

All reagents and solvents were purchased and used as received from commercial vendors or synthesized according to cited procedures. All intermediates and final compounds were purified using flash column chromatography on silica gel. NMR spectra were recorded on a Bruker or Varian 400 MHz spectrometer, and NMR data were collected in CDCl₃ at ambient temperature. Chemical shifts are reported in parts per million (ppm) relative to CDCl₃ (7.26). Data for 1H NMR are reported as follows: chemical shift, multiplicity (br=broad, s=singlet, d=doublet, t=triplet, q=quartet, dd=doublet of doublets, dt=doublet of triplets m=multiplet), coupling constant, and integration. MS data were recorded on a Waters SQD2 mass spectrometer with an electrospray ionization (ESI) source. Purity of the final compounds was determined by UPLC-MS-ELS using a Waters Acquity H-Class liquid chromatography instrument equipped with SQD2 mass spectrometer with photodiode array (PDA) and evaporative light scattering (ELS) detectors.

### Example 1—Compound 1

### Intermediate 1a: 4,4-bis(octyloxy)butanenitrile

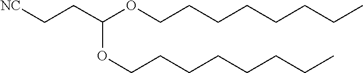 [SMILES: CCCCCCCCOC(CCC#N)OCCCCCCCC]

To a mixture of 4,4-dimethoxybutanenitrile (20 g, 1.0 equiv.) and octan-1-ol (2-3 equiv.) in toluene (0.5-2.0 M) was added 4-methylbenzenesulfonic acid monohydrate (0.1-0.25 equiv.). The mixture was stirred at 100-120° C. for at least 24 h under N₂ atmosphere. The reaction mixture was concentrated under reduced pressure to remove solvent. The residue was dried over Na₂SO₄, filtered, and the filtrate was concentrated under reduced pressure to afford product as a colorless oil (40%).

### Intermediate 1b: 4,4-bis(octyloxy)butanoic Acid

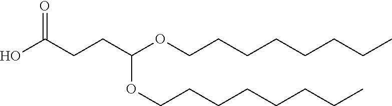 [SMILES: CCCCCCCCOC(CCC(=O)O)OCCCCCCCC]

To a solution of Intermediate 1a (20 g, 1.0 equiv.) in 1:1 H₂O/EtOH (0.1-1.0 M) was added KOH (4.0-15.0 equiv.). The mixture was stirred at 110° C. for at least 12 h under N2 atmosphere. The reaction mixture was then concentrated under reduced pressure to remove solvent. 6 N HCl was added to adjust the residue to pH 5-7 and extracted with 2-3× with EtOAc. The combined organic layers were concentrated under reduced pressure to give a residue as a colorless oil (35%). ¹H NMR (400 MHz, CDCl₃) δ 4.46 (t, J=5.4 Hz, 1H), 3.60-3.49 (m, 2H), 3.35 (dt, J=9.3, 6.7 Hz, 2H), 2.40 (t, J=7.2 Hz, 2H), 1.88 (td, J=7.2, 5.3 Hz, 2H), 1.50 (p, J=6.9 Hz, 4H), 1.33-1.12 (m, 21H), 0.81 (t, J=6.6 Hz, 6H).

### Intermediate 1c: 2-(hydroxymethyl)propane-1,3-diyl bis(4,4-bis(octyloxy)butanoate)

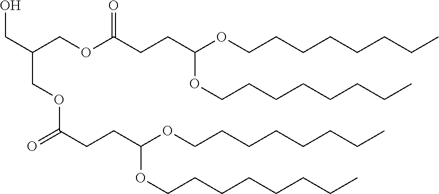 [SMILES: CCCCCCCCOC(CCC(=O)OCC(CO)COC(=O)CCC(OCCCCCCCC)OCCCCCCCC)OCCCCCCCC]

To a solution of Intermediate 1b (7.5 g, 2.0 equiv.) and 2-(hydroxymethyl)propane-1,3-diol (1.0 equiv.) in 3:1 DCM/DMF (0.1-0.5 M) was added DMAP (0.1 equiv.), DIPEA (2.5 equiv.) and EDCI (1.2 equiv.). The mixture was stirred at 20° C. for at least 12 h under N2 atmosphere. The reaction mixture was diluted with water and extracted 3× with DCM. The combined organic layers were dried over Na₂SO₄, filtered, and the filtrate was concentrated under reduced pressure to afford product as a colorless oil (36%).

### Intermediate 1d: 2-((((4-nitrophenoxy)carbonyl)oxy)methyl)propane-1,3-diyl bis(4,4-bis(octyloxy)butanoate)

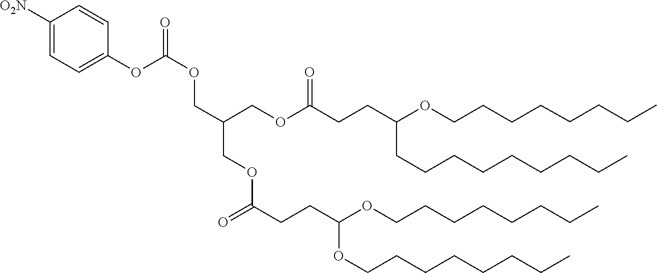 [SMILES: CCCCCCCCCC(CCC(=O)OCC(COC(=O)CCC(OCCCCCCCC)OCCCCCCCC)COC(=O)Oc1ccc([N+](=O)[O-])cc1)OCCCCCCCC]

To a mixture of Intermediate 1c (3 g, 1.0 equiv.), (4-nitrophenyl) carbonochloridate (1.5-2.0 equiv.) in DCM (0.1-0.5 M) was added pyridine (1.5 2.0 equiv.) dropwise at 0° C. Then the mixture was stirred at 15-20° C. for at least 1 h under N2 atmosphere. The reaction mixture was diluted with hexanes, filtered, and the filtrate was concentrated under reduced pressure to afford product as a colorless oil (68%). ¹H NMR (400 MHz, CDCl₃) δ 8.30-8.24 (m, 2H), 7.41-7.35 (m, 2H), 4.47 (t, J=5.5 Hz, 2H), 4.34 (d, J=5.7 Hz, 2H), 4.19 (dd, J=6.0, 2.7 Hz, 4H), 3.54 (dt, J=9.4, 6.7 Hz, 4H), 3.38 (dt, J=9.3, 6.7 Hz, 4H), 2.50 (p, J=6.0 Hz, 1H), 2.40 (t, J=7.6 Hz, 4H), 1.92 (td, J=7.6, 5.5 Hz, 4H), 1.54 (q, J=6.8 Hz, 8H), 1.32-1.20 (m, 40H), 0.93-0.78 (m, 12H).

### Compound 1: 2-((((3-(diethylamino)propoxy)carbonyl)oxy)methyl)propane-1,3-diyl bis(4,4-bis(octyloxy)butanoate)

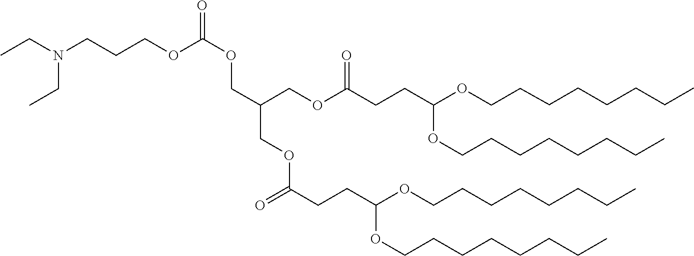 [SMILES: CCCCCCCCOC(CCC(=O)OCC(COC(=O)CCC(OCCCCCCCC)OCCCCCCCC)COC(=O)OCCCN(CC)CC)OCCCCCCCC]

To a mixture of Intermediate 1d (2.5 g, 0.75-1.0 equiv.), 3-(diethylamino)propan-1-ol (2.0 equiv.), DMAP (0.1-2.0 equiv.) and pyridine (1.0-3.0 equiv.) in MeCN (0.05-0.5 M) was degassed and purged 3× with N2, and then the mixture was stirred at 20° C. for at least 12 h under N2 atmosphere. The reaction mixture was concentrated under reduced pressure to remove MeCN. The residue was diluted with water and extracted 3× with EtOAc. The combined organic layers were washed 3× with aq. NaHCO₃, dried over Na₂SO₄, filtered, and the filtrate was concentrated under reduced pressure to give a residue and purified by column chromatography to afford product as a colorless oil. ¹H NMR (400 MHz, CDCl₃) δ 4.41 (t, J=5.6 Hz, 2H), 4.16-4.01 (m, 8H), 3.49 (dt, J=9.3, 6.7 Hz, 4H), 3.33 (dt, J=9.3, 6.7 Hz, 4H), 2.46 (q, J=7.0 Hz, 6H), 2.34 (q, J=7.3 Hz, 5H), 1.85 (td, J=7.6, 5.4 Hz, 4H), 1.75 (p, J=6.9 Hz, 2H), 1.48 (q, J=7.0 Hz, 8H), 1.33-1.06 (m, 44H), 0.95 (t, J=7.1 Hz, 6H), 0.81 (t, J=6.7 Hz, 12H). MS: 916.5 m/z [M+H].

### Example 2—Compound 2

### Intermediate 2a: heptadecan-9-ol

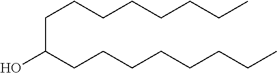 [SMILES: CCCCCCCCC(O)CCCCCCCC]

To a solution of heptadecan-9-one (45 g, 1.0 equiv.) in 5:1 THF/MeOH (0.1-0.5 M) was added NaBH₄ (1.5 equiv.) at 0° C. The mixture was stirred at 25° C. for at least 1 h. The reaction mixture was quenched with saturated NH₄Cl and diluted with H₂O. Then the mixture was extracted 3× with EtOAc, dried over Na₂SO₄, filtered, and the filtrate was concentrated under reduced pressure to give a residue. The residue was purified by column chromatography to afford product as a white solid. ¹H NMR (400 MHz, CDCl₃) δ 3.58 (dt, J=7.3, 4.3 Hz, 1H), 1.56-1.14 (m, 29H), 0.88 (t, J=6.7 Hz, 6H).

### Intermediate 2b: 5-(heptadecan-9-yloxy)-5-oxopentanoic Acid

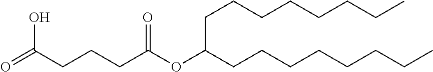 [SMILES: CCCCCCCCC(CCCCCCCC)OC(=O)CCCC(=O)O]

To a solution of glutaric acid (5.0 g, 1.0 equiv.) in THF (0.5-1.0 M) was added (COCl)₂ (1.0-1.2 equiv.) and DMF (0.05-0.1 equiv.) at 0° C. The mixture was stirred at 25° C. for 2 h. Then Intermediate 2a (1.0 equiv.) in THF (0.5-1.0 M) was added to the reaction mixture. The reaction mixture was stirred at 20-25° C. for at least 2 h. The reaction mixture was concentrated under reduced pressure to remove solvent. The residue was diluted with H₂O and extracted 3× with EtOAc or DCM, dried over Na₂SO₄, filtered, and the filtrate was concentrated under reduced pressure to give a residue. The residue was purified by column chromatography to afford product as a yellow oil (27%). ¹H NMR (400 MHz, CDCl₃) δ 4.87 (p, J=6.3 Hz, 1H), 2.40 (dt, J=21.5, 7.3 Hz, 4H), 1.97 (q, J=7.4 Hz, 2H), 1.51 (q, J=6.2 Hz, 4H), 1.25 (s, 23H), 0.97-0.76 (m, 6H).

### Intermediate 2c: di(heptadecan-9-yl) O,O′-(2-(hydroxymethyl)propane-1,3-diyl) diglutarate

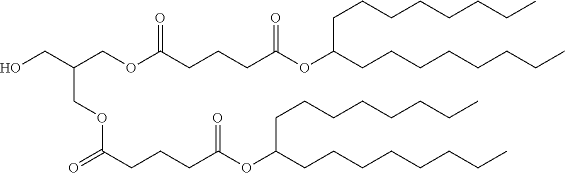 [SMILES: CCCCCCCCC(CCCCCCCC)OC(=O)CCCC(=O)OCC(CO)COC(=O)CCCC(=O)OC(CCCCCCCC)CCCCCCCC]

To a solution of Intermediate 2b (4 g, 0.5-1.0 equiv.) and 2-(hydroxymethyl)propane-1,3-diol (0.5-1.0 equiv.) in DCM (0.1-0.2 M) was added DMAP (0.2 equiv.), EDCI (1.0-2.0 equiv.) and DIPEA (2.0 equiv.). The reaction mixture was stirred at 15-25° C. for at least 5 h. The reaction mixture was concentrated under reduced pressure to remove solvent to get a residue. The residue was diluted with H₂O and extracted 3× with EtOAc or DCM, washed with brine, dried over Na₂SO₄, filtered, and the filtrate was concentrated under reduced pressure to give a residue. The residue was purified by column chromatography to afford product as a yellow oil. ¹H NMR (400 MHz, CDCl₃) δ 4.87 (p, J=6.3 Hz, 2H), 4.18 (dd, J=6.1, 2.2 Hz, 4H), 3.63 (d, J=5.5 Hz, 2H), 2.38 (dt, J=17.6, 7.3 Hz, 8H), 2.24-2.16 (m, 2H), 1.96 (p, J=7.4 Hz, 4H), 1.51 (q, J=6.1 Hz, 8H), 1.26 (s, 46H), 0.88 (t, J=6.7 Hz, 12H).

### Intermediate 2d: di(heptadecan-9-yl) O,O′-(2-((((4-nitrophenoxy)carbonyl)oxy)methyl)propane-1,3-diyl) diglutarate

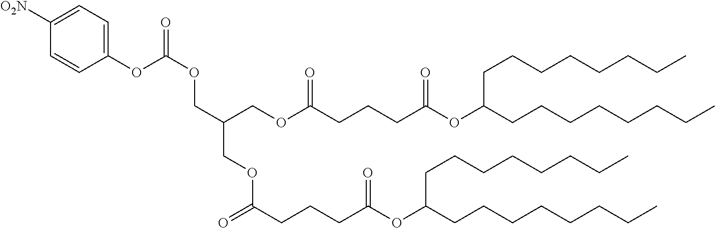 [SMILES: CCCCCCCCC(CCCCCCCC)OC(=O)CCCC(=O)OCC(COC(=O)CCCC(=O)OC(CCCCCCCC)CCCCCCCC)COC(=O)Oc1ccc([N+](=O)[O-])cc1]

To a solution of Intermediate 2c (1.1 g, 1.0 equiv.) and 4-nitrophenyl carbonochloridate (1.0-3.0 equiv.) in DCM (0.1-0.2 M) was added pyridine (2.0-3.0 equiv.). The reaction mixture was stirred at 25° C. for 1 h under N₂. The reaction mixture was concentrated under reduced pressure to remove solvent to get a residue. The residue was diluted with H₂O and extracted 3× with hexanes or EtOAc, dried over Na₂SO₄, filtered, and the filtrate was concentrated under reduced pressure to give a residue. The residue was purified by column chromatography to afford product as a colorless oil. ¹H NMR (400 MHz, CDCl₃) δ 8.31-8.23 (m, 2H), 7.43-7.35 (m, 2H), 4.86 (p, J=6.3 Hz, 2H), 4.35 (d, J=5.8 Hz, 2H), 4.22 (dd, J=6.1, 2.3 Hz, 4H), 2.51 (p, J=5.9 Hz, 1H), 2.38 (dt, J=22.6, 7.4 Hz, 8H), 1.96 (p, J=7.4 Hz, 4H), 1.50 (q, J=6.1 Hz, 9H), 1.25 (s, 46H), 0.87 (t, J=6.8 Hz, 12H).

### Compound 2: O,O′-(2-((((3-(diethylamino)propoxy)carbonyl)oxy)methyl)propane-1,3-diyl) di(heptadecan-9-yl) diglutarate

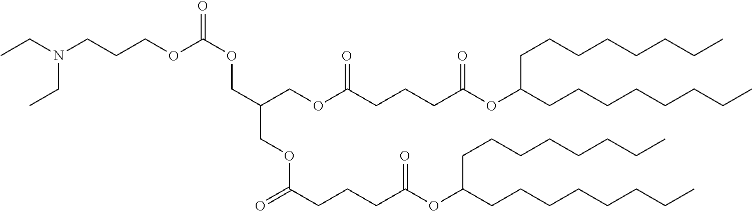 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(CC)CC]

To a solution of Intermediate 2d (1.8 g, 1.0 equiv.) and 3-(diethyl-1amino)propan-1-ol (1.0-3.0 equiv.) in DCM (0.05-0.2 M) was added pyridine (2.0-3.0 equiv.) and DMAP (0.1 equiv.). The reaction mixture was stirred at 25° C. for 2 h under N2. The reaction mixture was concentrated under reduced pressure to remove solvent to get a residue. The residue was diluted with H₂O and extracted 3× with EtOAc, dried over Na₂SO₄, filtered, and the filtrate was concentrated under reduced pressure to give a residue. The residue was purified by column chromatography to afford product as a colorless oil (95%). ¹H NMR (400 MHz, CDCl₃) δ 4.79 (tt, J=6.5, 3.2 Hz, 2H), 4.18-4.02 (m, 8H), 2.44 (qd, J=7.0, 2.5 Hz, 6H), 2.31 (tdd, J=15.2, 6.3, 2.0 Hz, 9H), 1.88 (pd, J=7.4, 2.0 Hz, 4H), 1.75 (qt, J=9.0, 4.6 Hz, 3H), 1.44 (dt, J=11.4, 5.5 Hz, 8H), 1.29-1.12 (m, 46H), 0.94 (td, J=7.1, 2.4 Hz, 6H), 0.81 (td, J=6.8, 2.3 Hz, 12H). MS: 968.7 m/z [M+H].

### Example 3—Compound 3

### Intermediate 3a: 7-(heptadecan-9-yloxy)-7-oxoheptanoic Acid

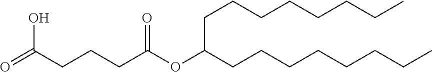 [SMILES: CCCCCCCCC(CCCCCCCC)OC(=O)CCCC(=O)O]

Intermediate 3a was synthesized (36%) from heptanedioic acid and heptadecan-9-ol using the method employed in the synthesis of Intermediate 2b. ¹H NMR (400 MHz, CDCl₃) δ 4.86 (p, J=6.3 Hz, 1H), 2.32 (dt, J=24.7, 7.5 Hz, 4H), 1.65 (dtt, J=11.7, 7.5, 4.0 Hz, 4H), 1.50 (q, J=6.0 Hz, 4H), 1.43-1.14 (m, 26H), 0.87 (t, J=6.8 Hz, 6H).

### Intermediate 3b: 7,7′-di(heptadecan-9-yl) O′1,O1-(2-(hydroxymethyl)propane-1,3-diyl) di(heptanedioate)

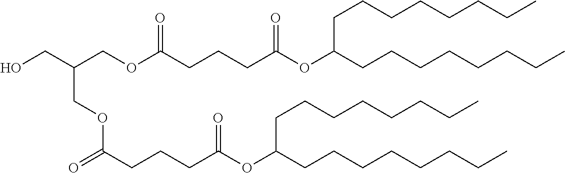 [SMILES: CCCCCCCCC(CCCCCCCC)OC(=O)CCCC(=O)OCC(CO)COC(=O)CCCC(=O)OC(CCCCCCCC)CCCCCCCC]

Intermediate 3b was synthesized (18%) from Intermediate 3a using the method employed in the synthesis of Intermediate 2c. ¹H NMR (400 MHz, CDCl₃) δ 4.79 (p, J=6.3 Hz, 2H), 4.10 (h, J=6.3, 5.9 Hz, 4H), 3.55 (d, J=5.5 Hz, 2H), 2.24 (dt, J=17.3, 7.5 Hz, 8H), 2.16-2.09 (m, 1H), 1.58 (pd, J=7.5, 5.1 Hz, 8H), 1.43 (d, J=6.0 Hz, 8H), 1.33-1.25 (m, 5H), 1.19 (s, 46H), 0.81 (t, J=6.7 Hz, 12H).

### Intermediate 3c: 7,7′-di(heptadecan-9-yl) O′1,O1-(2-((((4-nitrophenoxy)carbonyl)oxy)methyl)propane-1,3-diyl) di(heptanedioate)

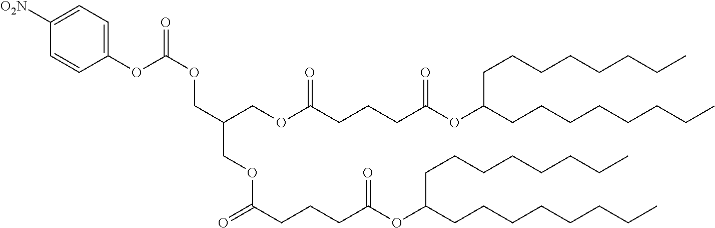 [SMILES: CCCCCCCCC(CCCCCCCC)OC(=O)CCCC(=O)OCC(COC(=O)CCCC(=O)OC(CCCCCCCC)CCCCCCCC)COC(=O)Oc1ccc([N+](=O)[O-])cc1]

Intermediate 3c was synthesized (84%) from Intermediate 3b using the method employed in the synthesis of Intermediate 2d. ¹H NMR (400 MHz, CDCl₃) δ 8.24-8.17 (m, 2H), 7.36-7.28 (m, 2H), 4.79 (p, J=6.3 Hz, 2H), 4.29 (d, J=5.9 Hz, 2H), 4.14 (dd, J=6.0, 2.3 Hz, 4H), 2.44 (p, J=6.0 Hz, 1H), 2.24 (dt, J=23.0, 7.5 Hz, 8H), 1.58 (h, J=7.3 Hz, 9H), 1.43 (q, J=6.1 Hz, 8H), 1.35-1.07 (m, 51H), 0.80 (t, J=6.8 Hz, 12H).

### Compound 3: O′1,O1-(2-((((3-(diethylamino)propoxy)carbonyl)oxy)methyl)propane-1,3-diyl) 7,7′-di(heptadecan-9-yl) di(heptanedioate)

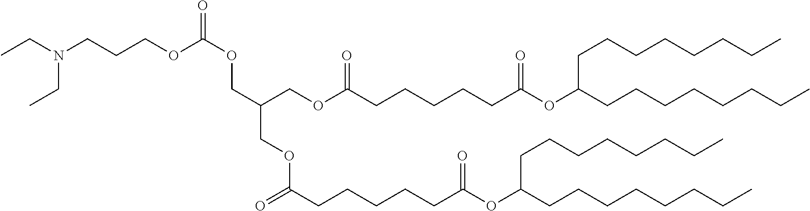 [SMILES: CCCCCCCCC(CCCCCCCC)OC(=O)CCCCCC(=O)OCC(COC(=O)CCCCCC(=O)OC(CCCCCCCC)CCCCCCCC)COC(=O)OCCCN(CC)CC]

Compound 3 was synthesized (57%) from Intermediate 3c using the method employed in the synthesis of Compound 2. ¹H NMR (400 MHz, CDCl₃) δ 4.79 (p, J=6.2 Hz, 2H), 4.15-4.04 (m, 8H), 2.43 (t, J=7.0 Hz, 6H), 2.35 (p, J=5.9 Hz, 1H), 2.23 (dt, J=12.9, 7.5 Hz, 8H), 1.75 (q, J=6.9 Hz, 2H), 1.57 (p, J=7.6 Hz, 9H), 1.43 (q, J=5.9 Hz, 8H), 1.32-1.11 (m, 53H), 0.94 (t, J=7.1 Hz, 6H), 0.81 (t, J=6.6 Hz, 12H). MS: 1024.8 m/z [M+H].

### Example 4—Compound 4

### Intermediate 4a: 7-((2-butyloctyl)oxy)-7-oxoheptanoic Acid

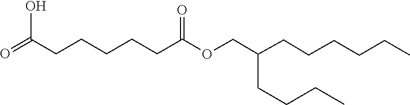 [SMILES: CCCCCCC(CCCC)COC(=O)CCCCCC(=O)O]

To a mixture of heptanedioic acid (20 g, 1.0 equiv.), DMF (0.2 equiv.) in THF (0.2-0.4 M) was added (COCl)₂ (1.0 equiv.) dropwise under N₂. The mixture was stirred at 20° C. for 2 h under N2 atmosphere. Then 2-butyloctan-1-ol (1.0 equiv.) was added to the reaction mixture dropwise and stirred at 20° C. for 2 h under N₂ atmosphere. The reaction mixture was concentrated under reduced pressure to remove solvent, poured into water, and extracted 3× with EtOAc. The combined organic phase was washed with brine, dried with anhydrous Na₂SO₄, filtered, and the filtrate was concentrated in vacuum. The residue was purified by column chromatography to afford product as a pale yellow oil (49%). ¹H NMR (400 MHz, CDCl₃) δ 3.96 (d, J=5.8 Hz, 2H), 2.33 (dt, J=16.4, 7.5 Hz, 4H), 1.66 (qd, J=7.6, 2.7 Hz, 5H), 1.42-1.33 (m, 2H), 1.27 (t, J=4.3 Hz, 16H), 0.88 (td, J=6.6, 3.8 Hz, 6H).

### Intermediate 4b: 3-hydroxy-2-(hydroxymethyl)propyl (9Z,12Z)-octadeca-9,12-dienoate

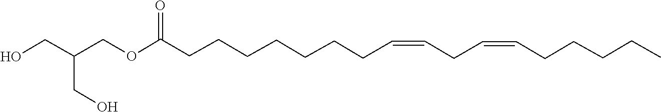 [SMILES: CCCCC/C=C\C/C=C\CCCCCCCC(=O)OCC(CO)CO]

To a solution of 2-(hydroxymethyl)propane-1,3-diol (2.0-3.0 equiv.) and (9Z,12Z)-octadeca-9,12-dienoic acid (20 g, 1.0 equiv.) in 2:1 DCM/DMF (0.2-0.5 M) was added EDCI (1.0-2.0 equiv.), DMAP (0.1-0.2 equiv.) and DIPEA (2.0-3.0equiv.). The reaction mixture was stirred at 15-25° C. for at least 5 h under N₂. The reaction mixture was concentrated under reduced pressure to remove solvent. The residue was diluted with H₂O, extracted 3× with DCM or EtOAc, dried over Na₂SO₄, filtered, and the filtrate was concentrated under reduced pressure to give a residue. The residue was purified by column chromatography to afford product as a yellow oil (76%). ¹H NMR (400 MHz, CDCl₃) δ 7.99 (s, 1H), 5.47-5.05 (m, 4H), 4.22 (d, J=6.3 Hz, 2H), 3.74 (qd, J=11.1, 5.2 Hz, 4H), 2.94 (s, 3H), 2.86 (s, 3H), 2.78-2.71 (m, 2H), 2.31 (t, J=7.5 Hz, 2H), 2.02 (p, J=6.5, 6.0 Hz, 5H), 1.60 (p, J=7.3 Hz, 2H), 1.41-1.12 (m, 13H), 0.91-0.75 (m, 3H).

### Intermediate 4c: 1-(2-butyloctyl) 7-(3-hydroxy-2-((((9Z,12Z)-octadeca-9,12-dienoyl)oxy)methyl)propyl) heptanedioate

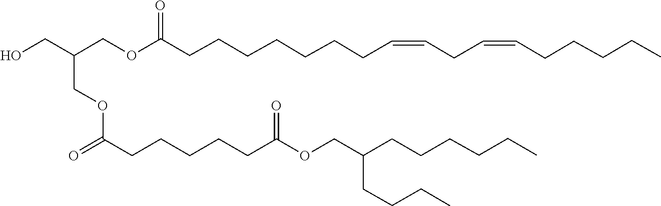 [SMILES: CCCCC/C=C\C/C=C\CCCCCCCC(=O)OCC(CO)COC(=O)CCCCCC(=O)OCC(CCCC)CCCCCC]

To a solution of Intermediate 4b (9 g, 1.0 equiv.) in DCM (0.1-0.3 M) was added Intermediate 10a (1.0 equiv.), EDCI (1.2 equiv.), DMAP (0.1 equiv.) and DIPEA (2.0-3.0 equiv.). The mixture was stirred at 15-25° C. for at least under N₂. The reaction mixture was concentrated under reduced pressure to remove solvent to afford a residue. The residue was diluted with H₂O and extracted 3× with EtOAc or DCM. The combined organic layers were dried over Na₂SO₄, filtered, and the filtrate was concentrated under reduced pressure to give a residue. The residue was purified by column chromatography to afford product as a colorless oil (42%). ¹H NMR (400 MHz, CDCl₃) δ 5.44-5.28 (m, 4H), 4.18 (pt, J=6.4, 3.7 Hz, 4H), 3.98 (d, J=5.8 Hz, 2H), 3.63 (d, J=5.6 Hz, 2H), 2.78 (t, J=6.4 Hz, 2H), 2.39-2.28 (m, 6H), 2.20 (hept, J=5.8 Hz, 2H), 2.06 (q, J=6.9 Hz, 4H), 1.66 (dtd, J=15.5, 7.6, 4.1 Hz, 9H), 1.41-1.20 (m, 31H), 0.90 (td, J=6.6, 3.1 Hz, 9H).

### Intermediate 4d: 1-(2-butyloctyl) 7-(3-(((4-nitrophenoxy)carbonyl)oxy)-2-((((9Z,12Z)-octadeca-9,12-dienoyl)oxy)methyl)propyl) heptanedioate

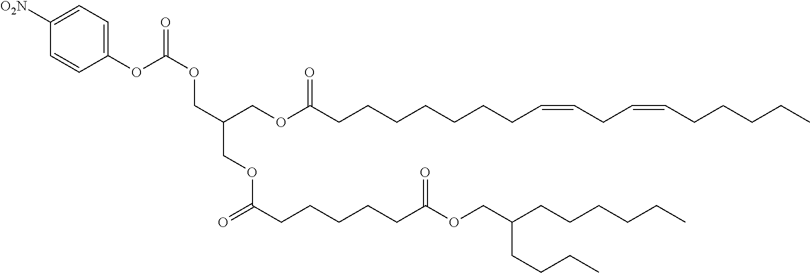 [SMILES: CCCCC/C=C\C/C=C\CCCCCCCC(=O)OCC(COC(=O)CCCCCC(=O)OCC(CCCC)CCCCCC)COC(=O)Oc1ccc([N+](=O)[O-])cc1]

To a solution of Intermediate 4c (5 g, 1.0 equiv.) in DCM (0.1-0.2 M) was added (4-nitrophenyl) carbonochloridate (3.0 equiv.) and pyridine (3.0 equiv.). The mixture was stirred at 25° C. for 2 h under N₂. The reaction mixture was concentrated under reduced pressure to remove DCM to afford a residue, and the residue was purified by column chromatography to afford product as a colorless oil (64%). ¹H NMR (400 MHz, CDCl₃) δ 8.32-8.23 (m, 2H), 7.42-7.34 (m, 2H), 5.43-5.23 (m, 4H), 4.36 (d, J=5.8 Hz, 2H), 4.27-4.13 (m, 4H), 3.97 (d, J=5.8 Hz, 2H), 2.77 (t, J=6.3 Hz, 2H), 2.52 (hept, J=6.0 Hz, 1H), 2.39-2.24 (m, 6H), 2.05 (q, J=6.9 Hz, 4H), 1.65 (dtd, J=15.2, 7.6, 5.0 Hz, 8H), 1.43-1.19 (m, 32H), 0.89 (td, J=6.6, 3.0 Hz, 9H).

### Compound 4: 1-(2-butyloctyl) 7-(3-(((3-(diethylamino)propoxy)carbonyl)oxy)-2-((((9Z,12Z)-octadeca-9,12-dienoyl)oxy)methyl)propyl) heptanedioate

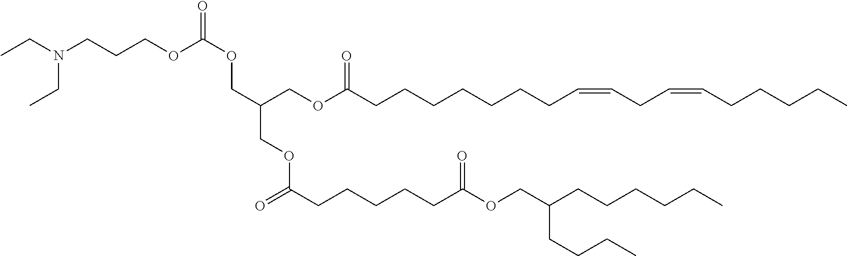 [SMILES: CCCCC/C=C\C/C=C\CCCCCCCC(=O)OCC(COC(=O)CCCCCC(=O)OCC(CCCC)CCCCCC)COC(=O)OCCCN(CC)CC]

Compound 4 was synthesized from Intermediate 4d using the method employed in the synthesis of Compound 2. ¹H NMR (400 MHz, CDCl₃) δ 5.37-5.20 (m, 4H), 4.17-4.03 (m, 8H), 3.90 (d, J=5.8 Hz, 2H), 2.70 (t, J=6.4 Hz, 2H), 2.44 (q, J=7.1 Hz, 6H), 2.35 (p, J=6.0 Hz, 1H), 2.24 (td, J=7.6, 4.7 Hz, 6H), 1.98 (q, J=6.9 Hz, 4H), 1.74 (p, J=6.7 Hz, 2H), 1.55 (dt, J=11.6, 6.0 Hz, 7H), 1.37-1.13 (m, 32H), 0.94 (t, J=7.1 Hz, 6H), 0.82 (dp, J=7.0, 3.1 Hz, 9H). MS: 836.6 m/z [M+H].

### Example 5—Compound 5

### Intermediate 5a: 7,7′-bis(2-butyloctyl) O′1,O1-(2-(hydroxymethyl)propane-1,3-diyl) di(heptanedioate)

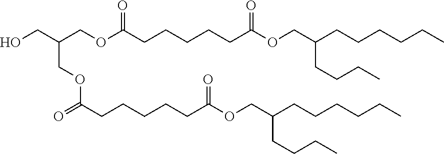 [SMILES: CCCCCCC(CCCC)COC(=O)CCCCCC(=O)OCC(CO)COC(=O)CCCCCC(=O)OCC(CCCC)CCCCCC]

Intermediate 5a was synthesized (68%) from Intermediate 4a using the method employed in the synthesis of Intermediate 2c. ¹H NMR (400 MHz, CDCl₃) δ 4.19 (h, J=5.9 Hz, 4H), 3.99 (d, J=5.8 Hz, 4H), 3.65 (t, J=5.9 Hz, 2H), 2.42-2.30 (m, 9H), 2.22 (p, J=5.9 Hz, 1H), 1.67 (ddp, J=11.5, 7.7, 3.8 Hz, 10H), 1.44-1.23 (m, 36H), 0.91 (td, J=6.6, 3.7 Hz, 12H).

### Intermediate 5b: 7,7′-bis(2-butyloctyl) O′1,O1-(2-((((4-nitrophenoxy)carbonyl)oxy)methyl)propane-1,3-diyl) di(heptanedioate)

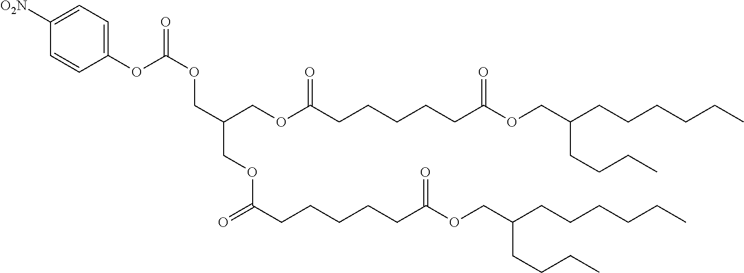 [SMILES: CCCCCCC(CCCC)COC(=O)CCCCCC(=O)OCC(COC(=O)CCCCCC(=O)OCC(CCCC)CCCCCC)COC(=O)Oc1ccc([N+](=O)[O-])cc1]

Intermediate 5b was synthesized (61%) from Intermediate 5a using the method employed in the synthesis of Intermediate 2d. ¹H NMR (400 MHz, CDCl₃) δ 8.24-8.17 (m, 2H), 7.36-7.28 (m, 2H), 4.29 (d, J=5.8 Hz, 2H), 4.20-4.08 (m, 4H), 3.95-3.84 (m, 4H), 2.45 (p, J=6.0 Hz, 1H), 2.26 (dt, J=14.7, 7.5 Hz, 8H), 1.58 (dtd, J=15.3, 7.6, 5.1 Hz, 10H), 1.36-1.09 (m, 36H), 0.81 (td, J=6.6, 3.9 Hz, 12H).

### Compound 5: 7,7′-bis(2-butyloctyl) O′1,O1-(2-((((3-(diethylamino)propoxy)carbonyl)oxy)methyl)propane-1,3-diyl) di(heptanedioate)

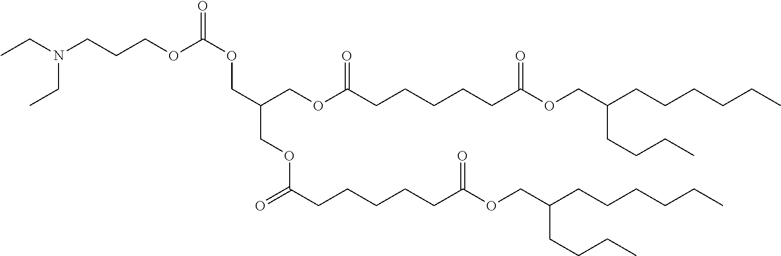 [SMILES: CCCCCCC(CCCC)COC(=O)CCCCCC(=O)OCC(COC(=O)CCCCCC(=O)OCC(CCCC)CCCCCC)COC(=O)OCCCN(CC)CC]

Compound 5 was synthesized from Intermediate 5b using the method employed in the synthesis of Compound 2. ¹H NMR (400 MHz, CDCl₃) δ 4.24-4.09 (m, 8H), 3.97 (dd, J=5.9, 2.2 Hz, 4H), 2.53 (q, J=7.0 Hz, 6H), 2.46-2.39 (m, 1H), 2.32 (dt, J=11.6, 5.7 Hz, 8H), 1.82 (p, J=6.9 Hz, 2H), 1.72-1.57 (m, 10H), 1.46-1.18 (m, 34H), 1.02 (t, J=7.2 Hz, 6H), 0.89 (dq, J=6.9, 3.3, 2.8 Hz, 12H). MS: 884.5 m/z [M+H].

### Example 6—Compound 6

### Compound 6: O,O′-(2-((((2-(diethylamino)ethyl)carbamoyl)oxy)methyl)propane-1,3-diyl) di(heptadecan-9-yl) diglutarate

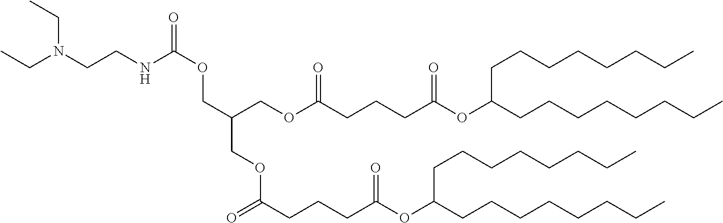 [SMILES: CCCCCCCCC(CCCCCCCC)OC(=O)CCCC(=O)OCC(COC(=O)CCCC(=O)OC(CCCCCCCC)CCCCCCCC)COC(=O)NCCN(CC)CC]

To a solution of Intermediate 2d (3 g, 1.0 equiv.) in MeCN (0.05-0.25 M) was added N′,N′-diethylethane-1,2-diamine (1.0-3.0 equiv.), pyridine (1.0-2.0 equiv.) and DMAP (0.1-1.0 equiv.). Then the mixture was stirred at 15-25° C. for at least 12 h under N₂ atmosphere. The reaction mixture was concentrated under reduced pressure to remove solvent. The residue was diluted with EtOAc and washed 2-5× with IN NaHCO₃ and 3× with H₂O. The organic layer was dried over Na₂SO₄, filtered and the filtrate was concentrated under reduced pressure to give a residue. The residue was purified by silica gel chromatography to afford product as a colorless oil. ¹H NMR (400 MHz, CDCl₃) δ 5.35 (s, 1H), 4.85 (p, J=6.3 Hz, 2H), 4.12 (t, J=6.0 Hz, 6H), 3.22 (q, J=5.8 Hz, 2H), 2.53 (d, J=6.8 Hz, 6H), 2.36 (dt, J=14.7, 7.4 Hz, 9H), 1.93 (p, J=7.5 Hz, 4H), 1.49 (q, J=5.9 Hz, 8H), 1.24 (s, 48H), 1.00 (t, J=7.1 Hz, 6H), 0.87 (t, J=6.7 Hz, 12H). MS: 953.6 m/z [M+H].

### Example 7—Compound 7

### Intermediate 7a: 2-butyloctyl 9-bromononanoate

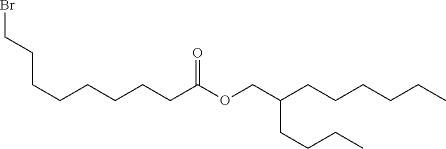 [SMILES: CCCCCCC(CCCC)COC(=O)CCCCCCCCBr]

A mixture of 9-bromononanoic acid (20 g, 1.0 equiv.), 2-butyloctan-1-ol (1.0 equiv.), EDCI (1.0-2.0 equiv.), DIPEA (2.0-3.0 equiv.) and DMAP (0.1-0.5 equiv.) in DCM (0.1-0.5 M) was stirred at 15-25° C. for at least 12 h under N₂ atmosphere. The reaction mixture was poured into water and extracted 3× with DCM or EtOAc. The combined organic phase was washed with brine, dried with anhydrous Na₂SO₄, filtered, and the filtrate was concentrated in vacuum. The residue was purified by column chromatography to afford product as a colorless oil (58%).

### Intermediate 7b: 1,1-dibenzyl 9-(2-butyloctyl) nonane-1,1,9-tricarboxylate

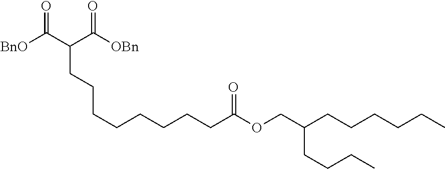 [SMILES: CCCCCCC(CCCC)COC(=O)CCCCCCCCC(C(=O)OCc1ccccc1)C(=O)OCc1ccccc1]

To a solution of Intermediate 7a (9 g, 1.0 equiv.), dibenzyl propanedioate (1.0-2.0 equiv.) in DMF (0.2-0.5 M) was added K₂CO₃ (1.0-5.0 equiv.) at 0° C., and the mixture was stirred at 15-60° C. for at least 12 h. The residue was poured into water and extracted 3× with DCM or EtOAc. The combined organic phase was washed with brine, dried with anhydrous Na₂SO₄, filtered, and the filtrate was concentrated in vacuum. The residue was purified by column chromatography to afford product as a colorless oil. ¹H NMR (400 MHz, CDCl₃) δ 7.42-7.32 (m, 10H), 5.19 (s, 4H), 4.01 (d, J=5.8 Hz, 2H), 3.47 (t, J=7.5 Hz, 1H), 2.33 (t, J=7.5 Hz, 2H), 1.96 (q, J=7.3 Hz, 2H), 1.70-1.54 (m, 6H), 1.30 (dh, J=10.1, 3.6, 3.2 Hz, 34H), 0.95-0.90 (m, 6H).

### Intermediate 7c: 2-(9-((2-butyloctyl)oxy)-9-oxononyl)malonic Acid

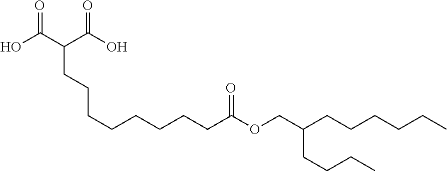 [SMILES: CCCCCCC(CCCC)COC(=O)CCCCCCCCC(C(=O)O)C(=O)O]

To a suspension of Pd/C (0.1-2.0 equiv.) in THF (0.1-0.5 M) was added Intermediate 7b (1.0 equiv.). The reaction mixture was stirred at 15-25° C. for at least 12 h under H2 atmosphere. The reaction mixture was filtered, and the filtrate was concentrated in vacuum and purified by column chromatography to afford product as a colorless oil (60%). ¹H NMR (400 MHz, CDCl₃) δ 3.91 (d, J=5.8 Hz, 2H), 3.37 (t, J=7.3 Hz, 1H), 2.24 (t, J=7.5 Hz, 2H), 1.89 (q, J=7.4 Hz, 2H), 1.54 (t, J=7.3 Hz, 3H), 1.33-1.18 (m, 24H), 0.82 (td, J=6.7, 3.7 Hz, 6H).

### Intermediate 7d: 2-butyloctyl 11-hydroxy-10-(hydroxymethyl)undecanoate

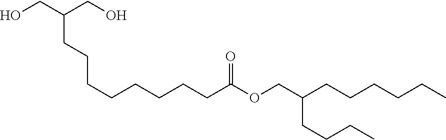 [SMILES: CCCCCCC(CCCC)COC(=O)CCCCCCCCC(CO)CO]

To a solution of Intermediate 7c (4 g, 1.0 equiv.) in THF (0.2 M) was added BH₃·THF (2.5 equiv.) dropwise at 0° C., and the mixture was stirred at 20° C. for 12 h under N₂ atmosphere. The reaction mixture was poured into aq. NH₄Cl and stirred at 5° C. for 0.5 h. The mixture was then extracted 3-5× with EtOAc. The organic layer was concentrated in vacuum, and the residue was purified by column chromatography to afford product as a colorless oil. ¹H NMR (400 MHz, CDCl₃) δ 3.90 (d, J=5.8 Hz, 2H), 3.75 (td, J=12.5, 11.6, 4.8 Hz, 1H), 3.59 (dt, J=9.5, 6.5 Hz, 2H), 3.34 (q, J=6.7, 6.1 Hz, 4H), 2.23 (t, J=7.5 Hz, 2H), 1.57 (dtt, J=10.2, 7.1, 3.5 Hz, 8H), 1.21 (d, J=4.8 Hz, 26H), 0.82 (td, J=8.1, 6.9, 5.2 Hz, 6H).

### Intermediate 7e: 11-((2-butyloctyl)oxy)-2-(hydroxymethyl)-11-oxoundecyl (9Z,12Z)-octadeca-9,12-dienoate

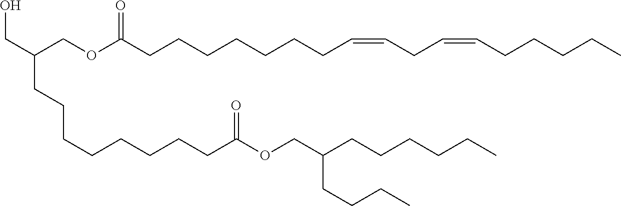 [SMILES: CCCCC/C=C\C/C=C\CCCCCCCC(=O)OCC(CO)CCCCCCCCC(=O)OCC(CCCC)CCCCCC]

To a mixture of Intermediate 7e (3.5 g, 1.0 equiv.), EDCI (1.0-2.0 equiv.), DMAP (0.1-0.5 equiv.) and DIPEA (2.0-4.0 equiv.) in DCM (0.1-0.5 M) was added (9Z,12Z)-octadeca-9,12-dienoic acid (1.0 equiv.). The reaction mixture was stirred at 15-25° C. for at least 12 h under N₂ atmosphere. The reaction mixture was concentrated under reduced pressure to remove solvent, and the residue was poured into water and extracted 3× with EtOAc or DCM. The combined organic phase was washed with brine, dried with anhydrous Na₂SO₄, filtered and the filtrate was concentrated in vacuum. The residue was purified by column chromatography to afford product as a colorless oil (43%). ¹H NMR (400 MHz, CDCl₃) δ 5.38-5.16 (m, 4H), 4.24 (t, J=6.7 Hz, 1H), 4.15 (dd, J=11.2, 4.3 Hz, 1H), 4.01 (dd, J=11.2, 6.7 Hz, 1H), 3.90 (d, J=5.8 Hz, 2H), 3.48 (dd, J=32.6, 9.4 Hz, 2H), 2.70 (t, J=6.4 Hz, 2H), 2.24 (dt, J=10.6, 7.5 Hz, 4H), 1.98 (q, J=6.9 Hz, 4H), 1.54 (ddd, J=19.9, 9.5, 5.9 Hz, 7H), 1.24 (ddq, J=13.9, 9.8, 5.9 Hz, 43H), 0.82 (td, J=6.7, 3.4 Hz, 9H).

### Intermediate 7f: 11-((2-butyloctyl)oxy)-2-((((4-nitrophenoxy)carbonyl)oxy)methyl)-11-oxoundecyl (9Z,12Z)-octadeca-9,12-dienoate

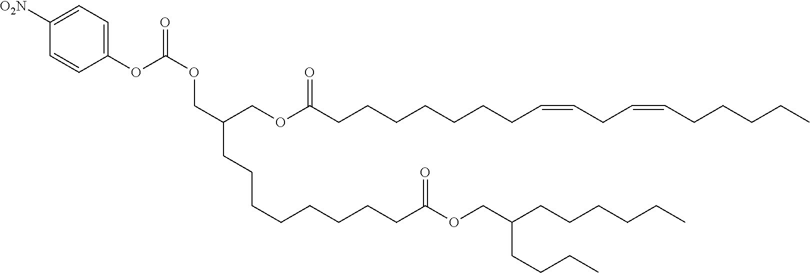 [SMILES: CCCCC/C=C\C/C=C\CCCCCCCC(=O)OCC(CCCCCCCCC(=O)OCC(CCCC)CCCCCC)COC(=O)Oc1ccc([N+](=O)[O-])cc1]

To a solution of (4-nitrophenyl) carbonochloridate (1.0-3.0 equiv.) and Intermediate 7e (1.0 equiv.) in DCM (0.05-0.5 M) was added pyridine (1.0-3.0 equiv.), and the mixture was stirred at 15-25° C. for at least 12 h under N₂ atmosphere. The reaction mixture was concentrated under reduced pressure to remove solvent. The residue was purified by silica gel chromatography to afford product as a colorless oil. ¹H NMR (400 MHz, CDCl₃) δ 8.25-8.17 (m, 2H), 7.36-7.28 (m, 2H), 5.38-5.16 (m, 4H), 4.31-4.14 (m, 3H), 4.10 (dd, J=11.2, 4.7 Hz, 1H), 4.02 (dd, J=11.3, 6.7 Hz, 1H), 3.95-3.88 (m, 2H), 2.70 (t, J=6.3 Hz, 2H), 2.24 (dt, J=9.7, 7.5 Hz, 4H), 2.07-1.91 (m, 5H), 1.55 (tt, J=7.5, 3.8 Hz, 5H), 1.39-1.12 (m, 42H), 0.81 (dh, J=6.1, 3.4, 2.9 Hz, 9H).

### Compound 7: 11-((2-butyloctyl)oxy)-2-((((3-(diethylamino)propoxy)carbonyl)oxy)methyl)-11-oxoundecyl (9Z,12Z)-octadeca-9,12-dienoate

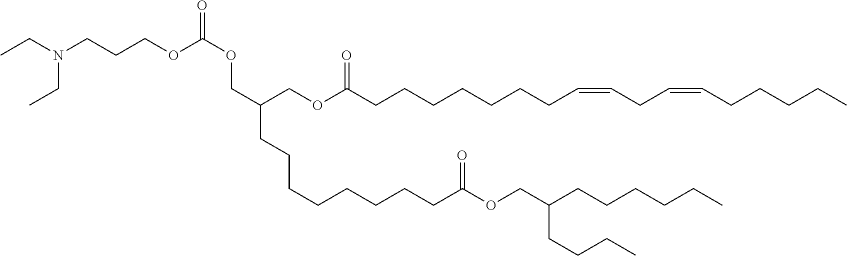 [SMILES: CCCCC/C=C\C/C=C\CCCCCCCC(=O)OCC(CCCCCCCCC(=O)OCC(CCCC)CCCCCC)COC(=O)OCCCN(CC)CC]

Compound 7 was synthesized (77%) from Intermediate 7f and 3-(diethylamino)propan-1-ol using the method employed in the synthesis of Compound 6. ¹H NMR (400 MHz, CDCl₃) δ 5.28 (tt, J=11.2, 5.4 Hz, 4H), 4.13 (t, J=6.4 Hz, 2H), 4.06-3.93 (m, 4H), 3.90 (d, J=5.8 Hz, 2H), 2.70 (t, J=6.4 Hz, 2H), 2.66-2.46 (m, 5H), 2.23 (td, J=7.6, 2.6 Hz, 4H), 1.98 (q, J=6.9 Hz, 4H), 1.94-1.82 (m, 2H), 1.54 (t, J=7.2 Hz, 6H), 1.35-1.14 (m, 45H), 1.04 (s, 6H), 0.82 (td, J=6.6, 3.3 Hz, 9H). MS: 820.7 m/z [M+H].

### Example 8—Compound 8

### Intermediate 8a: heptadecan-9-yl 7-bromoheptanoate

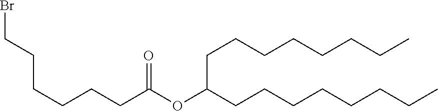 [SMILES: CCCCCCCCC(CCCCCCCC)OC(=O)CCCCCCBr]

Intermediate 8a was synthesized (58%) from 7-bromoheptanoic acid and heptadecan-9-ol using the method employed in the synthesis of Intermediate 7a. ¹H NMR (400 MHz, CDCl₃) δ 4.80 (p, J=6.3 Hz, 1H), 3.33 (t, J=6.8 Hz, 2H), 2.21 (t, J=7.5 Hz, 2H), 1.78 (p, J=7.0 Hz, 2H), 1.55 (t, J=7.2 Hz, 2H), 1.47-1.16 (m, 36H), 0.81 (t, J=6.7 Hz, 6H).

### Intermediate 8b: 1,1-dibenzyl 7-(heptadecan-9-yl) heptane-1,1,7-tricarboxylate

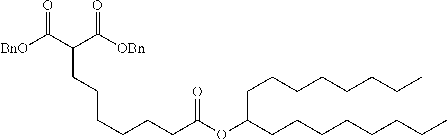 [SMILES: CCCCCCCCC(CCCCCCCC)OC(=O)CCCCCCC(C(=O)OCc1ccccc1)C(=O)OCc1ccccc1]

Intermediate 8b was synthesized from Intermediate 8a using the method employed in the synthesis of Intermediate 7b. ¹H NMR (400 MHz, CDCl₃) δ 7.30-7.19 (m, 10H), 5.07 (d, J=1.3 Hz, 4H), 4.79 (p, J=6.2 Hz, 1H), 3.36 (t, J=7.5 Hz, 1H), 2.18 (t, J=7.5 Hz, 2H), 1.85 (d, J=7.1 Hz, 2H), 1.45 (dd, J=16.0, 3.7 Hz, 8H), 1.28-1.05 (m, 30H), 0.80 (t, J=6.7 Hz, 6H).

### Intermediate 8c: 2-(7-(heptadecan-9-yloxy)-7-oxoheptyl)malonic Acid

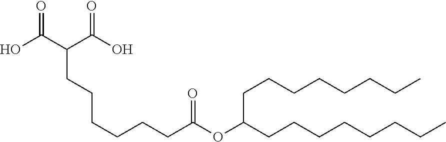 [SMILES: CCCCCCCCC(CCCCCCCC)OC(=O)CCCCCCC(C(=O)O)C(=O)O]

Intermediate 8c was synthesized (69%) from Intermediate 8b using the method employed in the synthesis of Intermediate 7c.

### Intermediate 8d: heptadecan-9-yl 9-hydroxy-8-(hydroxymethyl)nonanoate

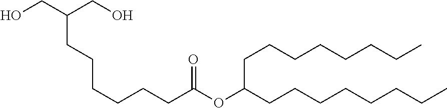 [SMILES: CCCCCCCCC(CCCCCCCC)OC(=O)CCCCCCC(CO)CO]

Intermediate 8d was synthesized (47%) from Intermediate 8c using the method employed in the synthesis of Intermediate 7d. ¹H NMR (400 MHz, CDCl₃) δ 4.79 (p, J=6.3 Hz, 1H), 3.90-3.69 (m, 2H), 3.59 (dt, J=10.7, 7.8 Hz, 2H), 2.21 (td, J=7.4, 1.7 Hz, 2H), 1.53 (q, J=7.3 Hz, 2H), 1.43 (q, J=6.1 Hz, 4H), 1.22 (d, J=23.1 Hz, 32H), 0.86-0.76 (m, 6H).

### Intermediate 8e: heptadecan-9-yl (9-(heptadecan-9-yloxy)-2-(hydroxymethyl)-9-oxononyl) glutarate

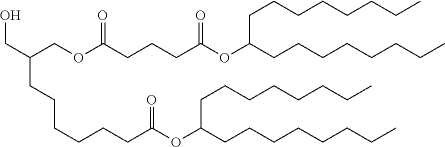 [SMILES: CCCCCCCCC(CCCCCCCC)OC(=O)CCCCCCC(CO)COC(=O)CCCC(=O)OC(CCCCCCCC)CCCCCCCC]

Intermediate 8e was synthesized (39%) from Intermediate 8d and Intermediate 2b using the method employed in the synthesis of Intermediate 7e.

### Intermediate 8f: heptadecan-9-yl (9-(heptadecan-9-yloxy)-2-((((4-nitrophenoxy)carbonyl)oxy)methyl)-9-oxononyl) glutarate

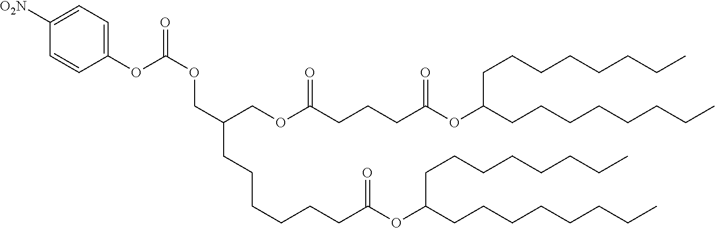 [SMILES: CCCCCCCCC(CCCCCCCC)OC(=O)CCCCCCC(COC(=O)CCCC(=O)OC(CCCCCCCC)CCCCCCCC)COC(=O)Oc1ccc([N+](=O)[O-])cc1]

Intermediate 8f was synthesized from Intermediate 8e using the method employed in the synthesis of Intermediate 7f. ¹H NMR (400 MHz, CDCl₃) δ 8.26-8.17 (m, 2H), 7.38-7.28 (m, 2H), 4.80 (p, J=6.2 Hz, 2H), 4.22-3.99 (m, 4H), 2.38-2.17 (m, 6H), 2.04 (q, J=6.0 Hz, 1H), 1.89 (p, J=7.4 Hz, 2H), 1.55 (q, J=7.0 Hz, 3H), 1.43 (t, J=6.2 Hz, 8H), 1.37-1.13 (m, 58H), 0.80 (t, J=6.7 Hz, 12H).

### Compound 8: 2-((((3-(diethylamino)propoxy)carbonyl)oxy)methyl)-9-(heptadecan-9-yloxy)-9-oxononyl heptadecan-9-yl glutarate

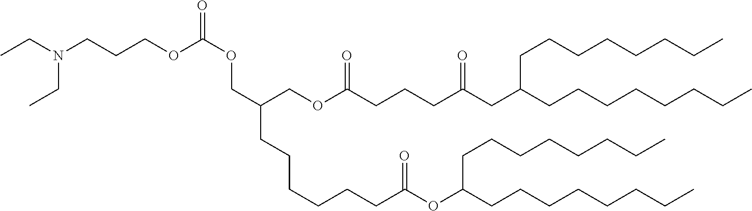 [SMILES: CCCCCCCCC(CCCCCCCC)CC(=O)CCCC(=O)OCC(CCCCCCC(=O)OC(CCCCCCCC)CCCCCCCC)COC(=O)OCCCN(CC)CC]

Compound 8 was synthesized from Intermediate 8f using the method employed in the synthesis of Compound 6. ¹H NMR (400 MHz, CDCl₃) δ 4.86 (pd, J=6.3, 2.2 Hz, 2H), 4.18 (t, J=6.5 Hz, 2H), 4.14-4.01 (m, 4H), 2.52 (s, 5H), 2.36 (dt, J=11.7, 7.4 Hz, 4H), 2.27 (t, J=7.6 Hz, 2H), 1.97 (dt, J=22.2, 7.9 Hz, 3H), 1.83 (s, 2H), 1.60 (dd, J=10.1, 4.5 Hz, 2H), 1.49 (t, J 3=6.1 Hz, 8H), 1.38-1.19 (m, 55H), 1.02 (d, J=7.5 Hz, 6H), 0.87 (t, J=6.7 Hz, 12H). MS: 952.8 m/z [M+H].

### Example 9—Compound 9

### Intermediate 9a: heptadecan-9-yl 9-bromononanoate

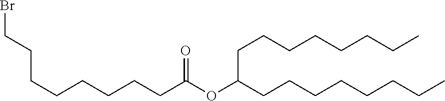 [SMILES: CCCCCCCCC(CCCCCCCC)OC(=O)CCCCCCCCBr]

Intermediate 9a was synthesized from 9-bromononanoic acid and heptadecan-9-ol using the method employed in the synthesis of Intermediate 7a. ¹H NMR (400 MHz, CDCl₃) δ 4.80 (p, J=6.3 Hz, 1H), 3.33 (t, J=6.8 Hz, 2H), 2.21 (t, J=7.5 Hz, 2H), 1.78 (p, J=7.0 Hz, 2H), 1.55 (t, J=7.2 Hz, 2H), 1.43 (q, J=6.0 Hz, 4H), 1.41-1.30 (m, 3H), 1.22 (d, J=23.8 Hz, 30H), 0.81 (t, J=6.7 Hz, 6H).

### Intermediate 9b: 1,1-dibenzyl 9-(heptadecan-9-yl) nonane-1,1,9-tricarboxylate

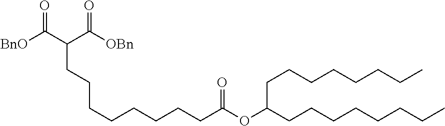 [SMILES: CCCCCCCCC(CCCCCCCC)OC(=O)CCCCCCCCC(C(=O)OCc1ccccc1)C(=O)OCc1ccccc1]

Intermediate 9b was synthesized (90%) from Intermediate 9a using the method employed in the synthesis of Intermediate 7b. ¹H NMR (400 MHz, CDCl₃) δ 7.34-7.20 (m, 10H), 5.07 (s, 3H), 4.80 (td, J=6.3, 2.4 Hz, 1H), 3.36 (t, J=7.5 Hz, 1H), 2.20 (d, J=14.9 Hz, 2H), 1.85 (q, J=7.3 Hz, 2H), 1.49 (dq, J=38.6, 6.7, 6.1 Hz, 7H), 1.20 (d, J=7.4 Hz, 36H), 0.81 (t, J=6.6 Hz, 6H).

### Intermediate 9c: 2-(9-(heptadecan-9-yloxy)-9-oxononyl)malonic Acid

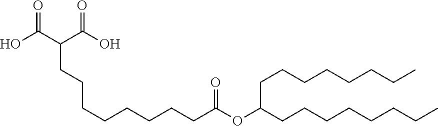 [SMILES: CCCCCCCCC(CCCCCCCC)OC(=O)CCCCCCCCC(C(=O)O)C(=O)O]

Intermediate 9c was synthesized (65%) from Intermediate 9b using the method employed in the synthesis of Intermediate 7c. ¹H NMR (400 MHz, CDCl₃) δ 4.80 (p, J=6.2 Hz, 1H), 3.35 (t, J=7.3 Hz, 1H), 2.22 (t, J=7.5 Hz, 2H), 1.87 (q, J=7.5 Hz, 2H), 1.54 (t, J=7.3 Hz, 2H), 1.44 (q, J=6.2 Hz, 4H), 1.21 (d, J=16.4 Hz, 36H), 0.81 (t, J=6.7 Hz, 6H).

### Intermediate 9d: heptadecan-9-yl 11-hydroxy-10-(hydroxymethyl)undecanoate

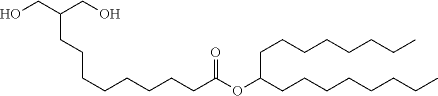 [SMILES: CCCCCCCCC(CCCCCCCC)OC(=O)CCCCCCCCC(CO)CO]

Intermediate 9d was synthesized (35%) from Intermediate 9c using the method employed in the synthesis of Intermediate 7d.

### Intermediate 9e: 11-(heptadecan-9-yloxy)-2-(hydroxymethyl)-11-oxoundecyl (9Z,12Z)-octadeca-9,12-dienoate

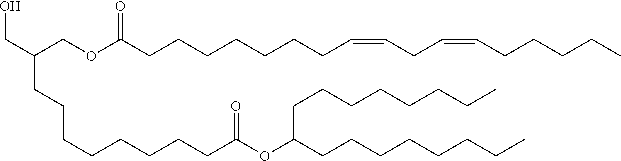 [SMILES: CCCCC/C=C\C/C=C\CCCCCCCC(=O)OCC(CO)CCCCCCCCC(=O)OC(CCCCCCCC)CCCCCCCC]

Intermediate 9e was synthesized (42%) from Intermediate 9d using the method employed in the synthesis of Intermediate 7d. ¹H NMR (400 MHz, CDCl₃) δ 5.38-5.21 (m, 4H), 4.80 (p, J=6.3 Hz, 1H), 4.15 (dd, J=11.2, 4.3 Hz, 1H), 4.01 (dd, J=11.2, 6.7 Hz, 1H), 3.56-3.44 (m, 1H), 3.45-3.38 (m, 1H), 2.70 (t, J=6.3 Hz, 2H), 2.23 (dt, J=18.4, 7.5 Hz, 4H), 1.98 (q, J=6.8 Hz, 4H), 1.72 (td, J=6.6, 3.3 Hz, 1H), 1.54 (tt, J=7.3, 3.6 Hz, 4H), 1.43 (q, J=6.0 Hz, 4H), 1.34-1.11 (m, 52H), 0.81 (q, J=6.6 Hz, 9H).

### Intermediate 9f: 11-(heptadecan-9-yloxy)-2-((((4-nitrophenoxy)carbonyl)oxy)methyl)-11-oxoundecyl (9Z,12Z)-octadeca-9,12-dienoate

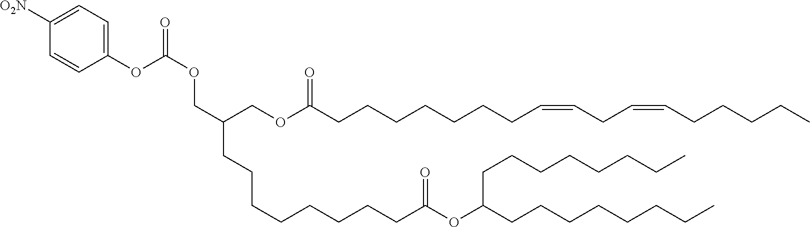 [SMILES: CCCCC/C=C\C/C=C\CCCCCCCC(=O)OCC(CCCCCCCCC(=O)OC(CCCCCCCC)CCCCCCCC)COC(=O)Oc1ccc([N+](=O)[O-])cc1]

Intermediate 9f was synthesized from Intermediate 9e using the method employed in the synthesis of Intermediate 7e. ¹H NMR (400 MHz, CDCl₃) δ 8.27-8.15 (m, 2H), 7.37-7.28 (m, 2H), 5.38-5.19 (m, 4H), 4.80 (p, J=6.3 Hz, 1H), 4.28-4.16 (m, 2H), 4.10 (dd, J=11.3, 4.7 Hz, 1H), 4.02 (dd, J=11.3, 6.6 Hz, 1H), 2.70 (t, J=6.3 Hz, 2H), 2.23 (dt, J=17.9, 7.5 Hz, 4H), 2.06-1.91 (m, 5H), 1.59-1.48 (m, 4H), 1.43 (t, J=6.2 Hz, 4H), 1.38-1.14 (m, 51H), 0.81 (td, J=6.8, 4.7 Hz, 9H).

### Compound 9: 2-((((3-(diethylamino)propoxy)carbonyl)oxy)methyl)-11-(heptadecan-9-yloxy)-11-oxoundecyl (9Z,12Z)-octadeca-9,12-dienoate

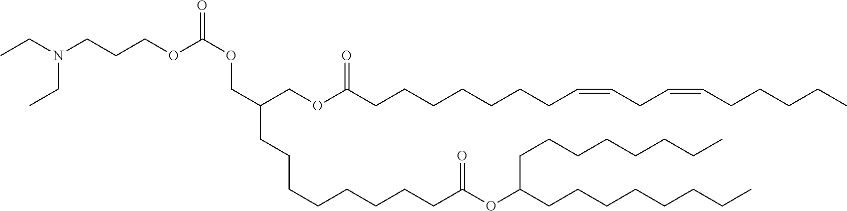 [SMILES: CCCCC/C=C\C/C=C\CCCCCCCC(=O)OCC(CCCCCCCCC(=O)OC(CCCCCCCC)CCCCCCCC)COC(=O)OCCCN(CC)CC]

Compound 9 was synthesized from Intermediate 9e using the method employed in the synthesis of Compound 7. ¹H NMR (400 MHz, CDCl₃) δ 5.38-5.18 (m, 4H), 4.79 (p, J=6.3 Hz, 1H), 4.11 (t, J=6.6 Hz, 2H), 4.07-3.91 (m, 4H), 2.70 (t, J=6.4 Hz, 2H), 2.47-2.40 (m, 5H), 2.22 (dt, J=10.6, 7.5 Hz, 4H), 1.96 (dq, J=11.9, 6.3, 5.9 Hz, 4H), 1.74 (p, J=6.8 Hz, 2H), 1.53 (d, J=7.4 Hz, 4H), 1.43 (q, J=5.9 Hz, 4H), 1.35-1.11 (m, 48H), 0.94 (t, J=7.1 Hz, 6H), 0.81 (h, J=5.4 Hz, 9H). MS: 890.7 m/z [M+H].

### Example 10—Compound 10

### Compound 10: 2-((((2-(diethylamino)ethyl)carbamoyl)oxy)methyl)-11-(heptadecan-9-yloxy)-11-oxoundecyl (9Z,12Z)-octadeca-9,12-dienoate

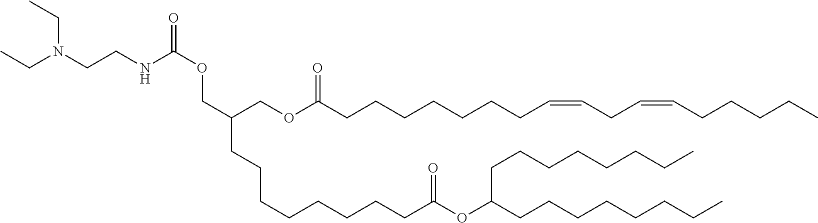 [SMILES: CCCCC/C=C\C/C=C\CCCCCCCC(=O)OCC(CCCCCCCCC(=O)OC(CCCCCCCC)CCCCCCCC)COC(=O)NCCN(CC)CC]

Compound 10 was synthesized from Intermediate 9f and N′,N′-diethylethane-1,2-diamine using the method employed in the synthesis of Compound 1. ¹H NMR (400 MHz, CDCl₃) δ 5.42-5.27 (m, 4H), 4.86 (p, J=6.3 Hz, 1H), 4.04 (tq, J=11.0, 5.5, 4.6 Hz, 4H), 3.31-3.11 (m, 2H), 2.77 (t, J=6.4 Hz, 2H), 2.53 (s, 5H), 2.28 (dt, J=10.7, 7.5 Hz, 4H), 2.04 (q, J=6.9 Hz, 4H), 1.61 (p, J=7.3 Hz, 4H), 1.50 (q, J=5.9 Hz, 4H), 1.40-1.19 (m, 52H), 1.01 (d, J=10.1 Hz, 6H), 0.90-0.83 (m, 9H). MS: 875.5 m/z [M+H].

### Example 11—Compound 11

### Compound 11: 2-((((3-(ethyl(methyl)amino)propoxy)carbonyl)oxy)methyl)-11-(heptadecan-9-yloxy)-11-oxoundecyl (9Z,12Z)-octadeca-9,12-dienoate

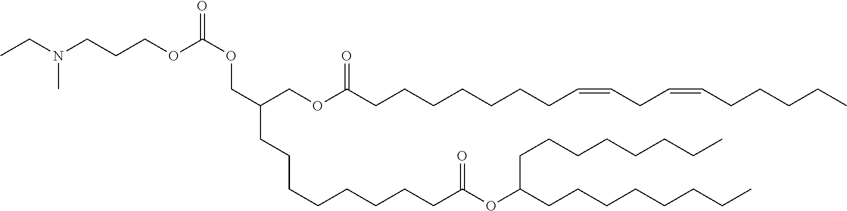 [SMILES: CCCCC/C=C\C/C=C\CCCCCCCC(=O)OCC(CCCCCCCCC(=O)OC(CCCCCCCC)CCCCCCCC)COC(=O)OCCCN(C)CC]

Compound 11 was synthesized from Intermediate 9f and 3-[ethyl(methyl)amino]pro pan-1-ol using the method employed in the synthesis of Compound 1. ¹H NMR (400 MHz, CDCl₃) δ 5.37-5.20 (m, 4H), 4.79 (p, J=6.3 Hz, 1H), 4.11 (t, J=6.5 Hz, 2H), 4.09-3.92 (m, 4H), 2.70 (t, J=6.4 Hz, 2H), 2.50-2.29 (m, 4H), 2.27-2.10 (m, 7H), 1.98 (q, J=6.8 Hz, 5H), 1.79 (p, J=7.1 Hz, 2H), 1.54 (p, J=7.1 Hz, 4H), 1.43 (q, J=5.8 Hz, 4H), 1.38-1.05 (m, 49H), 0.99 (t, J=7.2 Hz, 3H), 0.88-0.74 (m, 9H). MS: 876.5 m/z [M+H].

### Example 12—Compound 12

### Intermediate 12a: nonan-5-yl 5-bromopentanoate

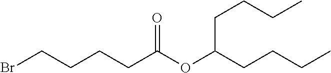 [SMILES: CCCCC(CCCC)OC(=O)CCCCBr]

Intermediate 12a was synthesized from 5-bromopentanoic acid and nonan-5-ol using the method employed in the synthesis of Intermediate 7a. ¹H NMR (400 MHz, CDCl₃) δ 4.86 (p, J=6.3 Hz, 1H), 3.39 (t, J=6.6 Hz, 2H), 2.31 (t, J=7.2 Hz, 2H), 1.94-1.84 (m, 2H), 1.82-1.71 (m, 2H), 1.57-1.43 (m, 4H), 1.26 (tdd, J=13.6, 10.2, 6.7 Hz, 8H), 0.87 (t, J=6.9 Hz, 6H).

### Intermediate 12b: 1,1-dibenzyl 5-(nonan-5-yl) pentane-1,1,5-tricarboxylate

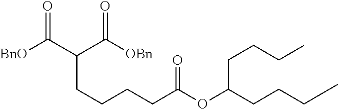 [SMILES: CCCCC(CCCC)OC(=O)CCCCC(C(=O)OCc1ccccc1)C(=O)OCc1ccccc1]

Intermediate 12b was synthesized from Intermediate 12a using the method employed in the synthesis of Intermediate 7b. ¹H NMR (400 MHz, CDCl₃) δ 7.40-7.08 (m, 10H), 5.11-4.94 (m, 4H), 4.78 (p, J=6.3 Hz, 1H), 3.36 (t, J=7.5 Hz, 1H), 2.16 (t, J=7.6 Hz, 2H), 1.87 (q, J=7.6 Hz, 2H), 1.54 (q, J=7.7 Hz, 2H), 1.47-1.34 (m, 4H), 1.32-1.08 (m, 10H), 0.81 (t, J=6.9 Hz, 6H).

### Intermediate 12c: 2-(5-(nonan-5-yloxy)-5-oxopentyl)malonic Acid

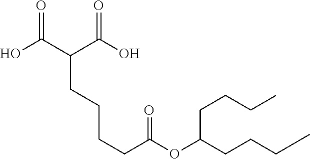 [SMILES: CCCCC(CCCC)OC(=O)CCCCC(C(=O)O)C(=O)O]

Intermediate 12c was synthesized from Intermediate 12b using the method employed in the synthesis of Intermediate 7c. ¹H NMR (400 MHz, CDCl₃) δ 4.87 (p, J=6.3 Hz, 1H), 3.43 (t, J=7.3 Hz, 1H), 2.32 (t, J=7.5 Hz, 2H), 1.96 (q, J=7.6 Hz, 2H), 1.68 (p, J=7.6 Hz, 2H), 1.58-1.39 (m, 6H), 1.27 (dddd, J=19.7, 9.6, 7.8, 5.6 Hz, 9H), 0.88 (t, J=7.0 Hz, 6H).

### Intermediate 12d: nonan-5-yl 7-hydroxy-6-(hydroxymethyl)heptanoate

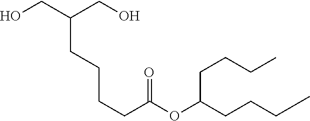 [SMILES: CCCCC(CCCC)OC(=O)CCCCC(CO)CO]

Intermediate 12d was synthesized from Intermediate 12c using the method employed in the synthesis of Intermediate 7d.

### Intermediate 12e: 2-(hydroxymethyl)-7-(nonan-5-yloxy)-7-oxoheptyl (9Z,12Z)-octadeca-9,12-dienoate (

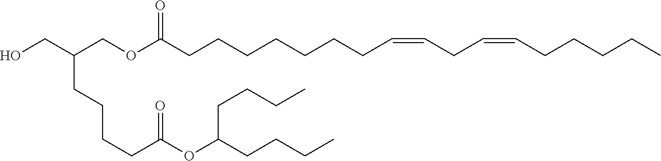 [SMILES: CCCCC/C=C\C/C=C\CCCCCCCC(=O)OCC(CO)CCCCC(=O)OC(CCCC)CCCC]

Intermediate 12e was synthesized from Intermediate 12d using the method employed in the synthesis of Intermediate 7e. ¹H NMR (400 MHz, CDCl₃) δ 5.44-5.26 (m, 4H), 4.86 (p, J=6.3 Hz, 1H), 4.20 (dd, J=11.3, 4.4 Hz, 1H), 4.07 (dd, J=11.3, 6.6 Hz, 1H), 3.58 (dd, J=11.3, 4.5 Hz, 1H), 3.49 (dd, J=11.3, 6.4 Hz, 1H), 2.76 (t, J=6.4 Hz, 2H), 2.30 (q, J=7.3 Hz, 4H), 2.04 (q, J=6.9 Hz, 4H), 1.84-1.74 (m, 1H), 1.70-1.57 (m, 4H), 1.50 (h, J=6.2 Hz, 4H), 1.44-1.20 (m, 26H), 0.96-0.81 (m, 9H).

### Intermediate 12f: 2-((((4-nitrophenoxy)carbonyl)oxy)methyl)-7-(nonan-5-yloxy)-7-oxoheptyl (9Z,12Z)-octadeca-9,12-dienoate

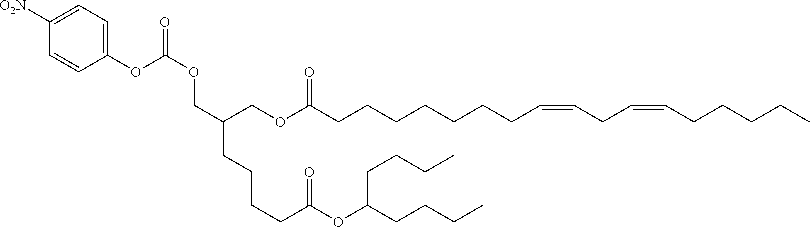 [SMILES: CCCCC/C=C\C/C=C\CCCCCCCC(=O)OCC(CCCCC(=O)OC(CCCC)CCCC)COC(=O)Oc1ccc([N+](=O)[O-])cc1]

Intermediate 12f was synthesized from Intermediate 12e using the method employed in the synthesis of Intermediate 7f. ¹H NMR (400 MHz, CDCl₃) δ 8.32-8.23 (m, 2H), 7.42-7.34 (m, 2H), 5.44-5.25 (m, 4H), 4.87 (p, J=6.3 Hz, 1H), 4.27 (h, J=5.9 Hz, 2H), 4.20-4.03 (m, 2H), 2.76 (t, J=6.4 Hz, 2H), 2.32 (td, J=7.5, 1.7 Hz, 4H), 2.11 (t, J=6.0 Hz, 1H), 2.04 (q, J=7.0 Hz, 4H), 1.63 (ddd, J=20.1, 16.1, 9.4 Hz, 4H), 1.56-1.41 (m, 8H), 1.29 (tdd, J=10.3, 7.6, 4.5 Hz, 23H), 0.88 (t, J=6.8 Hz, 9H).

### Compound 12: 2-((((2-(diethylamino)ethyl)carbamoyl)oxy)methyl)-7-(nonan-5-yloxy)-7-oxoheptyl (9Z,12Z)-octadeca-9,12-dienoate

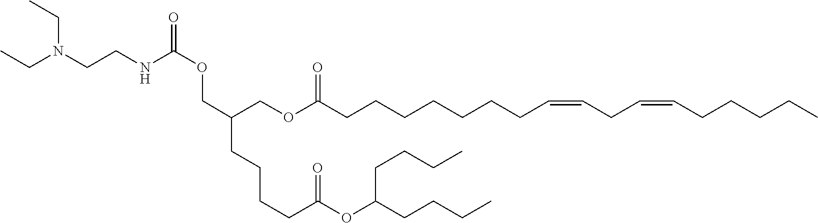 [SMILES: CCCCC/C=C\C/C=C\CCCCCCCC(=O)OCC(CCCCC(=O)OC(CCCC)CCCC)COC(=O)NCCN(CC)CC]

Compound 12 was synthesized from Intermediate 12f and N′,N′-diethylethane-1,2-diamine using the method employed in the synthesis of Compound 1. ¹H NMR (400 MHz, CDCl₃) δ 5.28 (tt, J=11.1, 5.6 Hz, 4H), 4.80 (p, J=6.3 Hz, 1H), 3.98 (td, J=13.6, 12.3, 6.6 Hz, 4H), 3.17 (s, 2H), 2.70 (t, J=6.5 Hz, 2H), 2.48 (s, 5H), 2.22 (td, J=7.6, 4.0 Hz, 4H), 1.98 (q, J=6.8 Hz, 4H), 1.54 (p, J=7.0 Hz, 4H), 1.50-1.41 (m, 4H), 1.39-1.15 (m, 27H), 0.96 (s, 6H), 0.82 (t, J=6.8 Hz, 9H). MS: 707.6 m/z [M+H].

### Example 13—Compound 13

### Compound 13: 2-((((3-(ethyl(methyl)amino)propoxy)carbonyl)oxy)methyl)-7-(nonan-5-yloxy)-7-oxoheptyl (9Z,12Z)-octadeca-9,12-dienoate

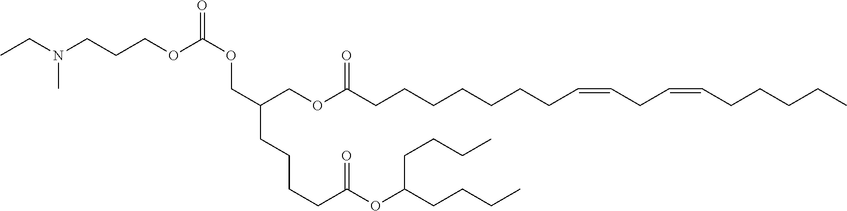 [SMILES: CCCCC/C=C\C/C=C\CCCCCCCC(=O)OCC(CCCCC(=O)OC(CCCC)CCCC)COC(=O)OCCCN(C)CC]

Compound 13 was synthesized from Intermediate 12f and 3-[ethyl(methyl)amino]propan-1-ol using the method employed in the synthesis of Compound 1. ¹H NMR (400 MHz, CDCl₃) δ 5.38-5.18 (m, 4H), 4.80 (p, J=6.3 Hz, 1H), 4.12 (t, J=6.5 Hz, 2H), 4.08-3.93 (m, 4H), 2.70 (t, J=6.5 Hz, 2H), 2.39 (s, 4H), 2.27-2.11 (m, 7H), 1.97 (p, J=8.1, 7.4 Hz, 5H), 1.55 (q, J=7.5 Hz, 4H), 1.50-1.41 (m, 4H), 1.39-1.14 (m, 27H), 1.00 (t, J=7.4 Hz, 3H), 0.82 (td, J=7.0, 1.6 Hz, 9H). MS: 708.6 m/z [M+H].

### Example 14—Compound 14

### Compound 14: 2-((((3-(diethylamino)propoxy)carbonyl)oxy)methyl)-7-(nonan-5-yloxy)-7-oxoheptyl (9Z,12Z)-octadeca-9,12-dienoate

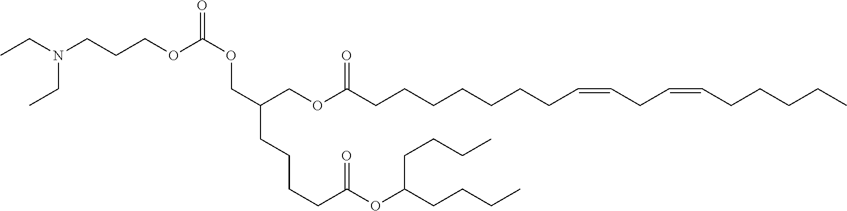 [SMILES: CCCCC/C=C\C/C=C\CCCCCCCC(=O)OCC(CCCCC(=O)OC(CCCC)CCCC)COC(=O)OCCCN(CC)CC]

Compound 14 was synthesized from Intermediate 12f using the method employed in the synthesis of Compound 1. ¹H NMR (400 MHz, CDCl₃) δ 5.37-5.21 (m, 4H), 4.80 (p, J=6.3 Hz, 1H), 4.11 (t, J=6.6 Hz, 2H), 4.07-3.94 (m, 4H), 2.70 (t, J=6.4 Hz, 2H), 2.50-2.39 (m, 6H), 2.22 (td, J=7.5, 3.1 Hz, 4H), 1.97 (p, J=8.0, 7.4 Hz, 5H), 1.75 (p, J=6.8 Hz, 2H), 1.54 (t, J=7.5 Hz, 4H), 1.44 (td, J=7.8, 6.9, 4.4 Hz, 4H), 1.39-1.15 (m, 27H), 0.95 (t, J=7.1 Hz, 6H), 0.87-0.74 (m, 9H). MS: 722.6 m/z [M+H].

### Example 15—Compound 15

### Intermediate 15a: heptadecan-9-yl 5-bromopentanoate

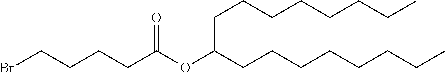 [SMILES: CCCCCCCCC(CCCCCCCC)OC(=O)CCCCBr]

Intermediate 15a was synthesized from 5-bromopentanoic and heptadecan-9-ol using the method employed in the synthesis of Intermediate 7a.

### Intermediate 15b: 1,1-dibenzyl 5-(heptadecan-9-yl) pentane-1,1,5-tricarboxylate

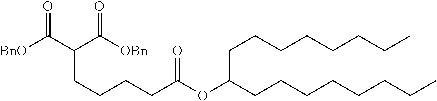 [SMILES: CCCCCCCCC(CCCCCCCC)OC(=O)CCCCC(C(=O)OCc1ccccc1)C(=O)OCc1ccccc1]

Intermediate 15b was synthesized from Intermediate 15a using the method employed in the synthesis of Intermediate 7b.

### Intermediate 15c: 2-(5-(heptadecan-9-yloxy)-5-oxopentyl)malonic Acid

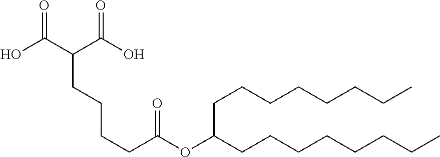 [SMILES: CCCCCCCCC(CCCCCCCC)OC(=O)CCCCC(C(=O)O)C(=O)O]

Intermediate 15c was synthesized from Intermediate 15b using the method employed in the synthesis of Intermediate 7c. ¹H NMR (400 MHz, CDCl₃) δ 4.86 (p, J=6.2 Hz, 1H), 3.42 (t, J=7.3 Hz, 1H), 2.31 (t, J=7.5 Hz, 2H), 1.96 (q, J=7.7 Hz, 2H), 1.72-1.60 (m, 2H), 1.46 (dq, J=25.4, 4.9, 3.4 Hz, 6H), 1.25 (s, 24H), 0.94-0.77 (m, 6H).

### Intermediate 15d: heptadecan-9-yl 7-hydroxy-6-(hydroxymethyl)heptanoate

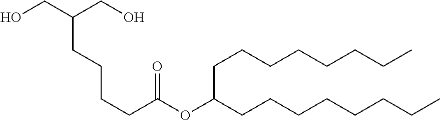 [SMILES: CCCCCCCCC(CCCCCCCC)OC(=O)CCCCC(CO)CO]

Intermediate 15d was synthesized from Intermediate 15c using the method employed in the synthesis of Intermediate 7d.

### Intermediate 15e: 7-(heptadecan-9-yloxy)-2-(hydroxymethyl)-7-oxoheptyl (9Z,12Z)-octadeca-9,12-dienoate

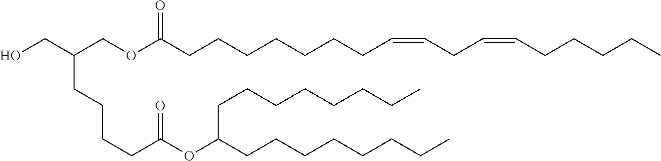 [SMILES: CCCCC/C=C\C/C=C\CCCCCCCC(=O)OCC(CO)CCCCC(=O)OC(CCCCCCCC)CCCCCCCC]

Intermediate 15e was synthesized from Intermediate 15d using the method employed in the synthesis of Intermediate 7e. ¹H NMR (400 MHz, CDCl₃) δ 5.45-5.24 (m, 4H), 4.86 (p, J=6.2 Hz, 1H), 4.20 (dd, J=11.2, 4.4 Hz, 1H), 4.08 (dd, J=11.3, 6.6 Hz, 1H), 3.58 (dd, J=11.3, 4.5 Hz, 1H), 3.49 (dd, J=11.3, 6.4 Hz, 1H), 2.77 (t, J=6.6 Hz, 2H), 2.30 (dt, J=8.9, 7.5 Hz, 4H), 2.05 (q, J=6.9 Hz, 4H), 1.79 (tt, J=6.4, 4.3 Hz, 1H), 1.62 (q, J=7.2 Hz, 4H), 1.49 (t, J=6.2 Hz, 4H), 1.43-1.20 (m, 44H), 0.88 (td, J=6.9, 5.1 Hz, 9H).

### Intermediate 15f: 7-(heptadecan-9-yloxy)-2-((((4-nitrophenoxy)carbonyl)oxy)methyl)-7-oxoheptyl (9Z,12Z)-octadeca-9,12-dienoate

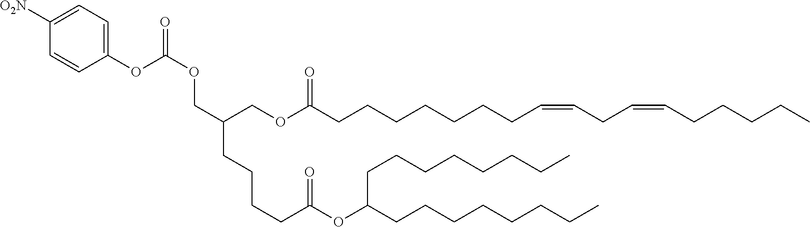 [SMILES: CCCCC/C=C\C/C=C\CCCCCCCC(=O)OCC(CCCCC(=O)OC(CCCCCCCC)CCCCCCCC)COC(=O)Oc1ccc([N+](=O)[O-])cc1]

Intermediate 15f was synthesized from Intermediate 15e using the method employed in the synthesis of Intermediate 7f. ¹H NMR (400 MHz, CDCl₃) δ 8.28-8.14 (m, 2H), 7.40-7.28 (m, 2H), 5.37-5.18 (m, 4H), 4.80 (p, J=6.3 Hz, 1H), 4.20 (h, J=5.9 Hz, 2H), 4.13-3.96 (m, 2H), 2.70 (t, J=6.4 Hz, 2H), 2.25 (td, J=7.5, 3.6 Hz, 4H), 2.10-1.91 (m, 5H), 1.64-1.48 (m, 5H), 1.48-1.34 (m, 8H), 1.34-1.12 (m, 39H), 0.94-0.65 (m, 9H).

### Compound 15: 2-((((3-(diethylamino)propoxy)carbonyl)oxy)methyl)-7-(heptadecan-9-yloxy)-7-oxoheptyl (9Z,12Z)-octadeca-9,12-dienoate

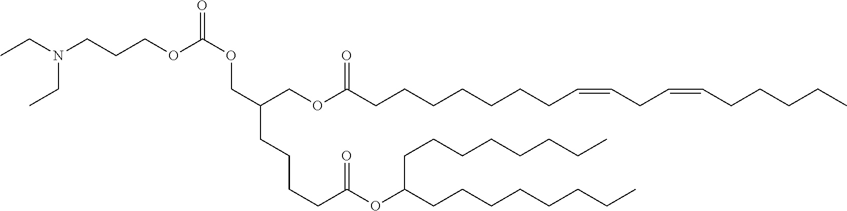 [SMILES: CCCCC/C=C\C/C=C\CCCCCCCC(=O)OCC(CCCCC(=O)OC(CCCCCCCC)CCCCCCCC)COC(=O)OCCCN(CC)CC]

Compound 15 was synthesized from Intermediate 15f using the method employed in the synthesis of Compound 1. 1H NMR (400 MHz, CDCl₃) δ 5.34 (dddd, J=12.1, 10.7, 8.8, 5.4 Hz, 4H), 4.85 (p, J=6.3 Hz, 1H), 4.17 (t, J=6.6 Hz, 2H), 4.12-3.98 (m, 4H), 2.76 (t, J=6.7 Hz, 2H), 2.56-2.46 (m, 6H), 2.28 (td, J=7.6, 5.0 Hz, 4H), 2.04 (q, J=6.8 Hz, 5H), 1.81 (p, J=6.7 Hz, 2H), 1.61 (dt, J=12.4, 6.8 Hz, 4H), 1.49 (q, J=6.2 Hz, 4H), 1.43-1.20 (m, 44H), 1.01 (t, J=7.1 Hz, 6H), 0.87 (td, J=6.8, 5.0 Hz, 9H). MS: 834.7 m/z [M+H].

### Example 16—Compound 16

### Intermediate 16a: heptadecan-9-yl 7-bromoheptanoate

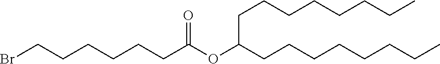 [SMILES: CCCCCCCCC(CCCCCCCC)OC(=O)CCCCCCBr]

Intermediate 16a was synthesized from Intermediate 2a and 7-bromoheptanoic acid using the method employed in the synthesis of Intermediate 7a. ¹H NMR (400 MHz, CDCl₃) δ 4.86 (p, J=6.3 Hz, 1H), 3.40 (t, J=6.8 Hz, 2H), 2.29 (t, J=7.4 Hz, 2H), 1.86 (dt, J=14.7, 6.9 Hz, 2H), 1.64 (p, J=7.4 Hz, 2H), 1.57-1.41 (m, 6H), 1.40-1.16 (m, 26H), 0.94-0.78 (m, 6H).

### Intermediate 16b: 1,1-dibenzyl 7-(heptadecan-9-yl) heptane-1,1,7-tricarboxylate

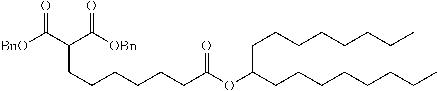 [SMILES: CCCCCCCCC(CCCCCCCC)OC(=O)CCCCCCC(C(=O)OCc1ccccc1)C(=O)OCc1ccccc1]

Intermediate 16b was synthesized from Intermediate 16a using the method employed in the synthesis of Intermediate 7b. ¹H NMR (400 MHz, CDCl₃) δ 7.46-7.14 (m, 10H), 5.15 (d, J=1.3 Hz, 4H), 4.87 (p, J=6.3 Hz, 1H), 3.43 (t, J=7.5 Hz, 1H), 2.25 (t, J=7.5 Hz, 2H), 1.93 (q, J=7.2 Hz, 2H), 1.54 (dt, J=31.6, 6.5 Hz, 6H), 1.28 (d, J=15.4 Hz, 31H), 0.91-0.86 (m, 6H).

### Intermediate 16c: 2-(7-(heptadecan-9-yloxy)-7-oxoheptyl)malonic Acid

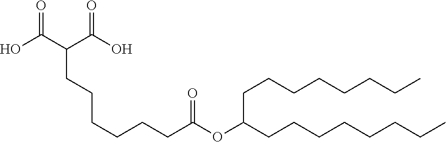 [SMILES: CCCCCCCCC(CCCCCCCC)OC(=O)CCCCCCC(C(=O)O)C(=O)O]

Intermediate 16c was synthesized from Intermediate 16b using the method employed in the synthesis of Intermediate 7c. ¹H NMR (400 MHz, CDCl₃) δ 4.80 (p, J=6.3 Hz, 1H), 3.35 (t, J=7.4 Hz, 1H), 2.22 (t, J=7.5 Hz, 2H), 1.88 (q, J=7.4 Hz, 2H), 1.54 (t, J=7.3 Hz, 2H), 1.44 (d, J=6.1 Hz, 4H), 1.36-1.13 (m, 32H), 0.81 (t, J=6.7 Hz, 6H).

### Intermediate 16d: heptadecan-9-yl 9-hydroxy-8-(hydroxymethyl)nonanoate

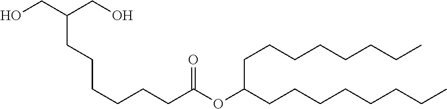 [SMILES: CCCCCCCCC(CCCCCCCC)OC(=O)CCCCCCC(CO)CO]

Intermediate 16d was synthesized from Intermediate 16c using the method employed in the synthesis of Intermediate 7c.

### Intermediate 16e: 9-(heptadecan-9-yloxy)-2-(hydroxymethyl)-9-oxononyl (9Z,12Z)-octadeca-9,12-dienoate (

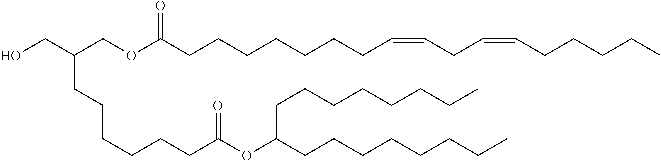 [SMILES: CCCCC/C=C\C/C=C\CCCCCCCC(=O)OCC(CO)CCCCCCC(=O)OC(CCCCCCCC)CCCCCCCC]

Intermediate 16e was synthesized from Intermediate 16d using the method employed in the synthesis of Intermediate 7e. ¹H NMR (400 MHz, CDCl₃) δ 5.29 (qd, J=10.9, 4.4 Hz, 4H), 4.79 (p, J=6.3 Hz, 1H), 4.14 (dd, J=11.2, 4.4 Hz, 1H), 4.01 (dd, J=11.2, 6.6 Hz, 1H), 3.51 (dd, J=11.2, 4.5 Hz, 1H), 3.42 (dd, J=11.3, 6.5 Hz, 1H), 2.70 (t, J=6.4 Hz, 2H), 2.23 (dt, J=17.1, 7.5 Hz, 4H), 1.98 (q, J=6.9 Hz, 4H), 1.71 (p, J=5.4 Hz, 1H), 1.55 (t, J=7.2 Hz, 4H), 1.43 (q, J=6.2 Hz, 5H), 1.37-1.14 (m, 48H), 0.81 (q, J=6.9, 6.4 Hz, 9H).

### Intermediate 16f: 9-(heptadecan-9-yloxy)-2-((((4-nitrophenoxy)carbonyl)oxy)methyl)-9-oxononyl (9Z,12Z)-octadeca-9,12-dienoate

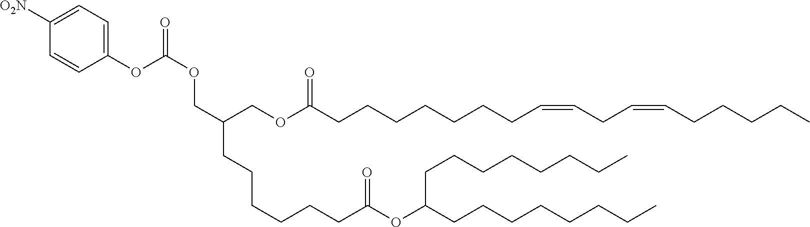 [SMILES: CCCCC/C=C\C/C=C\CCCCCCCC(=O)OCC(CCCCCCC(=O)OC(CCCCCCCC)CCCCCCCC)COC(=O)Oc1ccc([N+](=O)[O-])cc1]

Intermediate 16f was synthesized from Intermediate 16e using the method employed in the synthesis of Intermediate 7f. ¹H NMR (400 MHz, CDCl₃) δ 8.32-8.23 (m, 2H), 7.41-7.35 (m, 2H), 5.44-5.26 (m, 3H), 4.86 (q, J=6.3 Hz, 1H), 4.32-4.21 (m, 2H), 4.19-4.03 (m, 2H), 2.76 (dd, J=7.3, 5.9 Hz, 2H), 2.30 (dt, J=15.0, 7.5 Hz, 4H), 2.17-1.98 (m, 5H), 1.62 (p, J=6.9 Hz, 5H), 1.50 (q, J=6.1 Hz, 4H), 1.46-1.18 (m, 48H), 0.88 (td, J=6.8, 5.0 Hz, 9H).

### Compound 16: 2-((((3-(diethylamino)propoxy)carbonyl)oxy)methyl)-9-(heptadecan-9-yloxy)-9-oxononyl (9Z,12Z)-octadeca-9,12-dienoate

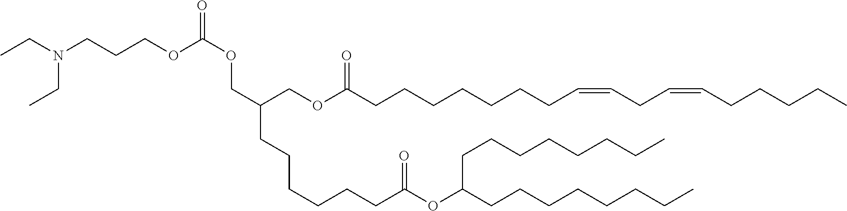 [SMILES: CCCCC/C=C\C/C=C\CCCCCCCC(=O)OCC(CCCCCCC(=O)OC(CCCCCCCC)CCCCCCCC)COC(=O)OCCCN(CC)CC]

Compound 16 was synthesized from Intermediate 16f using the method employed in the synthesis of Compound 1. ¹H NMR (400 MHz, CDCl₃) δ 5.37 (tt, J=11.3, 5.5 Hz, 4H), 4.88 (p, J=6.3 Hz, 1H), 4.20 (t, J=6.6 Hz, 2H), 4.17-4.00 (m, 4H), 2.79 (t, J=6.5 Hz, 2H), 2.53 (q, J=6.9 Hz, 6H), 2.30 (dt, J=11.0, 7.5 Hz, 4H), 2.05 (dq, J=12.4, 6.3, 5.8 Hz, 5H), 1.83 (p, J=6.8 Hz, 2H), 1.64 (q, J=7.1 Hz, 4H), 1.52 (q, J=6.0 Hz, 4H), 1.44-1.20 (m, 47H), 1.03 (t, J=7.1 Hz, 6H), 0.90 (q, J=6.5 Hz, 9H). MS: 862.4 m/z [M+H].

### Example 17—Compound 17

### Intermediate 17a: tridecan-7-yl 5-bromopentanoate

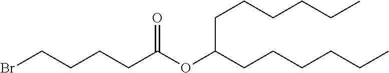 [SMILES: CCCCCCC(CCCCCC)OC(=O)CCCCBr]

Intermediate 17a was synthesized from 5-bromopentanoic acid and tridecan-7-ol using the method employed in the synthesis of Intermediate 7a.

### Intermediate 17b: 1,1-dibenzyl 5-(tridecan-7-yl) pentane-1,1,5-tricarboxylate

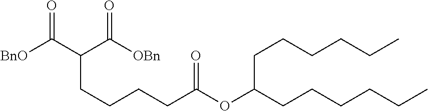 [SMILES: CCCCCCC(CCCCCC)OC(=O)CCCCC(C(=O)OCc1ccccc1)C(=O)OCc1ccccc1]

Intermediate 17b was synthesized from Intermediate 17a using the method employed in the synthesis of Intermediate 7b. ¹H NMR (400 MHz, CDCl₃) δ 7.46-7.22 (m, 10H), 5.20-5.08 (m, 4H), 4.85 (p, J=6.3 Hz, 1H), 3.43 (t, J=7.5 Hz, 1H), 2.23 (t, J=7.6 Hz, 2H), 2.00-1.91 (m, 2H), 1.62 (p, J=7.6 Hz, 2H), 1.50 (q, J=7.2, 6.7 Hz, 4H), 1.38-1.19 (m, 19H), 0.92-0.83 (m, 6H).

### Intermediate 17c: 2-(5-oxo-5-(tridecan-7-yloxy)pentyl)malonic Acid

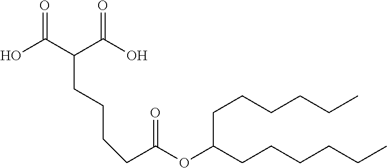 [SMILES: CCCCCCC(CCCCCC)OC(=O)CCCCC(C(=O)O)C(=O)O]

Intermediate 17c was synthesized from Intermediate 17b using the method employed in the synthesis of Intermediate 7c. ¹H NMR (400 MHz, CDCl₃) δ 4.87 (p, J=6.3 Hz, 1H), 3.43 (t, J=7.3 Hz, 1H), 2.32 (t, J=7.5 Hz, 2H), 1.96 (q, J=7.6 Hz, 2H), 1.68 (q, J=7.6 Hz, 2H), 1.57-1.38 (m, 6H), 1.26 (h, J=4.3, 3.5 Hz, 17H), 0.93-0.81 (m, 6H).

### Intermediate 17d: tridecan-7-yl 7-hydroxy-6-(hydroxymethyl)heptanoate

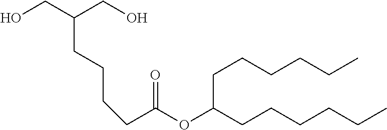 [SMILES: CCCCCCC(CCCCCC)OC(=O)CCCCC(CO)CO]

Intermediate 17d was synthesized from Intermediate 17c using the method employed in the synthesis of Intermediate 7d. ¹H NMR (400 MHz, CDCl₃) δ 4.86 (pd, J=6.3, 2.2 Hz, 1H), 4.04 (ddd, J=11.0, 7.8, 4.3 Hz, 1H), 3.69-3.56 (m, 1H), 2.36-2.24 (m, 2H), 1.97 (dddt, J=9.7, 7.4, 4.5, 2.5 Hz, 1H), 1.64 (ddt, J=15.1, 11.0, 7.6 Hz, 2H), 1.50 (d, J=6.4 Hz, 4H), 1.39-1.19 (m, 20H), 0.93-0.82 (m, 6H).

### Intermediate 17e: 2-(hydroxymethyl)-7-oxo-7-(tridecan-7-yloxy)heptyl (9Z,12Z)-octadeca-9,12-dienoate

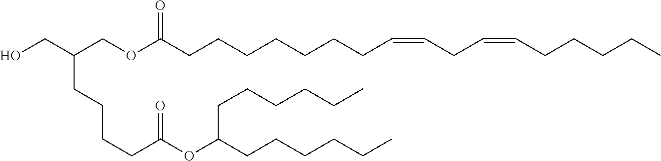 [SMILES: CCCCC/C=C\C/C=C\CCCCCCCC(=O)OCC(CO)CCCCC(=O)OC(CCCCCC)CCCCCC]

Intermediate 17e was synthesized from Intermediate 17d using the method employed in the synthesis of Intermediate 7e. ¹H NMR (400 MHz, CDCl₃) δ 5.42-5.28 (m, 4H), 4.86 (p, J=6.3 Hz, 1H), 4.20 (dd, J=11.2, 4.4 Hz, 1H), 4.08 (dd, J=11.2, 6.6 Hz, 1H), 3.58 (dd, J=11.3, 4.4 Hz, 1H), 3.49 (dd, J=11.3, 6.5 Hz, 1H), 2.77 (t, J=6.4 Hz, 2H), 2.30 (q, J=7.9 Hz, 4H), 2.04 (q, J=6.9 Hz, 4H), 1.79 (ddt, J=8.9, 6.5, 3.3 Hz, 1H), 1.62 (q, J=7.1 Hz, 4H), 1.50 (d, J=6.4 Hz, 4H), 1.42-1.22 (m, 34H), 0.92-0.83 (m, 9H).

### Intermediate 17f: 2-((((4-nitrophenoxy)carbonyl)oxy)methyl)-7-oxo-7-(tridecan-7-yloxy)heptyl (9Z,12Z)-octadeca-9,12-dienoate

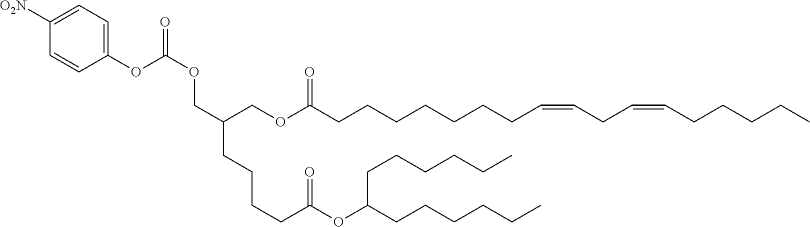 [SMILES: CCCCC/C=C\C/C=C\CCCCCCCC(=O)OCC(CCCCC(=O)OC(CCCCCC)CCCCCC)COC(=O)Oc1ccc([N+](=O)[O-])cc1]

Intermediate 17f was synthesized from Intermediate 17e using the method employed in the synthesis of Intermediate 7f. ¹H NMR (400 MHz, CDCl₃) δ 8.28-8.15 (m, 2H), 7.41-7.28 (m, 2H), 5.41-5.16 (m, 4H), 4.80 (p, J=6.3 Hz, 1H), 4.20 (h, J=5.9 Hz, 2H), 4.15-3.96 (m, 2H), 2.70 (t, J=6.3 Hz, 2H), 2.24 (tt, J=7.2, 3.7 Hz, 4H), 2.10-1.89 (m, 5H), 1.56 (dq, J=15.3, 7.5 Hz, 4H), 1.50-1.34 (m, 8H), 1.34-1.10 (m, 30H), 0.81 (q, J=6.5 Hz, 9H).

### Compound 17: 2-((((3-(diethylamino)propoxy)carbonyl)oxy)methyl)-7-oxo-7-(tridecan-7-yloxy)heptyl (9Z,12Z)-octadeca-9,12-dienoate

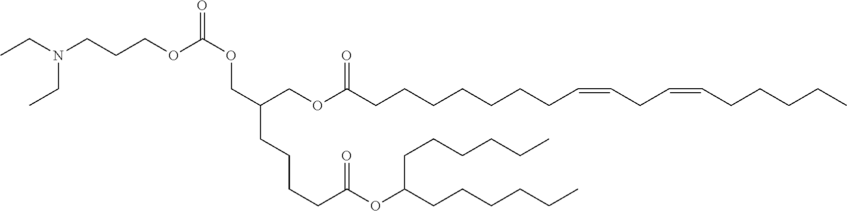 [SMILES: CCCCC/C=C\C/C=C\CCCCCCCC(=O)OCC(CCCCC(=O)OC(CCCCCC)CCCCCC)COC(=O)OCCCN(CC)CC]

Compound 17 was synthesized from Intermediate 17f using the method employed in the synthesis of Compound 1. ¹H NMR (400 MHz, CDCl₃) δ 5.37 (tt, J=11.2, 5.6 Hz, 4H), 4.88 (p, J=6.2 Hz, 1H), 4.20 (t, J=6.6 Hz, 2H), 4.16-4.01 (m, 4H), 2.79 (t, J=6.4 Hz, 2H), 2.55 (q, J=6.9 Hz, 5H), 2.31 (td, J=7.5, 4.7 Hz, 4H), 2.06 (p, J=8.0, 7.4 Hz, 4H), 1.85 (p, J=6.9 Hz, 2H), 1.63 (p, J=7.2 Hz, 4H), 1.52 (t, J=6.4 Hz, 4H), 1.47-1.21 (m, 34H), 1.04 (t, J=7.1 Hz, 6H), 0.90 (q, J=6.6 Hz, 9H). MS: 778.3 m/z [M+H].

### Example 18—Compound 18

### Intermediate 24a: tridecan-7-yl 7-bromoheptanoate

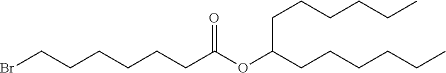 [SMILES: CCCCCCC(CCCCCC)OC(=O)CCCCCCBr]

Intermediate 18a was synthesized from 7-bromoheptanoic acid and tridecan-7-ol using the method employed in the synthesis of Intermediate 7a. ¹H NMR (400 MHz, CDCl₃) δ 4.80 (p, J=6.2 Hz, 1H), 3.46 (t, J=6.7 Hz, 1H), 3.34 (t, J=6.8 Hz, 1H), 2.22 (t, J=7.4 Hz, 2H), 1.79 (p, J=6.9 Hz, 1H), 1.71 (p, J=6.9 Hz, 1H), 1.57 (p, J=7.5 Hz, 2H), 1.42 (qd, J=16.2, 14.5, 9.2 Hz, 6H), 1.34-1.12 (m, 18H), 0.81 (t, J=6.7 Hz, 6H).

### Intermediate 18b: 1,1-dibenzyl 7-(tridecan-7-yl) heptane-1,1,7-tricarboxylate

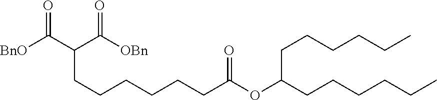 [SMILES: CCCCCCC(CCCCCC)OC(=O)CCCCCCC(C(=O)OCc1ccccc1)C(=O)OCc1ccccc1]

Intermediate 18b was synthesized from Intermediate 18a using the method employed in the synthesis of Intermediate 7b. ¹H NMR (400 MHz, CDCl₃) δ 7.43-7.18 (m, 10H), 5.14 (d, J=1.2 Hz, 4H), 4.86 (p, J=6.3 Hz, 1H), 3.43 (t, J=7.6 Hz, 1H), 2.25 (t, J=7.5 Hz, 2H), 1.92 (d, J=7.2 Hz, 2H), 1.63-1.43 (m, 7H), 1.27 (dd, J=10.7, 4.6 Hz, 23H), 0.90-0.84 (m, 6H).

### Intermediate 18c: 2-(7-oxo-7-(tridecan-7-yloxy)heptyl)malonic Acid

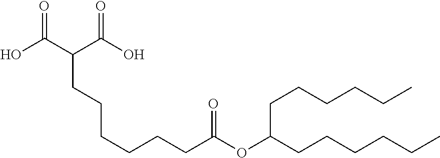 [SMILES: CCCCCCC(CCCCCC)OC(=O)CCCCCCC(C(=O)O)C(=O)O]

Intermediate 18c was synthesized from Intermediate 18b using the method employed in the synthesis of Intermediate 7c. ¹H NMR (400 MHz, CDCl₃) δ 4.87 (p, J=6.3 Hz, 1H), 3.42 (t, J=7.4 Hz, 1H), 2.29 (t, J=7.5 Hz, 2H), 1.94 (q, J=7.4 Hz, 2H), 1.61 (t, J=7.3 Hz, 2H), 1.56-1.46 (m, 4H), 1.44-1.21 (m, 23H), 0.93-0.83 (m, 6H).

### Intermediate 18d: tridecan-7-yl 9-hydroxy-8-(hydroxymethyl)nonanoate

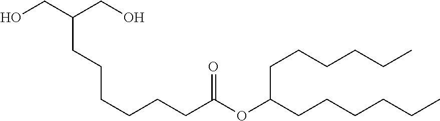 [SMILES: CCCCCCC(CCCCCC)OC(=O)CCCCCCC(CO)CO]

Intermediate 18d was synthesized from Intermediate 18c using the method employed in the synthesis of Intermediate 7d.

### Intermediate 18e: 2-(hydroxymethyl)-9-oxo-9-(tridecan-7-yloxy)nonyl (9Z,12Z)-octadeca-9,12-dienoate

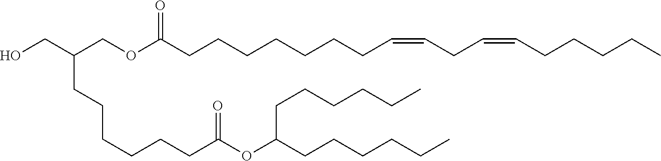 [SMILES: CCCCC/C=C\C/C=C\CCCCCCCC(=O)OCC(CO)CCCCCCC(=O)OC(CCCCCC)CCCCCC]

Intermediate 18e was synthesized from Intermediate 18d using the method employed in the synthesis of Intermediate 7e. ¹H NMR (400 MHz, CDCl₃) δ 5.44-5.26 (m, 4H), 4.86 (p, J=6.3 Hz, 1H), 4.21 (dd, J=11.2, 4.3 Hz, 1H), 4.07 (dd, J=11.2, 6.7 Hz, 1H), 3.58 (dd, J=11.3, 4.5 Hz, 1H), 3.49 (dd, J=11.3, 6.5 Hz, 1H), 2.77 (t, J=6.4 Hz, 2H), 2.30 (dt, J=17.4, 7.5 Hz, 4H), 2.05 (q, J=6.9 Hz, 4H), 1.78 (ddt, J=10.8, 6.7, 4.3 Hz, 1H), 1.67-1.57 (m, 4H), 1.50 (d, J=6.4 Hz, 4H), 1.30 (tt, J=17.5, 14.4, 6.2 Hz, 39H), 0.88 (q, J=6.8 Hz, 9H).

### Intermediate 18f: 2-((((4-nitrophenoxy)carbonyl)oxy)methyl)-9-oxo-9-(tridecan-7-yloxy)nonyl (9Z,12Z)-octadeca-9,12-dienoate

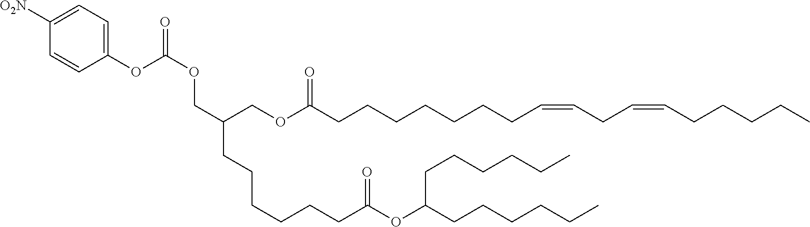 [SMILES: CCCCC/C=C\C/C=C\CCCCCCCC(=O)OCC(CCCCCCC(=O)OC(CCCCCC)CCCCCC)COC(=O)Oc1ccc([N+](=O)[O-])cc1]

Intermediate 18f was synthesized from Intermediate 18e using the method employed in the synthesis of Intermediate 7f. ¹H NMR (400 MHz, CDCl₃) δ 8.26-8.17 (m, 2H), 7.35-7.27 (m, 2H), 5.39-5.19 (m, 4H), 4.80 (p, J=6.3 Hz, 1H), 4.27-4.14 (m, 2H), 4.10 (dd, J=11.3, 4.7 Hz, 1H), 4.02 (dd, J=11.3, 6.6 Hz, 1H), 2.70 (t, J=6.4 Hz, 2H), 2.23 (dt, J=14.9, 7.5 Hz, 4H), 2.07-1.93 (m, 5H), 1.57 (q, J=7.1 Hz, 4H), 1.52-1.41 (m, 5H), 1.41-1.12 (m, 39H), 0.87-0.75 (m, 9H).

### Compound 18: 2-((((3-(diethylamino)propoxy)carbonyl)oxy)methyl)-9-oxo-9-(tridecan-7-yloxy)nonyl (9Z,12Z)-octadeca-9,12-dienoate

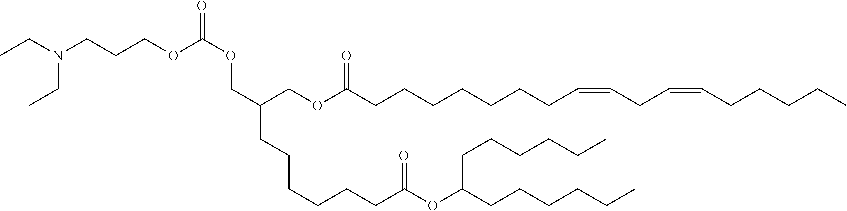 [SMILES: CCCCC/C=C\C/C=C\CCCCCCCC(=O)OCC(CCCCCCC(=O)OC(CCCCCC)CCCCCC)COC(=O)OCCCN(CC)CC]

Compound 18 was synthesized from Intermediate 18f using the method employed in the synthesis of Compound 1. ¹H NMR (400 MHz, CDCl₃) δ 5.35 (tt, J=11.2, 5.4 Hz, 4H), 4.86 (p, J=6.3 Hz, 1H), 4.18 (t, J=6.5 Hz, 2H), 4.15-4.06 (m, 3H), 4.02 (dd, J=11.2, 6.4 Hz, 1H), 2.77 (t, J=6.5 Hz, 2H), 2.53 (s, 5H), 2.28 (dt, J=10.6, 7.6 Hz, 4H), 2.03 (dq, J=12.3, 6.2, 5.6 Hz, 5H), 1.84 (s, 2H), 1.62 (q, J=7.2 Hz, 4H), 1.50 (q, J=6.3 Hz, 4H), 1.40-1.19 (m, 39H), 1.12-0.97 (m, 6H), 0.88 (q, J=6.6 Hz, 9H). MS: 806.3 m/z [M+H].

### Example 19—Compound 19

### Intermediate 19a: tridecan-7-yl 9-bromononanoate

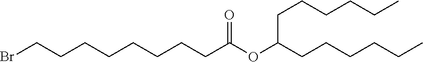 [SMILES: CCCCCCC(CCCCCC)OC(=O)CCCCCCCCBr]

Intermediate 19a was synthesized from 9-bromononanoic acid and tridecan-7-ol using the method employed in the synthesis of Intermediate 7a.

### Intermediate 19b: 1,1-dibenzyl 9-(tridecan-7-yl) nonane-1,1,9-tricarboxylate

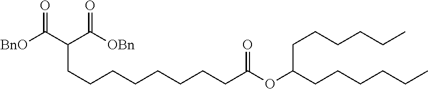 [SMILES: CCCCCCC(CCCCCC)OC(=O)CCCCCCCCC(C(=O)OCc1ccccc1)C(=O)OCc1ccccc1]

Intermediate 19b was synthesized from Intermediate 19a using the method employed in the synthesis of Intermediate 7b. ¹H NMR (400 MHz, CDCl₃) δ 7.36-7.14 (m, 10H), 5.07 (s, 4H), 4.80 (p, J=6.3 Hz, 1H), 3.36 (t, J=7.6 Hz, 1H), 2.19 (t, J=7.5 Hz, 2H), 1.84 (q, J=7.6, 5.2 Hz, 2H), 1.59-1.37 (m, 7H), 1.28-1.14 (m, 26H), 0.80 (t, J=6.7 Hz, 6H).

### Intermediate 19c: 2-(9-oxo-9-(tridecan-7-yloxy)nonyl)malonic Acid

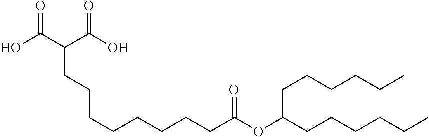 [SMILES: CCCCCCC(CCCCCC)OC(=O)CCCCCCCCC(C(=O)O)C(=O)O]

Intermediate 19c was synthesized from Intermediate 19b using the method employed in the synthesis of Intermediate 7c. ¹H NMR (400 MHz, CDCl₃) δ 4.81 (p, J=6.2 Hz, 1H), 3.35 (t, J=7.4 Hz, 1H), 2.22 (t, J=7.5 Hz, 2H), 1.87 (q, J=7.5 Hz, 2H), 1.54 (t, J=7.1 Hz, 2H), 1.50-1.38 (m, 4H), 1.39-0.97 (m, 26H), 0.80 (t, J=6.8 Hz, 6H).

### Intermediate 19d: tridecan-7-yl 11-hydroxy-10-(hydroxymethyl)undecanoate

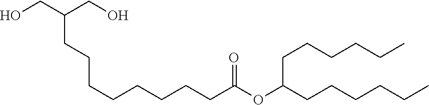 [SMILES: CCCCCCC(CCCCCC)OC(=O)CCCCCCCCC(CO)CO]

Intermediate 19d was synthesized from Intermediate 19c using the method employed in the synthesis of Intermediate 7d. ¹H NMR (400 MHz, CDCl₃) δ 4.80 (p, J=6.2 Hz, 1H), 3.75 (dd, J=10.6, 3.7 Hz, 2H), 3.59 (dd, J=10.6, 7.6 Hz, 2H), 2.21 (t, J=7.5 Hz, 2H), 1.69 (ddt, J=10.4, 6.9, 3.5 Hz, 1H), 1.54 (p, J=7.2, 6.6 Hz, 2H), 1.44 (q, J=6.4 Hz, 4H), 1.32-1.09 (m, 28H), 0.87-0.74 (m, 6H).

### Intermediate 19e: 2-(hydroxymethyl)-11-oxo-11-(tridecan-7-yloxy)undecyl (9Z,12Z)-octadeca-9,12-dienoate

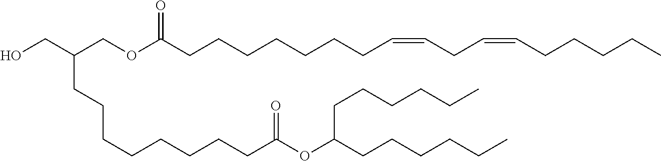 [SMILES: CCCCC/C=C\C/C=C\CCCCCCCC(=O)OCC(CO)CCCCCCCCC(=O)OC(CCCCCC)CCCCCC]

Intermediate 19e was synthesized from Intermediate 19d using the method employed in the synthesis of Intermediate 7e.

### Intermediate 19f: 2-((((4-nitrophenoxy)carbonyl)oxy)methyl)-11-oxo-11-(tridecan-7-yloxy)undecyl (9Z,12Z)-octadeca-9,12-dienoate

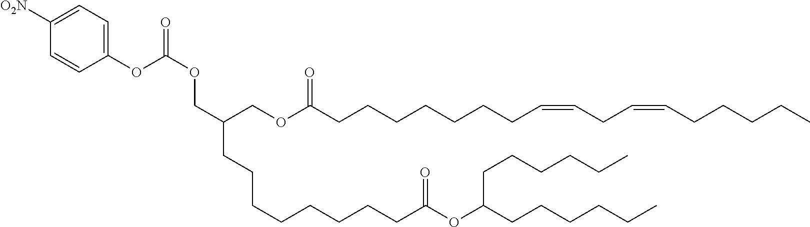 [SMILES: CCCCC/C=C\C/C=C\CCCCCCCC(=O)OCC(CCCCCCCCC(=O)OC(CCCCCC)CCCCCC)COC(=O)Oc1ccc([N+](=O)[O-])cc1]

Intermediate 19f was synthesized from Intermediate 19e using the method employed in the synthesis of Intermediate 7f. ¹H NMR (400 MHz, CDCl₃) δ 8.36-8.23 (m, 2H), 7.43-7.35 (m, 2H), 5.46-5.25 (m, 4H), 4.87 (p, J=6.3 Hz, 1H), 4.37-4.24 (m, 2H), 4.24-4.12 (m, 1H), 4.09 (dd, J=11.2, 6.6 Hz, 1H), 2.77 (t, J=6.3 Hz, 2H), 2.30 (dt, J=17.7, 7.5 Hz, 4H), 2.15-2.00 (m, 5H), 1.62 (tt, J=8.0, 4.0 Hz, 4H), 1.51 (q, J=6.3 Hz, 5H), 1.45-1.18 (m, 44H), 0.88 (td, J=6.8, 5.1 Hz, 9H).

### Compound 19: 2-((((3-(diethylamino)propoxy)carbonyl)oxy)methyl)-11-oxo-11-(tridecan-7-yloxy)undecyl (9Z,12Z)-octadeca-9,12-dienoate

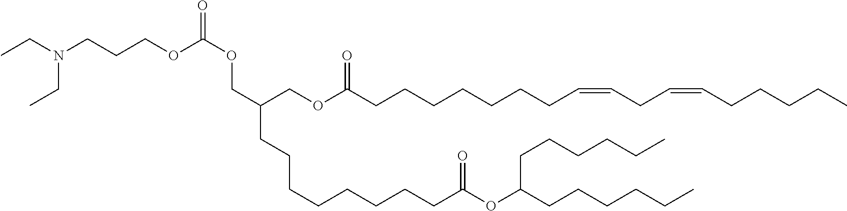 [SMILES: CCCCC/C=C\C/C=C\CCCCCCCC(=O)OCC(CCCCCCCCC(=O)OC(CCCCCC)CCCCCC)COC(=O)OCCCN(CC)CC]

Compound 19 was synthesized from Intermediate 19f using the method employed in the synthesis of Compound 1. ¹H NMR (400 MHz, CDCl₃) δ 5.48-5.24 (m, 4H), 4.86 (p, J=6.3 Hz, 1H), 4.18 (t, J=6.5 Hz, 2H), 4.14-4.00 (m, 4H), 2.77 (t, J=6.5 Hz, 2H), 2.31-2.22 (m, 4H), 2.03 (dq, J=12.1, 6.4, 6.0 Hz, 5H), 1.84 (s, 2H), 1.61 (p, J=7.2 Hz, 4H), 1.50 (q, J=6.2 Hz, 4H), 1.41-1.17 (m, 42H), 1.04 (s, 6H), 0.88 (q, J=6.7 Hz, 9H). MS: 834.7 m/z [M+H].

### Example 20—Compound 22

### Intermediate 20a: nonan-5-ol

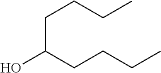

To a solution of nonan-5-one (25 g, 1.0 equiv.) in MeOH (0.5-1.0 M) was added NaBH₄ (2.0 equiv.) slowly under N₂ atmosphere at 0° C. The reaction mixture was stirred at 20° C. for 5 h under N₂ atmosphere. The reaction mixture was quenched with sat. NH₄C1 slowly under N₂ and stirred for another 60 min after addition. The reaction mixture was poured into water and extracted 2× with EtOAc. The organic phase was evaporated under reduced pressure to get a residue that was used directly in the next step without additional purification.

### Intermediate 20b: nonan-5-yl 7-bromoheptanoate

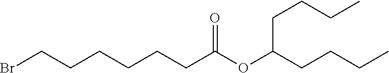 [SMILES: CCCCC(CCCC)OC(=O)CCCCCCBr]

Intermediate 20b was synthesized from Intermediate 20a and 7-bromoheptanoic acid using the method employed in the synthesis of Intermediate 7a.

### Intermediate 20c: 1,1-dibenzyl 7-(nonan-5-yl) heptane-1,1,7-tricarboxylate

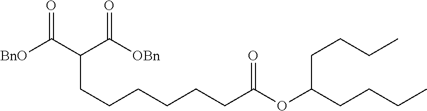 [SMILES: CCCCC(CCCC)OC(=O)CCCCCCC(C(=O)OCc1ccccc1)C(=O)OCc1ccccc1]

Intermediate 20c was synthesized from Intermediate 20b using the method employed in the synthesis of Intermediate 7b. ¹H NMR (400 MHz, CDCl₃) δ 7.32-7.10 (m, 10H), 5.07 (d, J=1.1 Hz, 4H), 4.79 (p, J=6.3 Hz, 1H), 3.36 (t, J=7.5 Hz, 1H), 2.18 (t, J=7.5 Hz, 2H), 1.85 (d, J=7.2 Hz, 2H), 1.54-1.39 (m, 6H), 1.30-1.13 (m, 14H), 0.81 (t, J=6.9 Hz, 6H).

### Intermediate 20d: 2-(7-(nonan-5-yloxy)-7-oxoheptyl)malonic Acid

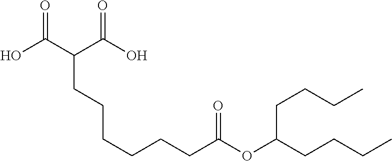 [SMILES: CCCCC(CCCC)OC(=O)CCCCCCC(C(=O)O)C(=O)O]

Intermediate 20d was synthesized from Intermediate 20c using the method employed in the synthesis of Intermediate 7c. ¹H NMR (400 MHz, CDCl₃) δ 4.88 (p, J=6.3 Hz, 1H), 3.43 (t, J=7.3 Hz, 1H), 2.29 (t, J=7.5 Hz, 2H), 1.99-1.91 (m, 2H), 1.63 (q, J=7.2 Hz, 2H), 1.56-1.48 (m, 4H), 1.44-1.20 (m, 15H), 0.88 (t, J=6.9 Hz, 6H).

### Intermediate 20e: nonan-5-yl 9-hydroxy-8-(hydroxymethyl)nonanoate

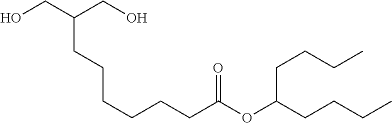 [SMILES: CCCCC(CCCC)OC(=O)CCCCCCC(CO)CO]

Intermediate 20e was synthesized from Intermediate 20d using the method employed in the synthesis of Intermediate 7d. ¹H NMR (400 MHz, CDCl₃) δ 4.87 (p, J=6.3 Hz, 1H), 3.86-3.77 (m, 1H), 3.69-3.60 (m, 1H), 2.28 (td, J=7.5, 1.3 Hz, 2H), 1.61 (t, J=7.2 Hz, 2H), 1.56-1.46 (m, 4H), 1.42-1.19 (m, 16H), 0.88 (t, J=6.9 Hz, 6H).

### Intermediate 26f: 2-(hydroxymethyl)-9-(nonan-5-yloxy)-9-oxononyl (9Z,12Z)-octadeca-9,12-dienoate

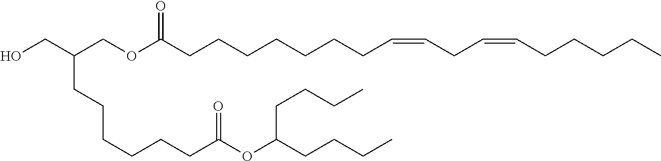 [SMILES: CCCCC/C=C\C/C=C\CCCCCCCC(=O)OCC(CO)CCCCCCC(=O)OC(CCCC)CCCC]

Intermediate 20f was synthesized from Intermediate 20e using the method employed in the synthesis of Intermediate 7e. 1H NMR (400 MHz, CDCl₃) δ 5.29 (tp, J=10.1, 5.0 Hz, 4H), 4.80 (p, J=6.4 Hz, 1H), 4.15 (dd, J=11.2, 4.3 Hz, 1H), 4.00 (dd, J=11.2, 6.7 Hz, 1H), 3.51 (dd, J=11.3, 4.5 Hz, 1H), 3.42 (dd, J=11.3, 6.5 Hz, 1H), 2.70 (t, J=6.4 Hz, 2H), 2.23 (dt, J=15.3, 7.5 Hz, 5H), 1.98 (q, J=6.9 Hz, 4H), 1.72 (t, J=5.9 Hz, 1H), 1.54 (d, J=7.3 Hz, 6H), 1.45 (q, J=7.0 Hz, 6H), 1.33-1.13 (m, 35H), 0.82 (t, J=6.8 Hz, 9H).

### Intermediate 20g: 2-((((4-nitrophenoxy)carbonyl)oxy)methyl)-9-(nonan-5-yloxy)-9-oxononyl (9Z,12Z)-octadeca-9,12-dienoate

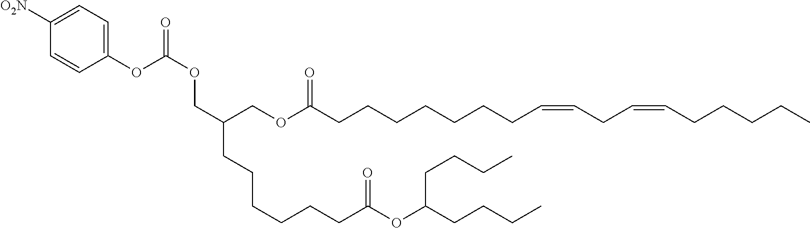 [SMILES: CCCCC/C=C\C/C=C\CCCCCCCC(=O)OCC(CCCCCCC(=O)OC(CCCC)CCCC)COC(=O)Oc1ccc([N+](=O)[O-])cc1]

Intermediate 20g was synthesized from Intermediate 20f using the method employed in the synthesis of Intermediate 7f. ¹H NMR (400 MHz, CDCl₃) δ 8.33-8.25 (m, 2H), 7.42-7.36 (m, 2H), 5.44-5.28 (m, 4H), 4.87 (p, J=6.3 Hz, 1H), 4.27 (h, J=6.0 Hz, 2H), 4.17 (dd, J=11.3, 4.8 Hz, 1H), 4.09 (dd, J=11.3, 6.7 Hz, 1H), 2.77 (t, J=6.3 Hz, 2H), 2.31 (dt, J=12.9, 7.5 Hz, 5H), 2.18-1.99 (m, 5H), 1.68-1.57 (m, 5H), 1.57-1.47 (m, 6H), 1.47-1.18 (m, 34H), 0.89 (t, J=6.8 Hz, 9H).

### Compound 20: 2-((((3-(diethylamino)propoxy)carbonyl)oxy)methyl)-9-(nonan-5-yloxy)-9-oxononyl (9Z,12Z)-octadeca-9,12-dienoate

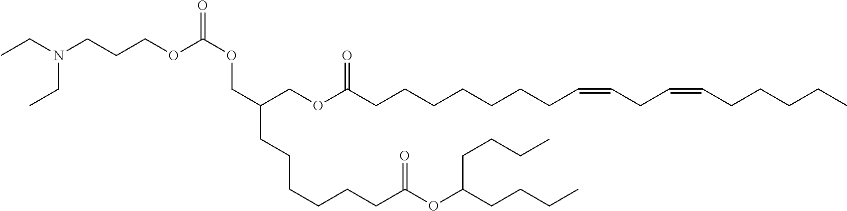 [SMILES: CCCCC/C=C\C/C=C\CCCCCCCC(=O)OCC(CCCCCCC(=O)OC(CCCC)CCCC)COC(=O)OCCCN(CC)CC]

Compound 20 was synthesized from Intermediate 20g using the method employed in the synthesis of Compound 1. ¹H NMR (400 MHz, CDCl₃) δ 5.36-5.21 (m, 4H), 4.80 (p, J=6.3 Hz, 1H), 4.11 (t, J=6.5 Hz, 2H), 4.07-3.92 (m, 4H), 2.70 (t, J=6.4 Hz, 2H), 2.46 (q, J=6.9 Hz, 5H), 2.22 (q, J=7.9 Hz, 4H), 2.03-1.90 (m, 5H), 1.77 (q, J=7.2 Hz, 2H), 1.53 (q, J=7.0 Hz, 5H), 1.44 (dq, J=13.2, 6.4 Hz, 5H), 1.33-1.09 (m, 31H), 0.96 (t, J=7.2 Hz, 6H), 0.82 (t, J=6.9 Hz, 9H). MS: 750.7 m/z [M+H].

### Example 21—Compound 21

### Intermediate 27a: nonan-5-yl 9-bromononanoate

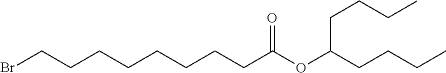 [SMILES: CCCCC(CCCC)OC(=O)CCCCCCCCBr]

Intermediate 21a was synthesized from 9-bromononanoic acid and Intermediate 20a using the method employed in the synthesis of Intermediate 7a.

### Intermediate 21b: 1,1-dibenzyl 9-(nonan-5-yl) nonane-1,1,9-tricarboxylate

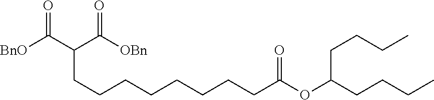 [SMILES: CCCCC(CCCC)OC(=O)CCCCCCCCC(C(=O)OCc1ccccc1)C(=O)OCc1ccccc1]

Intermediate 21b was synthesized from Intermediate 21a using the method employed in the synthesis of Intermediate 7b. ¹H NMR (400 MHz, CDCl₃) δ 7.34-7.16 (m, 10H), 5.07 (s, 4H), 4.80 (p, J=6.3 Hz, 1H), 3.36 (t, J=7.5 Hz, 1H), 2.20 (t, J=7.5 Hz, 2H), 1.85 (d, J=7.1 Hz, 2H), 1.48 (dq, J=29.2, 7.0 Hz, 7H), 1.20 (ttt, J=14.1, 11.2, 7.4 Hz, 19H), 0.81 (t, J=6.9 Hz, 6H).

### Intermediate 21c: 2-(9-(nonan-5-yloxy)-9-oxononyl)malonic Acid

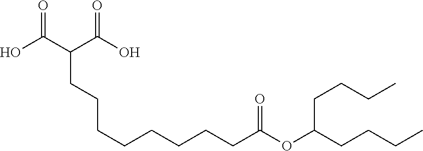 [SMILES: CCCCC(CCCC)OC(=O)CCCCCCCCC(C(=O)O)C(=O)O]

Intermediate 21c was synthesized from Intermediate 21b using the method employed in the synthesis of Intermediate 7c. ¹H NMR (400 MHz, CDCl₃) δ 4.88 (p, J=6.3 Hz, 1H), 3.43 (t, J=7.3 Hz, 1H), 2.29 (t, J=7.5 Hz, 2H), 1.94 (q, J=7.5 Hz, 2H), 1.61 (p, J=7.2 Hz, 2H), 1.57-1.46 (m, 4H), 1.43-1.21 (m, 19H), 0.88 (t, J=6.9 Hz, 6H).

### Intermediate 21d: nonan-5-yl 11-hydroxy-10-(hydroxymethyl)undecanoate

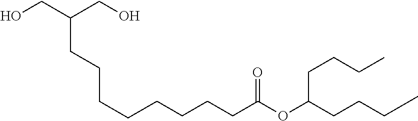 [SMILES: CCCCC(CCCC)OC(=O)CCCCCCCCC(CO)CO]

Intermediate 21d was synthesized from Intermediate 21c using the method employed in the synthesis of Intermediate 7d.

### Intermediate 21e: 2-(hydroxymethyl)-11-(nonan-5-yloxy)-11-oxoundecyl (9Z,12Z)-octadeca-9,12-dienoate

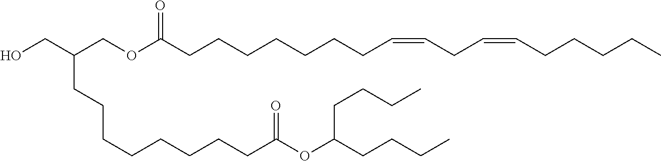 [SMILES: CCCCC/C=C\C/C=C\CCCCCCCC(=O)OCC(CO)CCCCCCCCC(=O)OC(CCCC)CCCC]

Intermediate 21e was synthesized from Intermediate 21d using the method employed in the synthesis of Intermediate 7e. ¹H NMR (400 MHz, CDCl₃) δ 5.48-5.23 (m, 4H), 4.87 (p, J=6.3 Hz, 1H), 4.22 (dd, J=11.2, 4.3 Hz, 1H), 4.08 (dd, J=11.2, 6.7 Hz, 1H), 3.59 (dd, J=11.3, 4.5 Hz, 1H), 3.49 (dd, J=11.3, 6.5 Hz, 1H), 2.77 (t, J=6.4 Hz, 2H), 2.30 (dt, J=16.8, 7.5 Hz, 4H), 2.05 (q, J=6.8 Hz, 4H), 1.83-1.74 (m, 1H), 1.63 (ddt, J=10.5, 7.2, 3.3 Hz, 5H), 1.58-1.46 (m, 5H), 1.40-1.21 (m, 38H), 0.89 (td, J=6.9, 1.8 Hz, 9H).

### Intermediate 21f: 2-((((4-nitrophenoxy)carbonyl)oxy)methyl)-11-(nonan-5-yloxy)-11-oxoundecyl (9Z,12Z)-octadeca-9,12-dienoate

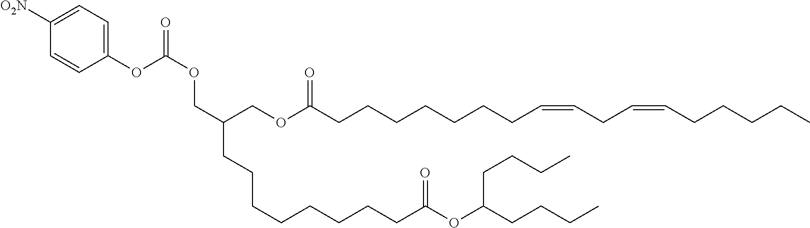 [SMILES: CCCCC/C=C\C/C=C\CCCCCCCC(=O)OCC(CCCCCCCCC(=O)OC(CCCC)CCCC)COC(=O)Oc1ccc([N+](=O)[O-])cc1]

Intermediate 21f was synthesized from Intermediate 21e using the method employed in the synthesis of Intermediate 7f. ¹H NMR (400 MHz, CDCl₃) δ 8.28-8.15 (m, 2H), 7.38-7.28 (m, 2H), 5.38-5.20 (m, 4H), 4.80 (p, J=6.3 Hz, 1H), 4.26-4.14 (m, 2H), 4.10 (dd, J=11.3, 4.8 Hz, 1H), 4.02 (dd, J=11.3, 6.7 Hz, 1H), 2.70 (t, J=6.3 Hz, 2H), 2.23 (dt, J=16.2, 7.5 Hz, 4H), 2.07-1.93 (m, 5H), 1.55 (tt, J=7.2, 3.6 Hz, 4H), 1.52-1.40 (m, 5H), 1.40-1.12 (m, 36H), 0.82 (t, J=6.8 Hz, 9H).

### Compound 21: 2-((((3-(diethylamino)propoxy)carbonyl)oxy)methyl)-11-(nonan-5-yloxy)-11-oxoundecyl (9Z,12Z)-octadeca-9,12-dienoate

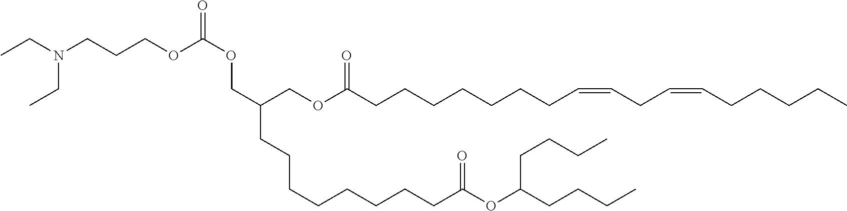 [SMILES: CCCCC/C=C\C/C=C\CCCCCCCC(=O)OCC(CCCCCCCCC(=O)OC(CCCC)CCCC)COC(=O)OCCCN(CC)CC]

Compound 21 was synthesized from Intermediate 21f using the method employed in the synthesis of Compound 1. ¹H NMR (400 MHz, CDCl₃) δ 5.43-5.27 (m, 4H), 4.87 (p, J=6.3 Hz, 1H), 4.19 (t, J=6.5 Hz, 2H), 4.15-4.00 (m, 4H), 2.77 (t, J=6.5 Hz, 2H), 2.57 (s, 5H), 2.33-2.22 (m, 4H), 2.03 (dq, J=12.2, 6.3, 5.8 Hz, 5H), 1.61 (p, J=7.1 Hz, 5H), 1.56-1.45 (m, 5H), 1.40-1.20 (m, 35H), 1.06 (s, 6H), 0.89 (td, J=6.9, 1.8 Hz, 9H). MS: 778.6 m/z [M+H].

### Example 22—Compound 22

### Compound 22: 2-((((2-(diethylamino)ethyl)carbamoyl)oxy)methyl)propane-1,3-diyl bis(4,4-bis(octyloxy)butanoate)

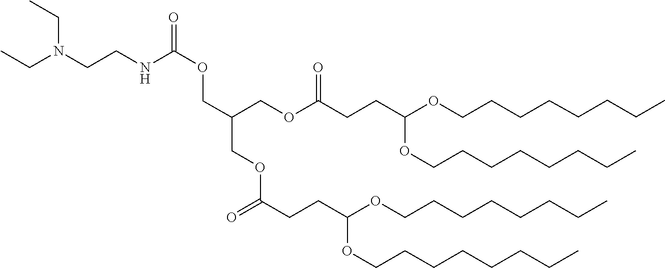 [SMILES: CCCCCCCCOC(CCC(=O)OCC(COC(=O)CCC(OCCCCCCCC)OCCCCCCCC)COC(=O)NCCN(CC)CC)OCCCCCCCC]

Compound 22 was synthesized from Intermediate 1d and N′,N′-diethylethane-1,2-diamine using the method employed in the synthesis of Compound 1. ¹H NMR (400 MHz, CDCl₃) δ 5.21 (d, J=5.4 Hz, 1H), 4.48 (t, J=5.5 Hz, 2H), 4.12 (d, J=5.7 Hz, 6H), 3.56 (dt, J=9.3, 6.7 Hz, 4H), 3.40 (dt, J=9.3, 6.7 Hz, 4H), 3.20 (q, J=5.9 Hz, 2H), 2.51 (q, J=7.1, 6.2 Hz, 6H), 2.39 (t, J=7.6 Hz, 5H), 1.92 (td, J=7.6, 5.5 Hz, 4H), 1.54 (q, J=6.9 Hz, 8H), 1.39-1.21 (m, 41H), 1.00 (t, J=7.1 Hz, 6H), 0.92-0.82 (m, 12H). MS: 901.3 m/z [M+H].

### Example 23—Compound 23

### Intermediate 23a: (9Z,12Z)-octadeca-9,12-dien-1-yl Methanesulfonate

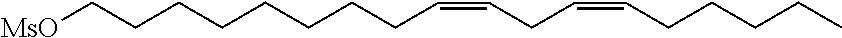 [SMILES: CCCCC/C=C\C/C=C\CCCCCCCCOS(C)(=O)=O]

A mixture of (9Z,12Z)-octadeca-9,12-dien-1-ol (16 g, 1.0 equiv.) and TEA (1.2 equiv.) in DCM (0.1-0.2 M) was degassed and purged 3× with N₂, and then the mixture was added MsCl (1.2 equiv.) slowly at 0° C. The mixture was stirred at 20° C. for 2 h under N₂ atmosphere. The reaction mixture was added sat. NaHCO₃ slowly under N₂ and stirred for 20 min after addition. The mixture was then poured into water and extracted with EtOAc. The organic phase was evaporated under reduced pressure to get a residue that was used directly in the next step without additional purification.

### Intermediate 23b: (6Z,9Z)-18-bromooctadeca-6,9-diene

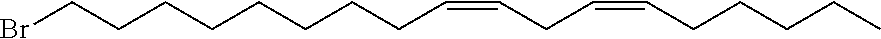 [SMILES: CCCCC/C=C\C/C=C\CCCCCCCCBr]

To a solution of Intermediate 23a (16 g, 1.0 equiv.) in THF (0.2-0.4 M) was added TBAB (1.5 equiv.). The mixture was stirred at 80° C. for 12 h. The reaction mixture was concentrated under reduced pressure to remove solvent. The residue was diluted with H₂O and extracted 2× with EtOAc. The combined organic layers were washed 2× with H₂O, dried over Na₂SO₄, filtered, and the filtrate was concentrated under reduced pressure to give a residue. The residue was purified by column chromatography to afford product as a colorless oil (98%). ¹H NMR (400 MHz, CDCl₃) δ 5.43-5.16 (m, 4H), 3.46 (t, J=6.8 Hz, 2H), 2.70 (t, J=6.6 Hz, 2H), 1.97 (t, J=6.9 Hz, 4H), 1.75-1.65 (m, 2H), 1.40-1.18 (m, 16H), 0.82 (t, J=6.8 Hz, 3H).

### Intermediate 23c: diethyl 2-((9Z,12Z)-octadeca-9,12-dien-1-yl)malonate

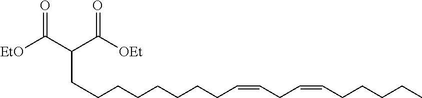 [SMILES: CCCCC/C=C\C/C=C\CCCCCCCCC(C(=O)OCC)C(=O)OCC]

To a solution of Intermediate 23c (8 g, 1.0 equiv.) in DMF (0.2-0.4 M) was added K₂CO₃ (3.0 equiv.) and diethyl propanedioate (1.5 equiv.). The mixture was stirred at 60° C. for 24 h. The reaction mixture was diluted with H₂O and extracted 3× with EtOAc. The combined organic layers were washed 3× with brine, dried over Na₂SO₄, filtered, and the filtrate was concentrated under reduced pressure to give a residue. The residue was purified by column chromatography to afford product as a colorless oil (60%). ¹H NMR (400 MHz, CDCl₃) δ 5.38-5.20 (m, 4H), 4.12 (qd, J=7.1, 1.1 Hz, 4H), 3.24 (t, J=7.6 Hz, 1H), 2.70 (t, J=6.5 Hz, 2H), 1.98 (qd, J=6.7, 2.4 Hz, 4H), 1.81 (q, J=7.5 Hz, 2H), 1.32-1.16 (m, 23H), 0.82 (t, J=6.8 Hz, 3H).

### Intermediate 23d: 2-((9Z,12Z)-octadeca-9,12-dien-1-yl)propane-1,3-

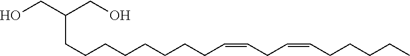 [SMILES: CCCCC/C=C\C/C=C\CCCCCCCCC(CO)CO]

To a solution of Intermediate 23c (5 g, 1.0 equiv.) in THF (0.1-0.4 M) was added LiAlH₄ (2.2 equiv.) slowly at 0° C. under N₂ atmosphere. The mixture was stirred at 20° C. for 12 h. The reaction mixture was quenched by pouring into sat. NH₄Cl and stirred for 0.5 h, followed by 3× extraction with EtOAc. The combined organic phase was washed 3× with brine, dried over Na₂SO₄, filtered, and the filtrate was concentrated under reduced pressure. The residue was purified by column chromatography to afford product as a colorless oil (53%). ¹H NMR (400 MHz, CDCl₃) δ 5.28 (tt, J=11.1, 4.8 Hz, 4H), 3.75 (dd, J=10.6, 3.7 Hz, 2H), 3.59 (dd, J=10.6, 7.6 Hz, 2H), 2.71 (t, J=6.5 Hz, 2H), 1.98 (q, J=7.0 Hz, 4H), 1.70 (ddq, J=10.6, 7.1, 3.5 Hz, 1H), 1.34-1.12 (m, 21H), 0.82 (t, J=6.7 Hz, 3H).

### Intermediate 23e: 1-(heptadecan-9-yl) 7-((11Z,14Z)-2-(hydroxymethyl)icosa-11,14-dien-1-yl) heptanedioate

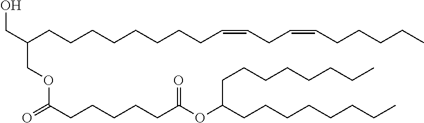 [SMILES: CCCCC/C=C\C/C=C\CCCCCCCCC(CO)COC(=O)CCCCCC(=O)OC(CCCCCCCC)CCCCCCCC]

A mixture of Intermediate 23d (4 g, 1.0 equiv.), DMAP (0.2 equiv.), EDCI (2.0 equiv.) and DIPEA (2.0 equiv.) in DCM (0.3 M) was degassed and purged 3× with N₂, then 7-(1-octylnonoxy)-7-oxo-heptanoic acid (1.0 equiv.) was added to the mixture slowly at 0° C. The mixture was stirred at 20° C. for 12 h under N₂ atmosphere. The reaction mixture was diluted with H₂O and extracted 2× with DCM. The combined organic layers were washed 2× with brine, dried over Na₂SO₄, filtered, and the filtrate was concentrated under reduced pressure to give a residue. The residue was purified by column chromatography to afford product as a colorless oil (48%). ¹H NMR (400 MHz, CDCl₃) δ 5.28 (tt, J=11.0, 4.9 Hz, 4H), 4.79 (p, J=6.3 Hz, 1H), 4.15 (dd, J=11.2, 4.3 Hz, 1H), 4.01 (dd, J=11.2, 6.7 Hz, 1H), 3.52 (dt, J=10.7, 5.0 Hz, 1H), 3.44 (q, J=5.7 Hz, 1H), 2.70 (t, J=6.5 Hz, 2H), 2.24 (dt, J=17.0, 7.5 Hz, 4H), 1.98 (q, J=7.0 Hz, 5H), 1.72 (ddt, J=11.1, 6.6, 4.4 Hz, 1H), 1.58 (pd, J=7.5, 5.1 Hz, 4H), 1.43 (q, J=6.0 Hz, 4H), 1.36-1.13 (m, 47H), 0.91-0.73 (m, 9H).

### Intermediate 23f: 1-(heptadecan-9-yl) 7-((11Z,14Z)-2-((((4-nitrophenoxy)carbonyl)oxy)methyl)icosa-11,14-dien-1-yl) heptanedioate

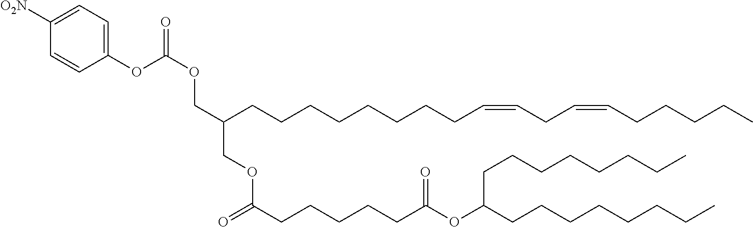 [SMILES: CCCCC/C=C\C/C=C\CCCCCCCCC(COC(=O)CCCCCC(=O)OC(CCCCCCCC)CCCCCCCC)COC(=O)Oc1ccc([N+](=O)[O-])cc1]

To a solution of Intermediate 23e (4 g, 1.0 equiv.), (4-nitrophenyl) carbonochloridate (2.0 equiv.) in DCM (0.1-0.2 M). Then pyridine (2.0 equiv.) was added slowly at 0° C. The mixture was stirred at 20° C. for 1 h. The reaction mixture was diluted with hexanes, filtered, and the filtrate was concentrated under reduced pressure to give a residue. The residue was purified by column chromatography to afford product as a colorless oil. ¹H NMR (400 MHz, CDCl₃) δ 8.26-8.17 (m, 2H), 7.36-7.27 (m, 2H), 5.37-5.22 (m, 4H), 4.79 (p, J=6.3 Hz, 1H), 4.26-4.16 (m, 2H), 4.10 (dd, J=11.2, 4.7 Hz, 1H), 4.02 (dd, J=11.3, 6.7 Hz, 1H), 2.70 (t, J=6.5 Hz, 2H), 2.24 (dt, J=20.3, 7.5 Hz, 4H), 2.00 (dq, J=13.9, 7.0 Hz, 5H), 1.56 (dq, J=16.5, 9.3, 8.3 Hz, 4H), 1.43 (q, J=6.2 Hz, 4H), 1.39-1.15 (m, 45H), 0.81 (q, J=6.7 Hz, 9H).

### Compound 23: 1-((11Z,14Z)-2-((((2-(diethylamino)ethyl)carbamoyl)oxy)methyl)icosa-11,14-dien-1-yl) 7-(heptadecan-9-yl) heptanedioate

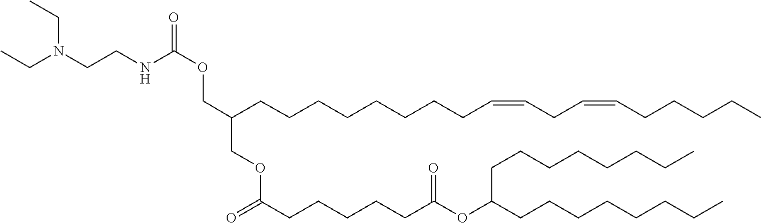 [SMILES: CCCCC/C=C\C/C=C\CCCCCCCCC(COC(=O)CCCCCC(=O)OC(CCCCCCCC)CCCCCCCC)COC(=O)NCCN(CC)CC]

Compound 23 was synthesized from Intermediate 23f and N′,N′-diethylethane-1,2-diamine using the method employed in the synthesis of Compound 1. ¹H NMR (400 MHz, CDCl₃) δ 5.29 (qd, J=11.0, 5.0 Hz, 4H), 5.10 (s, 1H), 4.79 (p, J=6.3 Hz, 1H), 3.97 (dq, J=16.0, 8.7, 5.8 Hz, 4H), 3.14 (q, J=5.9 Hz, 2H), 2.70 (t, J=6.5 Hz, 2H), 2.45 (q, J=7.1, 6.3 Hz, 6H), 2.23 (dt, J=10.4, 7.6 Hz, 4H), 1.98 (q, J=6.9 Hz, 4H), 1.88 (d, J=7.6 Hz, 1H), 1.62-1.52 (m, 5H), 1.43 (q, J=6.1 Hz, 4H), 1.33-1.17 (m, 46H), 0.93 (t, J=7.1 Hz, 6H), 0.85-0.77 (m, 9H). MS: 847.4 m/z [M+H].

### Example 24—Compound 24

### Intermediate 24a: octadecan-9-ol

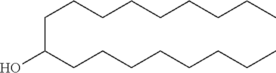 [SMILES: CCCCCCCCCC(O)CCCCCCCC]

To a solution of decanal (50 g, 1.0 equiv.) in THF (0.1-0.2 M) was added bromo(octyl)magnesium (1.0 equiv.) dropwise at −40° C. under N₂ atmosphere. The mixture was stirred at 20° C. for 2 h. The reaction mixture was quenched by adding H₂O and extracted 2× with EtOAc. The combined organic layers were dried over Na₂SO₄, filtered, and the filtrate was concentrated under reduced pressure to give a residue. The residue was purified by column chromatography to afford product as a white solid.

### Intermediate 24b: 9-bromononanoic Acid

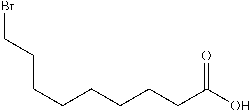 [SMILES: O=C(O)CCCCCCCCBr]

Intermediate 24b was synthesized (60%) from Intermediate 24a and 9-bromononanoic acid using the method employed in the synthesis of Intermediate 7a. ¹H NMR (400 MHz, CDCl₃) δ 4.80 (p, J=6.3 Hz, 1H), 3.46 (t, J=6.8 Hz, 1H), 3.33 (t, J=6.9 Hz, 1H), 2.21 (t, J=7.5 Hz, 2H), 1.78 (p, J=7.0 Hz, 1H), 1.69 (dt, J=14.4, 6.9 Hz, 1H), 1.55 (p, J=7.2 Hz, 2H), 1.43 (q, J=6.1 Hz, 4H), 1.39-1.31 (m, 2H), 1.22 (d, J=24.2 Hz, 32H), 0.81 (t, J=6.8 Hz, 6H).

### Intermediate 24c: 1,1-dibenzyl 9-(octadecan-9-yl) nonane-1,1,9-tricarboxylate

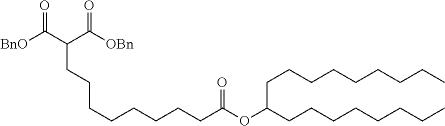 [SMILES: CCCCCCCCCC(CCCCCCCC)OC(=O)CCCCCCCCC(C(=O)OCc1ccccc1)C(=O)OCc1ccccc1]

Intermediate 24c was synthesized (74%) from Intermediate 24b using the method employed in the synthesis of Intermediate 7b. ¹H NMR (400 MHz, CDCl₃) δ 7.35-7.10 (m, 10H), 5.07 (s, 4H), 4.79 (p, J=6.3 Hz, 1H), 3.36 (t, J=7.5 Hz, 1H), 2.19 (t, J=7.5 Hz, 2H), 1.85 (d, J=7.2 Hz, 2H), 1.58-1.47 (m, 3H), 1.43 (q, J=6.1 Hz, 4H), 1.19 (d, J=7.9 Hz, 36H), 0.80 (t, J=6.7 Hz, 6H).

### Intermediate 24d: 2-(9-(octadecan-9-yloxy)-9-oxononyl)malonic Acid

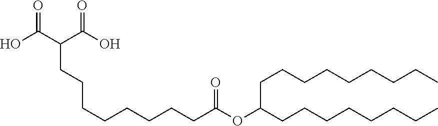 [SMILES: CCCCCCCCCC(CCCCCCCC)OC(=O)CCCCCCCCC(C(=O)O)C(=O)O]

Intermediate 24d was synthesized from Intermediate 24c using the method employed in the synthesis of Intermediate 24c.

### Intermediate 24e: octadecan-9-yl 11-hydroxy-10-(hydroxymethyl)undecanoate

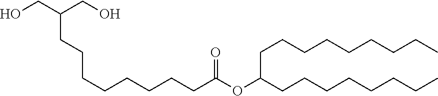 [SMILES: CCCCCCCCCC(CCCCCCCC)OC(=O)CCCCCCCCC(CO)CO]

Intermediate 24e was synthesized (30%) from Intermediate 24d using the method employed in the synthesis of Intermediate 7d. ¹H NMR (400 MHz, CDCl₃) δ 4.79 (p, J=6.3 Hz, 1H), 3.75 (dd, J=10.6, 3.8 Hz, 2H), 3.59 (dd, J=10.6, 7.6 Hz, 2H), 2.21 (t, J=7.5 Hz, 4H), 1.70 (ddq, J=10.5, 7.0, 3.4 Hz, 1H), 1.54 (t, J=7.2 Hz, 2H), 1.43 (q, J=6.1 Hz, 4H), 1.20 (d, J=12.6 Hz, 39H), 0.81 (t, J=6.7 Hz, 6H).

### Intermediate 24f: 2-(hydroxymethyl)-11-(octadecan-9-yloxy)-11-oxoundecyl (9Z,12Z)-octadeca-9,12-dienoate

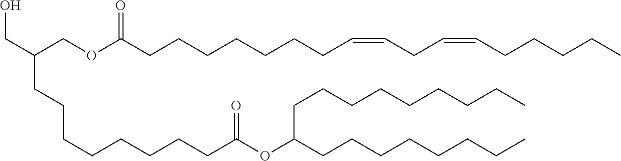 [SMILES: CCCCC/C=C\C/C=C\CCCCCCCC(=O)OCC(CO)CCCCCCCCC(=O)OC(CCCCCCCC)CCCCCCCCC]

Intermediate 24f was synthesized (58%) from Intermediate 24e using the method employed in the synthesis of Intermediate 7e. ¹H NMR (400 MHz, CDCl₃) δ 5.37-5.18 (m, 4H), 4.79 (p, J=6.3 Hz, 1H), 4.15 (dd, J=11.2, 4.3 Hz, 1H), 4.01 (dd, J=11.2, 6.7 Hz, 1H), 3.52 (ddd, J=11.0, 6.4, 4.4 Hz, 1H), 3.42 (dt, J=11.6, 6.1 Hz, 1H), 2.70 (t, J=6.4 Hz, 2H), 2.23 (dt, J=18.7, 7.5 Hz, 4H), 1.98 (q, J=6.8 Hz, 4H), 1.92 (t, J=6.2 Hz, 1H), 1.72 (ddd, J=11.0, 6.7, 4.3 Hz, 1H), 1.54 (qd, J=7.2, 4.1 Hz, 4H), 1.43 (q, J=5.9 Hz, 4H), 1.35-1.09 (m, 54H), 0.81 (td, J=6.8, 4.9 Hz, 9H).

### Intermediate 24g: sodium 4-hydroxydodecanoate

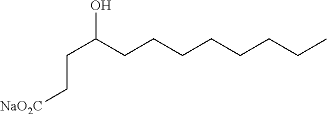

To a solution of 5-octyltetrahydrofuran-2-one (50 g, 1.0 equiv.) in H₂O (0.5 M) was added NaOH (1.0-1.1 equiv.). The mixture was degassed, purged 3× with N₂ and stirred at 20° C. for 12 h. The reaction mixture was concentrated to afford the crude product that was used directly in the next step without additional purification (83%).

### Intermediate 24h: benzyl 4-hydroxydodecanoate

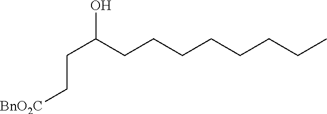

To a solution of Intermediate 24g (10 g, 1.0 equiv.) in DMSO (0.5-5.0 M) was added BnBr (1.0 equiv.) at 0° C. The mixture was stirred at 20° C. for 10 min. The reaction mixture was then diluted with 1:1 EtOAc/hexanes. The combined organic layers were washed 3× with NH₄Cl, dried over Na₂SO₄, filtered, and the filtrate was concentrated under reduced pressure to give a residue that was used directly in the next step without additional purification (78%).

### Intermediate 24i: benzyl 4-(((4-nitrophenoxy)carbonyl)oxy)dodecanoate

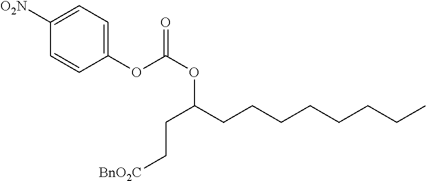

To a solution of Intermediate 24h (25 g, 1.0 equiv.), (4-nitrophenyl) carbonochloridate (1.5 equiv.) in DCM (0.2-0.5 M) was added pyridine (2.0 equiv.) dropwise at 0° C. The mixture was stirred at 20° C. for 1 h under N₂ atmosphere. The reaction mixture was diluted with hexanes and filtered. The filtrate was concentrated under reduced pressure to remove solvent. The residue was purified by column chromatography to afford product as a colorless oil (26%). ¹H NMR (400 MHz, CDCl₃) δ 8.24-8.14 (m, 2H), 7.38-7.27 (m, 7H), 5.06 (s, 2H), 4.80 (tdd, J=7.6, 5.5, 4.0 Hz, 1H), 2.49-2.39 (m, 2H), 2.04 (dtd, J=15.5, 7.7, 4.1 Hz, 1H), 1.97-1.87 (m, 1H), 1.70-1.60 (m, 1H), 1.60-1.48 (m, 1H), 1.36-1.14 (m, 12H), 0.94-0.69 (m, 3H).

### Intermediate 24j: benzyl 4-(((2-(ethyl(methyl)amino)ethyl)carbamoyl)oxy)dodecanoate

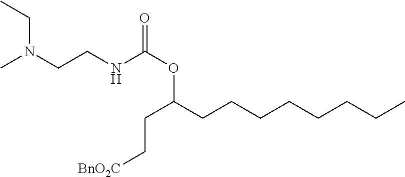

To a solution of Intermediate 24i (8 g, 1.0 equiv.), N′-ethyl-N′-methyl-ethane-1,2-diamine; hydrochloride (1.0 equiv.) in MeCN (0.1-0.2 M) was added pyridine (2.0 equiv.) and DMAP (0.1 equiv.). The mixture was stirred at 20° C. for 12 h under N₂ atmosphere. The reaction mixture was concentrated under reduced pressure to remove solvent. The residue was diluted with EtOAc and washed 3× with NaHCO₃ and 5× with H₂O. The organic layer was dried over Na₂SO₄, filtered, and the filtrate was concentrated under reduced pressure to give a residue. The residue was purified by column chromatography to afford product as a pale yellow oil (54%). ¹H NMR (400 MHz, CDCl₃) δ 7.34-7.21 (m, 5H), 5.07 (s, 2H), 4.69 (s, 1H), 3.17 (q, J=6.0 Hz, 2H), 2.44-2.30 (m, 6H), 2.15 (s, 3H), 1.86 (td, J=7.8, 4.1 Hz, 1H), 1.75 (dq, J=14.9, 7.9 Hz, 1H), 1.57-1.35 (m, 2H), 1.29-1.10 (m, 12H), 0.97 (t, J=7.1 Hz, 3H), 0.85-0.74 (m, 3H).

### Intermediate 24k: 4-(((2-(ethyl(methyl)amino)ethyl)carbamoyl)oxy)dodecanoic Acid

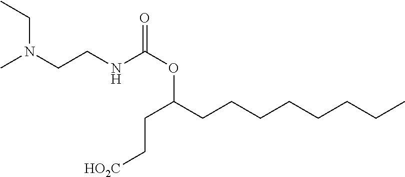 [SMILES: CCCCCCCCC(CCC(=O)O)OC(=O)NCCN(C)CC]

To a solution of Pd/C (1.0 equiv.) in THF (0.1-0.5 M) was added Intermediate 24j (4 g, 1.0 equiv.). The mixture was degassed and purged 3× with H₂ and stirred at 20° C. for 5 h under H₂ atmosphere. The reaction mixture was filtered, and the filtrate was concentrated under reduced pressure to give a residue. The residue was purified by column chromatography to afford product as a brown oil (63%). ¹H NMR (400 MHz, CDCl₃) δ 6.14 (dd, J=7.9, 4.0 Hz, 1H), 4.66 (qd, J=7.4, 3.9 Hz, 1H), 3.53 (dtd, J=15.1, 7.8, 7.4, 3.9 Hz, 1H), 3.13 (ddt, J=14.8, 7.4, 3.9 Hz, 1H), 2.87-2.57 (m, 4H), 2.38 (s, 3H), 2.31-2.13 (m, 2H), 1.95 (s, 1H), 1.73 (dt, J=14.4, 7.5 Hz, 1H), 1.59-1.47 (m, 1H), 1.41 (dt, J=13.8, 7.4 Hz, 1H), 1.30-0.97 (m, 15H), 0.80 (t, J=6.8 Hz, 3H).

### Compound 24: 3-methyl-15-(9-(octadecan-9-yloxy)-9-oxononyl)-9-octyl-7,12-dioxo-8,13-dioxa-3,6-diazahexadecan-16-yl (9Z,12Z)-octadeca-9,12-dienoate

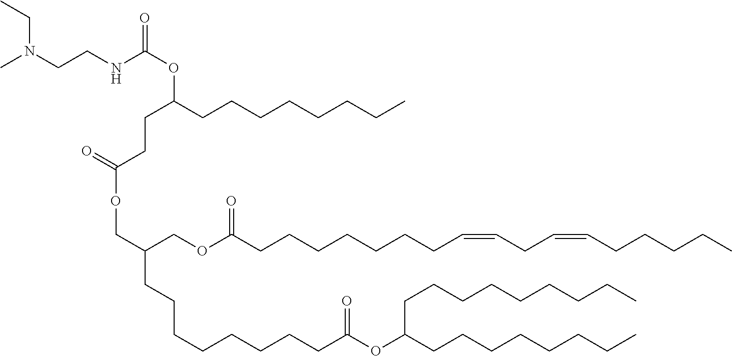 [SMILES: CCCCC/C=C\C/C=C\CCCCCCCC(=O)OCC(CCCCCCCCC(=O)OC(CCCCCCCC)CCCCCCCCC)COC(=O)CCC(CCCCCCCC)OC(=O)NCCN(C)CC]

A mixture of Intermediate 24k (2.0 g, 1.0 equiv.), Intermediate 24f (1.0 equiv.), EDCI (1.2 equiv.), DIPEA (2.5 equiv.), and DMAP (0.1 equiv.) in DCM (0.1-0.5 M) was degassed and purged 3× with N₂, and then the mixture was stirred at 20° C. for 12 h under N₂ atmosphere. The reaction mixture was diluted with H₂O and extracted 3× with DCM. The combined organic layers were dried over Na₂SO₄, filtered, and the filtrate was concentrated under reduced pressure to give a residue. The residue was purified by column chromatography, then diluted in MeCN and washed 3× with hexanes to afford product as a colorless oil. ¹H NMR (400 MHz, CDCl₃) δ 5.41-5.20 (m, 4H), 5.05 (d, J=6.0 Hz, 1H), 4.80 (q, J=6.2 Hz, 1H), 4.68 (s, 1H), 3.97 (qd, J=11.1, 6.3 Hz, 4H), 3.17 (q, J=6.0 Hz, 2H), 2.70 (t, J=6.4 Hz, 2H), 2.42-2.25 (m, 6H), 2.22 (dt, J=9.5, 7.5 Hz, 4H), 2.13 (s, 3H), 1.98 (q, J=6.9 Hz, 4H), 1.92-1.69 (m, 3H), 1.60-1.39 (m, 11H), 1.33-1.12 (m, 62H), 0.97 (t, J=7.1 Hz, 3H), 0.86-0.78 (m, 12H). MS: 1074.0 m/z [M+H].

### Example 25—Compound 25

### Intermediate 25a: benzyl 4-(((2-(pyrrolidin-1-yl)ethyl)carbamoyl)oxy)dodecanoate

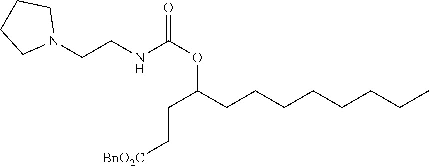

Intermediate 25a was synthesized from Intermediate 24i and 2-pyrrolidin-1-ylethanamine using the method employed in the synthesis of Intermediate 24j.

### Intermediate 25b: 4-(((2-(pyrrolidin-1-yl)ethyl)carbamoyl)oxy)dodecanoic Acid

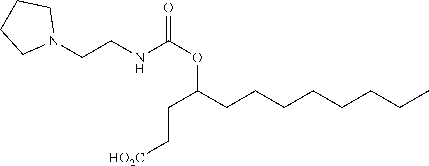 [SMILES: CCCCCCCCC(CCC(=O)O)OC(=O)NCCN1CCCC1]

Intermediate 25b was synthesized from Intermediate 25a using the method employed in the synthesis of Intermediate 24k. ¹H NMR (400 MHz, CDCl₃) δ 6.19 (s, 1H), 5.87 (s, 2H), 4.67 (p, J=6.4 Hz, 1H), 3.65-3.53 (m, 1H), 3.14-3.01 (m, 1H), 2.94 (qd, J=16.2, 13.5, 5.5 Hz, 4H), 2.76 (t, J=9.6 Hz, 1H), 2.20 (hept, J=7.3 Hz, 2H), 2.07-1.83 (m, 5H), 1.71 (dq, J=14.7, 7.4 Hz, 1H), 1.54 (dt, J=14.2, 6.8 Hz, 1H), 1.40 (dt, J=13.8, 6.9 Hz, 1H), 1.31-1.13 (m, 12H), 0.80 (t, J=6.7 Hz, 3H).

### Compound 25: 11-(octadecan-9-yloxy)-11-oxo-2-(((4-(((2-(pyrrolidin-1-yl)ethyl)carbamoyl)oxy)dodecanoyl)oxy)methyl)undecyl (9Z,12Z)-octadeca-9,12-dienoate

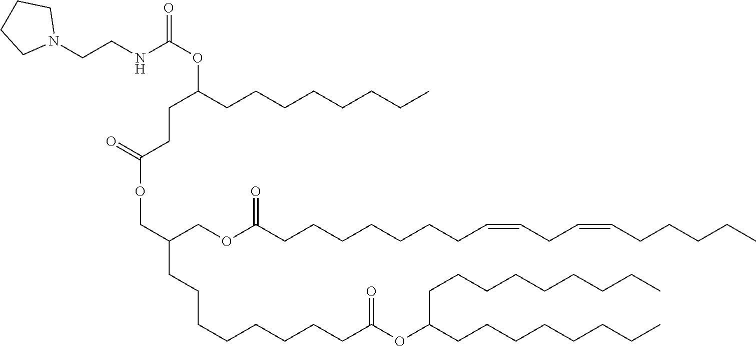 [SMILES: CCCCC/C=C\C/C=C\CCCCCCCC(=O)OCC(CCCCCCCCC(=O)OC(CCCCCCCC)CCCCCCCCC)COC(=O)CCC(CCCCCCCC)OC(=O)NCCN1CCCC1]

Compound 25 was synthesized from Intermediate 25b and Intermediate 24f using the method employed in the synthesis of Compound 24. ¹H NMR (400 MHz, CDCl₃) δ 5.28 (tt, J=11.0, 5.4 Hz, 4H), 5.09 (s, 1H), 4.79 (p, J=6.2 Hz, 1H), 4.70 (d, J=11.8 Hz, 1H), 3.97 (qd, J=11.2, 6.2 Hz, 4H), 3.18 (q, J=6.1 Hz, 2H), 2.70 (t, J=6.4 Hz, 2H), 2.38 (dt, J=14.2, 6.4 Hz, 4H), 2.30 (dt, J=8.8, 5.8 Hz, 2H), 2.22 (dt, J=9.7, 7.6 Hz, 4H), 2.14 (s, 3H), 1.99 (t, J=6.8 Hz, 4H), 1.91-1.69 (m, 3H), 1.49 (dq, J=45.3, 6.5, 6.0 Hz, 13H), 1.36-1.06 (m, 58H), 0.81 (q, J=6.8, 6.3 Hz, 12H). MS: 1085.9 m/z [M+H].

### Example 26—Compound 26

### Intermediate 26a: sodium 4-hydroxyoctanoate

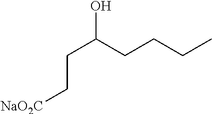

Intermediate 26a was synthesized from 5-butyltetrahydrofuran-2-one using the method employed in the synthesis of Intermediate 24g.

### Intermediate 26b: benzyl 4-hydroxyoctanoate

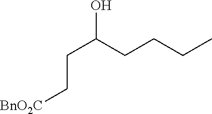

Intermediate 26b was synthesized from Intermediate 26a using the method employed in the synthesis of Intermediate 24h.

### Intermediate 26c: benzyl 4-(((4-nitrophenoxy)carbonyl)oxy)octanoate

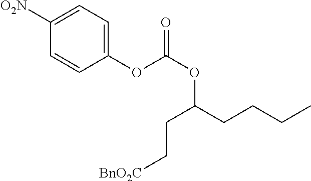

Intermediate 26c was synthesized from Intermediate 26b using the method employed in the synthesis of Intermediate 24i. ¹H NMR (400 MHz, CDCl₃) δ 8.38-8.19 (m, 2H), 7.51-7.29 (m, 7H), 5.15 (s, 2H), 4.97-4.86 (m, 1H), 2.53 (dd, J=8.1, 6.8 Hz, 2H), 2.14 (dtd, J=15.4, 7.7, 4.0 Hz, 1H), 2.08-1.95 (m, 1H), 1.82-1.72 (m, 1H), 1.66 (ddt, J=14.5, 8.9, 5.5 Hz, 1H), 1.48-1.32 (m, 4H), 1.04-0.85 (m, 3H).

### Intermediate 26d: benzyl 4-(((2-(ethyl(methyl)amino)ethyl)carbamoyl)oxy)octanoate

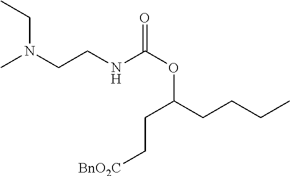

Intermediate 26d was synthesized from Intermediate 26c using the method employed in the synthesis of Intermediate 24j.

### Intermediate 26e: 4-(((2-(ethyl(methyl)amino)ethyl)carbamoyl)oxy)octanoic Acid

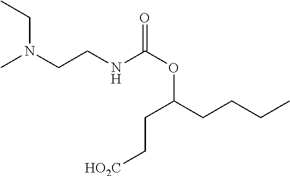 [SMILES: CCCCC(CCC(=O)O)OC(=O)NCCN(C)CC]

Intermediate 26e was synthesized from Intermediate 26d using the method employed in the synthesis of Intermediate 24k. ¹H NMR (400 MHz, CDCl₃) δ 6.16 (s, 1H), 4.76 (dq, J=11.5, 6.6 Hz, 1H), 3.64 (dtd, J=14.9, 7.5, 2.7 Hz, 1H), 3.20 (ddt, J=14.7, 7.1, 3.7 Hz, 1H), 2.93-2.71 (m, 4H), 2.47 (s, 3H), 2.41-2.20 (m, 2H), 2.05 (ddd, J=18.5, 9.7, 5.7 Hz, 1H), 1.81 (dq, J=14.7, 7.5 Hz, 1H), 1.75-1.58 (m, 1H), 1.50 (d, J=13.7 Hz, 1H), 1.40-1.25 (m, 4H), 1.21 (t, J=7.2 Hz, 3H), 1.01-0.73 (m, 3H).

### Compound 26: 9-butyl-3-methyl-15-(9-(octadecan-9-yloxy)-9-oxononyl)-7,12-dioxo-8,13-dioxa-3,6-diazahexadecan-16-yl (9Z,12Z)-octadeca-9,12-dienoate

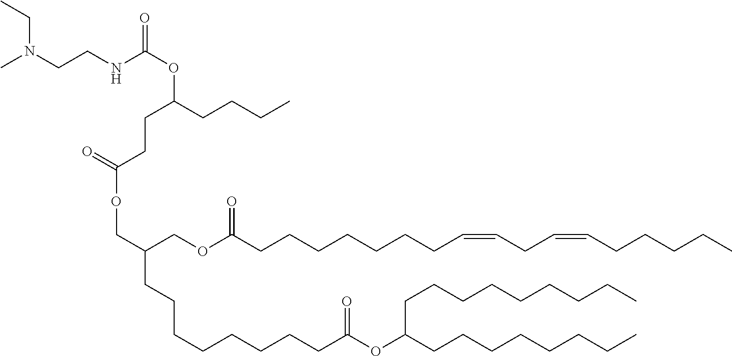 [SMILES: CCCCC/C=C\C/C=C\CCCCCCCC(=O)OCC(CCCCCCCCC(=O)OC(CCCCCCCC)CCCCCCCCC)COC(=O)CCC(CCCC)OC(=O)NCCN(C)CC]

Compound 26 was synthesized from Intermediate 26e and Intermediate 24f using the method employed in the synthesis of Compound 24. ¹H NMR (400 MHz, CDCl₃) δ 5.28 (tt, J=11.0, 5.4 Hz, 4H), 5.09 (s, 1H), 4.79 (p, J=6.2 Hz, 1H), 4.70 (d, J=11.8 Hz, 1H), 3.97 (qd, J=11.2, 6.2 Hz, 4H), 3.18 (q, J=6.1 Hz, 2H), 2.70 (t, J=6.4 Hz, 2H), 2.38 (dt, J=14.2, 6.4 Hz, 4H), 2.30 (dt, J=8.8, 5.8 Hz, 2H), 2.27-2.07 (m, 7H), 1.99 (t, J=6.8 Hz, 4H), 1.94-1.79 (m, 2H), 1.73 (t, J=7.8 Hz, 1H), 1.49 (dq, J=45.3, 6.5, 6.0 Hz, 13H), 1.37-1.09 (m, 57H), 0.98 (t, J=7.1 Hz, 3H), 0.81 (q, J=6.8, 6.3 Hz, 12H). MS: 1017.9 m/z [M+H].

### Example 27—Compound 27

### Intermediate 27a: benzyl 4-(((2-(pyrrolidin-1-yl)ethyl)carbamoyl)oxy)octanoate

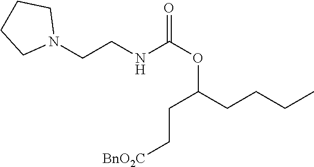

Intermediate 27a was synthesized from Intermediate 26c using the method employed in the synthesis of Intermediate 25a.

### Intermediate 27b: 4-(((2-(pyrrolidin-1-yl)ethyl)carbamoyl)oxy)octanoic Acid

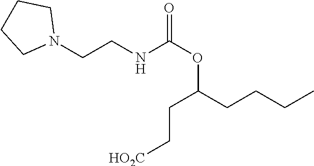 [SMILES: CCCCC(CCC(=O)O)OC(=O)NCCN1CCCC1]

Intermediate 27b was synthesized from Intermediate 27a using the method employed in the synthesis of Intermediate 24k. ¹H NMR (400 MHz, CDCl₃) δ 6.49 (dd, J=7.8, 4.0 Hz, 1H), 4.66 (tt, J=10.4, 5.1 Hz, 1H), 3.55 (dtd, J=14.5, 7.1, 3.4 Hz, 1H), 3.14 (ddt, J=15.1, 7.7, 3.6 Hz, 1H), 3.05-2.81 (m, 5H), 2.80-2.59 (m, 1H), 2.32-2.09 (m, 2H), 2.00-1.83 (m, 5H), 1.73 (dtd, J=14.6, 8.8, 6.2 Hz, 1H), 1.62-1.34 (m, 2H), 1.22 (dddd, J=14.2, 11.9, 8.9, 5.2 Hz, 4H), 0.80 (q, J=5.0, 3.4 Hz, 3H).

### Compound 27: 11-(octadecan-9-yloxy)-11-oxo-2-(((4-(((2-(pyrrolidin-1-yl)ethyl)carbamoyl)oxy)octanoyl)oxy)methyl)undecyl (9Z,12Z)-octadeca-9,12-dienoate

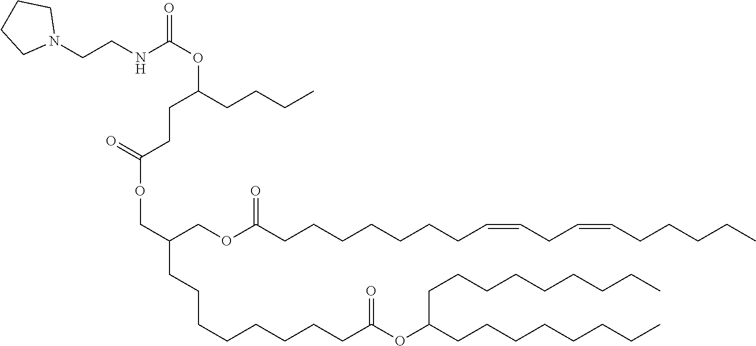 [SMILES: CCCCC/C=C\C/C=C\CCCCCCCC(=O)OCC(CCCCCCCCC(=O)OC(CCCCCCCC)CCCCCCCCC)COC(=O)CCC(CCCC)OC(=O)NCCN1CCCC1]

Compound 27 was synthesized from Intermediate 27b and Intermediate 24f using the method employed in the synthesis of Compound 24. ¹H NMR (400 MHz, CDCl₃) δ 5.38-5.20 (m, 4H), 5.06 (s, 1H), 4.79 (p, J=6.3 Hz, 1H), 4.69 (s, 1H), 3.97 (dtd, J=17.5, 10.7, 4.3 Hz, 4H), 3.21 (q, J=5.9 Hz, 2H), 2.70 (t, J=6.4 Hz, 2H), 2.51 (t, J=6.1 Hz, 2H), 2.43 (d, J=6.0 Hz, 4H), 2.34-2.26 (m, 2H), 2.22 (dt, J=9.7, 7.5 Hz, 4H), 1.98 (q, J=6.9 Hz, 4H), 1.94-1.79 (m, 2H), 1.72 (td, J=12.1, 10.3, 4.9 Hz, 5H), 1.54 (t, J=7.1 Hz, 6H), 1.43 (q, J=6.5 Hz, 5H), 1.34-1.13 (m, 58H), 0.81 (td, J=6.7, 4.7 Hz, 12H). MS: 1029.9 m/z [M+H].

### Example 28—Compound 28

### Compound 28: di(heptadecan-9-yl) O,O′-(2-((((2-(piperidin-1-yl)ethyl)carbamoyl)oxy)methyl)propane-1,3-diyl) diglutarate

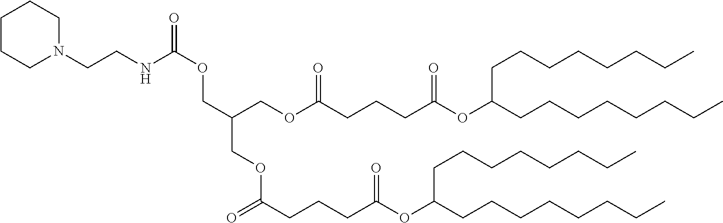 [SMILES: CCCCCCCCC(CCCCCCCC)OC(=O)CCCC(=O)OCC(COC(=O)CCCC(=O)OC(CCCCCCCC)CCCCCCCC)COC(=O)NCCN1CCCCC1]

Compound 28 was synthesized from Intermediate 2d and 2-(1-piperidyl) ethanamine using the method employed in the synthesis of Compound 6. ¹H NMR (400 MHz, CDCl₃) δ 5.18 (s, 1H), 4.80 (p, J=6.2 Hz, 2H), 4.06 (t, J=7.2 Hz, 6H), 3.19 (q, J=5.8 Hz, 2H), 2.30 (dt, J=14.9, 7.4 Hz, 15H), 1.87 (p, J=7.4 Hz, 4H), 1.55-1.31 (m, 14H), 1.19 (s, 50H), 0.81 (t, J=6.7 Hz, 12H). MS: 966.3 m/z [M+H].

### Example 29—Compound 29

### Compound 29: di(heptadecan-9-yl) O,O′-(2-((((2-(pyrrolidin-1-yl)ethyl)carbamoyl)oxy)methyl)propane-1,3-diyl) diglutarate

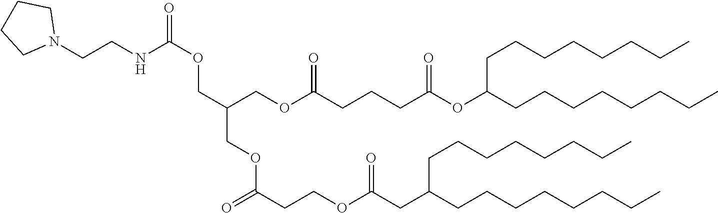 [SMILES: CCCCCCCCC(CCCCCCCC)CC(=O)OCCC(=O)OCC(COC(=O)CCCC(=O)OC(CCCCCCCC)CCCCCCCC)COC(=O)NCCN1CCCC1]

Compound 29 was synthesized from Intermediate 2d and 2-pyrrolidin-1-ylethanamine using the method employed in the synthesis of Compound 6. ¹H NMR (400 MHz, CDCl₃) δ 5.18 (s, 1H), 4.80 (p, J=6.3 Hz, 2H), 4.06 (t, J=6.6 Hz, 6H), 3.21 (q, J=5.8 Hz, 2H), 2.51 (t, J=6.1 Hz, 2H), 2.45 (d, J=7.2 Hz, 4H), 2.30 (dt, J=14.7, 7.4 Hz, 9H), 1.87 (p, J=7.5 Hz, 4H), 1.71 (q, J=3.4 Hz, 4H), 1.44 (q, J=6.0 Hz, 8H), 1.19 (s, 49H), 0.81 (t, J=6.8 Hz, 12H). MS: 952.3 m/z [M+H].

### Example 30—Compound 30

### Compound 30: O,O′-(2-((((3-(ethyl(methyl)amino)propoxy)carbonyl)oxy)methyl)propane-1,3-diyl) di(heptadecan-9-yl) diglutarate

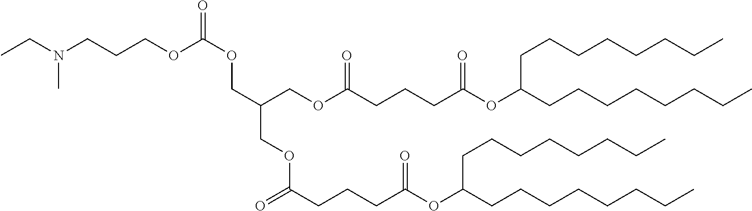 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(C)CC]

Compound 30 was synthesized from Intermediate 2d and 3-[ethyl(methyl)amino]propan-1-ol using the method employed in the synthesis of Compound 6. ¹H NMR (400 MHz, CDCl₃) δ 4.80 (p, J=6.3 Hz, 2H), 4.16-4.00 (m, 8H), 2.44-2.23 (m, 12H), 2.15 (s, 3H), 1.86 (q, J=7.4 Hz, 4H), 1.79 (q, J=7.3 Hz, 2H), 1.44 (q, J=5.9 Hz, 10H), 1.19 (s, 47H), 0.98 (t, J=7.2 Hz, 3H), 0.81 (t, J=6.7 Hz, 12H). MS: 955.3 m/z [M+H].

### Example 31—Compound 31

### Compound 31: O,O′-(2-((((3-(dimethylamino)propoxy)carbonyl)oxy)methyl)propane-1,3-diyl) di(heptadecan-9-yl) diglutarate

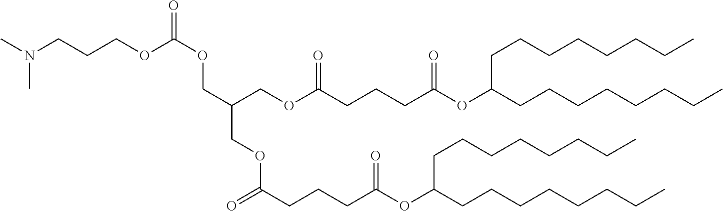 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN(C)C]

Compound 31 was synthesized from Intermediate 2d and 3-(dimethylamino)propan-1-ol using the method employed in the synthesis of Compound 6. ¹H NMR (400 MHz, CDCl₃) δ 4.80 (p, J=6.3 Hz, 2H), 4.17-4.04 (m, 8H), 2.38-2.26 (m, 11H), 2.16 (s, 6H), 1.86 (q, J=7.4 Hz, 4H), 1.77 (p, J=6.8 Hz, 2H), 1.44 (q, J=6.1 Hz, 8H), 1.19 (s, 50H), 0.81 (t, J=6.8 Hz, 12H). MS: 941.5 m/z [M+H].

### Example 32—Compound 32

### Compound 32: di(heptadecan-9-yl) O,O′-(2-((((3-(piperidin-1-yl)propoxy)carbonyl)oxy)methyl)propane-1,3-diyl) diglutarate

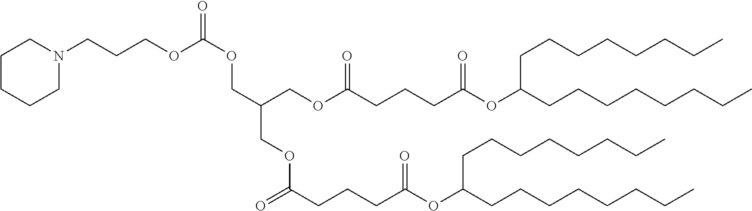 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN1CCCCC1]

Compound 32 was synthesized from Intermediate 2d and 3-(1-piperidyl)propan-1-ol using the method employed in the synthesis of Compound 6. ¹H NMR (400 MHz, CDCl₃) δ 4.80 (p, J=6.3 Hz, 2H), 4.10 (dd, J=14.7, 6.0 Hz, 8H), 2.30 (dt, J=15.3, 7.4 Hz, 14H), 1.87 (p, J=7.5 Hz, 4H), 1.79 (t, J=7.3 Hz, 2H), 1.47 (dd, J=29.0, 5.7 Hz, 15H), 1.19 (s, 49H), 0.81 (t, J=6.7 Hz, 12H). MS: 981.6 m/z [M+H].

### Example 33—Compound 33

### Compound 33: di(heptadecan-9-yl) O,O′-(2-((((3-(pyrrolidin-1-yl)propoxy)carbonyl)oxy)methyl)propane-1,3-diyl) diglutarate

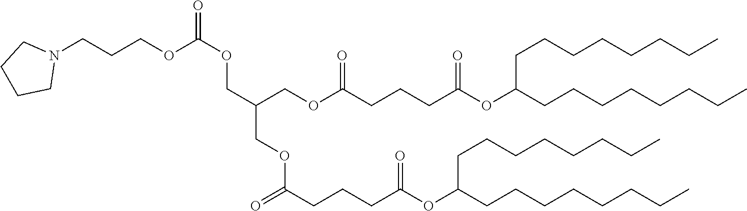 [SMILES: CCCCCCCCC(CCCCCCCC)OC(=O)CCCC(=O)OCC(COC(=O)CCCC(=O)OC(CCCCCCCC)CCCCCCCC)COC(=O)OCCCN1CCCC1]

Compound 33 was synthesized from Intermediate 2d and 3-pyrrolidin-1-ylpropan-1-ol using the method employed in the synthesis of Compound 6. ¹H NMR (400 MHz, CDCl₃) δ 4.89 (p, J=6.3 Hz, 2H), 4.27-4.12 (m, 8H), 2.62-2.46 (m, 6H), 2.39 (dt, J=15.3, 7.3 Hz, 9H), 1.95 (dp, J=12.8, 7.3 Hz, 6H), 1.53 (q, J=6.0 Hz, 8H), 1.28 (s, 48H), 0.90 (t, J=6.7 Hz, 12H). MS: 967.6 m/z [M+H].

### Example 34—Compound 34

### Compound 34: O,O′-(2-(((4-(dimethylamino)butanoyl)oxy)methyl)propane-1,3-diyl) di(heptadecan-9-yl) diglutarate

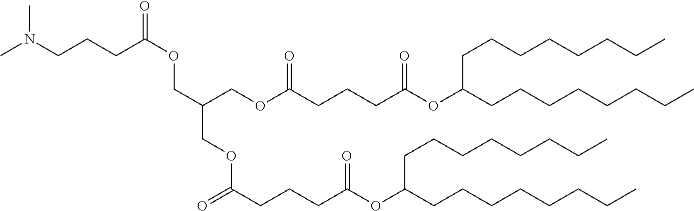 [SMILES: CCCCCCCCC(CCCCCCCC)OC(=O)CCCC(=O)OCC(COC(=O)CCCC(=O)OC(CCCCCCCC)CCCCCCCC)COC(=O)CCCN(C)C]

A mixture of Intermediate 2c (1.0 equiv.), 4-(dimethylamino)butanoic acid hydrochloride (2.0 equiv.), EDCI (1.2 equiv.), DMAP (0.1 equiv.), DIPEA (4.0 equiv.), and DCM (0.1-0.2 M) was degassed and purged 3× with N₂, and then, the mixture was stirred at 15° C. for 12 h under N₂ atmosphere. Upon completion, the reaction was concentrated under reduced pressure, and the resulting residue was diluted with water, extracted 2-3× with EtOAc, and the combined organic layers were washed 2× with sat. NaHCO₃, dried over Na₂SO₄, and filtered. The filtrate was concentrated under reduced pressure to give a residue that was purified by column chromatography to afford product. ¹H NMR (400 MHz, CDCl₃) δ 4.80 (p, J=6.3 Hz, 2H), 4.06 (d, J=6.0 Hz, 6H), 2.30 (dt, J=15.0, 7.3 Hz, 11H), 2.21 (t, J=7.2 Hz, 2H), 1.87 (p, J=7.4 Hz, 4H), 1.71 (p, J=7.4 Hz, 2H), 1.44 (q, J=6.1 Hz, 8H), 1.19 (s, 49H), 0.81 (t, J=6.7 Hz, 12H). MS: 925.4 m/z [M+H].

### Example 35—Compound 35

### Intermediate 35a: di(heptadecan-9-yl) O,O′-(2-oxopropane-1,3-diyl) diglutarate

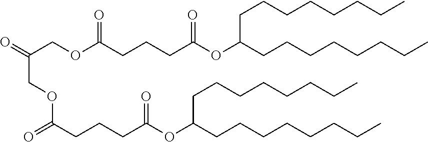 [SMILES: CCCCCCCCC(CCCCCCCC)OC(=O)CCCC(=O)OCC(=O)COC(=O)CCCC(=O)OC(CCCCCCCC)CCCCCCCC]

Intermediate 35a was synthesized from Intermediate 2b and 1,3-dihydroxypropan-2-one using the method employed in the synthesis of Intermediate 2c. ¹H NMR (400 MHz, CDCl₃) δ 4.80 (p, J=6.2 Hz, 2H), 4.69 (s, 4H), 2.44 (t, J=7.4 Hz, 4H), 2.32 (t, J=7.3 Hz, 4H), 1.94 (dt, J=14.7, 7.3 Hz, 4H), 1.43 (t, J=6.1 Hz, 8H), 1.19 (s, 49H), 0.81 (t, J=6.7 Hz, 12H).

### Intermediate 35b: di(heptadecan-9-yl) O,O′-(2-hydroxy)propane-1,3-diyl) diglutarate

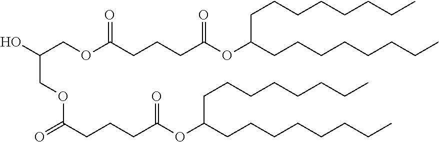 [SMILES: CCCCCCCCC(CCCCCCCC)OC(=O)CCCC(=O)OCC(O)COC(=O)CCCC(=O)OC(CCCCCCCC)CCCCCCCC]

To a solution of Intermediate 35b (1.0 equiv.) in THF/H₂O/toluene (30:15:8, 0.02 M) was added NaBH₄ (5.0 equiv.) under N₂ atmosphere at 0° C. The mixture was stirred at 5° C. for 5 h under N₂ atmosphere. The reaction was then poured into cold sat. NH₄Cl and stirred for an additional 30 min. The mixture was then poured into water and extracted 2× with EtOAc. The combined organic phase was evaporated under reduced pressure, and the resulting residue was purified by column chromatography to afford product as a white solid. ¹H NMR (400 MHz, CDCl₃) δ 4.80 (p, J=6.3 Hz, 2H), 4.18-3.95 (m, 5H), 2.33 (dt, J=25.4, 7.3 Hz, 8H), 1.90 (p, J=7.4 Hz, 4H), 1.44 (q, J=5.9 Hz, 8H), 1.19 (s, 48H), 0.81 (t, J=6.7 Hz, 12H).

### Intermediate 35c: di(heptadecan-9-yl) O,O′-(2-(((4-nitrophenoxy)carbonyl)oxy)propane-1,3-diyl) diglutarate

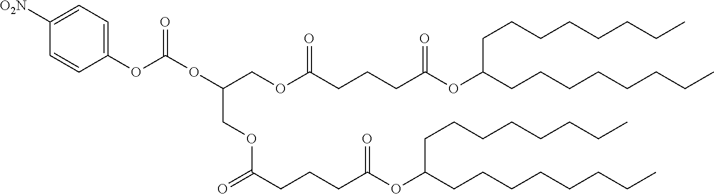 [SMILES: CCCCCCCCC(CCCCCCCC)OC(=O)CCCC(=O)OCC(COC(=O)CCCC(=O)OC(CCCCCCCC)CCCCCCCC)OC(=O)Oc1ccc([N+](=O)[O-])cc1]

Intermediate 35c was synthesized from Intermediate 35b using the method employed in the synthesis of Intermediate 1d. ¹H NMR (400 MHz, CDCl₃) δ 8.31-8.16 (m, 2H), 7.42-7.30 (m, 2H), 5.10 (td, J=6.0, 3.0 Hz, 1H), 4.80 (p, J=6.3 Hz, 2H), 4.44 (dd, J=12.3, 3.9 Hz, 2H), 4.18 (dd, J=12.3, 5.9 Hz, 2H), 2.33 (dt, J=30.6, 7.4 Hz, 8H), 1.90 (p, J=7.4 Hz, 4H), 1.43 (q, J=6.3 Hz, 8H), 1.18 (s, 51H), 0.80 (t, J=6.7 Hz, 12H).

### Compound 35: O,O′-(2-(((2-(diethylamino)ethyl)carbamoyl)oxy)propane-1,3-diyl) di(heptadecan-9-yl) diglutarate

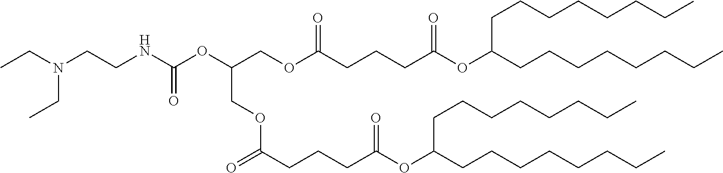 [SMILES: CCCCCCCCC(CCCCCCCC)OC(=O)CCCC(=O)OCC(COC(=O)CCCC(=O)OC(CCCCCCCC)CCCCCCCC)OC(=O)NCCN(CC)CC]

Compound 35 was synthesized from Intermediate 35c and N′,N′-diethylethane-1,2-diamine using the method employed in the synthesis of Compound 6. ¹H NMR (400 MHz, CDCl₃) δ 5.24 (t, J=5.2 Hz, 1H), 5.13-5.02 (m, 1H), 4.79 (p, J=6.3 Hz, 2H), 4.21 (dd, J=11.9, 4.4 Hz, 2H), 4.11 (dd, J=11.9, 5.7 Hz, 2H), 3.15 (q, J=5.9 Hz, 2H), 2.45 (q, J=7.2 Hz, 6H), 2.30 (dt, J=19.3, 7.4 Hz, 8H), 1.87 (p, J=7.4 Hz, 4H), 1.43 (q, J=5.9 Hz, 8H), 1.19 (s, 46H), 0.93 (t, J=7.1 Hz, 6H), 0.81 (t, J=6.7 Hz, 12H). MS: 940.4 m/z [M+H].

### Example 36—Compound 36

### Intermediate 36a: di(heptadecan-9-yl) O,O′-(2-(hydroxymethyl)-2-methylpropane-1,3-diyl) diglutarate

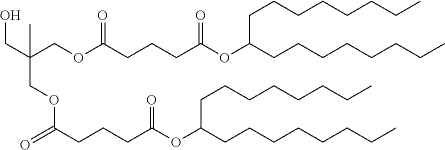 [SMILES: CCCCCCCCC(CCCCCCCC)OC(=O)CCCC(=O)OCC(C)(CO)COC(=O)CCCC(=O)OC(CCCCCCCC)CCCCCCCC]

Intermediate 36a was synthesized (36%) from 2-(hydroxymethyl)-2-methyl-propane-1,3-diol using the method employed in the synthesis of Intermediate 2c. ¹H NMR (400 MHz, CDCl₃) δ 4.80 (p, J=6.3 Hz, 2H), 3.96 (d, J=1.6 Hz, 4H), 3.33 (s, 2H), 2.31 (dt, J=21.4, 7.3 Hz, 8H), 1.90 (q, J=7.3 Hz, 4H), 1.44 (q, J=6.1 Hz, 8H), 1.19 (s, 49H), 0.88 (s, 3H), 0.81 (t, J=6.7 Hz, 12H).

### Intermediate 36b: di(heptadecan-9-yl) O,O′-(2-methyl-2-((((4-nitrophenoxy)carbonyl)oxy)methyl)propane-1,3-diyl) diglutarate

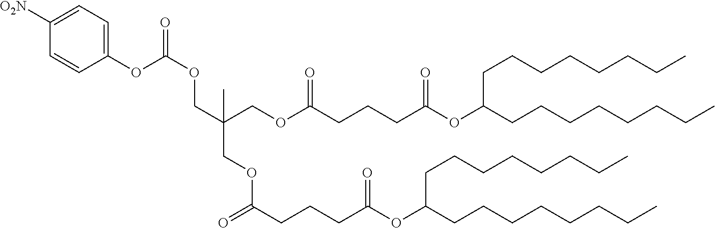 [SMILES: CCCCCCCCC(CCCCCCCC)OC(=O)CCCC(=O)OCC(C)(COC(=O)CCCC(=O)OC(CCCCCCCC)CCCCCCCC)COC(=O)Oc1ccc([N+](=O)[O-])cc1]

Intermediate 36b was synthesized (53%) from Intermediate 36a using the method employed in the synthesis of Intermediate 1d. ¹H NMR (400 MHz, CDCl₃) δ 8.28-8.17 (m, 2H), 7.40-7.27 (m, 2H), 4.80 (p, J=6.3 Hz, 2H), 4.14 (s, 2H), 4.02 (s, 4H), 2.35 (t, J=7.5 Hz, 4H), 2.29 (t, J=7.3 Hz, 4H), 1.89 (p, J=7.4 Hz, 4H), 1.44 (q, J=6.2 Hz, 8H), 1.28-1.12 (m, 49H), 1.02 (s, 3H), 0.80 (t, J=6.8 Hz, 12H).

### Compound 36: O,O′-(2-((((2-(diethylamino)ethyl)carbamoyl)oxy)methyl)-2-methylpropane-1,3-diyl) di(heptadecan-9-yl) diglutarate

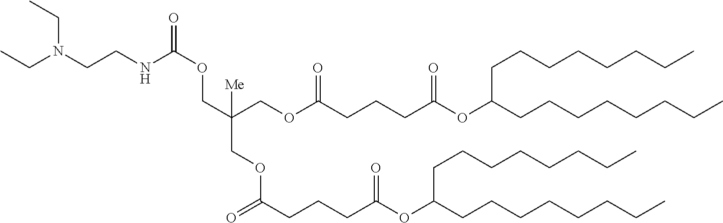 [SMILES: CCCCCCCCC(CCCCCCCC)OC(=O)CCCC(=O)OCC(C)(COC(=O)CCCC(=O)OC(CCCCCCCC)CCCCCCCC)COC(=O)NCCN(CC)CC]

Compound 36 was synthesized from Intermediate 36b and N′,N′-diethylethane-1,2-diamine using the method employed in the synthesis of Compound 6. ¹H NMR (400 MHz, CDCl₃) δ 5.14 (s, 1H), 4.80 (p, J=6.3 Hz, 2H), 3.93 (d, J=3.3 Hz, 6H), 3.14 (d, J=7.2 Hz, 2H), 2.45 (d, J=7.6 Hz, 6H), 2.30 (dt, J=17.6, 7.4 Hz, 8H), 1.87 (t, J=7.4 Hz, 4H), 1.43 (d, J=6.1 Hz, 10H), 1.19 (s, 50H), 0.93 (d, J=4.9 Hz, 9H), 0.81 (t, J=6.7 Hz, 12H). MS: 968.3 m/z [M+H].

### Example 37—Compound 37

### Intermediate 37a: 5-oxo-5-(tridecan-7-yloxy)pentanoic Acid

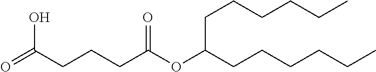 [SMILES: CCCCCCC(CCCCCC)OC(=O)CCCC(=O)O]

Intermediate 37a was synthesized (29%) from glutaric acid and tridecan-7-ol using the method employed in the synthesis of Intermediate 2b. ¹H NMR (400 MHz, CDCl₃) δ 4.81 (p, J=6.3 Hz, 1H), 2.34 (dt, J=22.0, 7.3 Hz, 4H), 1.90 (q, J=7.3 Hz, 2H), 1.44 (q, J=6.2 Hz, 4H), 1.26-1.15 (m, 16H), 0.86-0.75 (m, 6H).

### Intermediate 37b: O,O′-(2-(hydroxymethyl)propane-1,3-diyl) di(tridecan-7-yl) diglutarate

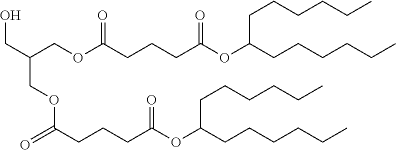 [SMILES: CCCCCCC(CCCCCC)OC(=O)CCCC(=O)OCC(CO)COC(=O)CCCC(=O)OC(CCCCCC)CCCCCC]

A mixture of Intermediate 37a (2.0 equiv.), 2-(hydroxymethyl)propane-1,3-diol (1.0 equiv.), EDCI (1.2 equiv.), DIPEA (2.5 equiv.), and DMAP (0.1 equiv.) in 1:1 DCM/DMF (0.1 M) was degassed and purged 3× with N₂, and the mixture was stirred at 20° C. for 24 h under N₂ atmosphere. The reaction mixture was then concentrated under reduced pressure, and the resulting residue was diluted with water and extracted 3× with EtOAc. The combined organic layers were washed with 3× with brine, dried over Na₂SO₄, filtered, and the filtrate was concentrated under reduced pressure to give a residue that was purified by column chromatography to afford product (23%) as a colorless oil. ¹H NMR (400 MHz, CDCl₃) δ 4.80 (p, J=6.3 Hz, 2H), 4.11 (dd, J=6.1, 2.6 Hz, 4H), 3.56 (t, J=5.9 Hz, 2H), 2.31 (dt, J=17.6, 7.3 Hz, 8H), 2.15 (q, J=5.8 Hz, 2H), 1.88 (p, J=7.4 Hz, 4H), 1.44 (q, J=6.2 Hz, 8H), 1.20 (p, J=7.1 Hz, 32H), 0.87-0.77 (m, 12H).

### Intermediate 37c: O,O′-(2-((((4-nitrophenoxy)carbonyl)oxy)methyl)propane-1,3-diyl) di(tridecan-7-yl) diglutarate

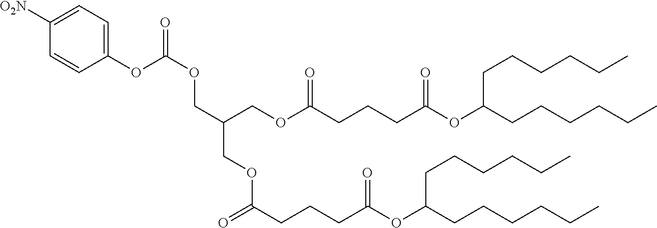 [SMILES: CCCCCCC(CCCCCC)OC(=O)CCCC(=O)OCC(COC(=O)CCCC(=O)OC(CCCCCC)CCCCCC)COC(=O)Oc1ccc([N+](=O)[O-])cc1]

Intermediate 37c was synthesized (65%) from Intermediate 37b using the method employed in the synthesis of Intermediate 2d.

### Compound 37: O,O′-(2-((((2-(diethylamino)ethyl)carbamoyl)oxy)methyl)propane-1,3-diyl) di(tridecan-7-yl) diglutarate

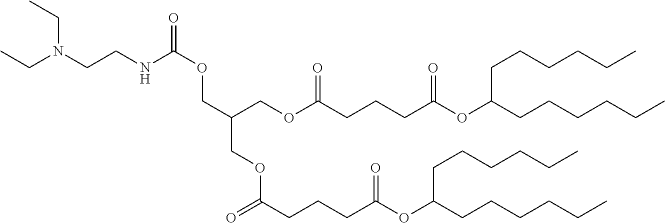 [SMILES: CCCCCCC(CCCCCC)OC(=O)CCCC(=O)OCC(COC(=O)CCCC(=O)OC(CCCCCC)CCCCCC)COC(=O)NCCN(CC)CC]

Compound 37 was synthesized from Intermediate 37c and N′,N′-diethylethane-1,2-diamine using the method employed in the synthesis of Compound 6. ¹H NMR (400 MHz, CDCl₃) δ 5.16 (s, 1H), 4.80 (p, J=6.3 Hz, 2H), 4.06 (t, J=6.3 Hz, 6H), 3.14 (d, J=6.3 Hz, 2H), 2.45 (q, J=7.5, 6.8 Hz, 6H), 2.30 (dt, J=14.7, 7.4 Hz, 9H), 1.87 (p, J=7.5 Hz, 4H), 1.44 (q, J=6.0 Hz, 8H), 1.20 (dd, J=9.9, 5.2 Hz, 32H), 0.94 (t, J=7.1 Hz, 6H), 0.81 (t, J=6.7 Hz, 12H). MS: 842.5 m/z [M+H].

### Example 38—Compound 38

### Intermediate 38a: 7-(heptadecan-9-yloxy)-7-oxoheptanoic Acid

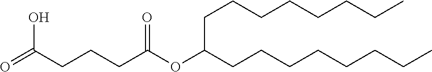 [SMILES: CCCCCCCCC(CCCCCCCC)OC(=O)CCCC(=O)O]

Intermediate 38a was synthesized (40%) from heptanedioic acid and Intermediate 2a using the method employed in the synthesis of Intermediate 2b. ¹H NMR (400 MHz, CDCl₃) δ 4.80 (p, J=6.3 Hz, 1H), 2.33-2.18 (m, 5H), 1.58 (ddt, J=11.1, 7.5, 3.6 Hz, 6H), 1.43 (q, J=6.1 Hz, 5H), 1.38-1.28 (m, 4H), 1.20 (d, J=7.6 Hz, 25H), 0.81 (t, J=6.8 Hz, 6H).

### Intermediate 38b: 7,7′-di(heptadecan-9-yl) O′1,O1-(2-(hydroxymethyl)propane-1,3-diyl) di(heptanedioate)

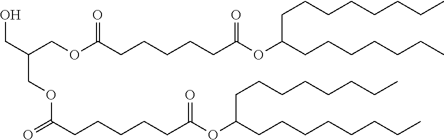 [SMILES: CCCCCCCCC(CCCCCCC)OC(=O)CCCCCC(=O)OCC(CO)COC(=O)CCCCCC(=O)OC(CCCCCCCC)CCCCCCCC]

Intermediate 38b was synthesized (16%) from Intermediate 38a using the method employed in the synthesis of Intermediate 2c. ¹H NMR (400 MHz, CDCl₃) δ 4.79 (p, J=6.3 Hz, 2H), 4.10 (h, J=6.3, 5.9 Hz, 4H), 3.55 (d, J=5.6 Hz, 2H), 2.24 (dt, J=17.1, 7.5 Hz, 9H), 2.12 (p, J=5.9 Hz, 1H), 1.57 (ddd, J=15.3, 7.6, 5.1 Hz, 8H), 1.43 (q, J=6.2 Hz, 8H), 1.33-1.14 (m, 54H), 0.81 (t, J=6.7 Hz, 12H).

### Intermediate 38c: 7,7′-di(heptadecan-9-yl) O′1,O1-(2-((((4-nitrophenoxy)carbonyl)oxy)methyl)propane-1,3-diyl) di(heptanedioate)

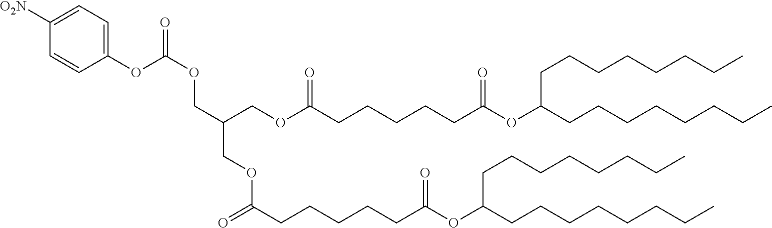 [SMILES: CCCCCCCCC(CCCCCCCC)OC(=O)CCCCCC(=O)OCC(COC(=O)CCCCCC(=O)OC(CCCCCCCC)CCCCCCCC)COC(=O)Oc1ccc([N+](=O)[O-])cc1]

Intermediate 38c was synthesized (54%) from Intermediate 38b using the method employed in the synthesis of Intermediate 8d. ¹H NMR (400 MHz, CDCl₃) δ 8.28-8.16 (m, 2H), 7.39-7.26 (m, 2H), 4.79 (p, J=6.3 Hz, 2H), 4.29 (d, J=5.8 Hz, 2H), 4.22-4.09 (m, 4H), 2.44 (h, J=6.0 Hz, 1H), 2.25 (dt, J=21.3, 7.5 Hz, 8H), 1.63-1.52 (m, 9H), 1.43 (q, J=6.2 Hz, 9H), 1.38-1.06 (m, 60H), 0.80 (t, J=6.7 Hz, 16H).

### Compound 38: O′1,O1-(2-((((2-(diethylamino)ethyl)carbamoyl)oxy)methyl)propane-1,3-diyl) 7,7′-di(heptadecan-9-yl) di(heptanedioate)

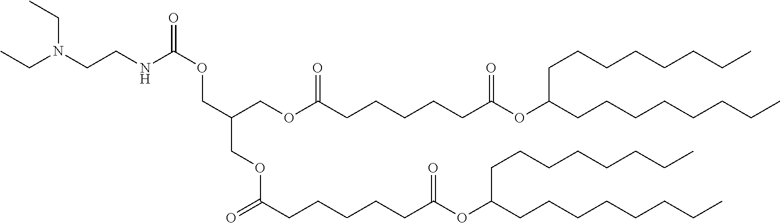 [SMILES: CCCCCCCCC(CCCCCCCC)OC(=O)CCCCCC(=O)OCC(COC(=O)CCCCCC(=O)OC(CCCCCCCC)CCCCCCCC)COC(=O)NCCN(CC)CC]

Compound 38 was synthesized from Intermediate 38c and N′,N′-diethylethane-1,2-diamine using the method employed in the synthesis of Compound 6. ¹H NMR (400 MHz, CDCl₃) δ 5.15 (s, 1H), 4.79 (p, J=6.2 Hz, 2H), 4.05 (d, J=6.0 Hz, 6H), 3.18-3.06 (m, 2H), 2.46 (t, J=6.8 Hz, 6H), 2.23 (dt, J=12.2, 7.5 Hz, 9H), 1.56 (q, J=7.3 Hz, 9H), 1.43 (q, J=5.9 Hz, 8H), 1.35-1.07 (m, 47H), 0.93 (t, J=7.1 Hz, 6H), 0.81 (t, J=6.7 Hz, 12H). MS: 1011.2 m/z [M+H].

### Example 39—Compound 39

### Intermediate 39a: ethyl 2-octyl-2-propionyldecanoate

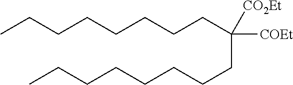

A solution of NaH (60% mineral oil, 1.2 equiv.) in THF (0.25-0.5 M) was cooled to 0° C. slowly under N₂ atmosphere. Then, diethyl propanedioate (0.4 equiv.) was added to the mixture, and the reaction was stirred at 0° C. for 30 min, followed by the addition of 1-iodooctane (1.0 equiv.). The mixture was stirred at 20° C. for 6 h under N₂ atmosphere, then quenched by the addition of water, extracted 2× with EtOAc, washed with brine, dried over anhydrous Na₂SO₄, and filtered. The filtrate was concentrated, and the resulting residue was purified by column chromatography to afford product (65%) as a yellow oil.

### Intermediate 39b: 2-octyldecanoic Acid

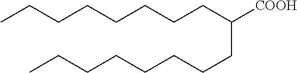 [SMILES: CCCCCCCCC(CCCCCCCC)C(=O)O]

To a solution of Intermediate 39a (70 g, 1.0 equiv.) in butan-1-ol (32 equiv.) was added 10 M NaOH (7.4 equiv.). The mixture was stirred at 120° C. for 6 h under N₂ atmosphere. The reaction mixture was then adjusted to pH=7 with 1 M HCl and extracted 2× with EtOAc, dried over Na₂SO₄, filtered, and the filtrate was concentrated to afford a residue. Then, acetic acid (19 equiv.) was added to the residue, and the mixture was stirred at 120° C. for 12 h. The mixture was then quenched with water, extracted 2× with EtOAc, dried over Na₂SO₄, filtered, and the filtrate was concentrated. The resulting residue was purified by column chromatography to afford product as a white solid (55%).

### Intermediate 39c: pent-4-en-1-yl 2-octyldecanoate

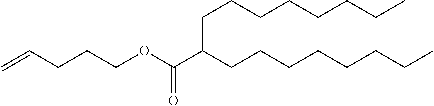 [SMILES: C=CCCCOC(=O)C(CCCCCCCC)CCCCCCCC]

To a solution of Intermediate 39b (1.0 equiv) in DCM (0.4 M) was added DIPEA (2.0 equiv.), EDCI (2.0 equiv.), and DMAP (0.2 equiv.). Then, pent-4-en-1-ol (1.0 equiv.) was added to the mixture, and the mixture was stirred at 25° C. for 12 h under N₂ atmosphere. The reaction mixture was then concentrated under reduced pressure, and the resulting residue was diluted with water, extracted 2× with EtOAc, and the combined organic layers were dried over Na₂SO₄ and filtered. The filtrate was concentrated, and the resulting residue purified by column chromatography to afford product as a colorless oil (71%).

### Intermediate 39d: 4-((2-octyldecanoyl)oxy)butanoic Acid

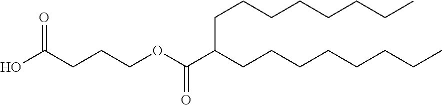 [SMILES: CCCCCCCCC(CCCCCCCC)C(=O)OCCCC(=O)O]

To a solution of Intermediate 39c (1.0 equiv.) in 1:1 DCM/MeCN (0.05 M) was added a solution of NaIO4 (5.0 equiv.) in H₂O (0.05 M). The mixture was cooled to 10° C. and stirred for 30 min under N₂ atmosphere. Then, RuCl₃ (0.04 equiv.) was added to the mixture, which was stirred at 25° C. for 12 h. The mixture was quenched by the addition of sat. NaHCO₃ at 0° C., diluted with water, and extracted 2× with EtOAc. The combined organic layers were dried over Na2_{S}O₄, filtered, and the filtrate was concentrated to afford a residue that was purified by column chromatography to afford product as a colorless oil (57%).

### Intermediate 39e: ((2-(hydroxymethyl)propane-1,3-diyl)bis(oxy))bis(4-oxobutane-4,1-diyl) bis(2-octyldecanoate)

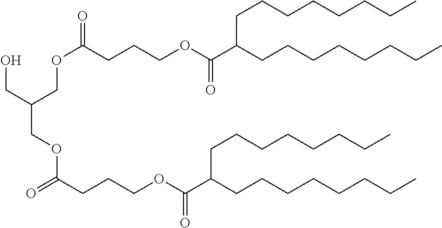 [SMILES: CCCCCCCCC(CCCCCCCC)C(=O)OCCCC(=O)OCC(CO)COC(=O)CCCOC(=O)C(CCCCCCCC)CCCCCCCC]

Intermediate 39e was synthesized (30%) from Intermediate 39d using the method employed in the synthesis of Intermediate 2c. ¹H NMR (400 MHz, CDCl₃) δ 4.19-4.08 (m, 4H), 4.04 (t, J=6.4 Hz, 4H), 3.57 (d, J=5.5 Hz, 2H), 2.34 (t, J=7.4 Hz, 4H), 2.25 (tt, J=8.7, 5.4 Hz, 3H), 2.17-2.11 (m, 1H), 1.90 (p, J=6.8 Hz, 4H), 1.51 (td, J=8.6, 4.2 Hz, 4H), 1.46-1.29 (m, 5H), 1.18 (s, 49H), 0.81 (t, J=6.8 Hz, 12H).

### Intermediate 39f: ((2-((((4-nitrophenoxy)carbonyl)oxy)methyl)propane-1,3-diyl)bis(oxy))bis(4-oxobutane-4,1-diyl) bis(2-octyldecanoate)

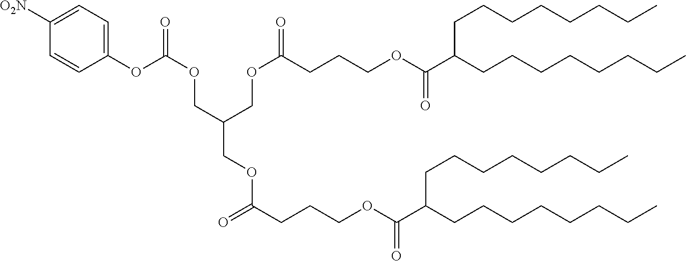 [SMILES: CCCCCCCCC(CCCCCCCC)C(=O)OCCCC(=O)OCC(COC(=O)CCCOC(=O)C(CCCCCCCC)CCCCCCCC)COC(=O)Oc1ccc([N+](=O)[O-])cc1]

Intermediate 39f was synthesized (62%) from Intermediate 39e using the method employed in the synthesis of Intermediate 2d. ¹H NMR (400 MHz, CDCl₃) δ 8.22 (d, J=9.0 Hz, 2H), 7.33 (d, J=8.9 Hz, 2H), 4.30 (d, J=5.7 Hz, 2H), 4.21-4.10 (m, 4H), 4.05 (t, J=6.4 Hz, 4H), 2.46 (hept, J=6.0 Hz, 1H), 2.37 (t, J=7.5 Hz, 4H), 2.25 (tt, J=8.9, 5.3 Hz, 2H), 1.91 (p, J=6.9 Hz, 4H), 1.51 (ddt, J=15.7, 12.2, 6.5 Hz, 4H), 1.44-1.29 (m, 5H), 1.18 (s, 49H), 0.80 (t, J=6.7 Hz, 12H).

### Compound 39: [4-[2-[2-(diethylamino)ethylcarbamoyloxymethyl]-3-[4-(2-octyldecanoyloxy)butanoyloxy]propoxy]-4-oxo-butyl]2-octyldecanoate

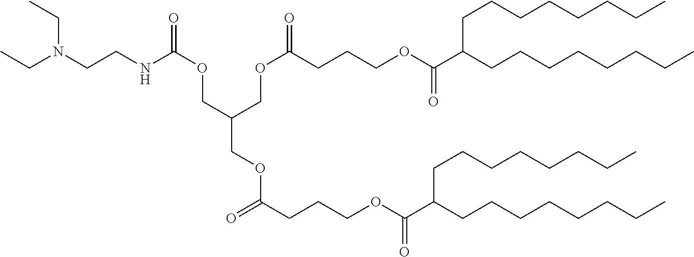 [SMILES: CCCCCCCCC(CCCCCCCC)C(=O)OCCCC(=O)OCC(COC(=O)CCCOC(=O)C(CCCCCCCC)CCCCCCCC)COC(=O)NCCN(CC)CC]

Compound 39 was synthesized from Intermediate 39f and N′,N′-diethylethane-1,2-diamine using the method employed in the synthesis of Compound 6. ¹H NMR (400 MHz, CDCl₃) δ 5.16 (s, 1H), 4.05 (dt, J=15.4, 6.4 Hz, 10H), 3.15 (q, J=6.0 Hz, 2H), 2.46 (t, J=6.7 Hz, 6H), 2.34 (t, J=7.5 Hz, 5H), 2.25 (tt, J=8.7, 5.4 Hz, 2H), 1.88 (q, J=7.1 Hz, 4H), 1.51 (tt, J=12.2, 5.0 Hz, 4H), 1.37 (p, J=6.9, 6.2 Hz, 5H), 1.18 (s, 49H), 0.93 (t, J=7.2 Hz, 6H), 0.81 (t, J=6.7 Hz, 12H). MS: 955.2 m/z [M+H].

### Example 40—Compound 40

### Intermediate 40a: 2-(hydroxymethyl)propane-1,3-diyl bis(2-octyldecanoate)

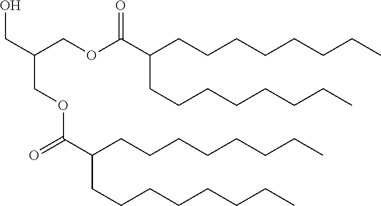 [SMILES: CCCCCCCCC(CCCCCCCC)C(=O)OCC(CO)COC(=O)C(CCCCCCCC)CCCCCCCC]

Intermediate 40a was synthesized from Intermediate 39b and 2-(hydroxymethyl) propane-1,3-diol using the method employed in the synthesis of Intermediate 2c. ¹H NMR (400 MHz, CDCl₃) δ 4.11 (qd, J=11.3, 6.0 Hz, 4H), 3.53 (t, J=6.0 Hz, 2H), 2.39-2.22 (m, 3H), 2.13 (p, J=5.9 Hz, 1H), 1.53 (ddd, J=14.0, 9.2, 5.6 Hz, 4H), 1.38 (td, J=8.1, 3.9 Hz, 5H), 1.19 (d, J=4.4 Hz, 47H), 0.81 (t, J=6.8 Hz, 12H).

### Intermediate 40b: 2-((((4-nitrophenoxy)carbonyl)oxy)methyl)propane-1,3-diyl bis(2-octyldecanoate)

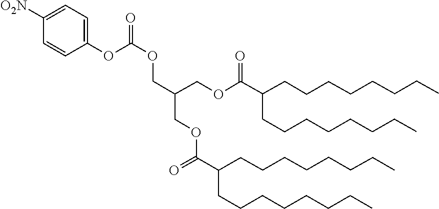 [SMILES: CCCCCCCCC(CCCCCCCC)C(=O)OCC(COC(=O)Oc1ccc([N+](=O)[O-])cc1)COC(=O)C(CCCCCCCC)CCCCCCCC]

Intermediate 40b was synthesized (68%) from Intermediate 40a using the method employed in the synthesis of Intermediate 2d.

### Compound 40: 2-((((2-(diethylamino)ethyl)carbamoyl)oxy)methyl)propane-1,3-diyl bis(2-octyldecanoate)

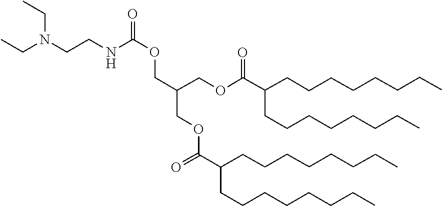 [SMILES: CCCCCCCCC(CCCCCCCC)C(=O)OCC(COC(=O)NCCN(CC)CC)COC(=O)C(CCCCCCCC)CCCCCCCC]

Compound 40 was synthesized from Intermediate 40b and N′,N′-diethylethane-1,2-diamine using the method employed in the synthesis of Compound 6. ¹H NMR (400 MHz, CDCl₃) δ 5.13 (s, 1H), 4.06 (dd, J=6.2, 3.3 Hz, 6H), 3.14 (d, J=6.7 Hz, 2H), 2.46 (dd, J=9.4, 4.9 Hz, 6H), 2.34-2.22 (m, 3H), 1.60 (s, 1H), 1.50 (dd, J=8.5, 5.3 Hz, 4H), 1.43-1.28 (m, 4H), 1.18 (s, 46H), 0.93 (t, J=7.1 Hz, 6H), 0.81 (t, J=6.7 Hz, 12H). MS: 782.3 m/z [M+H].

### Example 41—Compound 41

### Intermediate 41a: 3-hydroxy-2-(hydroxymethyl)propyl Dodecanoate

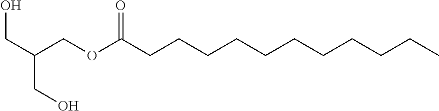 [SMILES: CCCCCCCCCCCC(=O)OCC(CO)CO]

Intermediate 41a was synthesized (13%) from dodecanoic acid using the method employed in the synthesis of Intermediate 4b.

### Intermediate 41b: 3-((4,4-bis(octyloxy)butanoyl)oxy)-2-(hydroxymethyl)propyl Dodecanoate

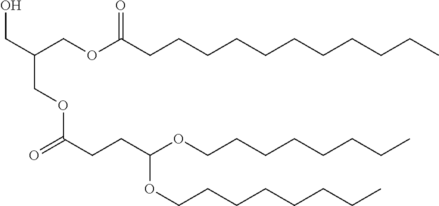 [SMILES: CCCCCCCCCCCC(=O)OCC(CO)COC(=O)CCC(OCCCCCCCC)OCCCCCCCC]

Intermediate 41b was synthesized (29%) from Intermediate 41a and Intermediate 1b using the method employed in the synthesis of Intermediate 4c. ¹H NMR (400 MHz, CDCl₃) δ 4.42 (t, J=5.5 Hz, 1H), 4.09 (ddd, J=14.7, 7.9, 5.1 Hz, 4H), 3.61-3.53 (m, 3H), 3.50 (dt, J=9.3, 6.7 Hz, 2H), 3.33 (dt, J=9.3, 6.7 Hz, 2H), 2.33 (q, J=7.9 Hz, 2H), 2.24 (q, J=9.3, 8.5 Hz, 3H), 2.13 (p, J=5.8 Hz, 1H), 1.87 (td, J=7.3, 5.3 Hz, 2H), 1.52 (dt, J=24.7, 6.6 Hz, 7H), 1.32-1.13 (m, 40H), 0.81 (t, J=6.7 Hz, 9H).

### Intermediate 41c: 3-((4,4-bis(octyloxy)butanoyl)oxy)-2-((((4-nitrophenoxy)carbonyl)oxy)methyl)propyl Dodecanoate

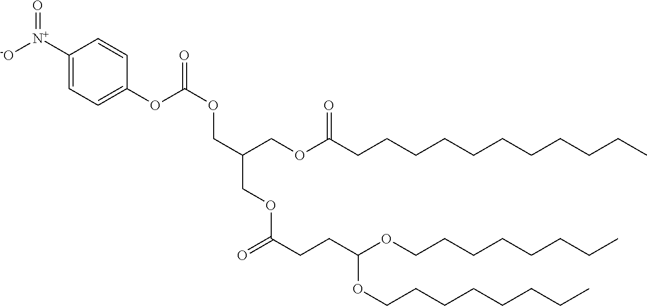

Intermediate 41c was synthesized (69%) from Intermediate 41b using the method employed in the synthesis of Intermediate 2d. ¹H NMR (400 MHz, CDCl₃) δ 8.31-8.25 (m, 2H), 7.43-7.35 (m, 2H), 4.49 (t, J=5.5 Hz, 1H), 4.36 (d, J=5.8 Hz, 2H), 4.21 (dt, J=6.1, 2.0 Hz, 4H), 3.56 (dt, J=9.3, 6.7 Hz, 2H), 3.40 (dt, J=9.3, 6.7 Hz, 2H), 2.52 (p, J=5.9 Hz, 1H), 2.42 (t, J=7.6 Hz, 2H), 2.39-2.30 (m, 2H), 1.98-1.92 (m, 2H), 1.67-1.51 (m, 8H), 1.34-1.23 (m, 38H), 0.91-0.85 (m, 9H).

### Compound 41: 3-((4,4-bis(octyloxy)butanoyl)oxy)-2-((((3-(diethylamino)propoxy)carbonyl)oxy)methyl)propyl Dodecanoate

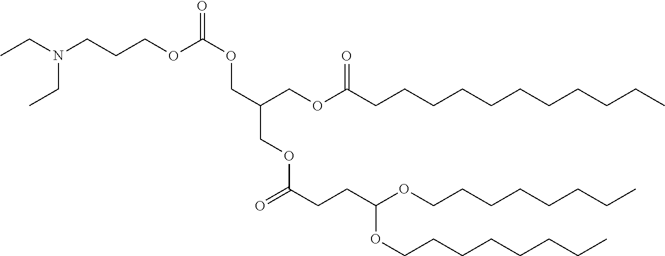 [SMILES: CCCCCCCCCCCC(=O)OCC(COC(=O)CCC(OCCCCCCCC)OCCCCCCCC)COC(=O)OCCCN(CC)CC]

Compound 41 was synthesized from Intermediate 41c and 3-(diethylamino)propan-1-ol using the method employed in the synthesis of Compound 6. ¹H NMR (400 MHz, CDCl₃) δ 4.41 (t, J=5.6 Hz, 1H), 4.12 (dt, J=6.5, 3.5 Hz, 4H), 4.08 (dd, J=6.1, 1.9 Hz, 4H), 3.49 (dt, J=9.3, 6.7 Hz, 2H), 3.33 (dt, J=9.3, 6.7 Hz, 2H), 2.40-2.28 (m, 4H), 2.23 (t, J=7.6 Hz, 2H), 1.85 (td, J=7.6, 5.5 Hz, 2H), 1.50 (dq, J=20.9, 7.1 Hz, 7H), 1.30-1.12 (m, 37H), 0.96 (s, 6H), 0.85-0.77 (m, 9H). MS: 773.0 m/z [M+H].

### Example 42—Compound 42

### Compound 42: 3-((4,4-bis(octyloxy)butanoyl)oxy)-2-((((2-(diethylamino)ethyl)carbamoyl)oxy)methyl)propyl Undecanoate

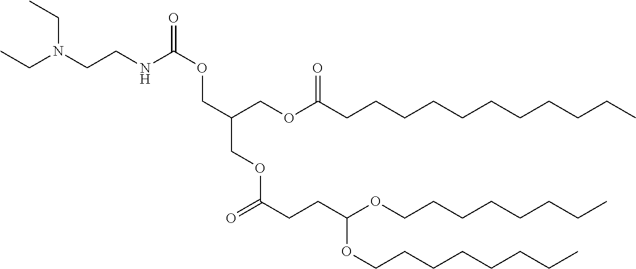 [SMILES: CCCCCCCCCCCC(=O)OCC(COC(=O)CCC(OCCCCCCCC)OCCCCCCCC)COC(=O)NCCN(CC)CC]

Compound 42 was synthesized from Intermediate 41c and N′,N′-diethylethane-1,2-diamine using the method employed in the synthesis of Compound 6. ¹H NMR (400 MHz, CDCl₃) δ 4.48 (t, J=5.6 Hz, 1H), 4.12 (dd, J=6.2, 2.9 Hz, 6H), 3.56 (dt, J=9.3, 6.7 Hz, 2H), 3.40 (dt, J=9.3, 6.7 Hz, 2H), 3.30 (s, 2H), 2.59 (d, J=40.7 Hz, 5H), 2.38 (q, J=6.8, 5.9 Hz, 3H), 2.30 (t, J=7.6 Hz, 2H), 1.92 (td, J=7.6, 5.4 Hz, 2H), 1.58 (dt, J=20.2, 7.3 Hz, 8H), 1.40-1.18 (m, 41H), 1.09 (s, 6H), 0.88 (t, J=6.7 Hz, 9H). MS: 758.6 m/z [M+H].

### Example 43—Compound 43

### Intermediate 43a: 2-butyloctyl Methanesulfonate

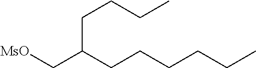 [SMILES: CCCCCCC(CCCC)COS(C)(=O)=O]

To a solution of 2-butyloctan-1-ol (20 g, 1.0 equiv.) and Et₃N (1.2 equiv.) in DCM (0.5 M) was added MsCl (1.5 equiv.) dropwise under N₂ at 0° C. The mixture was stirred at 0° C. for 2 h. The reaction mixture was quenched by added of sat. NaHCO₃, diluted with water, and extracted 3× with DCM. The combined organic layers were dried over Na₂SO₄, filtered, and the filtrate was concentrated under reduced pressure to give afford a white solid that was used directly in the next step without additional purification.

### Intermediate 43b: 3-butylnonanenitrile (

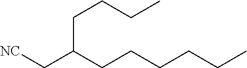

To a solution of Intermediate 43a (20 g, 1.0 equiv.) in DMF (0.3-0.4 M) was added NaCN (5.0 equiv.) under N₂. The reaction mixture was heated to 60° C. and stirred for 12 h. The reaction mixture was then poured into aq. NaOH to pH>11 and extracted 3× with EtOAc. The combined organic layers were concentrated under reduced pressure to remove solvent, dried over Na₂SO₄, filtered, and the filtrate was concentrated under reduced pressure to give a residue. The residue was purified by column chromatography to afford product (81%) as a colorless oil. ¹H NMR (400 MHz, CDCl₃) δ 2.32 (d, J=5.8 Hz, 2H), 1.67 (ddd, J=13.1, 7.1, 5.9 Hz, 1H), 1.48-1.18 (m, 16H), 0.89 (dt, J=8.6, 6.9 Hz, 6H).

### Intermediate 43c: 3-butylnonanoic Acid

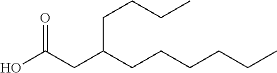

A mixture of Intermediate 43b (11 g, 1.0 equiv.) and KOH (10.0 equiv.) in 1:1 EtOH and H₂O (1 M) was degassed and purged 3× with N₂, and then the mixture was stirred at 120° C. for 12 h under N₂ atmosphere. The reaction mixture was concentrated under reduced pressure to remove solvent. The reaction mixture was adjusted to pH=5 by 1 M HCl, diluted with H₂O, and extracted 3× with EtOAc. The combined organic layers were dried over Na₂SO₄, filtered, and the filtrate was concentrated under reduced pressure to give a residue. The residue was purified by column chromatography to afford product (66%) as a yellow oil. ¹H NMR (400 MHz, CDCl₃) δ 2.21 (d, J=6.9 Hz, 2H), 1.78 (d, J=6.1 Hz, 1H), 1.29-1.16 (m, 16H), 0.82 (td, J=6.7, 4.0 Hz, 6H).

### Intermediate 43d: 3-hydroxy-2-(hydroxymethyl)propyl 4,4-bis(octyloxy)butanoate

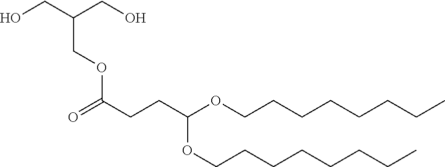 [SMILES: CCCCCCCCOC(CCC(=O)OCC(CO)CO)OCCCCCCCC]

A mixture of Intermediate 1b (10 g, 1.0 equiv.), 2-(hydroxymethyl)propane-1,3-diol (1.0 equiv.), EDCI (2.0 equiv.), DMAP (0.2 equiv.), and DIPEA (2.0 equiv.) in DCM (0.2 M) was degassed and purged 3× with N₂, and then the mixture was stirred at 15° C. for 12 h under N₂ atmosphere. The reaction mixture was diluted with H₂O, and extracted 3× with DCM, dried over Na₂SO₄, filtered, and the filtrate was concentrated under reduced pressure to give a residue. The residue was purified by column chromatography to afford product (52%) as a colorless oil. ¹H NMR (400 MHz, CDCl₃) δ 4.42 (t, J=5.5 Hz, 1H), 4.27-4.03 (m, 2H), 3.76-3.61 (m, 4H), 3.57 (t, J=6.7 Hz, 1H), 3.50 (dt, J=9.6, 6.7 Hz, 2H), 3.33 (dt, J=9.3, 6.7 Hz, 2H), 2.34 (q, J=7.4 Hz, 2H), 2.15-2.00 (m, 1H), 2.00-1.92 (m, 1H), 1.87 (td, J=7.2, 5.1 Hz, 2H), 1.49 (p, J=6.5 Hz, 4H), 1.32-1.11 (m, 20H), 0.81 (t, J=6.7 Hz, 6H).

### Intermediate 43e: 3-((4,4-bis(octyloxy)butanoyl)oxy)-2-(hydroxymethyl)propyl 3-butylnonanoate

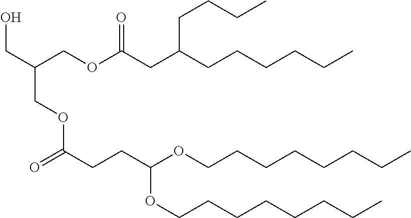 [SMILES: CCCCCCCCOC(CCC(=O)OCC(CO)COC(=O)CC(CCCC)CCCCCC)OCCCCCCCC]

A mixture of Intermediate 43d (1.0 equiv.), Intermediate 43c (1.0 equiv.), DMAP (0.2 equiv.), EDCI (2.0 equiv.) and DIPEA (1.0 equiv.) in DCM (0.25 M) was degassed and purged 3× with N₂, and then the mixture was stirred at 0° C. for 6 h under N₂ atmosphere. The reaction mixture was diluted with H₂O, extracted 3× with DCM, dried over Na₂SO₄, filtered, and the filtrate was concentrated under reduced pressure to give a residue. The residue was purified (53%) by column chromatography to afford product as a colorless oil. ¹H NMR (400 MHz, CDCl₃) δ 4.48 (t, J=5.5 Hz, 1H), 4.22-4.10 (m, 4H), 3.68-3.49 (m, 4H), 3.39 (dt, J=9.3, 6.7 Hz, 2H), 2.40 (q, J=7.9 Hz, 2H), 2.25 (d, J=6.8 Hz, 2H), 2.22-2.14 (m, 1H), 1.93 (td, J=7.4, 5.4 Hz, 2H), 1.83 (p, J=5.8 Hz, 1H), 1.56 (q, J=7.2, 6.4 Hz, 4H), 1.26 (dt, J=11.5, 6.8 Hz, 35H), 0.88 (td, J=6.8, 3.3 Hz, 12H).

### Intermediate 43f: 3-((4,4-bis(octyloxy)butanoyl)oxy)-2-((((4-nitrophenoxy)carbonyl)oxy)methyl)propyl 3-butylnonanoate

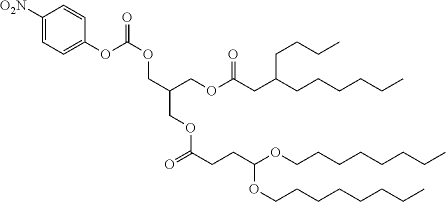 [SMILES: CCCCCCCCOC(CCC(=O)OCC(COC(=O)CC(CCCC)CCCCCC)COC(=O)Oc1ccc([N+](=O)[O-])cc1)OCCCCCCCC]

Intermediate 43f was synthesized (69%) from Intermediate 43e using the method employed in the synthesis of Intermediate 2d.

### Compound 43: 3-((4,4-bis(octyloxy)butanoyl)oxy)-2-((((3-(diethylamino)propoxy)carbonyl)oxy)methyl)propyl 3-butylnonanoate

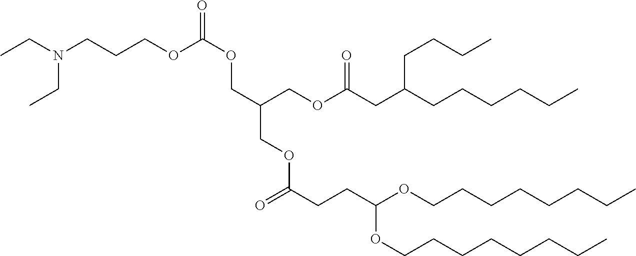 [SMILES: CCCCCCCCOC(CCC(=O)OCC(COC(=O)CC(CCCC)CCCCCC)COC(=O)OCCCN(CC)CC)OCCCCCCCC]

Compound 43 was synthesized from Intermediate 43f and 3-(diethylamino)propan-1-ol using the method employed in the synthesis of Compound 6. ¹H NMR (400 MHz, CDCl₃) δ 4.48 (t, J=5.5 Hz, 1H), 4.21-4.09 (m, 8H), 3.55 (dt, J=9.3, 6.7 Hz, 2H), 3.39 (dt, J=9.3, 6.7 Hz, 2H), 2.52 (q, J=7.0 Hz, 6H), 2.40 (q, J=7.6, 7.0 Hz, 3H), 2.24 (d, J=6.8 Hz, 2H), 1.92 (td, J=7.6, 5.4 Hz, 2H), 1.82 (p, J=6.8 Hz, 3H), 1.56 (q, J=6.9 Hz, 4H), 1.39-1.18 (m, 37H), 1.01 (t, J=7.1 Hz, 6H), 0.88 (td, J=6.9, 2.6 Hz, 12H). MS: 787.0 m/z [M+H].

### Example 44—Compound 44

### Compound 44: 3-((4,4-bis(octyloxy)butanoyl)oxy)-2-((((2-(diethylamino)ethyl)carbamoyl)oxy)methyl)propyl 3-butylnonanoate

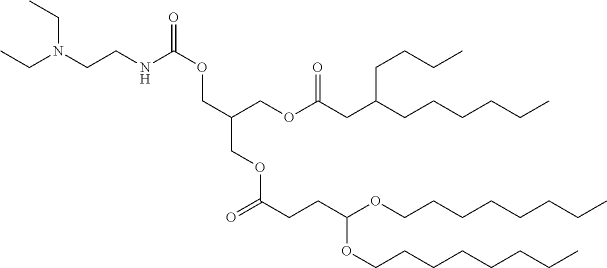 [SMILES: CCCCCCCCOC(CCC(=O)OCC(COC(=O)CC(CCCC)CCCCCC)COC(=O)NCCN(CC)CC)OCCCCCCCC]

Compound 44 was synthesized from Intermediate 43f and N′,N′-diethylethane-1,2-diamine using the method employed in the synthesis of Compound 6. ¹H NMR (400 MHz, CDCl₃) δ 5.14 (s, 1H), 4.41 (t, J=5.6 Hz, 1H), 4.06 (t, J=5.1 Hz, 6H), 3.49 (dt, J=9.3, 6.7 Hz, 2H), 3.33 (dt, J=9.3, 6.7 Hz, 2H), 3.14 (q, J=5.9 Hz, 2H), 2.45 (q, J=7.1, 6.1 Hz, 6H), 2.32 (q, J=7.9 Hz, 3H), 2.17 (d, J=6.8 Hz, 2H), 1.85 (td, J=7.6, 5.4 Hz, 2H), 1.55-1.43 (m, 6H), 1.22 (dd, J=18.6, 8.2 Hz, 35H), 0.93 (t, J=7.1 Hz, 6H), 0.81 (td, J=6.9, 2.4 Hz, 12H). MS: 772.5 m/z [M+H].

### Example 45—Compound 45

### Intermediate 45a: 3-hydroxy-2-(hydroxymethyl)propyl Nonanoate

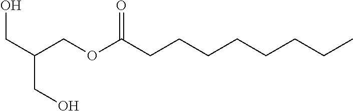 [SMILES: CCCCCCCCC(=O)OCC(CO)CO]

Intermediate 45a was synthesized (32%) from 2-(hydroxymethyl)propane-1,3-diol and nonanoic acid using the method employed in the synthesis of Intermediate 4b. ¹H NMR (400 MHz, CDCl₃) δ 4.28 (d, J=6.3 Hz, 2H), 3.79 (qd, J=11.1, 5.1 Hz, 4H), 2.35 (t, J=7.6 Hz, 2H), 2.28 (s, 2H), 2.05 (p, J=5.9 Hz, 1H), 1.68-1.56 (m, 2H), 1.39-1.24 (m, 10H), 0.96-0.83 (m, 3H).

### Intermediate 45b: 3-((4,4-bis(octyloxy)butanoyl)oxy)-2-(hydroxymethyl)propyl Nonanoate

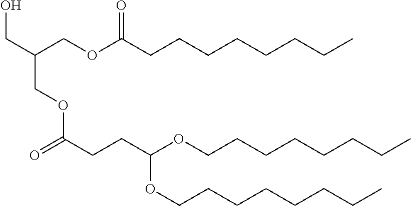 [SMILES: CCCCCCCCOC(CCC(=O)OCC(CO)COC(=O)CCCCCCCC)OCCCCCCCC]

Intermediate 45b was synthesized (76%) from Intermediate 45a and Intermediate 1b using the method employed in the synthesis of Intermediate 4c. ¹H NMR (400 MHz, CDCl₃) δ 4.42 (t, J=5.5 Hz, 1H), 4.18-4.01 (m, 4H), 3.60-3.46 (m, 4H), 3.33 (dt, J=9.3, 6.7 Hz, 2H), 2.33 (q, J=7.8 Hz, 2H), 2.28-2.18 (m, 3H), 2.13 (h, J=5.9 Hz, 1H), 1.87 (td, J=7.3, 5.3 Hz, 2H), 1.52 (dt, J=25.0, 7.1 Hz, 6H), 1.33-1.17 (m, 32H), 0.81 (t, J=6.7 Hz, 9H).

### Intermediate 45c: 3-((4,4-bis(octyloxy)butanoyl)oxy)-2-((((4-nitrophenoxy)carbonyl)oxy)methyl)propyl Nonanoate

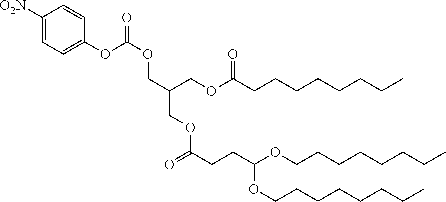 [SMILES: CCCCCCCCOC(CCC(=O)OCC(COC(=O)CCCCCCCC)COC(=O)Oc1ccc([N+](=O)[O-])cc1)OCCCCCCCC]

Intermediate 45c was synthesized from Intermediate 45b using the method employed in the synthesis of Intermediate 2d.

### Compound 45: 3-((4,4-bis(octyloxy)butanoyl)oxy)-2-((((3-(diethylamino)propoxy)carbonyl)oxy)methyl)propyl Nonanoate

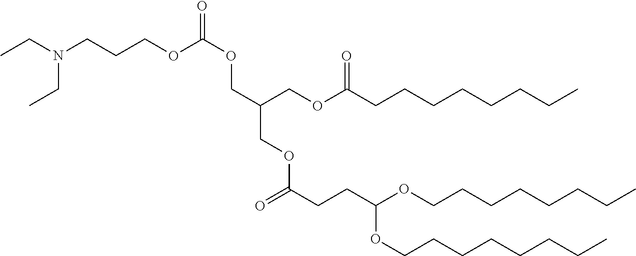 [SMILES: CCCCCCCCOC(CCC(=O)OCC(COC(=O)CCCCCCCC)COC(=O)OCCCN(CC)CC)OCCCCCCCC]

Compound 45 was synthesized from Intermediate 45c and 3-(diethylamino)propan-1-ol using the method employed in the synthesis of Compound 6. ¹H NMR (400 MHz, CDCl₃) δ 4.48 (t, J=5.5 Hz, 1H), 4.20 (t, J=6.0 Hz, 4H), 4.14 (dd, J=6.0, 1.9 Hz, 4H), 3.58-3.51 (m, 2H), 3.40 (dt, J=9.3, 6.7 Hz, 2H), 2.61 (s, 5H), 2.46-2.36 (m, 4H), 2.30 (t, J=7.6 Hz, 2H), 1.92 (td, J=7.6, 5.5 Hz, 4H), 1.63-1.49 (m, 7H), 1.34-1.25 (m, 31H), 1.08 (s, 6H), 0.89-0.85 (m, 9H). MS: 732.0 m/z [M+H].

### Example 46—Compound 46

### Compound 46: 3-((4,4-bis(octyloxy)butanoyl)oxy)-2-((((2-(diethylamino)ethyl)carbamoyl)oxy)methyl)propyl Nonanoate

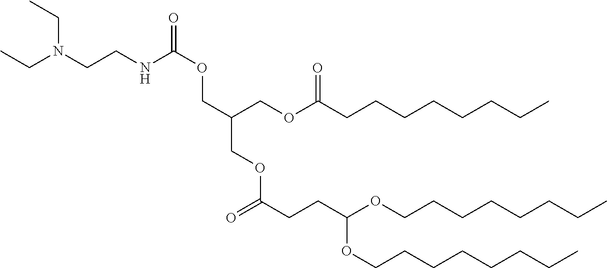 [SMILES: CCCCCCCCOC(CCC(=O)OCC(COC(=O)CCCCCCCC)COC(=O)NCCN(CC)CC)OCCCCCCCC]

Compound 46 was synthesized from Intermediate 45c and N′,N′-diethylethane-1,2-diamine using the method employed in the synthesis of Compound 6. ¹H NMR (400 MHz, CDCl₃) δ 5.25-5.11 (m, 1H), 4.42 (t, J=5.5 Hz, 1H), 4.13-4.00 (m, 6H), 3.49 (dt, J=9.3, 6.7 Hz, 2H), 3.33 (dt, J=9.1, 6.6 Hz, 2H), 3.15 (q, J=5.8 Hz, 2H), 2.46 (q, J=7.1, 6.1 Hz, 6H), 2.32 (q, J=7.6 Hz, 3H), 2.23 (t, J=7.6 Hz, 2H), 1.91-1.80 (m, 2H), 1.51 (dp, J=20.9, 7.1 Hz, 6H), 1.32-1.12 (m, 32H), 0.94 (t, J=7.1 Hz, 6H), 0.81 (t, J=6.7 Hz, 9H). MS: 717.0 m/z [M+H].

### Example 47—Compound 47

### Intermediate 47a: 3-hydroxy-2-(hydroxymethyl)propyl 2-butyloctanoate

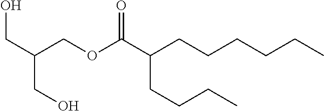 [SMILES: CCCCCCC(CCCC)C(=O)OCC(CO)CO]

Intermediate 47a was synthesized (35%) from 2-(hydroxymethyl)propane-1,3-diol and 2-butyloctanoic acid using the method employed in the synthesis of Intermediate 4b. ¹H NMR (400 MHz, CDCl₃) δ 4.18 (dd, J=6.2, 2.1 Hz, 2H), 3.69 (qt, J=10.7, 4.3 Hz, 4H), 2.55 (t, J=15.5 Hz, 2H), 2.28 (td, J=9.1, 4.7 Hz, 1H), 1.97 (h, J=5.5 Hz, 1H), 1.52 (dt, J=15.3, 7.3 Hz, 2H), 1.45-1.29 (m, 2H), 1.20 (dh, J=13.5, 6.6 Hz, 12H), 0.81 (td, J=6.9, 3.1 Hz, 6H).

### Intermediate 47b: 3-((4,4-bis(octyloxy)butanoyl)oxy)-2-(hydroxymethyl)propyl 2-butyloctanoate

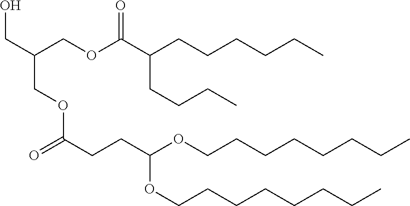 [SMILES: CCCCCCCCOC(CCC(=O)OCC(CO)COC(=O)C(CCCC)CCCCCC)OCCCCCCCC]

Intermediate 47b was synthesized (52%) from Intermediate 47a and Intermediate 1b using the method employed in the synthesis of Intermediate 4c. ¹H NMR (400 MHz, CDCl₃) δ 4.48 (t, J=5.5 Hz, 1H), 4.23-4.10 (m, 4H), 3.64-3.52 (m, 4H), 3.39 (dt, J=9.3, 6.7 Hz, 2H), 2.40 (t, J=7.5 Hz, 2H), 2.34 (td, J=8.9, 8.3, 4.3 Hz, 2H), 2.19 (p, J=5.9 Hz, 1H), 1.92 (td, J=7.4, 5.3 Hz, 2H), 1.54 (dq, J=13.9, 6.9, 6.1 Hz, 6H), 1.44 (ddt, J=14.9, 6.3, 4.1 Hz, 3H), 1.26 (tt, J=13.0, 6.8 Hz, 32H), 0.94-0.78 (m, 12H).

### Intermediate 47c: 3-((4,4-bis(octyloxy)butanoyl)oxy)-2-((((4-nitrophenoxy)carbonyl)oxy)methyl)propyl 2-butyloctanoate

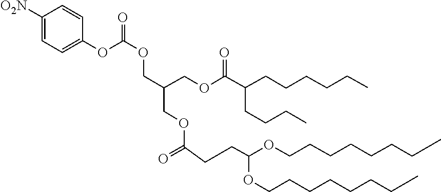 [SMILES: CCCCCCCCOC(CCC(=O)OCC(COC(=O)Oc1ccc([N+](=O)[O-])cc1)COC(=O)C(CCCC)CCCCCC)OCCCCCCCC]

Intermediate 47c was synthesized from Intermediate 47b using the method employed in the synthesis of Intermediate 2d.

### Compound 47: 3-((4,4-bis(octyloxy)butanoyl)oxy)-2-((((3-(diethylamino)propoxy)carbonyl)oxy)methyl)propyl 2-butyloctanoate

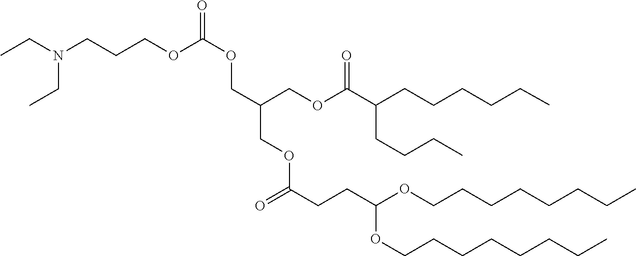 [SMILES: CCCCCCCCOC(CCC(=O)OCC(COC(=O)OCCCN(CC)CC)COC(=O)C(CCCC)CCCCCC)OCCCCCCCC]

Compound 47 was synthesized from Intermediate 47c and 3-(diethylamino)propan-1-ol using the method employed in the synthesis of Compound 6. ¹H NMR (400 MHz, CDCl₃) δ 4.46 (t, J=5.5 Hz, 1H), 4.21-4.05 (m, 8H), 3.54 (dt, J=9.3, 6.7 Hz, 2H), 3.38 (dt, J=9.3, 6.7 Hz, 2H), 2.54-2.45 (m, 6H), 2.45-2.35 (m, 3H), 2.31 (tt, J=8.8, 5.4 Hz, 1H), 1.90 (td, J=7.6, 5.4 Hz, 2H), 1.79 (p, J=6.8 Hz, 2H), 1.64-1.48 (m, 6H), 1.48-1.37 (m, 2H), 1.37-1.15 (m, 32H), 0.99 (t, J=7.2 Hz, 6H), 0.86 (td, J=6.9, 2.0 Hz, 12H). MS: 773.6 m/z [M+H].

### Example 48—Compound 48

### Compound 48: 3-((4,4-bis(octyloxy)butanoyl)oxy)-2-((((2-(diethylamino)ethyl)carbamoyl)oxy)methyl)propyl 2-butyloctanoate

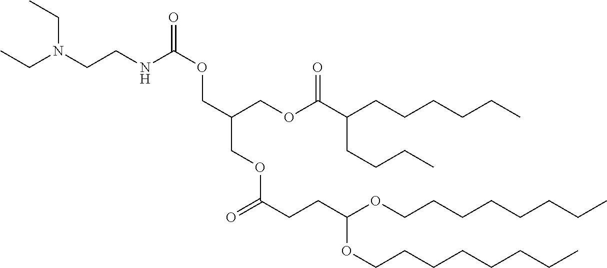 [SMILES: CCCCCCCCOC(CCC(=O)OCC(COC(=O)NCCN(CC)CC)COC(=O)C(CCCC)CCCCCC)OCCCCCCCC]

Compound 48 was synthesized from Intermediate 47c and N′,N′-diethylethane-1,2-diamine using the method employed in the synthesis of Compound 6. ¹H NMR (400 MHz, CDCl₃) δ 5.22 (t, J=5.3 Hz, 1H), 4.47 (t, J=5.6 Hz, 1H), 4.12 (q, J=5.2, 4.7 Hz, 6H), 3.55 (dt, J=9.3, 6.7 Hz, 2H), 3.39 (dt, J=9.3, 6.7 Hz, 2H), 3.20 (q, J=5.8 Hz, 2H), 2.51 (q, J=7.1, 5.9 Hz, 6H), 2.44-2.28 (m, 4H), 1.91 (td, J=7.6, 5.5 Hz, 2H), 1.55 (h, J=6.9 Hz, 6H), 1.49-1.39 (m, 2H), 1.39-1.16 (m, 33H), 0.99 (t, J=7.1 Hz, 6H), 0.86 (td, J=6.9, 2.2 Hz, 12H). MS: 758.0 m/z [M+H].

### Example 49—Compound 49

### Intermediate 49a: heptadecan-9-yl (3-hydroxy-2-((((9Z,12Z)-octadeca-9,12-dienoyl)oxy)methyl)propyl) glutarate

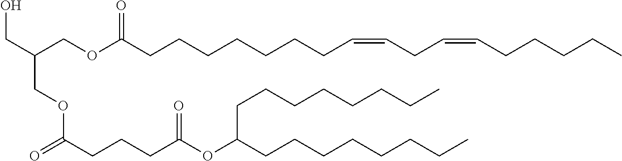 [SMILES: CCCCC/C=C\C/C=C\CCCCCCCC(=O)OCC(CO)COC(=O)CCCC(=O)OC(CCCCCCCC)CCCCCCCC]

To a solution of Intermediate 2b (1.0 equiv.) in DCM (0.25 M) was added EDCI (2.0 equiv.), DMAP (0.2 equiv.), DIPEA (2.0 equiv.) and Intermediate 10b (1.0 equiv.). The mixture was stirred at 15° C. for 12 h under N₂. The reaction mixture was concentrated under reduced pressure to remove solvent. The residue was diluted with water and extracted 3× with DCM. The combined organic layers were dried over Na₂SO₄, filtered, and the filtrate was concentrated under reduced pressure to give a residue. The residue was purified by column chromatography to afford product (32%) as a pale yellow oil. ¹H NMR (400 MHz, CDCl₃) δ 5.41-5.27 (m, 4H), 4.86 (p, J=6.3 Hz, 2H), 4.21-4.10 (m, 4H), 3.61 (d, J=5.5 Hz, 2H), 2.76 (t, J=6.4 Hz, 2H), 2.44-2.26 (m, 7H), 2.26-2.15 (m, 2H), 2.04 (q, J=6.9 Hz, 4H), 1.94 (q, J=7.4 Hz, 2H), 1.61 (t, J=7.2 Hz, 2H), 1.50 (d, J=6.1 Hz, 4H), 1.39-1.19 (m, 37H), 0.88 (td, J=6.7, 4.8 Hz, 9H).

### Intermediate 49b: heptadecan-9-yl (3-(((4-nitrophenoxy)carbonyl)oxy)-2-((((9Z,12Z)-octadeca-9,12-dienoyl)oxy)methyl)propyl) glutarate

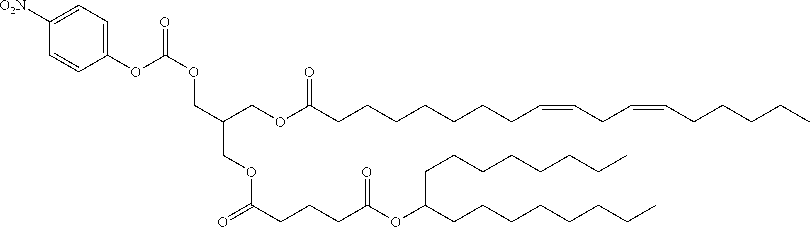 [SMILES: CCCCC/C=C\C/C=C\CCCCCCCC(=O)OCC(COC(=O)CCCC(=O)OC(CCCCCCCC)CCCCCCCC)COC(=O)Oc1ccc([N+](=O)[O-])cc1]

Intermediate 49b was synthesized (46%) from Intermediate 49a using the method employed in the synthesis of Intermediate 2d.

### Compound 49: 3-(((2-(diethylamino)ethyl)(methyl)carbamoyl)oxy)-2-((((9Z,12Z)-octadeca-9,12-dienoyl)oxy)methyl)propyl heptadecan-9-yl Glutarate

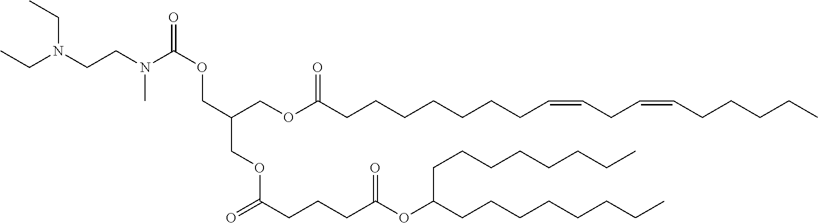 [SMILES: CCCCC/C=C\C/C=C\CCCCCCCC(=O)OCC(COC(=O)CCCC(=O)OC(CCCCCCCC)CCCCCCCC)COC(=O)N(C)CCN(CC)CC]

Compound 49 was synthesized from Intermediate 49b and N₁,N₁-diethyl-N₂-methylethane-1,2-diamine using the method employed in the synthesis of Compound 6. ¹H NMR (400 MHz, CDCl₃) δ 5.44-5.26 (m, 4H), 4.86 (p, J=6.2 Hz, 1H), 4.14 (t, J=5.3 Hz, 6H), 2.92 (d, J=10.7 Hz, 3H), 2.81-2.73 (m, 2H), 2.58-2.48 (m, 5H), 2.45-2.26 (m, 7H), 2.05 (q, J=6.8 Hz, 4H), 1.94 (p, J=7.5 Hz, 2H), 1.65-1.57 (m, 3H), 1.50 (d, J=6.2 Hz, 4H), 1.41-1.30 (m, 10H), 1.30-1.25 (m, 19H), 1.25 (s, 10H), 1.02 (q, J=6.9 Hz, 6H), 0.88 (td, J=6.9, 5.1 Hz, 9H). MS: 878.6 m/z [M+H].

### Example 50—Compound 50

### Compound 50: 3-(((2-(azepan-1-yl)ethyl)carbamoyl)oxy)-2-((((9Z,12Z)-octadeca-9,12-dienoyl)oxy)methyl)propyl heptadecan-9-yl Glutarate

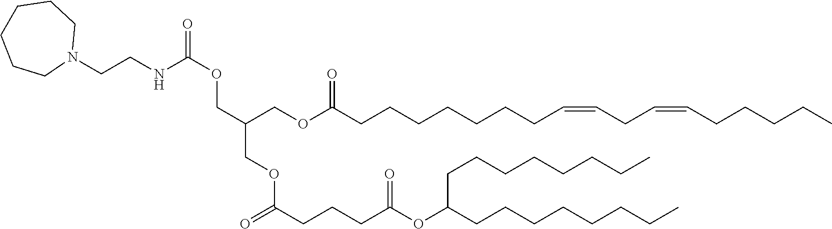 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1]

Compound 50 was synthesized from Intermediate 49b and 2-(azepan-1-yl)ethan-1-amine using the method employed in the synthesis of Compound 6. ¹H NMR (400 MHz, CDCl₃) δ 5.44-5.26 (m, 5H), 4.86 (p, J=6.3 Hz, 1H), 4.13 (t, J=5.8 Hz, 6H), 3.21 (t, J=6.0 Hz, 2H), 2.81-2.73 (m, 2H), 2.66-2.56 (m, 5H), 2.42-2.26 (m, 7H), 2.05 (q, J=6.8 Hz, 4H), 1.94 (p, J=7.5 Hz, 2H), 1.62 (d, J=16.8 Hz, 10H), 1.50 (d, J=6.2 Hz, 4H), 1.41-1.26 (m, 24H), 1.25 (s, 16H), 0.88 (td, J=6.9, 5.0 Hz, 9H). MS: 890.5 m/z [M+H].

### Example 51—Compound 51

### Intermediate 51a: 1-(11-((2-butyloctyl)oxy)-2-(hydroxymethyl)-11-oxoundecyl) 7-(heptadecan-9-yl) heptanedioate

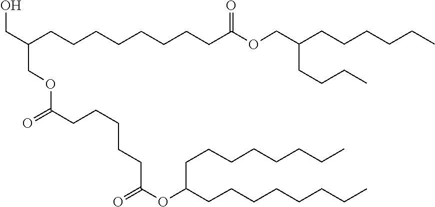 [SMILES: CCCCCCCCC(CCCCCCCC)OC(=O)CCCCCC(=O)OCC(CO)CCCCCCCCC(=O)OCC(CCCC)CCCCCC]

Intermediate 51a was synthesized from Intermediate 7d and Intermediate 3a using the method employed in the synthesis of Intermediate 7e. ¹H NMR (400 MHz, CDCl₃) δ 4.79 (p, J=6.3 Hz, 1H), 4.14 (dd, J=11.2, 4.3 Hz, 1H), 4.00 (dd, J=11.3, 6.7 Hz, 1H), 3.90 (d, J=5.8 Hz, 2H), 3.54-3.47 (m, 1H), 3.43 (dd, J=11.3, 6.5 Hz, 1H), 3.34 (q, J=6.5 Hz, 1H), 2.29-2.18 (m, 6H), 1.72 (td, J=6.7, 3.2 Hz, 1H), 1.57 (dq, J=10.3, 3.9, 2.7 Hz, 10H), 1.49-1.39 (m, 5H), 1.38-1.09 (m, 57H), 0.81 (td, J=6.9, 2.5 Hz, 12H).

### Intermediate 51b: 1-(11-((2-butyloctyl)oxy)-2-((((4-nitrophenoxy)carbonyl)oxy)methyl)-11-oxoundecyl) 7-(heptadecan-9-yl) heptanedioate

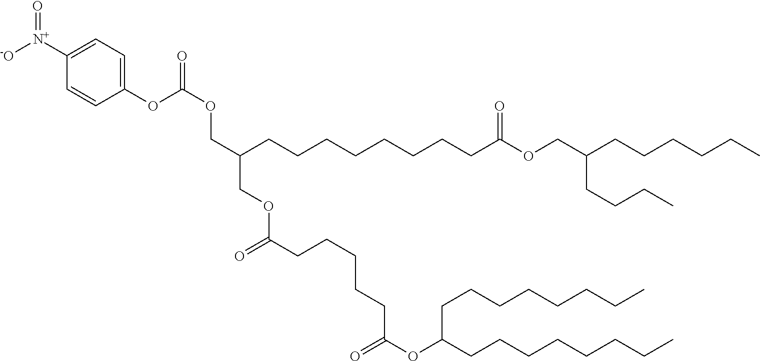

Intermediate 51b was synthesized from Intermediate 51a using the method employed in the synthesis of Intermediate 7f. ¹H NMR (400 MHz, CDCl₃) δ 8.24-8.17 (m, 2H), 7.36-7.29 (m, 2H), 4.79 (p, J=6.3 Hz, 1H), 4.28-4.15 (m, 2H), 4.09 (dt, J=11.9, 6.0 Hz, 1H), 4.02 (dd, J=11.3, 6.7 Hz, 1H), 3.90 (d, J=5.8 Hz, 2H), 2.24 (dq, J=14.1, 7.3 Hz, 6H), 2.03 (p, J=6.0 Hz, 1H), 1.63-1.48 (m, 8H), 1.43 (q, J=6.1 Hz, 5H), 1.38-1.10 (m, 56H), 0.81 (td, J=6.9, 2.8 Hz, 12H).

### Compound 51: 1-(11-((2-butyloctyl)oxy)-2-((((2-(diethylamino)ethyl)carbamoyl)oxy)methyl)-11-oxoundecyl) 7-(heptadecan-9-yl) heptanedioate

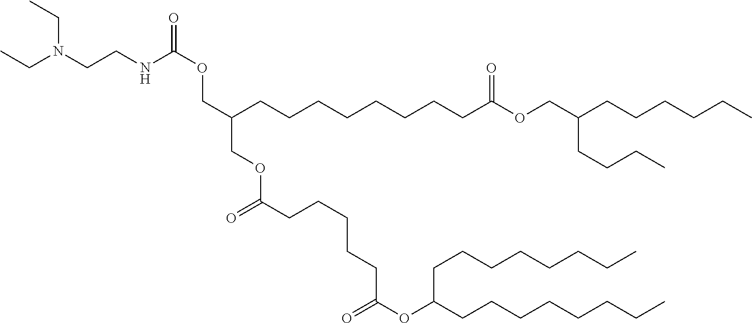 [SMILES: CCCCCCCCC(CCCCCCCC)OC(=O)CCCCCC(=O)OCC(CCCCCCCCC(=O)OCC(CCCC)CCCCCC)COC(=O)NCCN(CC)CC]

Compound 51 was synthesized from Intermediate 51b using the method employed in the synthesis of Compound 6. ¹H NMR (400 MHz, CDCl₃) δ 5.15 (s, 1H), 4.79 (p, J=6.3 Hz, 1H), 3.97 (td, J=14.7, 12.8, 9.0 Hz, 4H), 3.90 (d, J=5.7 Hz, 2H), 3.23-3.10 (m, 2H), 2.48 (s, 6H), 2.29-2.16 (m, 6H), 1.89 (s, 1H), 1.71-1.48 (m, 9H), 1.43 (q, J=6.1 Hz, 4H), 1.35-1.01 (m, 53H), 0.95 (t, J=7.2 Hz, 6H), 0.81 (dd, J=7.8, 5.6 Hz, 12H). MS: 923.5 m/z [M+H].

### Example 52—Compound 52

### Intermediate 52a: 8-(heptadecan-9-yloxy)-8-oxooctanoic Acid

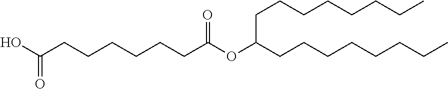 [SMILES: CCCCCCCCC(CCCCCCCC)OC(=O)CCCCCCC(=O)O]

Intermediate 52a was synthesized from octanedioic acid and Intermediate 2a using the method employed in the synthesis of Intermediate 2b. ¹H NMR (400 MHz, CDCl₃) δ 4.80 (p, J=6.3 Hz, 1H), 2.28 (t, J=7.5 Hz, 2H), 2.21 (t, J=7.5 Hz, 2H), 1.56 (ddq, J=11.6, 7.4, 4.2, 3.6 Hz, 4H), 1.34-1.11 (m, 29H), 0.86-0.75 (m, 6H).

### Intermediate 52b: 8,8′-di(heptadecan-9-yl) O′1,O1-(2-(hydroxymethyl)propane-1,3-diyl) di(octanedioate)

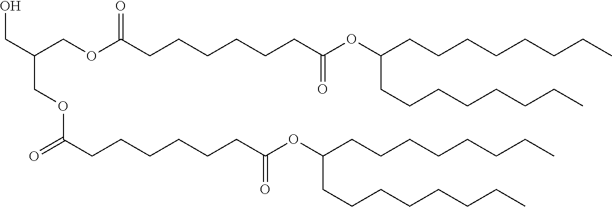 [SMILES: CCCCCCCCC(CCCCCCCC)OC(=O)CCCCCCC(=O)OCC(CO)COC(=O)CCCCCCC(=O)OC(CCCCCCCC)CCCCCCCC]

Intermediate 52b was synthesized from Intermediate 52a using the method employed in the synthesis of Intermediate 2c. ¹H NMR (400 MHz, CDCl₃) δ 4.88 (p, J=6.3 Hz, 2H), 4.24-4.12 (m, 4H), 3.64 (d, J=5.6 Hz, 2H), 2.32 (dt, J=17.5, 7.5 Hz, 8H), 2.21 (p, J=5.9 Hz, 1H), 1.65 (ddt, J=9.6, 4.8, 2.5 Hz, 8H), 1.52 (q, J=6.0 Hz, 8H), 1.40-1.20 (m, 58H), 0.89 (t, J=6.7 Hz, 12H).

### Intermediate 52c: 8,8′-di(heptadecan-9-yl) O′1,O1-(2-((((4-nitrophenoxy)carbonyl)oxy)methyl)propane-1,3-diyl) di(octanedioate)

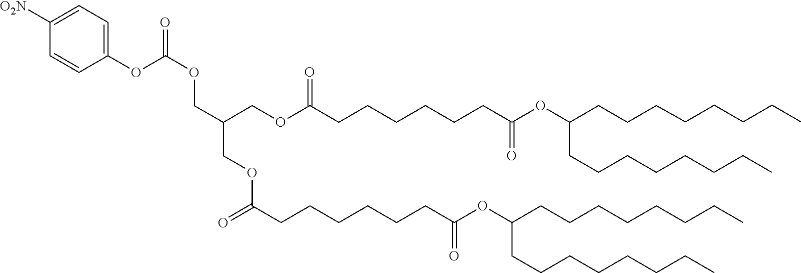 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)Oc1ccc([N+](=O)[O-])cc1]

Intermediate 52c was synthesized from Intermediate 52b using the method employed in the synthesis of Intermediate 2d. ¹H NMR (400 MHz, CDCl₃) δ 8.26-8.17 (m, 2H), 7.36-7.28 (m, 2H), 4.79 (p, J=6.3 Hz, 2H), 4.29 (d, J=5.9 Hz, 2H), 4.14 (dd, J=6.1, 1.9 Hz, 4H), 2.47-2.41 (m, 1H), 2.26 (t, J=7.5 Hz, 4H), 2.21 (t, J=7.5 Hz, 4H), 1.60-1.51 (m, 8H), 1.47-1.35 (m, 8H), 1.31-1.11 (m, 58H), 0.84-0.77 (m, 12H).

### Compound 52: O′1,O1-(2-((((2-(diethylamino)ethyl)carbamoyl)oxy)methyl)propane-1,3-diyl) 8,8′-di(heptadecan-9-yl) di(octanedioate)

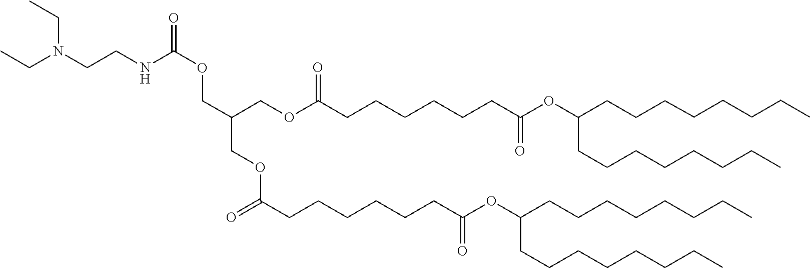 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)NCCN(CC)CC]

Compound 52 was synthesized from Intermediate 52c using the method employed in the synthesis of Compound 6. ¹H NMR (400 MHz, CDCl₃) δ 4.79 (p, J=6.3 Hz, 2H), 4.05 (d, J=6.0 Hz, 6H), 3.17 (s, 2H), 2.48 (s, 6H), 2.34-2.27 (m, 1H), 2.22 (dt, J=12.1, 7.5 Hz, 8H), 1.54 (dd, J=14.4, 7.6 Hz, 14H), 1.43 (q, J=6.3 Hz, 8H), 1.34-1.04 (m, 54H), 0.96 (s, 6H), 0.81 (t, J=6.7 Hz, 12H). MS: 1037.8 m/z [M+H].

### Example 53—Materials and Methods

### LNP Compositions for In Vivo Editing in Rats

The following additional ionizable lipids were used in the preparation of the LNPs:

LNPs were prepared using various amine lipids in a 4-component lipid system consisting of an ionizable lipid (e.g., an amine lipid), DSPC, cholesterol and a PEG lipid (e.g., PEG2K-DMG, C13 Ether, C14 Ether). In assays for percent liver editing in rats, Cas9 mRNA and chemically modified sgRNA targeting a rat sequence were formulated in LNPs, at either a 1:1 w/w ratio or a 1:2 w/w sgRNA:Cas9 mRNA ratio.

The lipid components were dissolved in 100% ethanol with the lipid component molar ratios described below. The chemically modified sgRNA and Cas9 mRNA were combined and dissolved in 25 mM citrate, 100 mM NaCl, pH 5.0, resulting in a concentration of total RNA cargo of approximately 0.45 mg/mL. The LNPs were formulated with an N:P ratio of about 6, with the ratio of chemically modified sgRNA:Cas9 mRNA at either a 1:1 or 1:2 w/w ratio as described below.

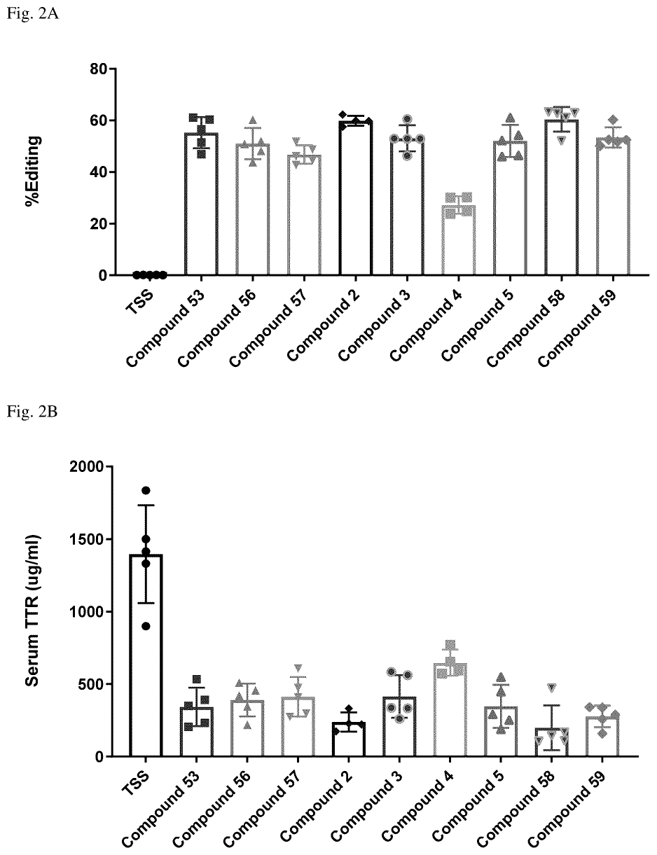

The LNPs were formed by an impinging jet mixing of the lipid in ethanol with two volumes of RNA solution and one volume of water. First, the lipid in ethanol is mixed through a mixing cross with the two volumes of RNA solution. Then, a fourth stream of water is mixed with the outlet stream of the cross through an inline tee. (See, e.g., WO2016010840, FIG. 2). The LNPs were held for 1 hour at room temperature, and further diluted with water (approximately 1:1 v/v). Diluted LNPs were buffer exchanged into 50 mM Tris, 45 mM NaCl, 5% (w/v) sucrose, pH 7.5 (TSS) and concentrated as needed by methods known in the art. The resulting mixture was then filtered using a 0.2 μm sterile filter. The final LNP was stored at 4° C. or −80° C. until further use.

The following additional ionizable lipids were used in the preparation of the LNPs:

| Com- | | Lipid |
|---|---|---|
| pound | Structure | pKa |
| 53 | 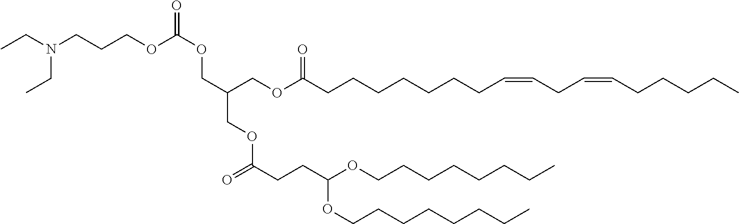 [SMILES: CCCCC/C=C\C/C=C\CCCCCCCC(=O)OCC(COC(=O)CCC(OCCCCCCCC)OCCCCCCCC)COC(=O)OCCCN(CC)CC] | 6.2 |
| 54 | 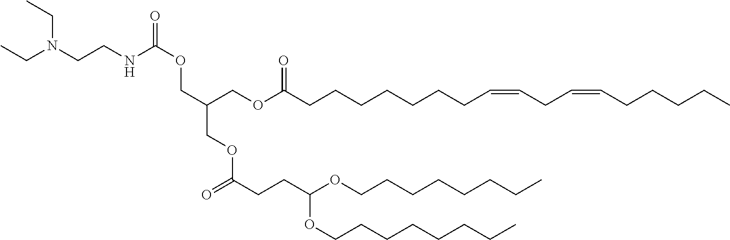 [SMILES: CCCCC/C=C\C/C=C\CCCCCCCC(=O)OCC(COC(=O)CCC(OCCCCCCCC)OCCCCCCCC)COC(=O)NCCN(CC)CC] | 6.5 |
| 55 | 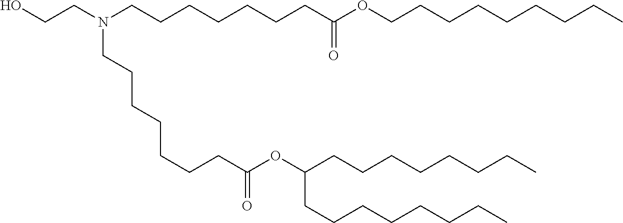 [SMILES: CCCCCCCCCOC(=O)CCCCCCCN(CCO)CCCCCCCC(=O)OC(CCCCCCCC)CCCCCCCC] | 6.6 |
| 56 | 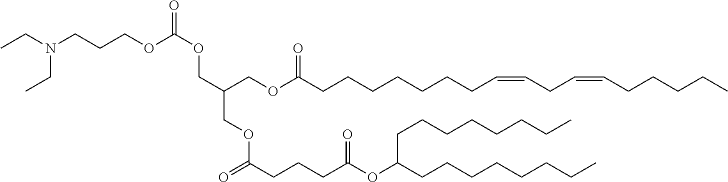 [SMILES: CCCCC/C=C\C/C=C\CCCCCCCC(=O)OCC(COC(=O)CCCC(=O)OC(CCCCCCCC)CCCCCCCC)COC(=O)OCCCN(CC)CC] | 6.1 |
| 57 | 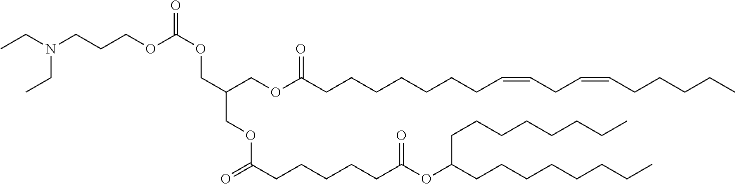 [SMILES: CCCCC/C=C\C/C=C\CCCCCCCC(=O)OCC(COC(=O)CCCCCC(=O)OC(CCCCCCCC)CCCCCCCC)COC(=O)OCCCN(CC)CC] | 6.1 |
| 58 | 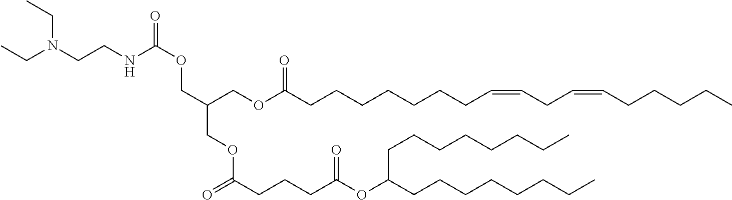 [SMILES: CCCCC/C=C\C/C=C\CCCCCCCC(=O)OCC(COC(=O)CCCC(=O)OC(CCCCCCCC)CCCCCCCC)COC(=O)NCCN(CC)CC] | 6.8 |
| 59 | 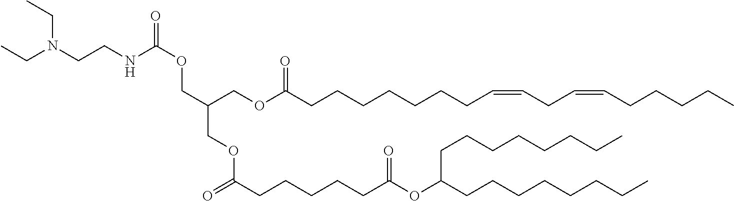 [SMILES: CCCCC/C=C\C/C=C\CCCCCCCC(=O)OCC(COC(=O)CCCCCC(=O)OC(CCCCCCCC)CCCCCCCC)COC(=O)NCCN(CC)CC] | 6.8 |
| 60 | 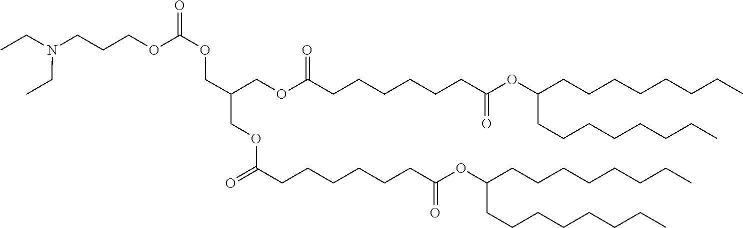 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN(CC)CC] | 6.1 |
| 61 | 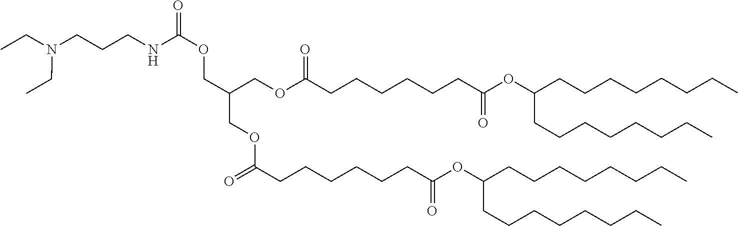 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)NCCCN(CC)CC] | 7.4 |

### LNP Composition Analytics

Dynamic Light Scattering (“DLS”) is used to characterize the polydispersity index (“pdi” or “PDI”) and size of the LNPs of the present disclosure. DLS measures the scattering of light that results from subjecting a sample to a light source. PDI, as determined from DLS measurements, represents the distribution of particle size (around the mean particle size) in a population, with a perfectly uniform population having a PDI of zero.

Electrophoretic light scattering is used to characterize the surface charge of the LNP at a specified pH. The surface charge, or the zeta potential, is a measure of the magnitude of electrostatic repulsion/attraction between particles in the LNP suspension.

Asymmetric-Flow Field Flow Fractionation-Multi-Angle Light Scattering (AF4-MALS) is used to separate particles in the composition by hydrodynamic radius and then measure the molecular weights, hydrodynamic radii and root mean square radii of the fractionated particles. This allows the ability to assess molecular weight and size distributions as well as secondary characteristics such as the Burchard-Stockmeyer Plot (ratio of root mean square (“rms”) radius to hydrodynamic radius over time suggesting the internal core density of a particle) and the rms conformation plot (log of rms radius vs log of molecular weight where the slope of the resulting linear fit gives a degree of compactness vs elongation).

Nanoparticle tracking analysis (NTA, Malvern Nanosight) can be used to determine particle size distribution as well as particle concentration. LNP samples are diluted appropriately and injected onto a microscope slide. A camera records the scattered light as the particles are slowly infused through field of view. After the movie is captured, the Nanoparticle Tracking Analysis processes the movie by tracking pixels and calculating a diffusion coefficient. This diffusion coefficient can be translated into the hydrodynamic radius of the particle. The instrument also counts the number of individual particles counted in the analysis to give particle concentration.

Cryo-electron microscopy (“cryo-EM”) can be used to determine the particle size, morphology, and structural characteristics of an LNP.

Lipid compositional analysis of the LNPs can be determined from liquid chromatography followed by charged aerosol detection (LC-CAD). This analysis can provide a comparison of the actual lipid content versus the theoretical lipid content.

LNP compositions are analyzed for average particle size, polydispersity index (pdi), total RNA content, encapsulation efficiency of RNA, and zeta potential. LNP compositions may be further characterized by lipid analysis, AF4-MALS, NTA, and/or cryo-EM.

Average particle size and polydispersity are measured by dynamic light scattering (DLS) using a Malvern Zetasizer DLS instrument. LNP samples were diluted with PBS buffer prior to being measured by DLS. Z-average diameter (“Z-avg”) which is an intensity-based measurement of average particle size was reported along with number average diameter (“number mean”) and PDI. A Malvern Zetasizer instrument is also used to measure the zeta potential of the LNP. Samples are diluted 1:17 (50 μL into 800 μL) in 0.1×PBS, pH 7.4 prior to measurement.

Encapsulation efficiency is calculated as (Total RNA−Free RNA)/Total RNA. A fluorescence-based assay (Ribogreen®, ThermoFisher Scientific) is used to determine total RNA concentration and free RNA. Encapsulation efficiency is calculated as (Total RNA−Free RNA)/Total RNA. LNP samples are diluted appropriately with 1×TE buffer containing 0.2% Triton-X 100 to determine total RNA or 1×TE buffer to determine free RNA. Standard curves are prepared by utilizing the starting RNA solution used to make the compositions and diluted in 1×TE buffer+/−0.2% Triton-X 100. Diluted RiboGreen® dye (according to the manufacturer's instructions) is then added to each of the standards and samples and allowed to incubate for approximately 10 minutes at room temperature, in the absence of light. A SpectraMax M5 Microplate Reader (Molecular Devices) is used to read the samples with excitation, auto cutoff and emission wavelengths set to 488 nm, 515 nm, and 525 nm respectively. Total RNA and free RNA are determined from the appropriate standard curves. Alternatively, the total RNA concentration can be determined by a reverse-phase ion-pairing (RP-IP) HPLC method. Triton X-100 is used to disrupt the LNPs, releasing the RNA. The RNA is then separated from the lipid components chromatographically by RP-IP HPLC and quantified against a standard curve using UV absorbance at 260 nm.

The same procedure may be used for determining the encapsulation efficiency of a DNA-based cargo component, in which case encapsulation efficiency is calculated as (Total DNA−Free DNA)/Total DNA. In a fluorescence-based assay, for single-strand DNA Oligreen Dye may be used, and for double-strand DNA, Picogreen Dye.

AF4-MALS is used to look at molecular weight and size distributions as well as secondary statistics from those calculations. LNPs are diluted as appropriate and injected into a AF4 separation channel using an HPLC autosampler where they are focused and then eluted with an exponential gradient in cross flow across the channel. All fluid is driven by an HPLC pump and Wyatt Eclipse Instrument. Particles eluting from the AF4 channel flow through a UV detector, multi-angle light scattering detector, quasi-elastic light scattering detector and differential refractive index detector. Raw data is processed by using a Debye model to determine molecular weight and rms radius from the detector signals.

Lipid components in LNPs are analyzed quantitatively by HPLC coupled to a charged aerosol detector (CAD). Chromatographic separation of 4 lipid components is achieved by reverse phase HPLC. CAD is a destructive mass based detector which detects all non-volatile compounds and the signal is consistent regardless of analyte structure.

The pKa of each amine lipid was determined according to the method in Jayaraman, et al. (Angew Chem Int Ed Engl 51(34), 2012, 8529-8533) with the following adaptations. The pKa was determined for unformulated amine lipid in ethanol. Lipid stock solutions (2.94 mM) were diluted into Sodium Phosphate Buffers (0.1 M, Boston Bioproducts) of different pH (pH-range: 4.5-9.0) yielding a final lipid concentration of approx. 100 μM. The test samples were supplemented with TNS {6-(p-Toluidino)-2-naphthalenesulfonic acid sodium salt}, incubated and the fluorescence intensity was measured using excitation and emission wavelengths of 321 nm and 448 nm, respectively. The recorded data were normalized and the respective pKa values were derived from sigmoidal fitting. The pKa values of the ionizable lipids are shown in Table 2.

TABLE 2

| Compound | Lipid pKa |
|---|---|
| 2 | 6.23 |
| 4 | 6.05 |
| 6 | 6.35 |
| 7 | 6.09 |
| 8 | 5.85 |
| 9 | 5.87 |
| 10 | 6.37 |
| 11 | 6.13 |
| 12 | 6.82 |
| 13 | 6.64 |
| 14 | 6.27 |
| 15 | 6.11 |
| 16 | 5.98 |
| 17 | 6.13 |
| 18 | 6.08 |
| 19 | 6.09 |
| 20 | 6.19 |
| 21 | 6.2 |
| 22 | 6.66 |
| 23 | 6.49 |
| 24 | 6.1 |
| 25 | 6.44 |
| 26 | 6.36 |
| 27 | 6.75 |
| 28 | 6.2 |
| 29 | 7.1 |
| 30 | 6.4 |
| 31 | 6.8 |
| 32 | 5.7 |
| 33 | 6.5 |
| 34 | 6.33 |
| 35 | 6.83 |
| 35 | 6.93 |
| 37 | 6.66 |
| 38 | 6.56 |
| 39 | 6.63 |
| 40 | 5.97 |
| 49 | 6.38 |
| 50 | 6.6 |
| 51 | 6.39 |
| 52 | 6.47 |

Cas9 mRNA and gRNA Cargos

The Cas9 mRNA cargo was prepared by in vitro transcription. Capped and polyadenylated Cas9 mRNA comprising 1×NLS (SEQ ID NO: 1) was generated by in vitro transcription using a linearized plasmid DNA template and T7 RNA polymerase. For example, plasmid DNA containing a T7 promoter and a 100 nt poly(A/T) region can be linearized by incubating at 37° C. for 2 hours with XbaI with the following conditions: 200 ng/μL plasmid, 2 U/μL XbaI (NEB), and 1× reaction buffer. The XbaI can be inactivated by heating the reaction at 65° C. for 20 min. The linearized plasmid can be purified from enzyme and buffer salts using a silica maxi spin column (Epoch Life Sciences) and analyzed by agarose gel to confirm linearization. The IVT reaction to generate Cas9 modified mRNA can be performed by incubating at 37° C. for 4 hours in the following conditions: 50 ng/μL linearized plasmid; 2 mM each of GTP, ATP, CTP, and N1-methyl pseudo-UTP (Trilink); 10 mM ARCA (Trilink); 5 U/μL T7 RNA polymerase (NEB); 1 U/μL Murine RNase inhibitor (NEB); 0.004 U/μL Inorganic E. coli pyrophosphatase (NEB); and 1× reaction buffer. After the 4 hr incubation, TURBO DNase (ThermoFisher) was added to a final concentration of 0.01 U/μL, and the reaction was incubated for an additional 30 minutes to remove the DNA template. The Cas9 mRNA (SEQ ID NO: 3) was purified with an LiCl precipitation-containing method.

The sgRNAs in the following examples were chemically synthesized by known methods using phosphoramidites.

### LNP Delivery In Vivo

Sprague-Dawley female rats, ranging from 6-10 weeks of age were used in each study. Animals were weighed and dosed based on individual body weight measured the morning of dosing. LNPs were dosed via the lateral tail vein in a volume of 2 mL per kilogram of animal body weight. The animals were periodically observed for adverse effects for at least 24 hours post dose.

For studies measuring in vivo editing in liver, Sprague-Dawley female rats were dosed at 0.1 mpk or 0.03 mpk, unless otherwise noted. Animals were euthanized at 6 or 7 days by carbon dioxide asphyxiation and a heart clip or by ex sanguination via cardiac puncture under isoflourane anesthesia. Blood was collected into serum separator tubes. Liver tissue was collected. Genomic DNA was isolated from the liver tissue for editing measurement by Next-Generation Sequencing (NGS).

### NGS Sequencing

In brief, to quantitatively determine the efficiency of editing at the target location in the genome, genomic DNA was isolated and deep sequencing was utilized to identify the presence of insertions and deletions introduced by gene editing.

PCR primers were designed around the target site (e.g., TTR), and the genomic area of interest was amplified. Additional PCR was performed according to the manufacturer's protocols (Illumina) to add the necessary chemistry for sequencing. The amplicons were sequenced on an Illumina MiSeq instrument. The reads were aligned to the human reference genome (e.g., hg38) after eliminating those having low quality scores. The resulting files containing the reads were mapped to the reference genome (BAM files), where reads that overlapped the target region of interest were selected and the number of wild type reads versus the number of reads which contain an insertion, substitution, or deletion was calculated.

The editing percentage (e.g., the “indel efficiency” or “percent indels” or “% editing” or “% indel”) can be defined as the total number of sequence reads with insertions or deletions over the total number of sequence reads, including wild type.

### Transthyretin (TTR) ELISA Analysis

Blood was collected and the serum was isolated as indicated. The total rat TTR serum levels were determined using a Rat Prealbumin (Transthyretin) ELISA Kit (Aviva Systems Biology, Cat. #OKIA00159). Briefly, sera were serial diluted with kit sample diluent to a final dilution of 10,000-fold. This diluted sample was then added to the ELISA plates and the assay was then carried out according to manufacturer's protocol.

### Example 54—Assessing Lipid Efficacy by In Vivo Editing

In vivo editing efficiency for materials delivered with formulations including various amine lipid (or ionizable lipid) compounds in Experiments 1 through 14 as described in Tables 3 and 4 was assessed. Editing was measured using G000534 (SEQ ID NO: 5) which targets the rat TTR gene. Lipids described above were assessed for efficacy through in vivo editing experiments. LNPs were formulated at a 1:2 w/w ratio of sgRNA (SEQ ID NO: 5) and Cas9 mRNA (SEQ ID NO: 3). In Experiments 1 through 13, lipid components of the LNP are formulated in a molar ratio of 50% ionizable lipid, 38% cholesterol, 9% DSPC, and 3% PEG2K-DMG. In Experiment 14, lipid components of the LNP are formulated in a molar ratio of 50% ionizable lipid, 38% cholesterol, 9% DSPC, and 3% C13 Ether. The final LNPs used in different in vivo editing experiments were characterized to determine the encapsulation efficiency (% E), polydispersity index (PDI), and average particle size (Z-avg and number mean) according to the analytical methods provided above. Results of the composition analysis are shown in Table 3. The pKa values of each of the following ionizable lipids were also measured to be in the range of about 5.8 to about 7.6.

TABLE 3
Composition Analytics.

| | | | | | | Number | |
|---|---|---|---|---|---|---|---|
| | | | | Z-avg | | mean | |
| LNP# | Ionizable Lipid | sgRNA:mRNA | % E | (nm) | PDI | (nm) | Experiment |
| 1 | Compound 53 | 1:2 | 97 | 95 | 0.01 | 78 | 1 |
| 2 | Compound 54 | 1:2 | 88 | 98 | 0.003 | 80 | 1 |
| 3 | Compound 58 | 1:2 | 90 | 94 | 0.01 | 77 | 1 |
| 4 | Compound 59 | 1:2 | 83 | 100 | 0.01 | 83 | 1 |
| 1 | Compound 53 | 1:2 | 98 | 94 | 0.03 | 77 | 2 |
| 5 | Compound 56 | 1:2 | 95 | 90 | 0.04 | 74 | 2 |
| 6 | Compound 57 | 1:2 | 96 | 91 | 0.004 | 75 | 2 |
| 7 | Compound 2 | 1:2 | 98 | 79 | 0.01 | 65 | 2 |
| 8 | Compound 3 | 1:2 | 96 | 86 | 0.004 | 72 | 2 |
| 9 | Compound 4 | 1:2 | 99 | 95 | 0.02 | 78 | 2 |
| 10 | Compound 5 | 1:2 | 97 | 109 | 0.01 | 90 | 2 |
| 3 | Compound 58 | 1:2 | 87 | 91 | 0.08 | 69 | 2 |
| 4 | Compound 59 | 1:2 | 82 | 95 | 0.02 | 78 | 2 |
| 1 | Compound 53 | 1:2 | 98 | 89 | 0.02 | 81 | 3 |
| 11 | Compound 1 | 1:2 | 98 | 80 | 0.06 | 70 | 3 |
| 12 | Compound 7 | 1:2 | 98 | 74 | 0.03 | 68 | 3 |
| 9 | Compound 4 | 1:2 | 99 | 91 | 0.04 | 83 | 3 |
| 13 | Compound 9 | 1:2 | 99 | 68 | 0.03 | 61 | 3 |
| 6 | Compound 57 | 1:2 | 96 | 88 | 0.03 | 80 | 3 |
| 14 | Compound 8 | 1:2 | 98 | 69 | 0.03 | 64 | 3 |
| 7 | Compound 2 | 1:2 | 98 | 76 | 0.01 | 72 | 3 |
| 1 | Compound 53 | 1:2 | 99 | 96 | 0.02 | 90 | 4 |
| 13 | Compound 9 | 1:2 | 99 | 73 | 0.03 | 65 | 4 |
| 15 | Compound 11 | 1:2 | 99 | 78 | 0.02 | 70 | 4 |
| 16 | Compound 10 | 1:2 | 97 | 90 | 0.04 | 83 | 4 |
| 17 | Compound 14 | 1:2 | 96 | 114 | 0.01 | 107 | 4 |
| 18 | Compound 13 | 1:2 | 98 | 99 | 0.04 | 87 | 4 |
| 19 | Compound 12 | 1:2 | 96 | 108 | 0.03 | 101 | 4 |
| 1 | Compound 53 | 1:2 | 98 | 92 | 0.04 | 79 | 5 |
| 20 | Compound 24 | 1:2 | 98 | 67 | 0.03 | 60 | 5 |
| 21 | Compound 25 | 1:2 | 98 | 72 | 0.03 | 64 | 5 |
| 22 | Compound 26 | 1:2 | 98 | 81 | 0.01 | 76 | 5 |
| 23 | Compound 27 | 1:2 | 98 | 82 | 0.01 | 77 | 5 |
| 1 | Compound 53 | 1:2 | 99 | 92 | 0.02 | 88 | 6 |
| 24 | Compound 23 | 1:2 | 92 | 90 | 0.02 | 86 | 6 |
| 1 | Compound 53 | 1:2 | 94 | 90 | 0.01 | 84 | 7 |
| 25 | Compound 41 | 1:2 | 98 | 83 | 0.05 | 75 | 7 |
| 26 | Compound 43 | 1:2 | 98 | 85 | 0.04 | 75 | 7 |
| 27 | Compound 45 | 1:2 | 98 | 98 | 0 | 91 | 7 |
| 28 | Compound 47 | 1:2 | 97 | 87 | 0.02 | 81 | 7 |
| 29 | Compound 42 | 1:2 | 98 | 86 | 0.01 | 80 | 7 |
| 30 | Compound 44 | 1:2 | 95 | 90 | 0.04 | 76 | 7 |
| 31 | Compound 46 | 1:2 | 97 | 96 | 0.01 | 90 | 7 |
| 32 | Compound 48 | 1:2 | 94 | 87 | 0.02 | 81 | 7 |
| 1 | Compound 53 | 1:2 | 99 | 91 | 0.06 | 84 | 8 |
| 33 | Compound 22 | 1:2 | 99 | 82 | 0 | 77 | 8 |
| 1 | Compound 53 | 1:2 | 99 | 95 | 0.04 | 85 | 9 |
| 13 | Compound 9 | 1:2 | 99 | 68 | 0.03 | 62 | 9 |
| 34 | Compound 15 | 1:2 | 98 | 66 | 0.06 | 58 | 9 |
| 35 | Compound 16 | 1:2 | 99 | 68 | 0.03 | 62 | 9 |
| 36 | Compound 17 | 1:2 | 99 | 74 | 0.03 | 67 | 9 |
| 37 | Compound 18 | 1:2 | 99 | 75 | 0.01 | 69 | 9 |
| 38 | Compound 19 | 1:2 | 99 | 71 | 0.02 | 66 | 9 |
| 39 | Compound 20 | 1:2 | 98 | 84 | 0.05 | 68 | 9 |
| 1 | Compound 53 | 1:2 | 98 | 90 | 0.002 | 86 | 10 |
| 5 | Compound 56 | 1:2 | 97 | 86 | 0.03 | 80 | 10 |
| 3 | Compound 58 | 1:2 | 87 | 92 | 0.02 | 84 | 10 |
| 40 | Compound 49 | 1:2 | 97 | 91 | 0.01 | 87 | 10 |
| 41 | Compound 50 | 1:2 | 98 | 74 | 0.02 | 66 | 10 |
| 1 | Compound 53 | 1:2 | 99 | 91 | 0.01 | 87 | 11 |
| 42 | Compound 55 | 1:2 | 84 | 77 | 0.05 | 65 | 11 |
| 24 | Compound 23 | 1:2 | 95 | 81 | 0.02 | 74 | 11 |
| 43 | Compound 51 | 1:2 | 99 | 90 | 0.03 | 81 | 11 |
| 44 | Compound 6 | 1:2 | 99 | 75 | 0.014 | 68 | 12 |
| 45 | Compound 28 | 1:2 | 99 | 65 | 0.022 | 60 | 12 |
| 46 | Compound 29 | 1:2 | 100 | 74 | 0.023 | 67 | 12 |
| 47 | Compound 30 | 1:2 | 99 | 71 | 0.02 | 64 | 12 |
| 48 | Compound 31 | 1:2 | 100 | 64 | 0.003 | 59 | 12 |
| 49 | Compound 32 | 1:2 | 99 | 63 | 0.008 | 59 | 12 |
| 50 | Compound 33 | 1:2 | 100 | 71 | 0.023 | 66 | 12 |
| 51 | Compound 36 | 1:2 | 98 | 103 | 0.056 | 87 | 12 |
| 52 | Compound 37 | 1:2 | 99 | 78 | 0.014 | 71 | 12 |
| 1 | Compound 53 | 1:2 | 98 | 90 | 0.02 | 81 | 13 |
| 44 | Compound 6 | 1:2 | 99 | 79 | 0.02 | 75 | 13 |
| 53 | Compound 60 | 1:2 | 97 | 87 | 0.02 | 78 | 13 |
| 54 | Compound 52 | 1:2 | 97 | 87 | 0.03 | 79 | 13 |
| 55 | Compound 61 | 1:2 | 100 | 74 | 0.16 | 39 | 13 |
| 56 | Compound 6 | 1:2 | 98 | 73 | 0.02 | 68 | 14 |
| 57 | Compound 2 | 1:2 | 99 | 80 | 0.017 | 74 | 14 |
| 58 | Compound 35 | 1:2 | 99 | 76 | 0.019 | 67 | 14 |
| 59 | Compound 39 | 1:2 | 98 | 71 | 0.086 | 61 | 14 |
| 60 | Compound 40 | 1:2 | 92 | 57 | 0.062 | 48 | 14 |
| 61 | Compound 38 | 1:2 | 95 | 85 | 0.034 | 74 | 14 |
| 62 | Compound 34 | 1:2 | 98 | 69 | 0.032 | 61 | 14 |

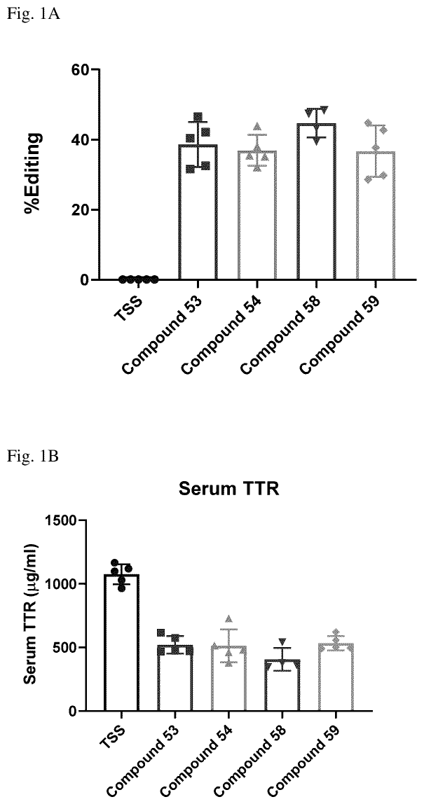

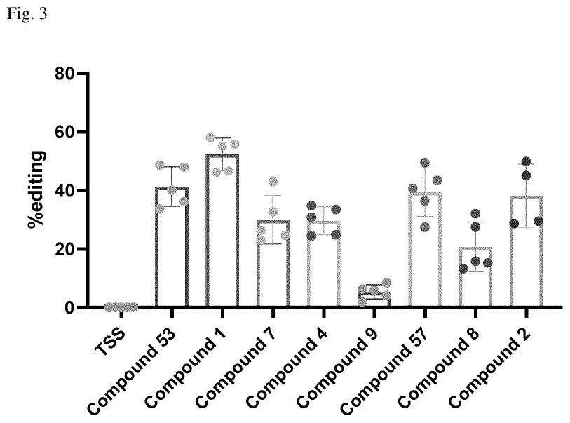

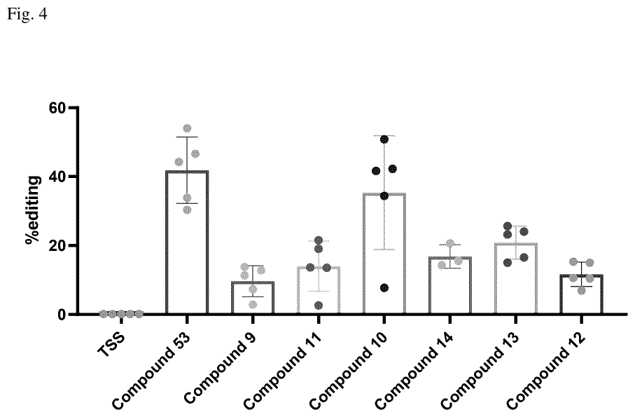

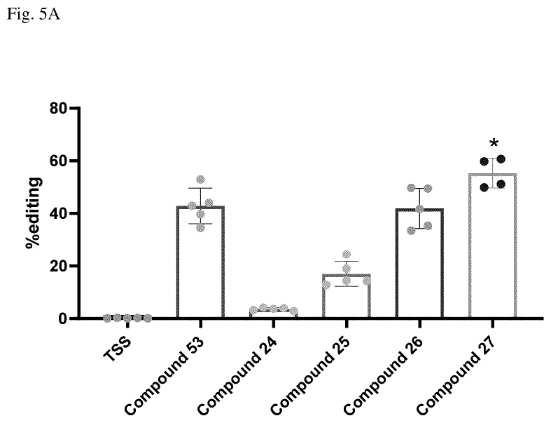

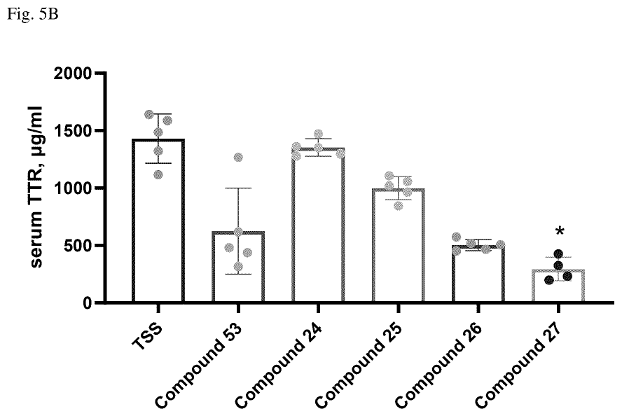

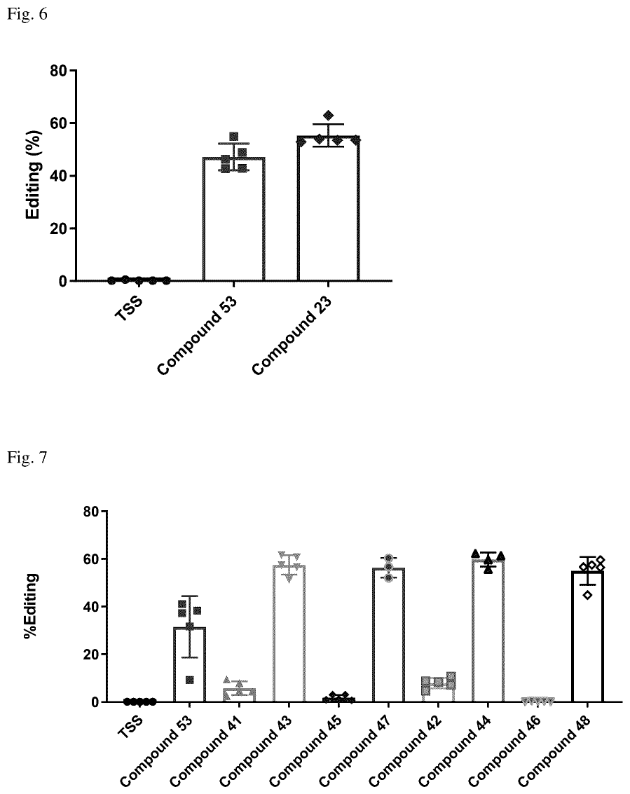

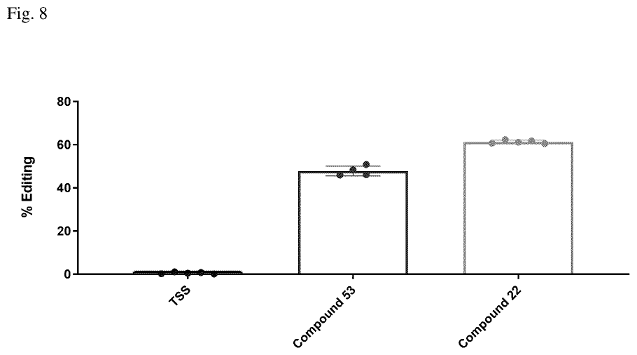

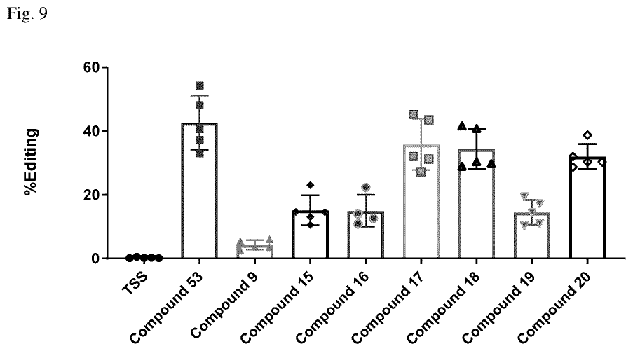

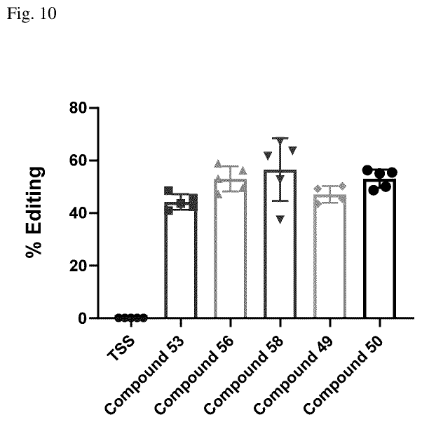

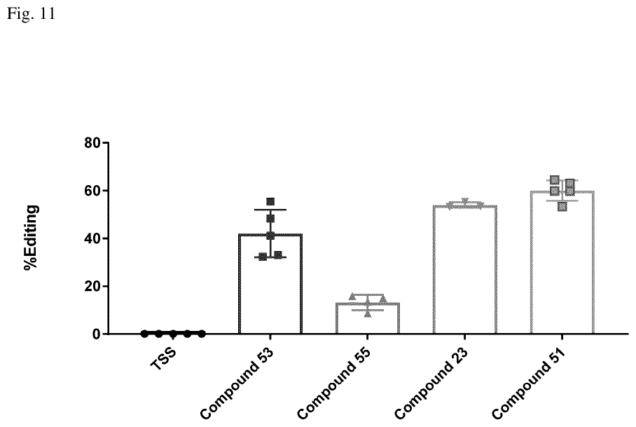

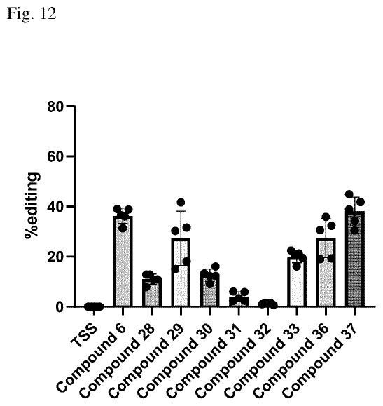

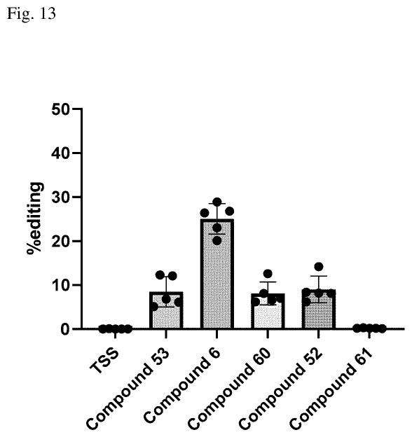

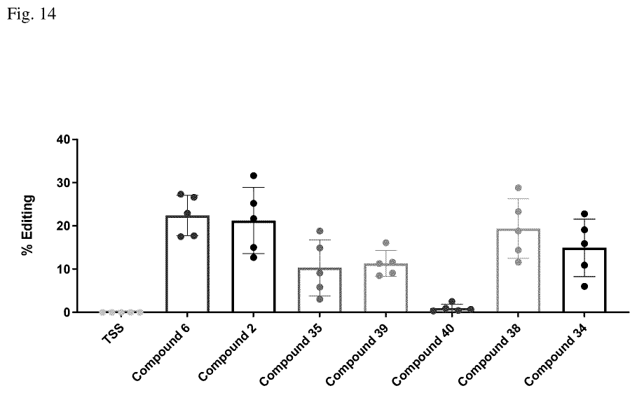

Table 4 shows editing percentages in rat liver as measured by NGS, and serum TTR levels as measured by ELISA when available, for Experiments 1 through 14. TSS was used as a vehicle-only negative control. The data are illustrated in FIGS. 1A through 14.

TABLE 4
Editing efficiency in rat liver measured by % editing and serum TTR levels.

| | | | | | Serum | | | | |
|---|---|---|---|---|---|---|---|---|---|
| Ionizable | Dose | Mean | | | TTR | | | | |
| Lipid | (mpk) | % editing | SD | N | (ug/ml) | SD | N | % TSS | Experiment |
| TSS | TSS | 0.1 | 0.0 | 5 | 1075.3 | 78.9 | 5 | 100% | 1 |
| Compound 53 | 0.1 | 38.6 | 6.4 | 5 | 519.9 | 69.7 | 5 | 48% | 1 |
| Compound 54 | 0.1 | 36.9 | 4.4 | 5 | 512.9 | 129.6 | 5 | 48% | 1 |
| Compound 58 | 0.1 | 44.7 | 4.1 | 4 | 406.3 | 90.0 | 4 | 38% | 1 |
| Compound 59 | 0.1 | 36.7 | 7.3 | 5 | 533.0 | 56.2 | 5 | 50% | 1 |
| TSS | TSS | 0.0 | 0.1 | 5 | 1396.0 | 337.5 | 5 | 100% | 2 |
| Compound 53 | 0.1 | 55.2 | 6.0 | 5 | 341.9 | 132.3 | 5 | 24% | 2 |
| Compound 56 | 0.1 | 50.9 | 6.1 | 5 | 390.1 | 112.4 | 5 | 28% | 2 |
| Compound 57 | 0.1 | 46.7 | 3.5 | 5 | 412.3 | 136.3 | 5 | 30% | 2 |
| Compound 2 | 0.1 | 59.8 | 1.9 | 4 | 238.5 | 66.4 | 4 | 17% | 2 |
| Compound 3 | 0.1 | 53.0 | 5.1 | 5 | 414.5 | 147.3 | 5 | 30% | 2 |
| Compound 4 | 0.1 | 27.2 | 3.4 | 4 | 647.4 | 89.6 | 4 | 46% | 2 |
| Compound 5 | 0.1 | 52.0 | 6.2 | 5 | 346.5 | 148.5 | 5 | 25% | 2 |
| Compound 58 | 0.1 | 60.4 | 4.8 | 5 | 198.8 | 154.2 | 5 | 14% | 2 |
| Compound 59 | 0.1 | 53.4 | 3.9 | 5 | 276.6 | 74.2 | 5 | 20% | 2 |
| TSS | TSS | 0.1 | 0.0 | 5 | | | | | 3 |
| Compound 53 | 0.1 | 41.4 | 6.8 | 5 | | | | | 3 |
| Compound 1 | 0.1 | 52.4 | 5.6 | 5 | | | | | 3 |
| Compound 7 | 0.1 | 30.0 | 8.2 | 5 | | | | | 3 |
| Compound 4 | 0.1 | 29.7 | 4.8 | 5 | | | | | 3 |
| Compound 9 | 0.1 | 5.3 | 2.4 | 5 | | | | | 3 |
| Compound 57 | 0.1 | 39.5 | 8.3 | 5 | | | | | 3 |
| Compound 8 | 0.1 | 20.8 | 8.5 | 5 | | | | | 3 |
| Compound 2 | 0.1 | 38.3 | 10.8 | 4 | | | | | 3 |
| TSS | TSS | 0.1 | 0.0 | 5 | | | | | 4 |
| Compound 53 | 0.1 | 41.8 | 9.6 | 5 | | | | | 4 |
| Compound 9 | 0.1 | 9.6 | 4.5 | 5 | | | | | 4 |
| Compound 11 | 0.1 | 14.0 | 7.3 | 5 | | | | | 4 |
| Compound 10 | 0.1 | 35.3 | 16.5 | 5 | | | | | 4 |
| Compound 14 | 0.1 | 16.8 | 3.4 | 3 | | | | | 4 |
| Compound 13 | 0.1 | 20.9 | 4.8 | 5 | | | | | 4 |
| Compound 12 | 0.1 | 11.6 | 3.5 | 5 | | | | | 4 |
| TSS | TSS | 0.2 | 0.1 | 5 | 1430.7 | 214.1 | 5 | 100% | 5 |
| Compound 53 | 0.1 | 42.8 | 6.7 | 5 | 624.1 | 375.5 | 5 | 44% | 5 |
| Compound 24 | 0.1 | 3.6 | 0.5 | 5 | 1352.4 | 76.2 | 5 | 95% | 5 |
| Compound 25 | 0.1 | 17.0 | 4.8 | 5 | 998.3 | 100.5 | 5 | 70% | 5 |
| Compound 26 | 0.1 | 41.9 | 7.6 | 5 | 502.9 | 48.7 | 5 | 35% | 5 |
| Compound 27 | 0.1 | 55.3 | 5.6 | 4 | 294.3 | 102.4 | 4 | 21% | 5 |
| TSS | TSS | 0.2 | 0.2 | 5 | | | | | 6 |
| Compound 53 | 0.1 | 47.1 | 5.1 | 5 | | | | | 6 |
| Compound 23 | 0.1 | 55.3 | 4.3 | 5 | | | | | 6 |
| TSS | TSS | 0.1 | 0.0 | 5 | | | | | 7 |
| Compound 53 | 0.1 | 31.5 | 12.9 | 5 | | | | | 7 |
| Compound 41 | 0.1 | 5.8 | 2.9 | 5 | | | | | 7 |
| Compound 43 | 0.1 | 57.5 | 4.1 | 5 | | | | | 7 |
| Compound 45 | 0.1 | 1.8 | 1.1 | 5 | | | | | 7 |
| Compound 47 | 0.1 | 56.3 | 4.2 | 3 | | | | | 7 |
| Compound 42 | 0.1 | 7.9 | 2.2 | 5 | | | | | 7 |
| Compound 44 | 0.1 | 59.8 | 2.9 | 4 | | | | | 7 |
| Compound 46 | 0.1 | 0.2 | 0.1 | 5 | | | | | 7 |
| Compound 48 | 0.1 | 55.0 | 5.8 | 5 | | | | | 7 |
| TSS | TSS | 0.5 | 0.4 | 5 | | | | | 8 |
| Compound 53 | 0.1 | 47.8 | 2.3 | 4 | | | | | 8 |
| Compound 22 | 0.1 | 61.3 | 0.8 | 5 | | | | | 8 |
| TSS | TSS | 0.2 | 0.2 | 5 | | | | | 9 |
| Compound 53 | 0.1 | 42.6 | 8.6 | 5 | | | | | 9 |
| Compound 9 | 0.1 | 4.3 | 1.5 | 5 | | | | | 9 |
| Compound 15 | 0.1 | 15.1 | 4.7 | 5 | | | | | 9 |
| Compound 16 | 0.1 | 14.9 | 5.1 | 4 | | | | | 9 |
| Compound 17 | 0.1 | 35.8 | 8.0 | 5 | | | | | 9 |
| Compound 18 | 0.1 | 34.4 | 6.3 | 5 | | | | | 9 |
| Compound 19 | 0.1 | 14.4 | 3.9 | 5 | | | | | 9 |
| Compound 20 | 0.1 | 32.0 | 4.0 | 5 | | | | | 9 |
| TSS | TSS | 0.1 | 0.0 | 5 | | | | | 10 |
| Compound 53 | 0.1 | 44.2 | 3.0 | 5 | | | | | 10 |
| Compound 56 | 0.1 | 53.0 | 4.8 | 5 | | | | | 10 |
| Compound 58 | 0.1 | 56.5 | 11.9 | 5 | | | | | 10 |
| Compound 49 | 0.1 | 47.1 | 3.2 | 4 | | | | | 10 |
| Compound 50 | 0.1 | 53.1 | 3.5 | 5 | | | | | 10 |
| TSS | TSS | 0.1 | 0.0 | 5 | | | | | 11 |
| Compound 53 | 0.1 | 42.0 | 9.9 | 5 | | | | | 11 |
| Compound 55 | 0.1 | 13.2 | 3.2 | 4 | | | | | 11 |
| Compound 23 | 0.1 | 54.0 | 1.2 | 3 | | | | | 11 |
| Compound 51 | 0.1 | 60.0 | 4.3 | 5 | | | | | 11 |
| TSS | TSS | 0.0 | 0.0 | 5 | | | | | 12 |
| Compound 6 | 0.04 | 36.3 | 3.1 | 5 | | | | | 12 |
| Compound 28 | 0.04 | 11.0 | 2.0 | 5 | | | | | 12 |
| Compound 29 | 0.04 | 27.3 | 10.9 | 5 | | | | | 12 |
| Compound 30 | 0.04 | 12.6 | 2.5 | 5 | | | | | 12 |
| Compound 31 | 0.04 | 4.0 | 1.9 | 5 | | | | | 12 |
| Compound 32 | 0.04 | 1.2 | 0.3 | 5 | | | | | 12 |
| Compound 33 | 0.04 | 20.0 | 2.4 | 5 | | | | | 12 |
| Compound 36 | 0.04 | 27.4 | 7.8 | 5 | | | | | 12 |
| Compound 37 | 0.04 | 38.1 | 5.8 | 5 | | | | | 12 |
| TSS | TSS | 0.0 | 0.0 | 5 | | | | | 13 |
| Compound 53 | 0.03 | 8.5 | 3.4 | 5 | | | | | 13 |
| Compound 6 | 0.03 | 25.0 | 3.5 | 5 | | | | | 13 |
| Compound 60 | 0.03 | 8.1 | 2.6 | 5 | | | | | 13 |
| Compound 52 | 0.03 | 9.0 | 3.0 | 5 | | | | | 13 |
| Compound 61 | 0.03 | 0.2 | 0.1 | 5 | | | | | 13 |
| TSS | TSS | 0.0 | 0.0 | 5 | | | | | 14 |
| Compound 6 | 0.03 | 22.4 | 4.7 | 5 | | | | | 14 |
| Compound 2 | 0.03 | 21.2 | 7.7 | 5 | | | | | 14 |
| Compound 35 | 0.03 | 10.3 | 6.5 | 5 | | | | | 14 |
| Compound 39 | 0.03 | 11.3 | 3.0 | 5 | | | | | 14 |
| Compound 40 | 0.03 | 1.0 | 0.9 | 5 | | | | | 14 |
| Compound 38 | 0.03 | 19.4 | 6.9 | 5 | | | | | 14 |
| Compound 34 | 0.03 | 14.9 | 6.6 | 5 | | | | | 14 |

### Example 55—Assessing Lipid Efficacy by In Vivo Editing

In vivo editing efficiency for materials delivered with formulations including various combinations of amine lipid (or ionizable lipid) and PEG lipid compounds was assessed as described in Tables 5 and 6. Editing was measured using G000534 (SEQ ID NO: 5) which targets the rat TTR gene. Lipids described above were assessed for efficacy through in vivo editing experiments. LNPs were formulated at a 1:2 w/w ratio of sgRNA (SEQ ID NO: 5) and Cas9 mRNA (SEQ ID NO:3). Lipid components of the LNP are formulated in a molar ratio of 50% ionizable lipid, 38% cholesterol, 9% DSPC, and 3% of various PEG lipids (e.g., PEG2K-DMG, C13 Ether, C14 Ether). The final LNPs used in different in vivo editing experiments were characterized to determine the encapsulation efficiency (% E), polydispersity index (PDI), and average particle size (Z-avg and number mean) according to the analytical methods provided above. Results of the composition analysis are shown in Table 5. The pKa values of each of the following ionizable lipids were also measured to be in the range of about 5.8 to about 7.6.

TABLE 5
Composition Analytics

| | | | | | | | Number |
|---|---|---|---|---|---|---|---|
| | | | | | Z-avg | | mean |
| LNP# | Ionizable Lipid | PEG lipid | sgRNA:mRNA | % E | (nm) | PDI | (nm) |
| 1 | Compound 53 | PEG2K-DMG | 1:2 | 97 | 87 | 0.01 | 82 |
| 63 | Compound 53 | C13 Ether | 1:2 | 97 | 87 | 0.02 | 80 |
| 64 | Compound 53 | C14 Ether | 1:2 | 99 | 96 | 0.01 | 90 |
| 44 | Compound 6 | PEG2K-DMG | 1:2 | 97 | 78 | 0.04 | 69 |
| 56 | Compound 6 | C13 Ether | 1:2 | 97 | 79 | 0.01 | 72 |
| 65 | Compound 6 | C14 Ether | 1:2 | 98 | 86 | 0.01 | 81 |
| 53 | Compound 60 | PEG2K-DMG | 1:2 | 96 | 89 | 0.03 | 77 |
| 66 | Compound 60 | C13 Ether | 1:2 | 95 | 87 | 0.03 | 80 |
| 67 | Compound 60 | C14 Ether | 1:2 | 96 | 95 | 0.03 | 83 |

Table 6 shows editing percentages in rat liver as measured by NGS. TSS was used as a vehicle-only negative control. The data are illustrated in FIG. 15.

TABLE 6
Editing efficiency in rat liver measured by % editing.

| Ionizable | | Dose | Mean % | | |
|---|---|---|---|---|---|
| Lipid | PEG lipid | (mpk) | editing | SD | N |
| TSS | — | TSS | 0.1 | 0.0 | 5 |
| Compound 53 | PEG2K-DMG | 0.03 | 9.6 | 3.8 | 5 |
| Compound 53 | C13 Ether | 0.03 | 7.5 | 2.3 | 5 |
| Compound 53 | C14 Ether | 0.03 | 4.2 | 2.8 | 5 |
| Compound 6 | PEG2K-DMG | 0.03 | 26.8 | 4.9 | 5 |
| Compound 6 | C13 Ether | 0.03 | 27.4 | 5.4 | 5 |
| Compound 6 | C14 Ether | 0.03 | 18.9 | 5.8 | 5 |
| Compound 60 | PEG2K-DMG | 0.03 | 12.0 | 4.1 | 5 |
| Compound 60 | C13 Ether | 0.03 | 10.1 | 3.4 | 5 |
| Compound 60 | C14 Ether | 0.03 | 6.4 | 1.6 | 5 |

### Example 56—Compound 62

### Compound 62: 3-((((1-ethylpiperidin-3-yl)methoxy)carbonyl)oxy)-2-((((9Z,12Z)-octadeca-9,12-dienoyl)oxy)methyl)propyl heptadecan-9-yl glutarate

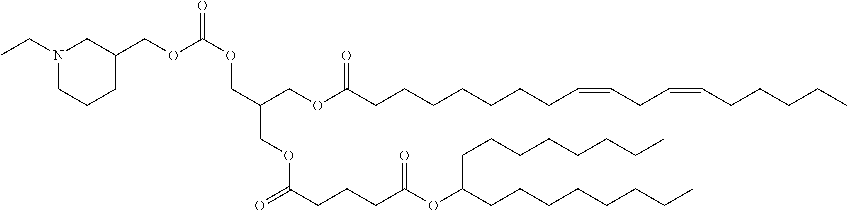 [SMILES: CCCCC/C=C\C/C=C\CCCCCCCC(=O)OCC(COC(=O)CCCC(=O)OC(CCCCCCCC)CCCCCCCC)COC(=O)OCC1CCCN(CC)C1]

Compound 62 was synthesized from Intermediate 49b and (1-ethylpiperidin-3-yl)methanol using the method employed in the synthesis of Compound 6. ¹H NMR (500 MHz, CDCl₃) δ 5.43-5.28 (m, 4H), 4.87 (p, J=6.3 Hz, 1H), 4.19 (d, J=5.9 Hz, 2H), 4.15 (t, J=6.1 Hz, 4H), 2.77 (t, J=6.7 Hz, 2H), 2.48-2.27 (m, 8H), 2.05 (q, J=7.0 Hz, 4H), 1.94 (p, J=7.4 Hz, 3H), 1.80-1.48 (m, 11H), 1.43-1.01 (m, 43H), 0.88 (q, J=6.8 Hz, 9H). MS: 891.7 m/z [M+H].

### Example 57—Compound 63

### Intermediate 63a: ethyl (E)-undec-2-enoate

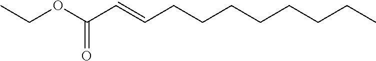

A solution of ethyl 2-(diethoxyphosphoryl)acetate (6.24 mL, 1.5 equiv.) in THF (0.5 M) was cooled to 0° C. and stirred for 20 min. Then, a solution of nonanal (3.62 mL, 1.0 equiv.) in THF (0.5 M) was added to the mixture, and the reaction was allowed to stir at 0° C. for an additional 30 min, followed by 2 h at room temperature. Next, a second portion of ethyl 2-(diethoxyphosphoryl)acetate (0.5 equiv.) in THF (1.0 M) was added, and the reaction was stirred overnight at room temperature. The resulting reaction was quenched by the addition of water, extracted 3× with EtOAc, and the combined organic layers were dried over Na₂SO₄, filtered, and concentrated. The crude residue was purified by column chromatography (EtOAc/hexanes) to afford product as a colorless oil (2.77 g, 62%). ¹H NMR (500 MHz, CDCl₃) δ 7.01 (dt, J=15.7, 7.0 Hz, 1H), 5.86 (dd, J=15.6, 1.8 Hz, 1H), 4.23 (q, J=7.1 Hz, 2H), 2.24 (qd, J=7.3, 1.4 Hz, 2H), 1.50 (p, J=7.3 Hz, 2H), 1.39-1.26 (m, J=5.5, 5.1 Hz, 14H), 0.93 (t, J=6.8 Hz, 3H).

### Intermediate 63b: ethyl 3-octylundecanoate

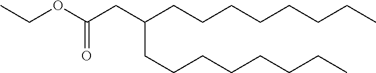 [SMILES: CCCCCCCCC(CCCCCCCC)CC(=O)OCC]

To a mixture of CuBr (143 mg, 0.1 equiv.) and LiCl (84 mg, 0.2 equiv.) was added THF (27 mL, 0.375 M), and the mixture was stirred for 10 min at room temperature. Then, the reaction was cooled to 0° C., and Intermediate 63a (2.13 g, 1.0 equiv.) in 1.6 mL THF, followed by chlorotrimethylsilane (1.51 mL, 1.2 equiv.). The mixture was stirred for 20 min at 0° C., followed by the addition of octyl magnesium bromide in THF (1.0 M, 13.3 mL, 1.2 equiv.). The resulting mixture was stirred for 2 hours at 0° C. The reaction was quenched by the addition of sat. NH₄Cl, and the mixture was extracted 3× with EtOAc. The resulting organic layers were washed with brine, dried over Na₂SO₄, filtered, and concentrated under reduced pressure. The resulting crude residue was purified by column chromatography (EtOAc/hexanes) to afford product as a colorless oil (3.12 g, 95%). ¹H NMR (400 MHz, CDCl₃) δ 4.12 (q, J=7.2 Hz, 2H), 2.21 (d, J=6.9 Hz, 2H), 1.85 (d, J=6.6 Hz, 1H), 1.25 (d, J=1.7 Hz, 29H), 0.92-0.84 (m, 6H).

### Intermediate 63c: 3-octylundecanoic Acid

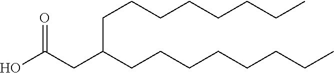 [SMILES: CCCCCCCCC(CCCCCCCC)CC(=O)O]

To Intermediate 63b (1.04 g, 1.0 equiv.) was added 1 M NaOH (15.9 mL, 5.0 equiv.), and the mixture was stirred at 50° C. for 16 h. The resulting mixture was then concentrated in vacuo to remove EtOH, followed by the addition of 1 M HCl (until pH=1). The aqueous mixture was diluted with brine and extracted 3× with EtOAc. The resulting organic layers were washed with brine, dried over Na₂SO₄, filtered, and concentrated under reduced pressure. The resulting crude residue was purified by column chromatography (MeOH/DCM) to afford product as a pale yellow oil (584 mg, 62%). ¹H NMR (400 MHz, CDCl₃) δ 2.27 (d, J=6.9 Hz, 2H), 1.92-1.79 (m, 1H), 1.26 (s, 28H), 0.94-0.82 (m, 6H).

### Intermediate 63d: 3-(benzyloxy)-3-oxopropyl 3-octylundecanoate

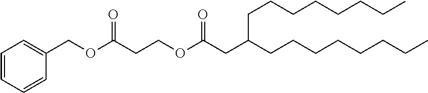 [SMILES: CCCCCCCCC(CCCCCCCC)CC(=O)OCCC(=O)OCc1ccccc1]

To a solution of Intermediate 63c (1.93 g, 1 equiv.) in THF (1.0 M) was added oxalyl chloride (609 μL, 1.1 equiv. and 60 μL of DMF (0.01 M). After 5 min, benzyl 3-hydroxypropanoate (1.39 g, 1.2 equiv. 0 was added, and the reaction was stirred at room temperature for 4 h. The mixture was then concentrated in vacuo, diluted with sat. NaHCO₃, and extracted 3× with EtOAc. The resulting organic layers were washed with brine, dried over Na₂SO₄, filtered, and concentrated under reduced pressure. The resulting crude residue was purified by column chromatography (EtOAc/hexanes) to afford product as a colorless oil (1.15 g, 39%). ¹H NMR (400 MHz, CDCl₃) δ 7.43-7.31 (m, 5H), 5.18 (s, 2H), 4.38 (t, J=6.4 Hz, 2H), 2.72 (t, J=6.4 Hz, 2H), 2.22 (d, J=6.9 Hz, 2H), 1.40-1.15 (m, 29H), 0.90 (t, J=6.8 Hz, 6H).

### Intermediate 63e: 3-(3-hydroxy-2-(hydroxymethyl)propoxy)-3-oxopropyl 3-octylundecanoate

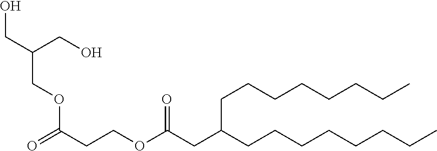 [SMILES: CCCCCCCCC(CCCCCCCC)CC(=O)OCCC(=O)OCC(CO)CO]

To a solution of Intermediate 63d (12.0 g, 1.0 equiv.) and 2-(hydroxymethyl)propane-1,3-diol (17.0 g, 5.0 equiv.) in DCM (0.25 M) was added triethylamine (13.4 mL, 3.0 equiv.), EDCI (9.27 g, 1.5 equiv.), and DMAP (3.94 g, 1.0 equiv.) in sequence, and the reaction was stirred overnight at room temperature. The reaction was diluted with water and extracted 3× with DCM. The resulting organic layers were washed with brine, dried over Na₂SO₄, filtered, and concentrated under reduced pressure. The resulting crude residue was purified by column chromatography (EtOAc/hexanes) to afford product as a colorless oil (2.79 g, 19%). ¹H NMR (400 MHz, CDCl₃) δ 4.39-4.28 (m, 4H), 3.85-3.72 (m, 4H), 2.68 (t, J=6.3 Hz, 2H), 2.22 (d, J=6.9 Hz, 2H), 2.11-1.99 (m, 2H), 1.81 (s, 1H), 1.34-1.28 (m, 3H), 1.25 (t, J=2.2 Hz, 24H), 0.92-0.84 (m, 6H).

### Intermediate 63f: heptadecan-9-yl (3-hydroxy-2-(((3-((3-octylundecanoyl)oxy)propanoyl)oxy)methyl)propyl) glutarate

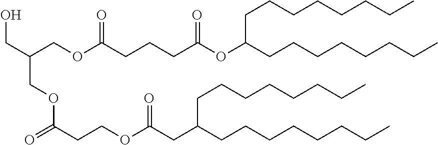 [SMILES: CCCCCCCCC(CCCCCCCC)CC(=O)OCCC(=O)OCC(CO)COC(=O)CCCC(=O)OC(CCCCCCCC)CCCCCCCC]

Intermediate 63f was synthesized (41%) from Intermediate 63e and Intermediate 2b using the methods employed in Intermediate 63e. ¹H NMR (400 MHz, CDCl₃) δ 4.86 (p, J=6.3 Hz, 1H), 4.34 (t, J=6.3 Hz, 2H), 4.20 (ddd, J=12.3, 6.1, 1.8 Hz, 4H), 3.63 (d, J=5.5 Hz, 2H), 2.66 (t, J=6.3 Hz, 2H), 2.38 (dt, J=18.0, 7.4 Hz, 4H), 2.21 (dd, J=9.3, 6.4 Hz, 4H), 1.95 (p, J=7.4 Hz, 2H), 1.81 (q, J=5.9, 5.3 Hz, 1H), 1.60-1.46 (m, 6H), 1.25 (s, 52H), 0.88 (t, J=6.7 Hz, 12H).

### Intermediate 63g: heptadecan-9-yl (3-(((4-nitrophenoxy)carbonyl)oxy)-2-(((3-((3-octylundecanoyl)oxy)propanoyl)oxy)methyl)propyl) glutarate

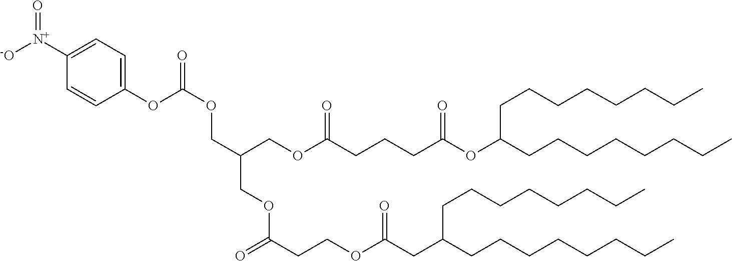

To a solution of Intermediate 63f (935 mg, 1.0 equiv.) in DCM (0.1-0.2 M) was added (4-nitrophenyl) carbonochloridate (1.5 equiv.) and pyridine (2.0 equiv.). The mixture was stirred at 25° C. for 16-20 h under N₂. The reaction mixture was diluted with water and extracted 3× with DCM. The resulting organic layers were washed with brine, dried over Na₂SO₄, filtered, and concentrated under reduced pressure. The resulting crude residue was purified by column chromatography (EtOAc/hexanes) to afford product as a white slurry (1.06 g, 95%). ¹H NMR (400 MHz, CDCl₃) δ 8.31-8.26 (m, 2H), 7.44-7.35 (m, 2H), 4.86 (p, J=6.3 Hz, 1H), 4.40-4.31 (m, 4H), 4.24 (ddd, J=13.1, 6.0, 2.2 Hz, 4H), 2.68 (t, J=6.4 Hz, 2H), 2.54 (h, J=6.0 Hz, 1H), 2.39 (dt, J=23.1, 7.4 Hz, 4H), 2.22 (d, J=6.9 Hz, 2H), 1.96 (p, J=7.4 Hz, 2H), 1.50 (d, J=6.3 Hz, 4H), 1.25 (s, 53H), 0.87 (t, J=6.8 Hz, 12H).

### Compound 63: 3-(((2-(diethylamino)ethyl)carbamoyl)oxy)-2-(((3-((3-octylundecanoyl)oxy)propanoyl)oxy)methyl)propyl heptadecan-9-yl Glutarate

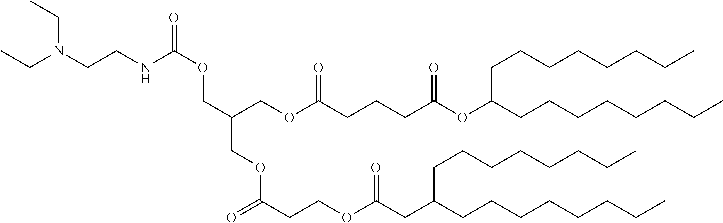 [SMILES: CCCCCCCCC(CCCCCCCC)CC(=O)OCCC(=O)OCC(COC(=O)CCCC(=O)OC(CCCCCCCC)CCCCCCCC)COC(=O)NCCN(CC)CC]

To a solution of Intermediate 63g (200 mg, 1.0 equiv.) in MeCN (0.1 M) was added (2-aminoethyl)diethylamine (36 mg, 1.5 equiv.), pyridine (49 uL, 3.0 equiv.), and DMAP (2.5 mg, 0.1 equiv.) in sequence. The reaction was allowed to stir at room temperature for 16 h. Heptane was added to the mixture, and the mixture was washed 3× with MeCN. The combined MeCN layers were combined, extracted 2× with heptane, and all heptane layers were combined, concentrated, and purified by column chromatography (MeOH/DC) to afford product (127 mg, 65%) as a colorless oil. ¹H NMR (400 MHz, CDCl₃) δ 4.86 (p, J=6.3 Hz, 1H), 4.32 (t, J=6.5 Hz, 2H), 4.21-4.08 (m, 6H), 3.42 (s, 2H), 2.82 (s, 4H), 2.66 (t, J=6.5 Hz, 3H), 2.36 (dt, J=14.8, 7.4 Hz, 5H), 2.22 (d, J=6.9 Hz, 2H), 1.94 (p, J=7.5 Hz, 2H), 1.82 (s, 1H), 1.68-1.44 (m, 9H), 1.25 (s, 59H), 0.91-0.84 (m, 12H). MS: 954.8 m/z [M+H].

### Example 58—Assessing Lipid Efficacy by In Vivo Editing

In vivo editing efficiency for materials delivered with formulations including various amine lipid (or ionizable lipid) compounds as described in Tables 7 and 8 was assessed. Editing was measured using G000534 (SEQ ID NO: 5) which targets the rat TTR gene. Lipids described above were assessed for efficacy through in vivo editing experiments. LNPs were formulated at a 1:2 w/w ratio of sgRNA (SEQ ID NO: 5) and Cas9 mRNA (SEQ ID NO: 3). Lipid components of the LNP were formulated in a molar ratio of 50% ionizable lipid, 38% cholesterol, 9% DSPC, and 3% C13 Ether. The final LNPs used in different in vivo editing experiments were characterized to determine the encapsulation efficiency (% E), polydispersity index (PDI), and average particle size (Z-avg and number mean) according to the analytical methods provided above. Results of the composition analysis are shown in Table 7. The pKa values of each of the following ionizable lipids were also measured to be in the range of about 5.8 to about 7.6.

TABLE 7
Composition Analytics.

| | | | | | | Number |
|---|---|---|---|---|---|---|
| | | | | Z-avg | | mean |
| LNP# | Ionizable Lipid | sgRNA:mRNA | % E | (nm) | PDI | (nm) |
| 56 | Compound 6 | 1:2 | 98 | 78 | 0.01 | 71 |
| 68 | Compound 52 | 1:2 | 97 | 85 | 0.01 | 79 |
| 69 | Compound 62 | 1:2 | 99 | 71 | 0.04 | 62 |
| 70 | Compound 63 | 1:2 | 96 | 80 | 0.02 | 73 |

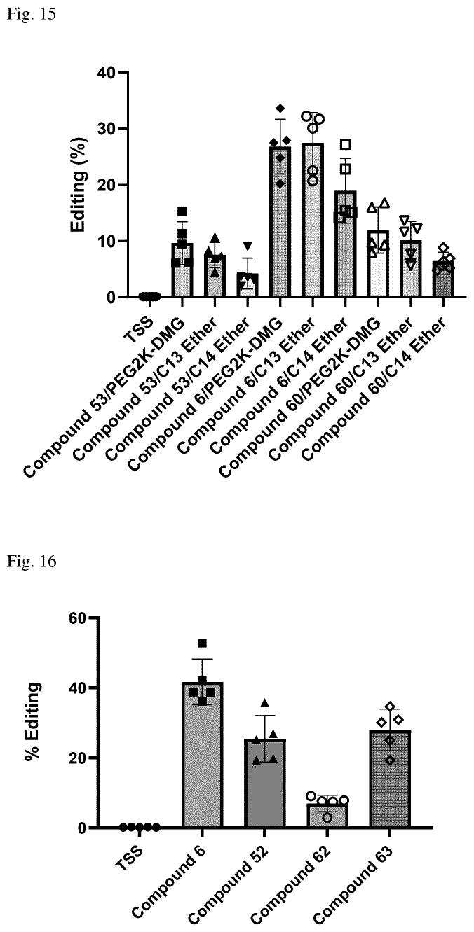

Table 8 shows editing percentages in rat liver as measured by NGS. TSS was used as a vehicle-only negative control. The data are illustrated in FIG. 16.

TABLE 8
Editing efficiency in rat liver measured by % editing.

| Ionizable | Dose | Mean % | | |
|---|---|---|---|---|
| Lipid | (mpk) | editing | SD | N |
| TSS | TSS | 0.1 | 0.1 | 5 |
| Compound 6 | 0.03 | 41.7 | 6.6 | 5 |
| Compound 52 | 0.03 | 7.0 | 2.4 | 5 |
| Compound 62 | 0.03 | 28.0 | 5.9 | 5 |
| Compound 63 | 0.03 | 0.1 | 0.1 | 5 |

### Example 59—Compound 64

### Compound 64: 7,7′-di(heptadecan-9-yl) O′1,O1-(2-((((3-(pyrrolidin-1-yl)propoxy)carbonyl)oxy)methyl)propane-1,3-diyl) di(heptanedioate)

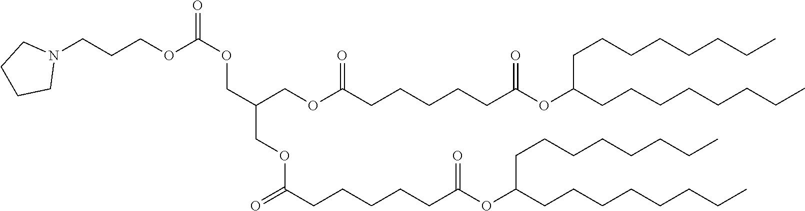 [SMILES: CCCCCCCCC(CCCCCCCC)OC(=O)CCCCCC(=O)OCC(COC(=O)CCCCCC(=O)OC(CCCCCCCC)CCCCCCCC)COC(=O)OCCCN1CCCC1]

Compound 64 was synthesized from Intermediate 38c and 3-pyrrolidin-1-ylpropan-1-ol (4.0 equiv.) using the method employed in the synthesis of Compound 6. ¹H NMR (500 MHz, CDCl₃) δ 64.86 (p, J=6.3 Hz, 2H), 4.24-4.08 (m, 7H), 2.55-2.47 (m, 5H), 2.42 (p, J=6.0 Hz, 1H), 2.30 (dt, J=15.4, 7.5 Hz, 8H), 1.89 (dq, J=8.3, 6.7 Hz, 2H), 1.80-1.75 (m, 3H), 1.66 (d, J=7.0 Hz, 3H), 1.64-1.60 (m, 5H), 1.57 (s, 2H), 1.50 (d, J=6.2 Hz, 8H), 1.39-1.32 (m, 5H), 1.32-1.19 (m, 53H), 0.88 (t, J=6.9 Hz, 15H). MS: 1023.9 m/z [M+H]. pKa: 6.4.

### Example 60—Compound 65

### Compound 65: 8,8′-di(heptadecan-9-yl) O′1,O1-(2-((((3-(pyrrolidin-1-yl)propoxy)carbonyl)oxy)methyl)propane-1,3-diyl) di(octanedioate)

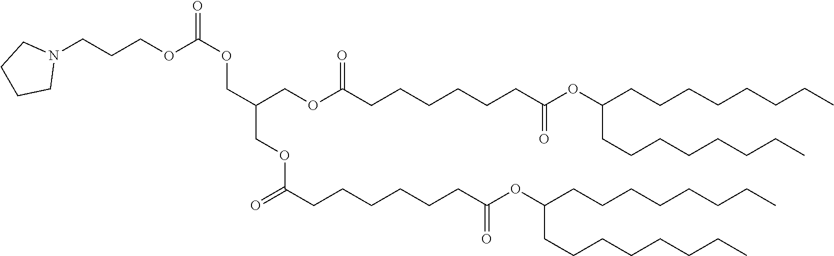 [SMILES: CCCCCCCCC(CCCCCCCC)OC(=O)CCCCCCC(=O)OCC(COC(=O)CCCCCCC(=O)OC(CCCCCCCC)CCCCCCCC)COC(=O)OCCCN1CCCC1]

Compound 65 was synthesized from Intermediate 52c and 3-pyrrolidin-1-ylpropan-1-ol (4.0 equiv.) using the method employed in the synthesis of Compound 6. ¹H NMR (500 MHz, CDCl₃) δ 4.86 (p, J=6.3 Hz, 2H), 4.19 (dt, J=7.7, 5.8 Hz, 4H), 4.18-4.07 (m, 4H), 2.56-2.47 (m, 5H), 2.42 (p, J=6.0 Hz, 1H), 2.29 (dt, J=15.3, 7.5 Hz, 7H), 1.92-1.85 (m, 2H), 1.80-1.74 (m, 3H), 1.61 (d, J=7.1 Hz, 8H), 1.50 (d, J=8.3 Hz, 8H), 1.37-1.19 (m, 62H), 0.88 (t, J=6.9 Hz, 15H). MS: 1052.2 m/z [M+H]. pKa: 6.4.

### Example 61—Compound 66

### Intermediate 66a: O′1,O1-(2-ethyl-2-(hydroxymethyl)propane-1,3-diyl) 8,8′-di(heptadecan-9-yl) di(octanedioate)

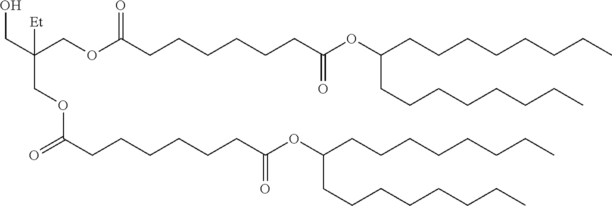 [SMILES: CCCCCCCCC(CCCCCCCC)OC(=O)CCCCCCC(=O)OCC(CC)(CO)COC(=O)CCCCCCC(=O)OC(CCCCCCCC)CCCCCCCC]

Intermediate 66a was synthesized from Intermediate 52a and 2-ethyl-2-(hydroxymethyl)propane-1,3-diol using the method employed in the synthesis of Intermediate 2c. ¹H NMR (500 MHz, CDCl₃) δ 4.86 (p, J=6.3 Hz, 2H), 4.03 (s, 3H), 3.40 (s, 2H), 2.30 (dt, J=26.0, 7.5 Hz, 8H), 1.67-1.46 (m, 22H), 1.43-1.32 (m, 11H), 1.26 (d, J=7.4 Hz, 49H), 0.88 (td, J=7.3, 4.6 Hz, 15H).

### Intermediate 66b: O′1,O1-(2-ethyl-2-((((4-nitrophenoxy)carbonyl)oxy)methyl)propane-1,3-diyl) 8,8′-di(heptadecan-9-yl) di(octanedioate)

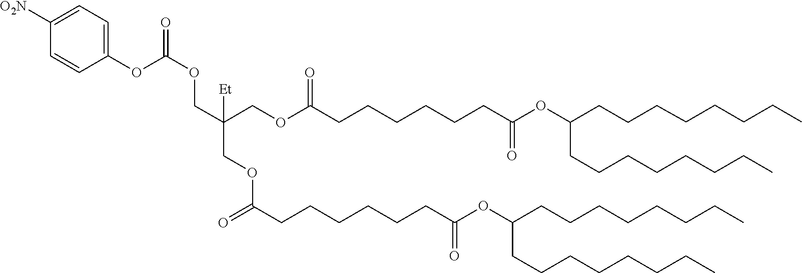 [SMILES: CCCCCCCCC(CCCCCCCC)OC(=O)CCCCCCC(=O)OCC(CC)(COC(=O)CCCCCCC(=O)OC(CCCCCCCC)CCCCCCCC)COC(=O)Oc1ccc([N+](=O)[O-])cc1]

Intermediate 66b was synthesized from Intermediate 66a using the method employe in Example 52c. ¹H NMR (400 MHz, CDCl₃) δ 8.33-8.25 (m, 2H), 7.43-7.35 (m, 2H), 4.86 (p, J=6.3 Hz, 2H), 4.23 (s, 2H), 4.09 (s, 3H), 2.30 (dt, J=23.5, 7.5 Hz, 7H), 1.68-1.45 (m, 23H), 1.38-1.19 (m, 49H), 0.98-0.83 (m, 13H).

### Compound 66: O′1,O1-(2-((((2-(diethylamino)ethyl)carbamoyl)oxy)methyl)-2-ethylpropane-1,3-diyl) 8,8′-di(heptadecan-9-yl) di(octanedioate)

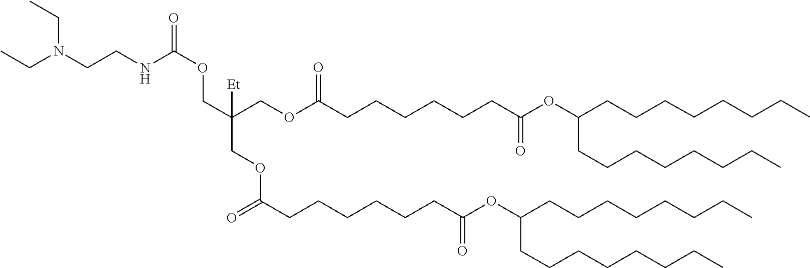 [SMILES: CCCCCCCCC(CCCCCCCC)OC(=O)CCCCCCC(=O)OCC(CC)(COC(=O)CCCCCCC(=O)OC(CCCCCCCC)CCCCCCCC)COC(=O)NCCN(CC)CC]

Compound 66 was synthesized from Intermediate 66b using the method employed in the synthesis of Compound 52. ¹H NMR (500 MHz, CDCl₃) δ 5.16 (d, J=5.3 Hz, 1H), 4.86 (p, J=6.3 Hz, 2H), 4.01 (d, J=4.9 Hz, 5H), 3.20 (d, J=6.2 Hz, 2H), 2.54-2.49 (m, 5H), 2.29 (dt, J=14.9, 7.6 Hz, 7H), 1.53 (d, J=20.3 Hz, 17H), 1.36-1.22 (m, 66H), 1.00 (t, J=7.1 Hz, 6H), 0.90-0.85 (m, 18H). MS: 1066.3 m/z [M+H]. pKa: 6.1.

### Example 62—Compound 67

### Compound 67: O′1,O1-(2-ethyl-2-((((3-(pyrrolidin-1-yl)propoxy)carbonyl)oxy)methyl)propane-1,3-diyl) 8,8′-di(heptadecan-9-yl) di(octanedioate)

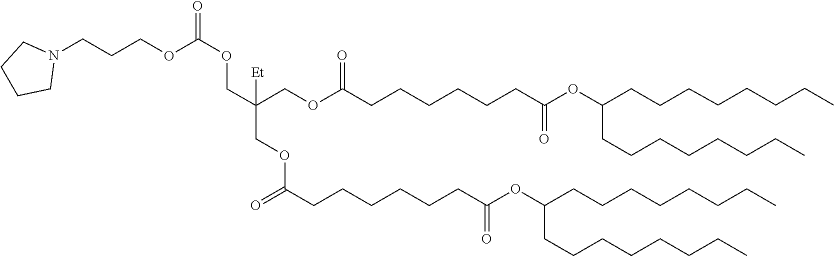 [SMILES: CCCCCCCCC(CCCCCCCC)OC(=O)CCCCCCC(=O)OCC(CC)(COC(=O)CCCCCCC(=O)OC(CCCCCCCC)CCCCCCCC)COC(=O)OCCCN1CCCC1]

Compound 67 was synthesized from Intermediate 66b and 3-pyrrolidin-1-ylpropan-1-ol (4.0 equiv.) using the method employed in the synthesis of Compound 6. ¹H NMR (500 MHz, CDCl₃) δ 4.86 (p, J=6.3 Hz, 2H), 4.19 (t, J=6.6 Hz, 2H), 4.08 (s, 1H), 4.02 (s, 4H), 2.51 (dt, J=13.3, 6.8 Hz, 5H), 2.29 (dt, J=15.1, 7.5 Hz, 7H), 1.88 (p, J=6.9 Hz, 2H), 1.77 (q, J=3.3, 2.9 Hz, 3H), 1.66-1.55 (m, 12H), 1.49 (p, J=8.0, 6.9 Hz, 10H), 1.36-1.18 (m, 65H), 0.88 (q, J=6.9 Hz, 19H). MS: 1079.2 m/z [M+H]. pKa: 6.2.

### Example 63—Compound 68

### Intermediate 68a: 6-(heptadecan-9-yloxy)-6-oxohexanoic Acid

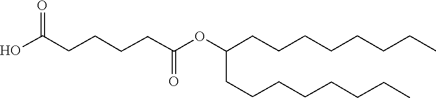 [SMILES: CCCCCCCCC(CCCCCCCC)OC(=O)CCCCC(=O)O]

To a solution of adipic acid (0.5 equiv.) in DCM (0.05 M) was added heptadecan-9-ol (1.0 equiv.), triethylamine (3.0 equiv.), EDCI (1.5 equiv.), and DMAP (0.5 equiv.) in sequence. The reaction was stirred at room temperature for 16 h, after which point the reaction was quenched by the addition of water. The biphasic mixture was extracted 3× with DCM and concentrated in vacuo before purification by column chromatography (EtOAc/hexanes) to afford product. ¹H NMR (400 MHz, CDCl₃) δ 4.87 (p, J=6.3 Hz, 1H), 2.43-2.36 (m, 2H), 2.31 (ddt, J=7.2, 5.0, 2.6 Hz, 2H), 1.68 (h, J=3.9 Hz, 4H), 1.25 (s, 24H), 0.93-0.82 (m, 6H).

### Intermediate 68b: di(heptadecan-9-yl) O,O′-(2-(hydroxymethyl)propane-1,3-diyl) diadipate

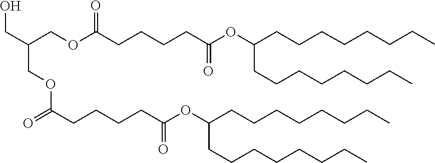 [SMILES: CCCCCCCCC(CCCCCCCC)OC(=O)CCCCC(=O)OCC(CO)COC(=O)CCCCC(=O)OC(CCCCCCCC)CCCCCCCC]

Intermediate 68b was synthesized from Intermediate 68a (1.0 equiv.) and 2-(hydroxymethyl)propane-1,3-diol (5.0 equiv.) using the method employed in the synthesis of Intermediate 2b. ¹H NMR (500 MHz, CDCl₃) δ 4.86 (p, J=6.3 Hz, 2H), 4.23-4.12 (m, 4H), 3.62 (d, J=5.4 Hz, 2H), 2.40-2.23 (m, 9H), 2.24-2.15 (m, 1H), 1.71-1.62 (m, 8H), 1.56-1.48 (m, 9H), 1.26 (d, J=7.2 Hz, 50H), 0.88 (t, J=6.9 Hz, 12H).

### Intermediate 68c: di(heptadecan-9-yl) O,O′-(2-((((4-nitrophenoxy)carbonyl)oxy)methyl)propane-1,3-diyl) diadipate

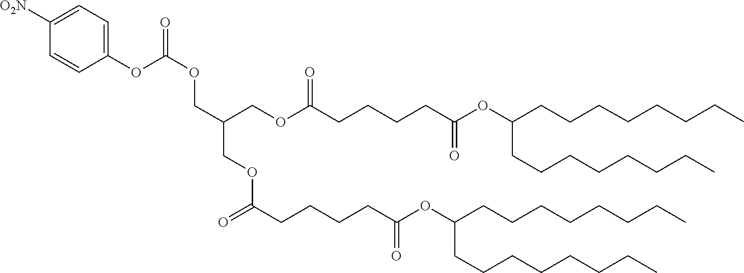 [SMILES: CCCCCCCCC(CCCCCCCC)OC(=O)CCCCC(=O)OCC(COC(=O)CCCCC(=O)OC(CCCCCCCC)CCCCCCCC)COC(=O)Oc1ccc([N+](=O)[O-])cc1]

Intermediate 68c was synthesized from Intermediate 68b using the method employed in Example 52c. ¹H NMR (500 MHz, CDCl₃) δ 8.32-8.26 (m, 2H), 7.43-7.36 (m, 2H), 4.86 (p, J=6.3 Hz, 2H), 4.36 (d, J=5.8 Hz, 2H), 4.22-4.16 (m, 3H), 2.51 (p, J=6.0 Hz, 1H), 2.40-2.27 (m, 8H), 1.71-1.62 (m, 8H), 1.50 (d, J=6.2 Hz, 8H), 1.34-1.19 (m, 49H), 0.87 (t, J=6.9 Hz, 12H).

### Compound 68: O,O′-(2-((((2-(diethylamino)ethyl)carbamoyl)oxy)methyl)propane-1,3-diyl) di(heptadecan-9-yl) diadipate

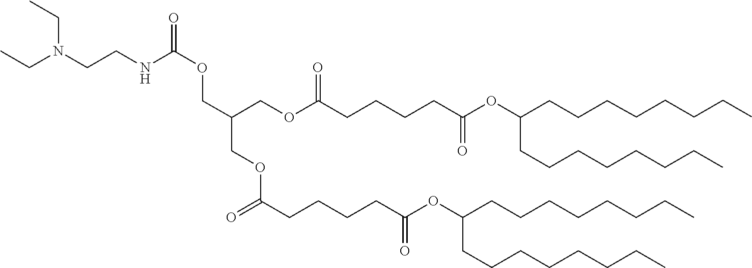 [SMILES: CCCCCCCCC(CCCCCCCC)OC(=O)CCCCC(=O)OCC(COC(=O)CCCCC(=O)OC(CCCCCCCC)CCCCCCCC)COC(=O)NCCN(CC)CC]

Compound 68 was synthesized from Intermediate 68c using the method employed in the synthesis of Compound 52. ¹H NMR (500 MHz, CDCl₃) δ 5.22 (s, 1H), 4.86 (p, J=6.3 Hz, 2H), 4.12 (d, J=5.8 Hz, 6H), 3.21 (d, J=6.3 Hz, 2H), 2.52 (q, J=7.4, 6.9 Hz, 5H), 2.37-2.27 (m, 8H), 1.65 (t, J=3.6 Hz, 7H), 1.55 (s, 4H), 1.50 (d, J=6.1 Hz, 8H), 1.25 (s, 58H), 1.00 (t, J=7.1 Hz, 6H), 0.88 (t, J=6.9 Hz, 15H). MS: 982.9 m/z [M+H]. pKa: 6.4.

### Example 64—Compound 69

### Compound 69: di(heptadecan-9-yl) O,O′-(2-((((3-(pyrrolidin-1-yl)propoxy)carbonyl)oxy)methyl)propane-1,3-diyl) diadipate

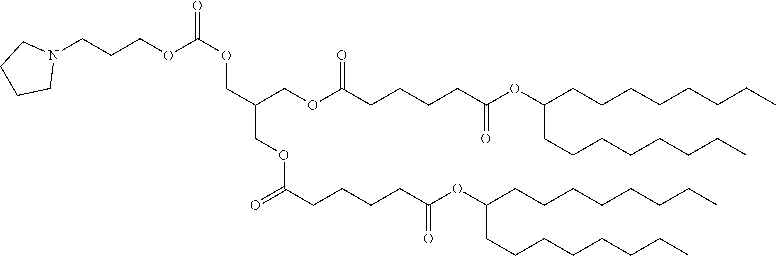 [SMILES: CCCCCCCCC(CCCCCCCC)OC(=O)CCCCC(=O)OCC(COC(=O)CCCCC(=O)OC(CCCCCCCC)CCCCCCCC)COC(=O)OCCCN1CCCC1]

Compound 69 was synthesized from Intermediate 68c and 3-pyrrolidin-1-ylpropan-1-ol (4.0 equiv.) using the method employed in the synthesis of Compound 6. ¹H NMR (500 MHz, CDCl₃) δ 4.86 (p, J=6.2 Hz, 2H), 4.24-4.13 (m, 7H), 2.55-2.47 (m, 5H), 2.42 (p, J=6.0 Hz, 1H), 2.36-2.28 (m, 7H), 1.89 (dq, J=8.4, 6.6 Hz, 2H), 1.80-1.75 (m, 3H), 1.69-1.63 (m, 8H), 1.56 (s, 4H), 1.50 (q, J=6.3 Hz, 9H), 1.25 (s, 54H), 0.93-0.81 (m, 16H). MS: 996.3 m/z [M+H]. pKa: 6.4.

### Example 65—Assessing Lipid Efficacy by In Vivo Editing

In vivo editing efficiency for materials delivered with formulations including various amine lipid (or ionizable lipid) compounds as described in Tables 9 and 10 was assessed. Editing was measured using G000534 (SEQ ID NO: 5) which targets the rat TTR gene. Lipids described above were assessed for efficacy through in vivo editing experiments. LNPs were formulated at a 1:2 w/w ratio of sgRNA (SEQ ID NO: 5) and Cas9 mRNA (SEQ ID NO: 3). Lipid components of the LNP were formulated in a molar ratio of 50% ionizable lipid, 38% cholesterol, 9% DSPC, and 3% C13 Ether, except LNP #1 was formulated with 3% PEG2K-DMG instead of 3% C13 Ether. The final LNPs used in different in vivo editing experiments were characterized to determine the encapsulation efficiency (% E), polydispersity index (PDI), and average particle size (Z-avg and number mean) according to the analytical methods provided above. Results of the composition analysis are shown in Table 9. The pKa values of each of the following ionizable lipids were also measured to be in the range of about 5.8 to about 7.6.

TABLE 9
Composition Analytics.

| | | | | | | Number |
|---|---|---|---|---|---|---|
| | | | | Z-avg | | mean |
| LNP# | Ionizable Lipid | sgRNA:mRNA | % E | (nm) | PDI | (nm) |
| 1 | Compound 53 | 1:2 | 98 | 87 | 0.04 | 71 |
| 56 | Compound 6 | 1:2 | 99 | 81 | 0.002 | 67 |
| 71 | Compound 68 | 1:2 | 99 | 75 | 0.01 | 63 |
| 72 | Compound 66 | 1:2 | 99 | 78 | 0.01 | 64 |
| 73 | Compound 33 | 1:2 | 99 | 73 | 0.01 | 60 |
| 74 | Compound 69 | 1:2 | 99 | 71 | 0.004 | 59 |
| 75 | Compound 64 | 1:2 | 99 | 63 | 0.01 | 52 |
| 76 | Compound 65 | 1:2 | 99 | 74 | 0.01 | 62 |
| 77 | Compound 67 | 1:2 | 99 | 65 | 0.03 | 53 |
| 68 | Compound 52 | 1:2 | 97 | 88 | 0.02 | 74 |

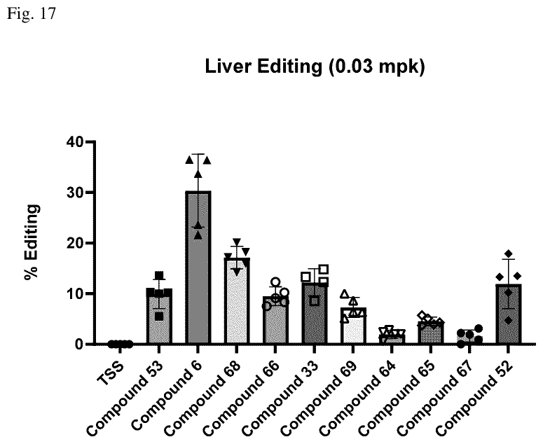

Table 10 shows editing percentages in rat liver as measured by NGS. TSS was used as a vehicle-only negative control. The data are illustrated in FIG. 17.

TABLE 10
Editing efficiency in rat liver measured by % editing.

| | Dose | Mean % | | |
|---|---|---|---|---|
| Ionizable Lipid | (mpk) | editing | SD | N |
| TSS | TSS | 0.0 | 0.0 | 5 |
| Compound 53 | 0.03 | 9.9 | 2.9 | 5 |
| Compound 6 | 0.03 | 30.4 | 7.2 | 5 |
| Compound 68 | 0.03 | 17.1 | 2.2 | 5 |
| Compound 66 | 0.03 | 9.5 | 1.9 | 5 |
| Compound 33 | 0.03 | 12.2 | 2.7 | 4 |
| Compound 69 | 0.03 | 7.3 | 2.0 | 5 |
| Compound 64 | 0.03 | 2.0 | 0.8 | 5 |
| Compound 65 | 0.03 | 4.5 | 0.8 | 5 |
| Compound 67 | 0.03 | 1.6 | 1.2 | 5 |
| Compound 52 | 0.03 | 11.9 | 4.9 | 5 |

In the following table and throughout, the terms “mA,” “mC,” “mU,” or “mG” are used to denote a nucleotide that has been modified with 2′-O-Me.

In the following table, a “*” is used to depict a PS modification. In this application, the terms A*, C*, U*, or G* may be used to denote a nucleotide that is linked to the next (e.g., 3′) nucleotide with a PS bond.

It is understood that if a DNA sequence (comprising Ts) is referenced with respect to an RNA, then Ts should be replaced with Us (which may be modified or unmodified depending on the context), and vice versa.

In the following table, single amino acid letter code is used to provide peptide sequences.

TABLE 11
List of sequences

| | SEQ ID | |
|---|---|---|
| Description | NO | Sequence |
| 1X NLS | SEQ ID | CCCAAGAAGAAGCGGAAGGUG |
| nucleotide | NO: 1 | |
| sequence | | |
| G000534 full | SEQ ID | ACGCAAAUAUCAGUCCAGCGGUUUUAGAGCUA |
| sequence | NO: 2 | GAAAUAGCAAGUUAAAAUAAGGCUAGUCCGU |
| | | UAUCAACUUGAAAAAGUGGCACCGAGUCGGU |
| | | GCUUUU |
| Cas9 mRNA | SEQ ID | GGGAAGCUCAGAAUAAACGCUCAACUUUGGCC |
| sequence | NO: 3 | GGAUCUGCCACCAUGGACAAGAAGUACUCCAU |
| | | CGGCCUGGACAUCGGCACCAACUCCGUGGGCU |
| | | GGGCCGUGAUCACCGACGAGUACAAGGUGCCC |
| | | UCCAAGAAGUUCAAGGUGCUGGGCAACACCGA |
| | | CCGGCACUCCAUCAAGAAGAACCUGAUCGGCG |
| | | CCCUGCUGUUCGACUCCGGCGAGACCGCCGAG |
| | | GCCACCCGGCUGAAGCGGACCGCCCGGCGGCG |
| | | GUACACCCGGCGGAAGAACCGGAUCUGCUACC |
| | | UGCAGGAGAUCUUCUCCAACGAGAUGGCCAAG |
| | | GUGGACGACUCCUUCUUCCACCGGCUGGAGGA |
| | | GUCCUUCCUGGUGGAGGAGGACAAGAAGCACG |
| | | AGCGGCACCCCAUCUUCGGCAACAUCGUGGAC |
| | | GAGGUGGCCUACCACGAGAAGUACCCCACCAU |
| | | CUACCACCUGCGGAAGAAGCUGGUGGACUCCA |
| | | CCGACAAGGCCGACCUGCGGCUGAUCUACCUG |
| | | GCCCUGGCCCACAUGAUCAAGUUCCGGGGCCA |
| | | CUUCCUGAUCGAGGGCGACCUGAACCCCGACA |
| | | ACUCCGACGUGGACAAGCUGUUCAUCCAGCUG |
| | | GUGCAGACCUACAACCAGCUGUUCGAGGAGAA |
| | | CCCCAUCAACGCCUCCGGCGUGGACGCCAAGG |
| | | CCAUCCUGUCCGCCCGGCUGUCCAAGUCCCGG |
| | | CGGCUGGAGAACCUGAUCGCCCAGCUGCCCGG |
| | | CGAGAAGAAGAACGGCCUGUUCGGCAACCUGA |
| | | UCGCCCUGUCCCUGGGCCUGACCCCCAACUUC |
| | | AAGUCCAACUUCGACCUGGCCGAGGACGCCAA |
| | | GCUGCAGCUGUCCAAGGACACCUACGACGACG |
| | | ACCUGGACAACCUGCUGGCCCAGAUCGGCGAC |
| | | CAGUACGCCGACCUGUUCCUGGCCGCCAAGAA |
| | | CCUGUCCGACGCCAUCCUGCUGUCCGACAUCC |
| | | UGCGGGUGAACACCGAGAUCACCAAGGCCCCC |
| | | CUGUCCGCCUCCAUGAUCAAGCGGUACGACGA |
| | | GCACCACCAGGACCUGACCCUGCUGAAGGCCC |
| | | UGGUGCGGCAGCAGCUGCCCGAGAAGUACAAG |
| | | GAGAUCUUCUUCGACCAGUCCAAGAACGGCUA |
| | | CGCCGGCUACAUCGACGGCGGCGCCUCCCAGG |
| | | AGGAGUUCUACAAGUUCAUCAAGCCCAUCCUG |
| | | GAGAAGAUGGACGGCACCGAGGAGCUGCUGG |
| | | UGAAGCUGAACCGGGAGGACCUGCUGCGGAAG |
| | | CAGCGGACCUUCGACAACGGCUCCAUCCCCCA |
| | | CCAGAUCCACCUGGGCGAGCUGCACGCCAUCC |
| | | UGCGGCGGCAGGAGGACUUCUACCCCUUCCUG |
| | | AAGGACAACCGGGAGAAGAUCGAGAAGAUCC |
| | | UGACCUUCCGGAUCCCCUACUACGUGGGCCCC |
| | | CUGGCCCGGGGCAACUCCCGGUUCGCCUGGAU |
| | | GACCCGGAAGUCCGAGGAGACCAUCACCCCCU |
| | | GGAACUUCGAGGAGGUGGUGGACAAGGGCGC |
| | | CUCCGCCCAGUCCUUCAUCGAGCGGAUGACCA |
| | | ACUUCGACAAGAACCUGCCCAACGAGAAGGUG |
| | | CUGCCCAAGCACUCCCUGCUGUACGAGUACUU |
| | | CACCGUGUACAACGAGCUGACCAAGGUGAAGU |
| | | ACGUGACCGAGGGCAUGCGGAAGCCCGCCUUC |
| | | CUGUCCGGCGAGCAGAAGAAGGCCAUCGUGGA |
| | | CCUGCUGUUCAAGACCAACCGGAAGGUGACCG |
| | | UGAAGCAGCUGAAGGAGGACUACUUCAAGAA |
| | | GAUCGAGUGCUUCGACUCCGUGGAGAUCUCCG |
| | | GCGUGGAGGACCGGUUCAACGCCUCCCUGGGC |
| | | ACCUACCACGACCUGCUGAAGAUCAUCAAGGA |
| | | CAAGGACUUCCUGGACAACGAGGAGAACGAGG |
| | | ACAUCCUGGAGGACAUCGUGCUGACCCUGACC |
| | | CUGUUCGAGGACCGGGAGAUGAUCGAGGAGC |
| | | GGCUGAAGACCUACGCCCACCUGUUCGACGAC |
| | | AAGGUGAUGAAGCAGCUGAAGCGGCGGCGGU |
| | | ACACCGGCUGGGGCCGGCUGUCCCGGAAGCUG |
| | | AUCAACGGCAUCCGGGACAAGCAGUCCGGCAA |
| | | GACCAUCCUGGACUUCCUGAAGUCCGACGGCU |
| | | UCGCCAACCGGAACUUCAUGCAGCUGAUCCAC |
| | | GACGACUCCCUGACCUUCAAGGAGGACAUCCA |
| | | GAAGGCCCAGGUGUCCGGCCAGGGCGACUCCC |
| | | UGCACGAGCACAUCGCCAACCUGGCCGGCUCC |
| | | CCCGCCAUCAAGAAGGGCAUCCUGCAGACCGU |
| | | GAAGGUGGUGGACGAGCUGGUGAAGGUGAUG |
| | | GGCCGGCACAAGCCCGAGAACAUCGUGAUCGA |
| | | GAUGGCCCGGGAGAACCAGACCACCCAGAAGG |
| | | GCCAGAAGAACUCCCGGGAGCGGAUGAAGCGG |
| | | AUCGAGGAGGGCAUCAAGGAGCUGGGCUCCCA |
| | | GAUCCUGAAGGAGCACCCCGUGGAGAACACCC |
| | | AGCUGCAGAACGAGAAGCUGUACCUGUACUAC |
| | | CUGCAGAACGGCCGGGACAUGUACGUGGACCA |
| | | GGAGCUGGACAUCAACCGGCUGUCCGACUACG |
| | | ACGUGGACCACAUCGUGCCCCAGUCCUUCCUG |
| | | AAGGACGACUCCAUCGACAACAAGGUGCUGAC |
| | | CCGGUCCGACAAGAACCGGGGCAAGUCCGACA |
| | | ACGUGCCCUCCGAGGAGGUGGUGAAGAAGAU |
| | | GAAGAACUACUGGCGGCAGCUGCUGAACGCCA |
| | | AGCUGAUCACCCAGCGGAAGUUCGACAACCUG |
| | | ACCAAGGCCGAGCGGGGCGGCCUGUCCGAGCU |
| | | GGACAAGGCCGGCUUCAUCAAGCGGCAGCUGG |
| | | UGGAGACCCGGCAGAUCACCAAGCACGUGGCC |
| | | CAGAUCCUGGACUCCCGGAUGAACACCAAGUA |
| | | CGACGAGAACGACAAGCUGAUCCGGGAGGUGA |
| | | AGGUGAUCACCCUGAAGUCCAAGCUGGUGUCC |
| | | GACUUCCGGAAGGACUUCCAGUUCUACAAGGU |
| | | GCGGGAGAUCAACAACUACCACCACGCCCACG |
| | | ACGCCUACCUGAACGCCGUGGUGGGCACCGCC |
| | | CUGAUCAAGAAGUACCCCAAGCUGGAGUCCGA |
| | | GUUCGUGUACGGCGACUACAAGGUGUACGACG |
| | | UGCGGAAGAUGAUCGCCAAGUCCGAGCAGGAG |
| | | AUCGGCAAGGCCACCGCCAAGUACUUCUUCUA |
| | | CUCCAACAUCAUGAACUUCUUCAAGACCGAGA |
| | | UCACCCUGGCCAACGGCGAGAUCCGGAAGCGG |
| | | CCCCUGAUCGAGACCAACGGCGAGACCGGCGA |
| | | GAUCGUGUGGGACAAGGGCCGGGACUUCGCCA |
| | | CCGUGCGGAAGGUGCUGUCCAUGCCCCAGGUG |
| | | AACAUCGUGAAGAAGACCGAGGUGCAGACCGG |
| | | CGGCUUCUCCAAGGAGUCCAUCCUGCCCAAGC |
| | | GGAACUCCGACAAGCUGAUCGCCCGGAAGAAG |
| | | GACUGGGACCCCAAGAAGUACGGCGGCUUCGA |
| | | CUCCCCCACCGUGGCCUACUCCGUGCUGGUGG |
| | | UGGCCAAGGUGGAGAAGGGCAAGUCCAAGAA |
| | | GCUGAAGUCCGUGAAGGAGCUGCUGGGCAUCA |
| | | CCAUCAUGGAGCGGUCCUCCUUCGAGAAGAAC |
| | | CCCAUCGACUUCCUGGAGGCCAAGGGCUACAA |
| | | GGAGGUGAAGAAGGACCUGAUCAUCAAGCUG |
| | | CCCAAGUACUCCCUGUUCGAGCUGGAGAACGG |
| | | CCGGAAGCGGAUGCUGGCCUCCGCCGGCGAGC |
| | | UGCAGAAGGGCAACGAGCUGGCCCUGCCCUCC |
| | | AAGUACGUGAACUUCCUGUACCUGGCCUCCCA |
| | | CUACGAGAAGCUGAAGGGCUCCCCCGAGGACA |
| | | ACGAGCAGAAGCAGCUGUUCGUGGAGCAGCAC |
| | | AAGCACUACCUGGACGAGAUCAUCGAGCAGAU |
| | | CUCCGAGUUCUCCAAGCGGGUGAUCCUGGCCG |
| | | ACGCCAACCUGGACAAGGUGCUGUCCGCCUAC |
| | | AACAAGCACCGGGACAAGCCCAUCCGGGAGCA |
| | | GGCCGAGAACAUCAUCCACCUGUUCACCCUGA |
| | | CCAACCUGGGCGCCCCCGCCGCCUUCAAGUAC |
| | | UUCGACACCACCAUCGACCGGAAGCGGUACAC |
| | | CUCCACCAAGGAGGUGCUGGACGCCACCCUGA |
| | | UCCACCAGUCCAUCACCGGCCUGUACGAGACC |
| | | CGGAUCGACCUGUCCCAGCUGGGCGGCGACGG |
| | | CGGCGGCUCCCCCAAGAAGAAGCGGAAGGUGU |
| | | GACUAGCACCAGCCUCAAGAACACCCGAAUGG |
| | | AGUCUCUAAGCUACAUAAUACCAACUUACACU |
| | | UUACAAAAUGUUGUCCCCCAAAAUGUAGCCAU |
| | | UCGUAUCUGCUCCUAAUAAAAAGAAAGUUUC |
| | | UUCACAUUCUCUCGAGAAAAAAAAAAAAAAA |
| | | AAAAAAAAAAAAAAAAAAAAAAAAAAAAAAA |
| | | AAAAAAAAAAAAAAAAAAAAAAAAAAAAAAA |
| | | AAAAAAAAAAAAAAAAUCUAG |
| amino acid | SEQ ID | PKKKRKV |
| sequence | NO: 4 | |
| encoded by SEQ | | |
| ID NO: 1 | | |
| G000534 full | SEQ ID | mA*mC*mG*CAAAUAUCAGUCCAGCGGUUUUA |
| modified | NO: 5 | GAmGmCmUmAmGmAmAmAmUmAmGmCAAGU |
| sequence | | UAAAAUAAGGCUAGUCCGUUAUCAmAmCmUm |
| | | UmGmAmAmAmAmAmGmUmGmGmCmAmCmCm |
| | | GmAmGmUmCmGmGmUmGmCmU*mU*mU*mU |
| G000534 target | SEQ ID | ACGCAAAUAUCAGUCCAGCG |
| sequence | NO: 6 | |
| SpyCas9 | SEQ ID | auggacaagaaguacuccaucggccuggacaucggcaccaacuccgug |
| mRNA42 - | NO: 7 | ggcugggccgugaucaccgacgaguacaaggugcccuccaagaaguuc |
| PINT416 ORF | | aaggugcugggcaacaccgaccggcacuccaucaagaagaaccugauc |
| sequence | | ggcgcccugcuguucgacuccggcgagaccgccgaggccacccggcug |
| | | aagcggaccgcccggcggcgguacacccggcggaagaaccggaucugc |
| | | uaccugcaggagaucuucuccaacgagauggccaagguggacgacucc |
| | | uucuuccaccggcuggaggaguccuuccugguggaggaggacaagaa |
| | | gcacgagcggcaccccaucuucggcaacaucguggacgagguggccua |
| | | ccacgagaaguaccccaccaucuaccaccugcggaagaagcuggugga |
| | | cuccaccgacaaggccgaccugcggcugaucuaccuggcccuggccca |
| | | caugaucaaguuccggggccacuuccugaucgagggcgaccugaaccc |
| | | cgacaacuccgacguggacaagcuguucauccagcuggugcagaccua |
| | | caaccagcuguucgaggagaaccccaucaacgccuccggcguggacgc |
| | | caaggccauccuguccgcccggcuguccaagucccggggcuggagaa |
| | | ccugaucgcccagcugcccggcgagaagaagaacggccuguucggcaa |
| | | ccugaucgcccugucccugggccugacccccaacuucaaguccaacuu |
| | | cgaccuggccgaggacgccaagcugcagcuguccaaggacaccuacga |
| | | cgacgaccuggacaaccugcuggcccagaucggcgaccaguacgccga |
| | | ccuguuccuggccgccaagaaccuguccgacgccauccugcuguccga |
| | | cauccugcgggugaacaccgagaucaccaaggccccccuguccgccuc |
| | | caugaucaagcgguacgacgagcaccaccaggaccugacccugcugaa |
| | | ggcccuggugcggcagcagcugcccgagaaguacaaggagaucuucuu |
| | | cgaccaguccaagaacggcuacgccggcuacaucgacggggcgccuc |
| | | ccaggaggaguucuacaaguucaucaagcccauccuggagaagaugga |
| | | cggcaccgaggagcugcuggugaagcugaaccgggaggaccugcugc |
| | | ggaagcagcggaccuucgacaacggcuccaucccccaccagauccaccu |
| | | gggcgagcugcacgccauccugcggcggcaggaggacuucuaccccuu |
| | | ccugaaggacaaccgggagaagaucgagaagauccugaccuuccggau |
| | | ccccuacuacgugggcccccuggcccggggcaacucccgguucgccug |
| | | gaugacccggaaguccgaggagaccaucacccccuggaacuucgagga |
| | | ggugguggacaagggcgccuccgcccaguccuucaucgagcggaugac |
| | | caacuucgacaagaaccugcccaacgagaaggugcugcccaagcacucc |
| | | cugcuguacgaguacuucaccguguacaacgagcugaccaaggugaag |
| | | uacgugaccgagggcaugcggaagcccgccuuccuguccggcgagcag |
| | | aagaaggccaucguggaccugcuguucaagaccaaccggaaggugacc |
| | | gugaagcagcugaaggaggacuacuucaagaagaucgagugcuucgac |
| | | uccguggagaucuccggcguggaggaccgguucaacgccucccuggg |
| | | caccuaccacgaccugcugaagaucaucaaggacaaggacuuccugga |
| | | caacgaggagaacgaggacauccuggaggacaucgugcugacccugac |
| | | ccuguucgaggaccgggagaugaucgaggagcggcugaagaccuacgc |
| | | ccaccuguucgacgacaaggugaugaagcagcugaagcggcggcggua |
| | | caccggcuggggccggcugucccggaagcugaucaacggcauccggga |
| | | caagcaguccggcaagaccauccuggacuuccugaaguccgacggcuu |
| | | cgccaaccggaacuucaugcagcugauccacgacgacucccugaccuu |
| | | caaggaggacauccagaaggcccagguguccggccagggcgacucccu |
| | | gcacgagcacaucgccaaccuggccggcucccccgccaucaagaagggc |
| | | auccugcagaccgugaaggugguggacgagcuggugaaggugauggg |
| | | ccggcacaagcccgagaacaucgugaucgagauggcccgggagaacca |
| | | gaccacccagaagggccagaagaacucccgggagcggaugaagcggau |
| | | cgaggagggcaucaaggagcugggcucccagauccugaaggagcaccc |
| | | cguggagaacacccagcugcagaacgagaagcuguaccuguacuaccu |
| | | gcagaacggccgggacauguacguggaccaggagcuggacaucaaccg |
| | | gcuguccgacuacgacguggaccacaucgugccccaguccuuccugaa |
| | | ggacgacuccaucgacaacaaggugcugacccgguccgacaagaaccg |
| | | gggcaaguccgacaacgugcccuccgaggagguggugaagaagaugaa |
| | | gaacuacuggcggcagcugcugaacgccaagcugaucacccagcggaa |
| | | guucgacaaccugaccaaggccgagcggggggccuguccgagcugga |
| | | caaggccggcuucaucaagcggcagcugguggagacccggcagaucac |
| | | caagcacguggcccagauccuggacucccggaugaacaccaaguacga |
| | | cgagaacgacaagcugauccgggaggugaaggugaucacccugaaguc |
| | | caagcugguguccgacuuccggaaggacuuccaguucuacaaggugcg |
| | | ggagaucaacaacuaccaccacgcccacgacgccuaccugaacgccgug |
| | | gugggcaccgcccugaucaagaaguaccccaagcuggaguccgaguuc |
| | | guguacggcgacuacaagguguacgacgugcggaagaugaucgccaag |
| | | uccgagcaggagaucggcaaggccaccgccaaguacuucuucuacucc |
| | | aacaucaugaacuucuucaagaccgagaucacccuggccaacggcgag |
| | | auccggaagcggccccugaucgagaccaacggcgagaccggcgagauc |
| | | gugugggacaagggccgggacuucgccaccgugcggaaggugcuguc |
| | | caugccccaggugaacaucgugaagaagaccgaggugcagaccggcgg |
| | | cuucuccaaggaguccauccugcccaagcggaacuccgacaagcugau |
| | | cgcccggaagaaggacugggaccccaagaaguacggcggcuucgacuc |
| | | ccccaccguggccuacuccgugcuggugguggccaagguggagaagg |
| | | gcaaguccaagaagcugaaguccgugaaggagcugcugggcaucacca |
| | | ucauggagcgguccuccuucgagaagaaccccaucgacuuccuggagg |
| | | ccaagggcuacaaggaggugaagaaggaccugaucaucaagcugccca |
| | | aguacucccuguucgagcuggagaacggccggaagcggaugcuggcc |
| | | uccgccggcgagcugcagaagggcaacgagcuggcccugcccuccaag |
| | | uacgugaacuuccuguaccuggccucccacuacgagaagcugaagggc |
| | | ucccccgaggacaacgagcagaagcagcuguucguggagcagcacaag |
| | | cacuaccuggacgagaucaucgagcagaucuccgaguucuccaagcgg |
| | | gugauccuggccgacgccaaccuggacaaggugcuguccgccuacaac |
| | | aagcaccgggacaagcccauccgggagcaggccgagaacaucauccacc |
| | | uguucacccugaccaaccuggggcccccgccgccuucaaguacuucg |
| | | acaccaccaucgaccggaagcgguacaccuccaccaaggaggugcugg |
| | | acgccacccugauccaccaguccaucaccggccuguacgagacccggau |
| | | cgaccugucccagcugggcggcgac |
| SpyCas9 | SEQ ID | MDKKYSIGLDIGTNSVGWAVITDEYKVPSKKFKV |
| mRNA42 - AA | NO: 8 | LGNTDRHSIKKNLIGALLFDSGETAEATRLKRTAR |
| sequence | | RRYTRRKNRICYLQEIFSNEMAKVDDSFFHRLEES |
| | | FLVEEDKKHERHPIFGNIVDEVAYHEKYPTIYHLR |
| | | KKLVDSTDKADLRLIYLALAHMIKFRGHFLIEGDL |
| | | NPDNSDVDKLFIQLVQTYNQLFEENPINASGVDA |
| | | KAILSARLSKSRRLENLIAQLPGEKKNGLFGNLIAL |
| | | SLGLTPNFKSNFDLAEDAKLQLSKDTYDDDLDNL |
| | | LAQIGDQYADLFLAAKNLSDAILLSDILRVNTEIT |
| | | KAPLSASMIKRYDEHHQDLTLLKALVRQQLPEKY |
| | | KEIFFDQSKNGYAGYIDGGASQEEFYKFIKPILEK |
| | | MDGTEELLVKLNREDLLRKQRTFDNGSIPHQIHL |
| | | GELHAILRRQEDFYPFLKDNREKIEKILTFRIPYYV |
| | | GPLARGNSRFAWMTRKSEETITPWNFEEVVDKGA |
| | | SAQSFIERMTNFDKNLPNEKVLPKHSLLYEYFTVY |
| | | NELTKVKYVTEGMRKPAFLSGEQKKAIVDLLFKT |
| | | NRKVTVKQLKEDYFKKIECFDSVEISGVEDRFNAS |
| | | LGTYHDLLKIIKDKDFLDNEENEDILEDIVLTLTLF |
| | | EDREMIEERLKTYAHLFDDKVMKQLKRRRYTGW |
| | | GRLSRKLINGIRDKQSGKTILDFLKSDGFANRNFM |
| | | QLIHDDSLTFKEDIQKAQVSGQGDSLHEHIANLAG |
| | | SPAIKKGILQTVKVVDELVKVMGRHKPENIVIEM |
| | | ARENQTTQKGQKNSRERMKRIEEGIKELGSQILKE |
| | | HPVENTQLQNEKLYLYYLQNGRDMYVDQELDIN |
| | | RLSDYDVDHIVPQSFLKDDSIDNKVLTRSDKNRG |
| | | KSDNVPSEEVVKKMKNYWRQLLNAKLITQRKFD |
| | | NLTKAERGGLSELDKAGFIKRQLVETRQITKHVA |
| | | QILDSRMNTKYDENDKLIREVKVITLKSKLVSDFR |
| | | KDFQFYKVREINNYHHAHDAYLNAVVGTALIKK |
| | | YPKLESEFVYGDYKVYDVRKMIAKSEQEIGKATA |
| | | KYFFYSNIMNFFKTEITLANGEIRKRPLIETNGETG |
| | | EIVWDKGRDFATVRKVLSMPQVNIVKKTEVQTG |
| | | GFSKESILPKRNSDKLIARKKDWDPKKYGGFDSPT |
| | | VAYSVLVVAKVEKGKSKKLKSVKELLGITIMERS |
| | | SFEKNPIDFLEAKGYKEVKKDLIIKLPKYSLFELEN |
| | | GRKRMLASAGELQKGNELALPSKYVNFLYLASH |
| | | YEKLKGSPEDNEQKQLFVEQHKHYLDEIIEQISEFS |
| | | KRVILADANLDKVLSAYNKHRDKPIREQAENIIHL |
| | | FTLTNLGAPAAFKYFDTTIDRKRYTSTKEVLDATL |
| | | IHQSITGLYETRIDLSQLGGD |

While this invention has been particularly shown and described with references to example embodiments thereof, it will be understood by those skilled in the art that various changes in form and details may be made therein without departing from the scope of the invention encompassed by the appended claims.

1.-134. (canceled)

135. A compound selected from the group consisting of: 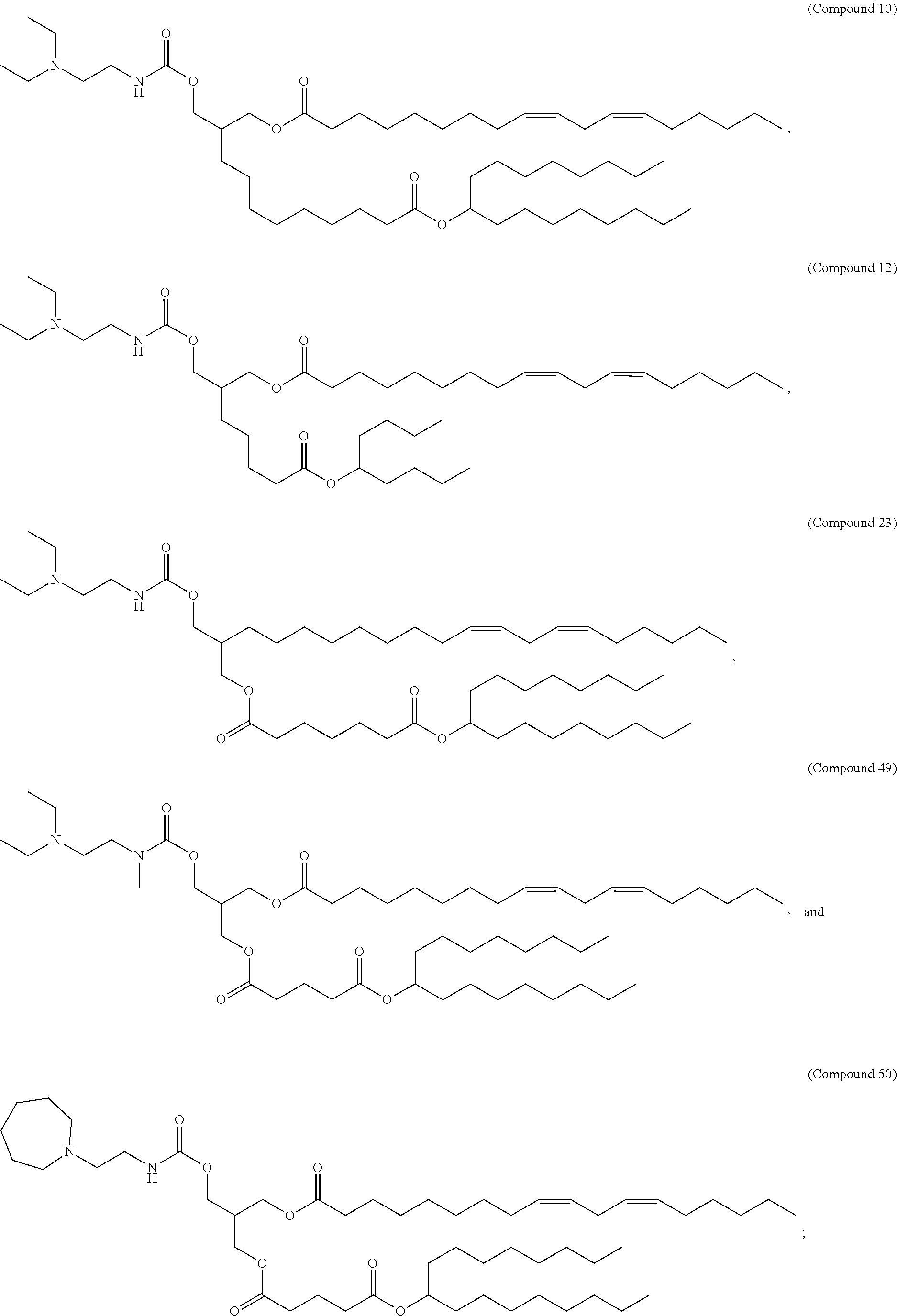
or a salt thereof.

136. The compound of claim 135, wherein the compound is Compound 10: 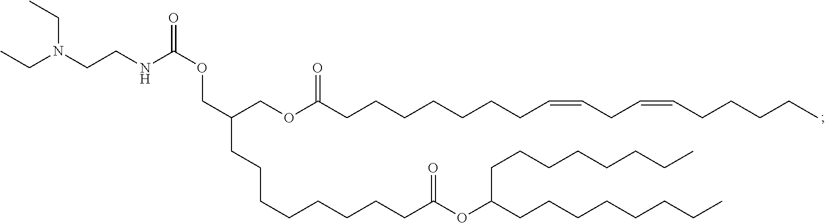 [SMILES: CCCCC/C=C\C/C=C\CCCCCCCC(=O)OCC(CCCCCCCCC(=O)OC(CCCCCCCC)CCCCCCCC)COC(=O)NCCN(CC)CC]
or a salt thereof.

137. The compound of claim 135, wherein the compound is Compound 12: 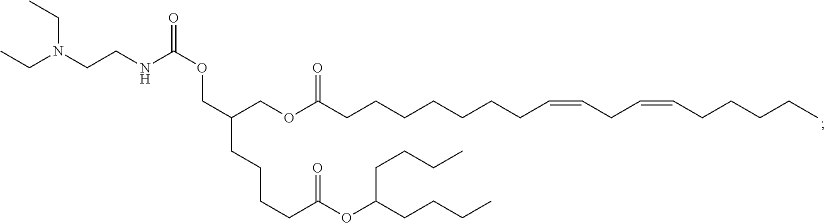 [SMILES: CCCCC/C=C\C/C=C\CCCCCCCC(=O)OCC(CCCCC(=O)OC(CCCC)CCCC)COC(=O)NCCN(CC)CC]
or a salt thereof.

138. The compound of claim 135, wherein the compound is Compound 23: 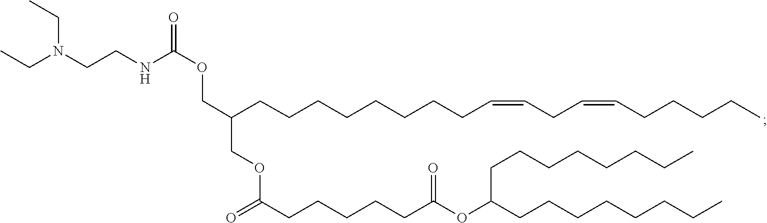 [SMILES: CCCCC/C=C\C/C=C\CCCCCCCCC(COC(=O)CCCCCC(=O)OC(CCCCCCCC)CCCCCCCC)COC(=O)NCCN(CC)CC]
or a salt thereof.

139. The compound of claim 135, wherein the compound is Compound 49: 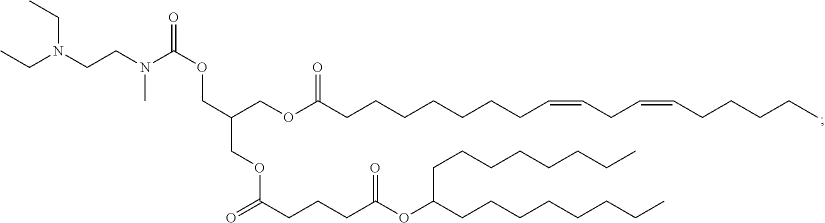 [SMILES: CCCCC/C=C\C/C=C\CCCCCCCC(=O)OCC(COC(=O)CCCC(=O)OC(CCCCCCCC)CCCCCCCC)COC(=O)N(C)CCN(CC)CC]
or a salt thereof.

140. The compound of claim 135, wherein the compound is Compound 50: 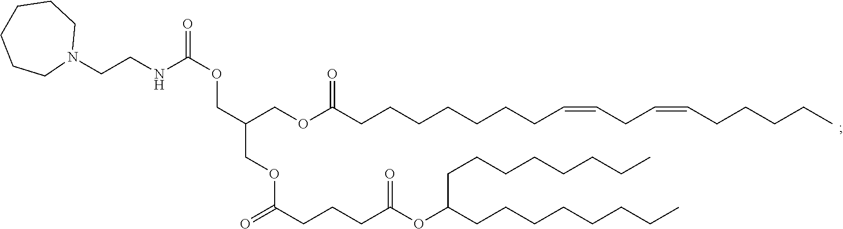 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1]
or a salt thereof.

141. A lipid nanoparticle (LNP) composition comprising the compound of claim 135, encapsulating a nucleic acid.

142. The LNP composition of claim 141, wherein the nucleic acid is a messenger RNA (mRNA).

143. The LNP composition of claim 142, wherein the mRNA encodes an RNA-guided DNA-binding agent.

144. The LNP composition of claim 143, wherein the RNA-guided DNA-binding agent comprises a Class 2 Cas nuclease.

145. The LNP composition of claim 144, wherein the Class 2 Cas nuclease is an S. pyogenes Cas9.

146. The LNP composition of claim 143, further comprising a guide RNA (gRNA).

147. The LNP composition of claim 141, further comprising: (a) a neutral lipid; (b) a helper lipid; and (c) a PEG lipid.

148. The LNP composition of claim 147, wherein:
(a) the neutral lipid is DSPC;
(b) the helper lipid is cholesterol; and
(c) the PEG lipid is 1,2-dimyristoyl-rac-glycero-3-[methoxy(polyethylene glycol)-2000](PEG2K-DMG), methoxy-PEG2000-carbamoyl-1,2-tridecyoxypropylamine (C13 ether), or methoxy-PEG2000-carbamoyl-1,2-tetradecyoxypropylamine (C14 ether).

149. The LNP composition of claim 141, wherein the compound is Compound 10: 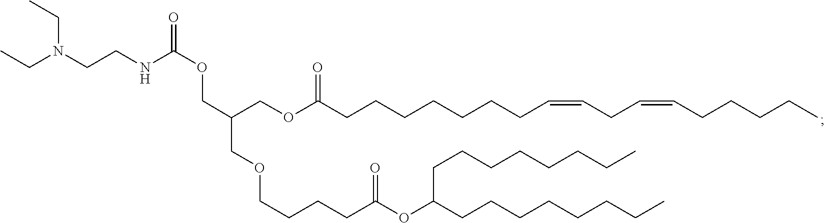 [SMILES: CCCCC/C=C\C/C=C\CCCCCCCC(=O)OCC(COCCCCC(=O)OC(CCCCCCCC)CCCCCCCC)COC(=O)NCCN(CC)CC]
or a salt thereof.

150. The LNP composition of claim 141, wherein the compound is Compound 12: 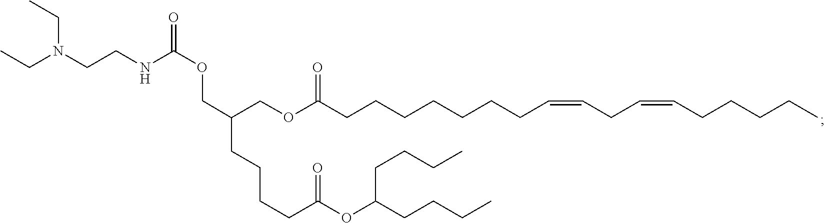 [SMILES: CCCCC/C=C\C/C=C\CCCCCCCC(=O)OCC(CCCCC(=O)OC(CCCC)CCCC)COC(=O)NCCN(CC)CC]
or a salt thereof.

151. The LNP composition of claim 141, wherein the compound is Compound 23: 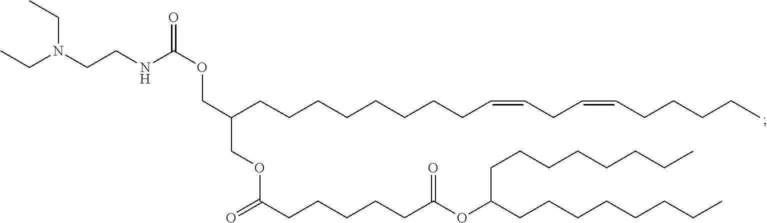 [SMILES: CCCCC/C=C\C/C=C\CCCCCCCCC(COC(=O)CCCCCC(=O)OC(CCCCCCCC)CCCCCCCC)COC(=O)NCCN(CC)CC]
or a salt thereof.

152. The LNP composition of claim 141, wherein the compound is Compound 49: 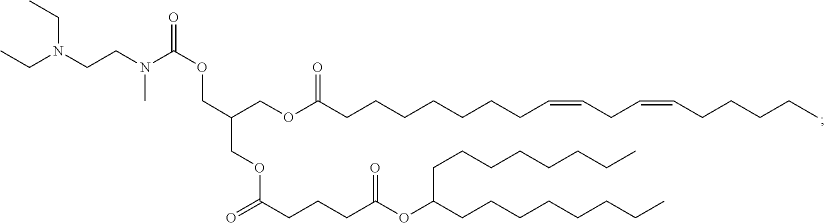 [SMILES: CCCCC/C=C\C/C=C\CCCCCCCC(=O)OCC(COC(=O)CCCC(=O)OC(CCCCCCCC)CCCCCCCC)COC(=O)N(C)CCN(CC)CC]
or a salt thereof.

153. The LNP composition of claim 141, wherein the compound is Compound 50: 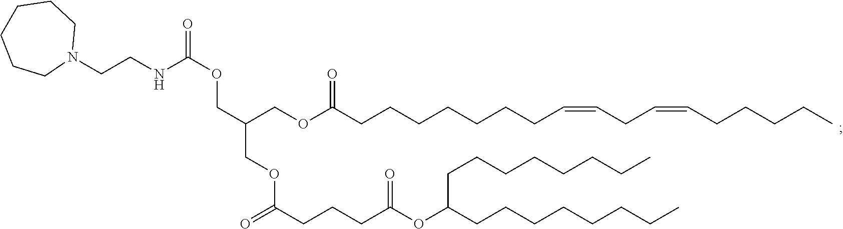 [SMILES: CCCCC/C=C\C/C=C\CCCCCCCC(=O)OCC(COC(=O)CCCC(=O)OC(CCCCCCCC)CCCCCCCC)COC(=O)NCCN1CCCCCC1]
or a salt thereof.

154. The LNP composition of claim 141, further comprising:
(a) a neutral lipid, wherein the neutral lipid is DSPC;
(b) a helper lipid, wherein the helper lipid is cholesterol; and
(c) a PEG lipid, wherein the PEG lipid is 1,2-dimyristoyl-rac-glycero-3-[methoxy(polyethylene glycol)-2000] (PEG2K-DMG), methoxy-PEG2000-carbamoyl-1,2-tridecyoxypropylamine (C13 ether), or methoxy-PEG2000-carbamoyl-1,2-tetradecyoxypropylamine (C14 ether);
wherein the nucleic acid comprises an mRNA encoding a Class 2 Cas nuclease and a guide RNA.

155. A method of cleaving a DNA, comprising contacting a cell with the LNP composition of claim 146.

156. A method of gene editing, comprising contacting a cell with the LNP composition of claim 146.

157. The method of claim 156, comprising administering the LNP composition to a human.


SEQUENCE LISTING (8 sequences)

SEQ ID NO: 1 - RNA, 21 nt, synthetic construct
  source 1..21: mol_type = other RNA
cccaagaagaagcggaaggtg

SEQ ID NO: 2 - RNA, 100 nt, synthetic construct
  source 1..100: mol_type = other RNA
acgcaaatatcagtccagcggttttagagctagaaatagcaagttaaaataaggctagtccgttatcaacttgaaaaagtggcaccgagtcggtgctttt

SEQ ID NO: 3 - RNA, 4423 nt, synthetic construct
  source 1..4423: mol_type = other RNA
gggaagctcagaataaacgctcaactttggccggatctgccaccatggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgacggcggcggctcccccaagaagaagcggaaggtgtgactagcaccagcctcaagaacacccgaatggagtctctaagctacataataccaacttacactttacaaaatgttgtcccccaaaatgtagccattcgtatctgctcctaataaaaagaaagtttcttcacattctctcgagaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaatctag

SEQ ID NO: 4 - protein, 7 aa, synthetic construct
PKKKRKV

SEQ ID NO: 5 - RNA, 100 nt, synthetic construct; identical to SEQ ID NO: 2
  source 1..100: mol_type = other RNA
  modified_base 1: mod_base = OTHER; note = 2'-O-methyl modified nucleotide with phosphorothioate linkage
  modified_base 2: mod_base = OTHER; note = 2'-O-methyl modified nucleotide with phosphorothioate linkage
  modified_base 3: mod_base = OTHER; note = 2'-O-methyl modified nucleotide with phosphorothioate linkage
  modified_base 29: mod_base = OTHER; note = 2'-O-methyl modified nucleotide
  modified_base 30: mod_base = OTHER; note = 2'-O-methyl modified nucleotide
  modified_base 31: mod_base = OTHER; note = 2'-O-methyl modified nucleotide
  modified_base 32: mod_base = OTHER; note = 2'-O-methyl modified nucleotide
  modified_base 33: mod_base = OTHER; note = 2'-O-methyl modified nucleotide
  modified_base 34: mod_base = OTHER; note = 2'-O-methyl modified nucleotide
  modified_base 35: mod_base = OTHER; note = 2'-O-methyl modified nucleotide
  modified_base 36: mod_base = OTHER; note = 2'-O-methyl modified nucleotide
  modified_base 37: mod_base = OTHER; note = 2'-O-methyl modified nucleotide
  modified_base 38: mod_base = OTHER; note = 2'-O-methyl modified nucleotide
  modified_base 39: mod_base = OTHER; note = 2'-O-methyl modified nucleotide
  modified_base 40: mod_base = OTHER; note = 2'-O-methyl modified nucleotide
  modified_base 69: mod_base = OTHER; note = 2'-O-methyl modified nucleotide
  modified_base 70: mod_base = OTHER; note = 2'-O-methyl modified nucleotide
  modified_base 71: mod_base = OTHER; note = 2'-O-methyl modified nucleotide
  modified_base 72: mod_base = OTHER; note = 2'-O-methyl modified nucleotide
  modified_base 73: mod_base = OTHER; note = 2'-O-methyl modified nucleotide
  modified_base 74: mod_base = OTHER; note = 2'-O-methyl modified nucleotide
  modified_base 75: mod_base = OTHER; note = 2'-O-methyl modified nucleotide
  modified_base 76: mod_base = OTHER; note = 2'-O-methyl modified nucleotide
  modified_base 77: mod_base = OTHER; note = 2'-O-methyl modified nucleotide
  modified_base 78: mod_base = OTHER; note = 2'-O-methyl modified nucleotide
  modified_base 79: mod_base = OTHER; note = 2'-O-methyl modified nucleotide
  modified_base 80: mod_base = OTHER; note = 2'-O-methyl modified nucleotide
  modified_base 81: mod_base = OTHER; note = 2'-O-methyl modified nucleotide
  modified_base 82: mod_base = OTHER; note = 2'-O-methyl modified nucleotide
  modified_base 83: mod_base = OTHER; note = 2'-O-methyl modified nucleotide
  modified_base 84: mod_base = OTHER; note = 2'-O-methyl modified nucleotide
  modified_base 85: mod_base = OTHER; note = 2'-O-methyl modified nucleotide
  modified_base 86: mod_base = OTHER; note = 2'-O-methyl modified nucleotide
  modified_base 87: mod_base = OTHER; note = 2'-O-methyl modified nucleotide
  modified_base 88: mod_base = OTHER; note = 2'-O-methyl modified nucleotide
  modified_base 89: mod_base = OTHER; note = 2'-O-methyl modified nucleotide
  modified_base 90: mod_base = OTHER; note = 2'-O-methyl modified nucleotide
  modified_base 91: mod_base = OTHER; note = 2'-O-methyl modified nucleotide
  modified_base 92: mod_base = OTHER; note = 2'-O-methyl modified nucleotide
  modified_base 93: mod_base = OTHER; note = 2'-O-methyl modified nucleotide
  modified_base 94: mod_base = OTHER; note = 2'-O-methyl modified nucleotide
  modified_base 95: mod_base = OTHER; note = 2'-O-methyl modified nucleotide
  modified_base 96: mod_base = OTHER; note = 2'-O-methyl modified nucleotide
  modified_base 97: mod_base = OTHER; note = 2'-O-methyl modified nucleotide with phosphorothioate linkage
  modified_base 98: mod_base = OTHER; note = 2'-O-methyl modified nucleotide with phosphorothioate linkage
  modified_base 99: mod_base = OTHER; note = 2'-O-methyl modified nucleotide with phosphorothioate linkage
  modified_base 100: mod_base = OTHER; note = 2'-O-methyl modified nucleotide

SEQ ID NO: 6 - RNA, 20 nt, synthetic construct
  source 1..20: mol_type = other RNA
acgcaaatatcagtccagcg

SEQ ID NO: 7 - RNA, 4104 nt, synthetic construct
  source 1..4104: mol_type = other RNA
atggacaagaagtactccatcggcctggacatcggcaccaactccgtgggctgggccgtgatcaccgacgagtacaaggtgccctccaagaagttcaaggtgctgggcaacaccgaccggcactccatcaagaagaacctgatcggcgccctgctgttcgactccggcgagaccgccgaggccacccggctgaagcggaccgcccggcggcggtacacccggcggaagaaccggatctgctacctgcaggagatcttctccaacgagatggccaaggtggacgactccttcttccaccggctggaggagtccttcctggtggaggaggacaagaagcacgagcggcaccccatcttcggcaacatcgtggacgaggtggcctaccacgagaagtaccccaccatctaccacctgcggaagaagctggtggactccaccgacaaggccgacctgcggctgatctacctggccctggcccacatgatcaagttccggggccacttcctgatcgagggcgacctgaaccccgacaactccgacgtggacaagctgttcatccagctggtgcagacctacaaccagctgttcgaggagaaccccatcaacgcctccggcgtggacgccaaggccatcctgtccgcccggctgtccaagtcccggcggctggagaacctgatcgcccagctgcccggcgagaagaagaacggcctgttcggcaacctgatcgccctgtccctgggcctgacccccaacttcaagtccaacttcgacctggccgaggacgccaagctgcagctgtccaaggacacctacgacgacgacctggacaacctgctggcccagatcggcgaccagtacgccgacctgttcctggccgccaagaacctgtccgacgccatcctgctgtccgacatcctgcgggtgaacaccgagatcaccaaggcccccctgtccgcctccatgatcaagcggtacgacgagcaccaccaggacctgaccctgctgaaggccctggtgcggcagcagctgcccgagaagtacaaggagatcttcttcgaccagtccaagaacggctacgccggctacatcgacggcggcgcctcccaggaggagttctacaagttcatcaagcccatcctggagaagatggacggcaccgaggagctgctggtgaagctgaaccgggaggacctgctgcggaagcagcggaccttcgacaacggctccatcccccaccagatccacctgggcgagctgcacgccatcctgcggcggcaggaggacttctaccccttcctgaaggacaaccgggagaagatcgagaagatcctgaccttccggatcccctactacgtgggccccctggcccggggcaactcccggttcgcctggatgacccggaagtccgaggagaccatcaccccctggaacttcgaggaggtggtggacaagggcgcctccgcccagtccttcatcgagcggatgaccaacttcgacaagaacctgcccaacgagaaggtgctgcccaagcactccctgctgtacgagtacttcaccgtgtacaacgagctgaccaaggtgaagtacgtgaccgagggcatgcggaagcccgccttcctgtccggcgagcagaagaaggccatcgtggacctgctgttcaagaccaaccggaaggtgaccgtgaagcagctgaaggaggactacttcaagaagatcgagtgcttcgactccgtggagatctccggcgtggaggaccggttcaacgcctccctgggcacctaccacgacctgctgaagatcatcaaggacaaggacttcctggacaacgaggagaacgaggacatcctggaggacatcgtgctgaccctgaccctgttcgaggaccgggagatgatcgaggagcggctgaagacctacgcccacctgttcgacgacaaggtgatgaagcagctgaagcggcggcggtacaccggctggggccggctgtcccggaagctgatcaacggcatccgggacaagcagtccggcaagaccatcctggacttcctgaagtccgacggcttcgccaaccggaacttcatgcagctgatccacgacgactccctgaccttcaaggaggacatccagaaggcccaggtgtccggccagggcgactccctgcacgagcacatcgccaacctggccggctcccccgccatcaagaagggcatcctgcagaccgtgaaggtggtggacgagctggtgaaggtgatgggccggcacaagcccgagaacatcgtgatcgagatggcccgggagaaccagaccacccagaagggccagaagaactcccgggagcggatgaagcggatcgaggagggcatcaaggagctgggctcccagatcctgaaggagcaccccgtggagaacacccagctgcagaacgagaagctgtacctgtactacctgcagaacggccgggacatgtacgtggaccaggagctggacatcaaccggctgtccgactacgacgtggaccacatcgtgccccagtccttcctgaaggacgactccatcgacaacaaggtgctgacccggtccgacaagaaccggggcaagtccgacaacgtgccctccgaggaggtggtgaagaagatgaagaactactggcggcagctgctgaacgccaagctgatcacccagcggaagttcgacaacctgaccaaggccgagcggggcggcctgtccgagctggacaaggccggcttcatcaagcggcagctggtggagacccggcagatcaccaagcacgtggcccagatcctggactcccggatgaacaccaagtacgacgagaacgacaagctgatccgggaggtgaaggtgatcaccctgaagtccaagctggtgtccgacttccggaaggacttccagttctacaaggtgcgggagatcaacaactaccaccacgcccacgacgcctacctgaacgccgtggtgggcaccgccctgatcaagaagtaccccaagctggagtccgagttcgtgtacggcgactacaaggtgtacgacgtgcggaagatgatcgccaagtccgagcaggagatcggcaaggccaccgccaagtacttcttctactccaacatcatgaacttcttcaagaccgagatcaccctggccaacggcgagatccggaagcggcccctgatcgagaccaacggcgagaccggcgagatcgtgtgggacaagggccgggacttcgccaccgtgcggaaggtgctgtccatgccccaggtgaacatcgtgaagaagaccgaggtgcagaccggcggcttctccaaggagtccatcctgcccaagcggaactccgacaagctgatcgcccggaagaaggactgggaccccaagaagtacggcggcttcgactcccccaccgtggcctactccgtgctggtggtggccaaggtggagaagggcaagtccaagaagctgaagtccgtgaaggagctgctgggcatcaccatcatggagcggtcctccttcgagaagaaccccatcgacttcctggaggccaagggctacaaggaggtgaagaaggacctgatcatcaagctgcccaagtactccctgttcgagctggagaacggccggaagcggatgctggcctccgccggcgagctgcagaagggcaacgagctggccctgccctccaagtacgtgaacttcctgtacctggcctcccactacgagaagctgaagggctcccccgaggacaacgagcagaagcagctgttcgtggagcagcacaagcactacctggacgagatcatcgagcagatctccgagttctccaagcgggtgatcctggccgacgccaacctggacaaggtgctgtccgcctacaacaagcaccgggacaagcccatccgggagcaggccgagaacatcatccacctgttcaccctgaccaacctgggcgcccccgccgccttcaagtacttcgacaccaccatcgaccggaagcggtacacctccaccaaggaggtgctggacgccaccctgatccaccagtccatcaccggcctgtacgagacccggatcgacctgtcccagctgggcggcgac

SEQ ID NO: 8 - protein, 1368 aa, synthetic construct [sequence omitted]